(19) United States
(12) Patent Application Publication
(10) Pub. No.: US 2024/0116910 A1
(43) Pub. Date: Apr. 11, 2024

(54) Heterocycles as WIP1 Inhibitors

(71) Applicant: PMV Pharmaceuticals, Inc., Princeton, NJ (US)

(72) Inventors: Binh Vu, North Caldwell, NJ (US); Romyr Dominique, East Brunswick, NJ (US); Yi Chen, Nutley, NJ (US); Hongju Li, Monroe, NJ (US)

(21) Appl. No.: 18/368,467

(22) Filed: Sep. 14, 2023

Related U.S. Application Data
(60) Provisional application No. 63/406,543, filed on Sep. 14, 2022.

(51) Int. Cl. C07D 409/12; C07D 277/28; C07D 333/22; C07D 409/04; C07D 409/14; C07D 417/12; C07D 487/04

(52) U.S. Cl. CPC C07D 409/12; C07D 277/28; C07D 333/22; C07D 409/04; C07D 409/14; C07D 417/12; C07D 487/04

(57) ABSTRACT

The present disclosure relates to the use of substituted heterocycles for the inhibition of the activity of WIP1. Suitably, the present disclosure relates to the use of substituted heterocycles for the treatment of cancer.

### CROSS-REFERENCE TO RELATED APPLICATIONS

This application claims the benefit of U.S. Provisional Application No. 63/406,543, filed Sep. 14, 2022, which is incorporated herein by reference in its entirety.

### FIELD OF THE INVENTION

This invention relates to the substituted heterocycles for the inhibition and/or induced destabilization of the WIP1 (PPM1D) phosphatase. The invention relates to the use of the substituted heterocycles for the treatment of cancer.

### BACKGROUND OF THE INVENTION

Wild-type P53 inducible Phosphatase 1 (WIP1/PPMID) is a serin threonine phosphatase that plays a key role in the homeostatic down-regulation of various stress response and DNA repair signaling pathways. WIP1 is a member of the type 2C family protein phosphatase family that are dependent on Mg²⁺ or Mn²⁺ for their catalytic activity. As its name suggests, WIP1 can be induced by various DNA damaging treatments including ionizing or UV irradiation in a P53 dependent manner (Fiscella et al. PNAS 94, 6048-6053 (1997)). Several groups have explored different WIP1 inhibitors, including Bang, et al. ChemMedChem, 2008, 3, 230-232; Gilmarten et al, Nature Chem. Biol. 10, 2014, 181-187; Esfandari et al. Mol. Cancer Ther. 15, 2016, 379-391, and PCT Publication WO2012149102.

Several lines of evidence have suggested an oncogenic role for WIP1 but there has not been success in developing WIP1 inhibitors for the treatment of cancer.

### INCORPORATION BY REFERENCE

Each patent, publication, and non-patent literature cited in the application is hereby incorporated by reference in its entirety as if each was incorporated by reference individually.

### SUMMARY OF THE INVENTION

In some embodiments, the present disclosure provides a compound of Formula 1:

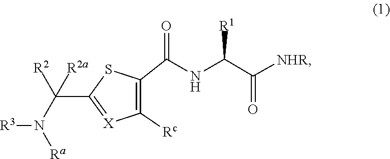

wherein
X is CR^{b} or N;
R is selected from H, unsubstituted or substituted C₁-C₄ alkoxy, unsubstituted or substituted C₁-C₄ alkyl, C₃-C₇cycloalkyl, C₅-C₁₀ bicycloalkyl, C₅-C₁₀ spirocycloalkyl, C₆-C₁₀ aryl, 5-10 membered heteroaryl, 4-10 membered heterocyclyl, 5-10 membered bicycloheterocyclyl, and 7-12 membered spiroheterocyclyl;
   wherein each of said cycloalkyl, bicycloalkyl and spirocycloalkyl is independently unsubstituted or substituted with one, two, or three substituents selected from heterocyclyl, C₁-C₄ alkyl, carboxy, aminocarbonyl, C₁-C₄ alkylaminocarbonyl, C₁-C₄ dialkylaminocarbonyl, C₁-C₄ alkoxycarbonyl, hydroxyl, amino, and C₁C alkoxy, each of which is substituted or unsubstituted, and halo, hydroxyl, and cyano;
   wherein each of said heterocyclyl, bicycloheterocyclyl, and spiroheterocyclyl is independently unsubstituted or substituted with one, two, or three substituents selected from heterocyclyl, C₁-C₄ alkyl, carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, hydroxyl, and cyano;
   wherein said heteroaryl is unsubstituted or substituted with one, two, or three substituents selected from heterocyclyl, C₁-C₄ alkyl, C₁-C₄ alkylsulfonyl, carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, hydroxyl, and cyano, and
   wherein said aryl is unsubstituted or substituted with one, two, or three substituents selected from heterocyclyl, C₁-C₄ alkyl, C₁-C₄ alkylsulfonyl, carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, hydroxyl, and cyano;
R¹ is selected from C₃-C₈alkyl, cyclohexyl, cyclopentyl-C₁₋₂-alkyl, cyclohexyl-C₁₋₂-alkyl, bicyclo[2.2.2]octan-1-yl-C₁₋₂-alkyl, bicyclo[3.1.0]hexan-2-yl-C₁₋₂-alkyl and bicyclo[1.1.1]pentan-1-yl-C₁₋₂-alkyl, each of which is unsubstituted;
R^{2a} is H or substituted or unsubstituted C₁-C₄ alkyl;
R³ is selected from H, C₆-C₁₀ aryl, and 5-10 membered heteroaryl, wherein each heteroaryl and aryl is unsubstituted or substituted with one, two, or three substituents selected from C₁C-alkyl, C₃-C₆ heterocycloalkyl, aralkyl, C₁-C₄ alkylsulfonyl, carboxy, amino, substituted amino, C₃-C₇ cycloalkyl, phenyl, aminocarbonyl, C₁-C₄ alkylaminocarbonyl, C₁-C₄ alkoxycarbonyl, and C₁-C₄ alkoxy, each of which is substituted or unsubstituted, and halo, hydroxyl, and cyano, R² is selected from H, substituted or unsubstituted C₁-C₄ alkyl, C₁-C₂ haloalkyl, substituted or unsubstituted C₁-C₄ alkoxy-C₁-C₄ alkyl, substituted or unsubstituted 5-6 membered heterocyclyl-C₁-C₄ alkyl, substituted or unsubstituted aryl-C₁-C₄ alkyl, and substituted or unsubstituted 5-6 membered heteroaryl-C₁₋₂ alkyl, and R^{a} is H or C₁-C₆ alkyl; or
   R³ and R² together with the nitrogen atom to which R³ is bound and the carbon atom to which R² is bound form 9-10 membered heterocyclyl that is unsubstituted or substituted with one or two substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, hydroxyl, and C₁-C₄ alkoxy, each of which is substituted or unsubstituted, and halo, hydroxyl, and cyano, and R^{a} is H or C₁-C₆ alkyl; or
R³ and R^{a} together with the nitrogen atom to which R³ and R^{a} are bound form 5-10 membered heterocyclyl that is unsubstituted or substituted with one or two substituents selected from C₁-C₄ alkyl, C₁-C₄ alkylsulfonyl, carboxy, aminocarboyl, C₁-C₄ alkylaminocarbonyl, C₁-C₄ alkoxycarbonyl, and C₁-C₄ alkoxy, each of which is substituted or unsubstituted, and halo, hydroxyl, and cyano, and R² is selected from H, substituted or unsubstituted C₁-C₄ alkyl, C₁-C₂ haloalkyl, substituted or unsubstituted C₁-C₄ alkoxy-C₁-C₄ alkyl, substituted or unsubstituted 5-6 merbered heterocyclyl-C₁-C₄ alkyl, substituted or unsubstituted aryl-C₁-C₄ alkyl, and substituted or unsubstituted 5-6 membered heteroaryl-C₁₋₂ alkyl;
R^{b} and R^{c} are independently selected from C₁-C₄ alkyl, C₃-C₆ heterocycloalkyl, aralkyl, C₁-C₄ alkylsulfonyl, carboxy, amino, C₃-C₇ cycloalkyl, aminocarbonyl, C₁-C₄ alkylaminocarbonyl, C₁-C₄ alkoxycarbonyl and C₁-C₄ alkoxy, each of which is substituted or unsubstituted, and H, halo, hydroxyl, and cyano,
provided R² and R^{2a} are not both H when:
(i) X is CH, R^{c} is H, R¹ is cyclopentylmethyl or cyclohexylmethyl, R^{a} is H or ethyl, R³ is 3-chloropyridin-5-yl, 3-chloro-6-methyl-pyridin-5-yl, 4-chloropyridin-2-yl, or 3-chlorophenyl, and R is H, methyl, ethyl, methoxy, hydroxyethyl, hydroxypropyl, methoxyethyl, cyclopropyl, 3-hydroxycyclobutyl, 3-(1,5-diaminopent-1-ylcarbonyloxy)cyclobutyl, 3-(N²,N⁶-bis(tert-butoxycarbonyl)lysyl)oxycyclobutyl, 3-methylcarbonyloxycyclobutyl, or oxetan-3-yl, or
(ii) X is N, R^{c} is H, R¹ is cyclohexyl or cyclohexylethyl, R^{a} is H, R³ is H or 2-fluoro-4-(3-(4-methylpiperazin-1-yl)-3-oxo-2-propionamidopropyl)phenyl, and R is H or methyl, or a pharmaceutically-acceptable salt thereof.

In some embodiments, the present disclosure provides a compound of Formula 2:

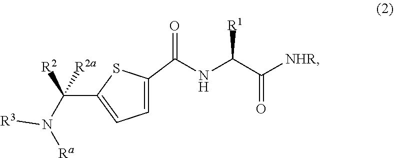

wherein
R is selected from H, unsubstituted or substituted C₁-C₄ alkoxy, unsubstituted or substituted C₁-C₄ alkyl, C₃-C₇ cycloalkyl, C₅-C₁₀ bicycloalkyl, C₅-C₁₀ spirocycloalkyl, C₆-C₁₀ aryl, 5-10 membered heteroaryl, 4-10 membered heterocyclyl, 5-10 membered bicycloheterocyclyl, and 7-12 membered spiroheterocyclyl;
   wherein each of said cycloalkyl, bicycloalkyl and spirocycloalkyl is independently unsubstituted or substituted with one, two, or three substituents selected from heterocyclyl, C₁-C₄ alkyl, carboxy, aminocarbonyl, C₁-C₄ alkylaminocarbonyl, C₁-C₄ dialkylaminocarbonyl, C₁-C₄ alkoxycarbonyl, hydroxyl, amino, and C₁-C₄ alkoxy, each of which is substituted or unsubstituted, and halo, cyano, and hydroxyl;
   wherein each of said heterocyclyl, bicycloheterocyclyl, and spiroheterocyclyl is independently unsubstituted or substituted with one, two, or three substituents selected from heterocyclyl, C₁-C₄ alkyl, carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, cyano, and hydroxyl;
   wherein said heteroaryl is unsubstituted or substituted with one, two, or three substituents selected from heterocyclyl, C₁-C₄ alkyl, C₁-C₄ alkylsulfonyl, carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, cyano, and hydroxyl; and
   wherein said aryl is unsubstituted or substituted with one, two, or three substituents selected from heterocyclyl, C₁-C₄ alkyl, C₁-C₄ alkylsulfonyl, carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, cyano, and hydroxyl;
R¹ is selected from C₃-C₈ alkyl, cyclohexyl, cyclopentyl-C₁₋₂-alkyl, cyclohexyl-C₁₋₂-alkyl, bicyclo[2.2.2]octan-1-yl-C₁₋₂-alkyl, bicyclo[3.1.0]hexan-2-yl-C₁₋₂-alkyl and bicyclo[1.1.1]pentan-1-yl-C₁₋₂-alkyl, each of which is unsubstituted;
R² is selected from substituted or unsubstituted C₁-C₄ alkyl, C₁-C₂ haloalkyl, substituted or unsubstituted C₁-C₄ alkoxy-C₁-C₄ alkyl, substituted or unsubstituted 5-6 membered heterocyclyl-C₁-C₄ alkyl, substituted or unsubstituted aryl-C₁-C₄ alkyl, and substituted or unsubstituted 5-6 membered heteroaryl-C₁₋₂ alkyl;
R^{2a} is H or substituted or unsubstituted C₁-C₄ alkyl;
R³ is selected from H, C₆-C₁₀ aryl, and 5-10 membered heteroaryl, wherein each heteroaryl and aryl is unsubstituted or substituted with one, two, or three substituents selected from C₁-C₁ alkyl, C₃-C₆ heterocycloalkyl, aralkyl, C₁-C₄ alkylsulfonyl, carboxy, amino, substituted amino, C₃-C₇ cycloalkyl, phenyl, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl; and R^{a} is H or substituted or unsubstituted C₁-C₆ alkyl; or R³ and R^{a} together with the nitrogen atom to which R³ and R^{a} are bound form 5-10 membered heterocyclyl that is unsubstituted or substituted with one or two substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, or C₁-C₄ alkoxy, each of which is substituted or unsubstituted, and halo, cyano, and hydroxyl,
or a pharmaceutically acceptable salt thereof.

In some embodiments, the present disclosure provides a compound of Formula 3

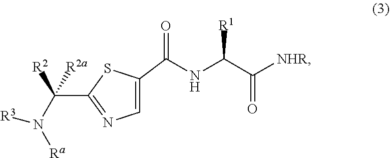

wherein
R is selected from H, unsubstituted or substituted C₁-C₄ alkoxy, unsubstituted or substituted C₁-C₄ alkyl, C₃-C₇ cycloalkyl, C₅-C₁₀ bicycloalkyl, C₅-C₁₀ spirocycloalkyl, C₆-C₁₀ aryl, 5-10 membered heteroaryl, 4-10 membered heterocyclyl, 5-10 membered bicycloheterocyclyl, and 7-12 membered spiroheterocyclyl;
   wherein each of said cycloalkyl, bicycloalkyl and spirocycloalkyl is independently unsubstituted or substituted with one, two, or three substituents selected from heterocyclyl, C₁-C₄ alkyl, carboxy, aminocarbonyl, C₁-C₄ alkylaminocarbonyl, C₁-C₄ dialkylaminocarbonyl, C₁-C₄ alkoxycarbonyl, hydroxyl, amino, and C₁-C₄ alkoxy, each of which is substituted or unsubstituted, and halo, cyano, and hydroxyl,
   wherein each of said heterocyclyl, bicycloheterocyclyl, and spiroheterocyclyl is independently unsubstituted or substituted with one, two, or three substituents selected from heterocyclyl, C₁-C₄ alkyl, carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, cyano, and hydroxyl;
   wherein said heteroaryl is unsubstituted or substituted with one, two, or three substituents selected from heterocyclyl, C₁-C₄ alkyl, C₁-C₄ alkylsulfonyl, carboxy, aminocarbonyl, C₁-C₄ alkylaminocarbonyl, C₁C dialkylaminocarbonyl, C₁-C₄ alkoxycarbonyl, amino, and C₁-C₄ alkoxy, each of which is substituted or unsubstituted, and halo, cyano, and hydroxyl; and
   wherein said aryl is unsubstituted or substituted with one, two, or three substituents selected from heterocyclyl, C₁-C₄ alkyl, C₁-C₄ alkylsulfonyl, carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, cyano, and hydroxyl;
R¹ is selected from C₃-C₈ alkyl, cyclohexyl, cyclopentyl-C₁₋₂-alkyl, cyclohexyl-C₁₋₂-alkyl, bicyclo[2.2.2]octan-1-yl-C₁₋₂-alkyl, bicyclo[3.1.0]hexan-2-yl-C₁₋₂-alkyl and bicyclo[1.1.1]pentan-1-yl-C₁₋₂-alkyl, each of which is unsubstituted;
R² is selected from H, substituted or unsubstituted C₁-C₄ alkyl, (C₁-C₂ haloalkyl, substituted or unsubstituted C₁-C₄ alkoxy-C₁-C₄ alkyl, substituted or unsubstituted 5-6 membered heterocyclyl-C₁-C₄ alkyl, substituted or unsubstituted aryl-C₁-C₄ alkyl and substituted or unsubstituted 5-6 membered heteroaryl-C₁-C₂ alkyl;
R^{2a} is H or substituted or unsubstituted C₁-C₄ alkyl;
R³ is selected from H, C₆-C₁₀ aryl, and 5-10 membered heteroaryl, wherein each heteroaryl and aryl is unsubstituted or substituted with one, two, or three substituents selected from C₁-C₄ alkyl, C₃-C₆ heterocycloalkyl, aralkyl, C₁-C₄ alkylsulfonyl, carboxy, amino, substituted amino, C₃-C₇ cycloalkyl, phenyl, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl; and R^{a} is H or substituted or unsubstituted C₁-C₆ alkyl; or R³ and R^{a} together with the nitrogen atom to which R³ and R^{a} are bound form 5-10 membered heterocyclyl that is unsubstituted or substituted with one or two substituents selected from C₁-C₄ alkyl, C₁-C₄ alkylsulfonyl, carboxy, aminocarbonyl, C₁-C₄ alkylaminocarbonyl, C₁—C alkoxycarbonyl, or C₁-C₄alkoxy, each of which is substituted or unsubstituted, and halo, cyano, and hydroxyl,
or a pharmaceutically acceptable salt thereof.

In some embodiments, described herein is a method of treating a cancer, the method comprising administering to a subject in need thereof a therapeutically-effective amount of a compound described herein.

### DETAILED DESCRIPTION OF THE INVENTION

The present disclosure relates to the use of substituted heterocycles for the inhibition of the activity of WIP1. Suitably, the present disclosure relates to the use of substituted heterocycles for the treatment of cancer.

In some embodiments, the present disclosure provides a compound of Formula 1:

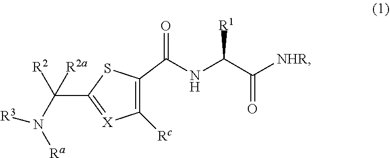

wherein
X is CR^{b} or N;
R is selected from H, unsubstituted or substituted C₁-C₄ alkoxy, unsubstituted or substituted C₁-C₄ alkyl, C₃-C₇ cycloalkyl, C₅-C₁₀ bicycloalkyl, C₅-C₁₀ spirocycloalkyl, C₆-C₁₀ aryl, 5-10 membered heteroaryl, 4-10 membered heterocyclyl, 5-10 membered bicycloheterocyclyl, and 7-12 membered spiroheterocyclyl;
   wherein each of said cycloalkyl, bicycloalkyl and spirocycloalkyl is independently unsubstituted or substituted with one, two, or three substituents selected from heterocyclyl, C₁-C₄ alkyl, carboxy, aminocarbonyl, C₁-C₄ alkylaminocarbonyl, C₁-C₄ dialkylaminocarbonyl, C₁-C₄ alkoxycarbonyl, hydroxyl, amino, and C₁-C₄ alkoxy, each of which is substituted or unsubstituted, and halo, hydroxyl, and cyano;
   wherein each of said heterocyclyl, bicycloheterocyclyl, and spiroheterocyclyl is independently unsubstituted or substituted with one, two, or three substituents selected from heterocyclyl, C₁-C₄ alkyl, carboxy, aminocarbonyl, C₁-C₄ acylaminocarbonyl, C₁-C₄ dialkylaminocarbonyl, C₁-C₄ alkoxycarbonyl, amino, and C₁-C₄ alkoxy, each of which is substituted or unsubstituted, and halo, hydroxyl, and cyano;
   wherein said heteroaryl is unsubstituted or substituted with one, two, or three substituents selected from heterocyclyl, C₁-C₄ alkyl, C₁-C₄ alkylsulfonyl, carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, hydroxyl, and cyano, and
   wherein said aryl is unsubstituted or substituted with one, two, or three substituents selected from heterocyclyl, C₁-C₄ alkyl, C₁-C₄ alkylsulfonyl, carboxy, aminocarbonyl, C₁₋₄ alkylaminocarbonyl, C₁C dialkylaminocarbonyl, C₁-C₆ alkoxycarbonyl, amino, and C₁-C₄ alkoxy, each of which is substituted or unsubstituted, and halo, hydroxyl, and cyano;
R¹ is selected from C₃-C₈ alkyl, cyclohexyl, cyclopentyl-C₁₋₂-alkyl, cyclohexyl-C₁₋₂-alkyl, bicyclo[2.2.2]octan-1-yl-C₁₋₂-alkyl, bicyclo[3.1.0]hexan-2-yl-C₁₋₂-alkyl and bicyclo[1.1.1]pentan-1-yl-C₁₋₂-alkyl, each of which is unsubstituted;
R^{2a} is H or substituted or unsubstituted C₁-C₄ alkyl;
R³ is selected from H, C₆-C₁₀ aryl, and 5-10 membered heteroaryl, wherein each heteroaryl and aryl is unsubstituted or substituted with one, two, or three substituents selected from C₁-C₄ alkyl, C₃-C₆ heterocycloalkyl, aralkyl, C₁-C₄ alkylsulfonyl, carboxy, amino, substituted amino, C₃-C₇ cycloalkyl, phenyl, aminocarbonyl, C₁-C₄ alkylaminocarbonyl, C₁-C₄ alkoxycarbonyl, and C₁-C₄ alkoxy, each of which is substituted or unsubstituted, and halo, hydroxyl, and cyano, R² is selected from H, substituted or unsubstituted C₁-C₄ alkyl, C₁-C₂ haloalkyl, substituted or unsubstituted C₁-C₄ alkoxy-C₁-C₄ alkyl, substituted or unsubstituted 5-6 membered heterocyclo-C₁-C₄ alkyl, substituted or unsubstituted aryl-C₁-C₄ alkyl, and substituted or unsubstituted 5-6 membered heteroaryl-C₁₋₂ alkyl, and R^{a} is H or C₁C alkyl; or
R³ and R² together with the nitrogen atom to which R³ is bound and the carbon atom to which R² is bound form 9-10 membered heterocyclyl that is unsubstituted or substituted with one or two substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, hydroxyl, and C₁-C₄ alkoxy, each of which is substituted or unsubstituted, and halo, hydroxyl, and cyano, and R^{a} is H or C₁-C₆ alkyl; or
R³ and R^{a} together with the nitrogen atom to which R³ and R^{a} are bound form 5-10 membered heterocyclyl that is unsubstituted or substituted with one or two substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, hydroxyl, and cyano, and R² is selected from H, substituted or unsubstituted C₁-C₄ alkyl, C₁-C₂ haloalkyl, substituted or unsubstituted C₁-C₄ alkoxy-C₁-C₄ alkyl, substituted or unsubstituted 5-6 merbered heterocyclyl-C₁-C₄ alkyl, substituted or unsubstituted aryl-C₁-C₄ alkyl, and substituted or unsubstituted 5-6 membered heteroaryl-C₁₋₂ alkyl;
R^{b} and R^{c} are independently selected from C₁-C₄ alkyl, C₃-C₆ heterocycloalkyl, aralkyl, C₁-C₄ alkylsulfonyl, carboxy, amino, C₃-C₇ cycloalkyl, aminocarbonyl, C₁-C₄ alkylaminocarbonyl, C₁-C₄ alkoxycarbonyl, and C₁-C₄ alkoxy, each of which is substituted or unsubstituted, and H, halo, hydroxyl, and cyano,
provided R² and R^{2a} are not both H when:
(i) X is CH, R^{c} is H, R¹ is cyclopentylmethyl or cyclohexylmethyl, R^{a} is H or ethyl, R³ is 3-chloropyridin-5-yl, 3-chloro-6-methyl-pyridin-5-yl, 4-chloropyridin-2-yl, or 3-chlorophenyl, and R is H, methyl, ethyl, methoxy, hydroxyethyl, hydroxypropyl, methoxyethyl, cyclopropyl, 3-hydroxycyclobutyl, 3-(1,5-diaminopent-1-ylcarbonyloxy)cyclobutyl, 3-(N²,N⁶-bis(tert-butoxycarbonyl)lysyl)oxycyclobutyl, 3-methylcarbonyloxycyclobutyl, or oxetan-3-yl, or
(ii) X is N, R^{c} is H, R¹ is cyclohexyl or cyclohexylethyl, R^{a} is H, R³ is H or 2-fluoro-4-(3-(4-methylpiperazin-1-yl)-3-oxo-2-propionamidopropyl)phenyl, and R is H or methyl,
or a pharmaceutically-acceptable salt thereof.

In some embodiments, R is selected from H, substituted or unsubstituted C₁-C₄ alkyl, C₃-C₇ cycloalkyl, phenyl, 4-10 membered heteroaryl, 5-10 membered heterocyclyl, C₅-C₁₀ bicycloalkyl, C₅-C₁₀ spirocycloalkyl, 5-10 membered bicycloheterocyclyl, and 7-12 membered spiroheterocyclyl;
wherein each of said cycloalkyl, bicycloalkyl and spirocycloalkyl is independently unsubstituted or substituted with one, two, or three substituents selected from C₁-C₄ alkyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl;
wherein each of said heterocyclyl, bicycloheterocyclyl and spiroheterocyclyl is independently unsubstituted or substituted with one, two, or three substituents selected from C₁-C₄ alkyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl;
wherein said heteroaryl is unsubstituted or substituted with one, two, or three substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl; and
wherein said phenyl is unsubstituted or substituted with one, two, or three substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl.

In some embodiments, R is selected from H, methyl, ethyl, fluoroethyl, isopropyl, 1-cyano-1-methylethyl, CH₃OCH₂CH₂OCH₂—, CH₃O(C═O)CH₂—, carboxymethyl, 3-fluorophenylmethyl, 4-fluorophenylmethyl, 3-pyridylmethyl, and 4-pyridylmethyl.

In some embodiments, R is selected from 3-pyrazolyl, 4-pyrazolyl, 1H-1,2,3-triazol-4-yl, 3-pyridinyl, 4-pyridinyl, 2-pyridinyl, 3-thienyl, phenyl, 5-indolyl, 6-indolyl, 5-indazolyl, 5-benzimidazolyl, 1H-pyrrolo[2,3-b]pyridin-5-yl, 1,2,3,4-tetrahydroquinolin-7-yl, 5,6,7,8-tetrahydroquinolin-6-yl, 1,2,3,4-tetrahydroquinolin-6-yl, 6-isoquinolinyl, and quinolin-6-yl; each of which is unsubstituted or substituted with one, two, or three substituents selected from methyl, methoxy, ethoxy, isopropoxy, carboxy, (2-methoxyeth-1-yl), methoxycarbonyl, methylaminocarbonyl, aminocarbonyl, methylsulfonyl, hydroxy, chloro, fluoro, and cyano.

In some embodiments, R is bicyclo[1.1.1]pentan-1-yl that is unsubstituted or substituted with one, two, or three substituents selected from methyl, methoxy, carboxy, methoxycarbonyl, methylaminocarbonyl, aminocarbonyl hydroxy, fluoro, and cyano.

In some embodiments, R is selected from bicyclo[1.1.1]pentan-1-yl, 3-methyl-bicyclo[1.1.1]pentan-1-yl, 3-methoxycarbonyl-bicyclo[1.1.1]pentan-1-yl, 3-carboxyl-bicyclo[1.1.1]pentan-1-yl, 3-aminocarbonyl-bicyclo[1.1.1]pentan-1-yl, 3-methylaminocarbonyl-bicyclo[1.1.1]pentan-1-yl, 3-hydroxy-bicyclo[1.1.1]pentan-1-yl, 3-fluoro-bicyclo[1.1.1]pentan-1-yl, 3-cyano-bicyclo[1.1.1]pentan-1-yl, and bicyclo[2.2.2]octan-1-yl.

In some embodiments, R is spiro[3.3]heptan-2-yl or 2-oxaspiro[3.3]heptan-6-yl.

In some embodiments, R is selected from 1,1-dioxo-1λ⁶-thian-4-yl, 2-oxabicyclo[2.1.1]hexan-4-yl, and 8-oxabicyclo[3.2.1]octan-3-yl.

In some embodiments, R is selected from cyclopropyl, 1-cyanocyclopropyl, 1-trifluoromethyl-cyclopropyl, 2-fluorocyclopropyl, 2,2-difluorocyclopropyl, cyclobutyl, 3-fluorocyclobutyl, 2-methylcyclobutyl, 3-methylcyclobutyl, 3,3-difluorocyclobutyl, 3-methoxycarbonylcyclobutyl, 3-carboxy-cyclobutyl, 3,3-difluorocyclopentyl, 3-cyanocyclobutyl, 3-hydroxycyclopentyl, cyclopentyl, 3-fluorocyclopentyl, and 3-cyanocyclopentyl.

In some embodiments, R is selected from piperidin-4-yl, 1-methylpiperidin-4-yl, and 3-oxetanyl.

In some embodiments, R is selected from 1-methyl-3-pyrazolyl, 3-pyrazolyl, 4-pyrazolyl, 1-methyl-4-pyrazolyl, 1-(2-methoxyeth-1-yl)-4-pyrazolyl, 1-ethyl-4-pyrazolyl, 1-fluoroethyl-4-pyrazolyl, 1-trifluoroethyl-4-pyrazolyl, 1-dimethylaminoethyl-4-pyrazolyl, 1-tert-butyl-4-pyrazolyl, 1-(1-methyl-4-piperidinyl)-4-pyrazolyl, 1-oxan-4-yl-4-pyrazolyl, 1-oxetan-3-yl-4-pyrazolyl, 1H-1,2,3-triazol-4-yl, 2-methylpyridin-4-yl, 2,6-dimethylpyridin-4-yl, 2-methylpyridin-5-yl, 2-methylpyridin-6-yl, 2-hydroxy-4-pyridinyl, 2-methoxy-4-pyridinyl, 2-chloro-4-pyridinyl, 2-aminocarbonyl-4-pyridinyl, 2-methoxy-5-pyridinyl, 2-aminocarbonyl-5-pyridinyl, 4-amino-2-pyridinyl, 3-thienyl, 4-cyanophenyl, 3-cyanophenyl, 3-methylsulfonylphenyl, 4-methylsulfonylphenyl, 3,4-difluorophenyl, 4-fluorophenyl, 3-chlorophenyl, 4-chiorophenyl, 3-methoxyphenyl, 3-ethoxyphenyl, 3-isopropoxyphenyl, 4-methoxyethoxyphenyl, 4-fluorophenyl, 4-fluoro-3-methoxyphenyl, 5-indolyl, 1-methyl-5-indolyl, 6-indolyl, 1-methyl-indol-6-yl, 4-chloro-6-indolyl, 7-fluoro-5-indolyl, 3-cyano-5-indolyl, 1-(2-methoxyeth-1-yl)-5-indolyl, 5-indazolyl, 5-benzimidazolyl, 2-methyl-5-benzimidazolyl, 1H-pyrrolo[2,3-b]pyridin-5-yl, 1-methyl-1,2,3,4-tetrahydroquinolin-7-yl, 5,6,7,8-tetrahydroquinolin-6-yl, 1-methyl-1,2,3,4-tetrahydroquinolin-6-yl, 1-(2-methoxyeth-1-yl)-1,2,3,4-tetrahydroquinolin-6-yl, 6-isoquinolinyl, 3-cyano-quinolin-6-yl, 3-fluoroquinolin-6-yl, and 3-methoxyquinolin-6-yl.

In some embodiments, R² is selected from methyl, ethyl, benzyl, trifluoromethyl, 4-fluorobenzyl, methoxymethyl, 1-methyl-4-pteridinylmethyl, 4-pyridinylmethyl, 1-pyrazolylmethyl, and 1-methyl-4-pyrazolylmethyl; and R^{2a} is H.

In some embodiments, R² is methyl; and R^{2a} is H.

In some embodiments, R² is methy; and R^{2a} is methyl.

In some embodiments, R² is H; and R^{2a} is H.

In some embodiments, R³ is pyrazolo[1,5-a]pyrimidin-7-yl, phenyl or pyridyl, each of which is unsubstituted or substituted with one, two, or three selected from chloro, fluoro, bromo, cyano, C₁₋₃ alkyl, hydroxymethyl, C₁₋₃ haloalkyl, cyclopropyl, aminocarbonyl, di[CC₁₋₃ alkyl]aminocarbonyl, di[C₁₋₃alkyl]aminocarbonyl, methoxycarbonyl, carboxy, hydroxyl, C₁₋₃ alkoxy, C₁₋₃ alkoxyalkyl, pyridyl, and phenyl that is unsubstituted or substituted with one or two substituents selected from chloro, fluoro, bromo, cyano, methyl, ethyl, hydroxymethyl, trifluoromethyl, methoxymethyl, methoxypropyl, cyanopropyl, cyclopropyl, carboxy, methoxycarbonyl, aminocarbonyl, methylaminocarbonyl, dimethylaminocarbonyl, hydroxyl, and methoxy.

In some embodiments, R³ is 3-pyridyl that is unsubstituted or substituted with one or two substituents selected from chloro, fluoro, bromo, cyano, methyl, ethyl, hydroxymethyl, 2-hydroxy-2-methylethyl, trifluoromethyl, methoxymethyl, methoxypropyl, 2-cyano-2-methylethyl, cyclopropyl, carboxy, methoxycarbonyl, aminocarbonyl, methylaminocarbonyl, dimethylaminocarbonyl, hydroxyl, methoxy, pyridyl and phenyl that is unsubstituted or substituted with one or two substituents selected from chloro, fluoro, and methoxy.

In some embodiments, R¹ is selected from tert-butyl, 1-methylpropyl, 2-methylbutyl, 2,2-dimethylbutyl, cyclohexyl, bicyclo[2.2.2]octan-1-yl-CH₂—, bicyclo[3.1.0]hexan-2-yl-CH₂— and bicyclo[1.1.1]pentan-1-yl-CH₂—.

In some embodiments, R¹ is cyclopentyl-CH₂— or cyclohexyl-CH₂—.

In some embodiments, R³ and R^{a} together with the nitrogen atom to which R³ and R^{a} are bound form dihydroindol-1-yl, 4-methyl-7-chloro-tetrahydroquinoxalin-1-yl, 5-chloro-1,2,3,4-tetrahydroquinolin-2-yl or 7-chloro-1,2,3,4-tetrahydroquinolin-1-yl, wherein the dihydroindol-1-yl or tetrahydroquinolin-1-yl each are independently unsubstituted of substituted with one or two substituents selected from halo or methoxy.

In some embodiments, R^{a} is H.

In some embodiments, R⁴ is H.

In some embodiments, R³ and R² together with the nitrogen atom to which R³ is bound and the carbon atom to which R² is bound form a 9 or 10 membered heterocyclyl that is a fused ring system of an aromatic ring fused to a non-aromatic ring, wherein said heterocyclyl is unsubstituted or substituted with one or two substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, hydroxyl, and C₁-C₄ alkoxy, each of which is substituted or unsubstituted, and halo, hydroxyl, and cyano.

In some embodiments, R³ and R together with the nitrogen atom to which R³ and R are bound form a 9 or 10 membered heterocyclyl that is a fused ring system of an aromatic ring fused to a non-aromatic ring, wherein said heterocyclyl is unsubstituted or substituted with one or two substituents selected from C₁-C₄ alkyl, C₁-C₄ alkylsulfonyl, carboxy, aminocarbonyl, C₁-C₄ alkylaminocarbonyl. C₁-C₄ alkoxycarbonyl, and C₁-C₁ alkoxy, each of which is substituted or unsubstituted, and halo, hydroxyl, and cyano.

In some embodiments, R³ and R^{a} together with the nitrogen atom to which R³ and R^{a} are bound form a 5, 9, or 10 membered heteroaryl, wherein said heteroaryl is unsubstituted or substituted with one or two substituents selected from C₁-C₄ alkyl, C₁-C₄ alkylsulfonyll, carboxy, aminocarbonyl, C₁-C₄ alkylaminocarbonyl, C₁-C₄ alkoxycarbonyl, and C₁-C₄ alkoxy, each of which is substituted or unsubstituted, and halo, hydroxyl, and cyano.

In some embodiments, R³, and R^{a} together with the nitrogen atom to which R³ and R^{a} are bound form 1,2,3,4-tetrahydroquinolin-1-yl that is unsubstituted or substituted with one or two substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl.

In some embodiments, R³ and R² together with the nitrogen atom to which R³ is bound and the carbon atom to which R² is bound form 1,2,3,4-tetrahydroquinolin-1,2-diyl that is unsubstituted or substituted with one or two substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl.

In some embodiments, the present disclosure provides a compound of Formula 2:

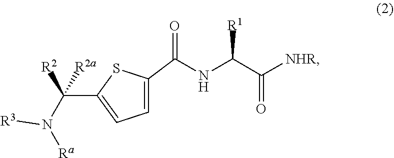

wherein
R is selected from H, unsubstituted or substituted C₁-C₄ alkoxy, unsubstituted or substituted C₁-C₄ alkyl, C₃-C₇ cycloalkyl, C₅-C₁₀ bicycloalkyl, C₅-C₁₀ spirocycloalkyl, C₆-C₁₀ aryl, 5-10 membered heteroaryl, 4-10 membered heterocyclyl, 5-10 membered bicycloheterocyclyl, and 7-12 membered spiroheterocyclyl;
   wherein each of said cycloalkyl, bicycloalkyl and spirocycloalkyl is independently unsubstituted or substituted with one, two, or three substituents selected from heterocyclyl, C₁-C₄ alkyl, carboxy, aminocarbonyl, C₁-C₄ alkylaminocarbonyl, C₁-C₄ dialkylaminocarbonyl, C₁-C₄ alkoxycarbonyl, hydroxyl, amino, and C₁-C₄ alkoxy, each of which is substituted or unsubstituted, and halo, cyano, and hydroxyl;
   wherein each of said heterocyclyl, bicycloheterocyclyl, and spiroheterocyclyl is independently unsubstituted or substituted with one, two, or three substituents selected from heterocyclyl, C₁-C₄ alkyl, carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, cyano, and hydroxyl;
   wherein said heteroaryl is unsubstituted or substituted with one, two, or three substituents selected from heterocyclyl, C₁-C₄ alkyl, C₁-C₄ alkylsulfonyl, carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, cyano, and hydroxyl; and
   wherein said aryl is unsubstituted or substituted with one, two, or three substituents selected from heterocyclyl, C₁-C₄ alkyl, C₁-C₄ alkylsulfonyl, carboxy, aminocarbonyl, C₁-C₄ alkylaminocarbonyl, C₁-C₄ dialkylaminocarboiyl, C₁-C₄ alkoxycarbonyl, amino, and C₁-C₄ alkoxy, each of which is substituted or unsubstituted, and halo, cyano, and hydroxyl,
R¹ is selected from C₃-C₈ alkyl, cyclohexyl, cyclopentyl-C₁₋₂-alkyl, cyclohexyl-C₁₋₂-alkyl, bicyclo[2.2.2]octan-1-yl-C₁₋₂-alkyl, bicyclo[3.1.0]hexan-2-yl-C₁₋₂-alkyl and bicyclo[1.1.1]pentan-1-yl-C₁₋₂-alkyl, each of which is unsubstituted;
R² is selected from substituted or unsubstituted C₁-C₄ alkyl, C₁-C₂ haloalkyl, substituted or unsubstituted C₁-C₄ alkoxy-C₁-C₄ alkyl, substituted or unsubstituted 5-6 membered heterocyclyl-C₁-C₄ alkyl, substituted or unsubstituted aryl-C₁-C₄ alkyl, and substituted or unsubstituted 5-6 membered heteroaryl-C₁₋₂ alkyl;
R^{2a} is H or substituted or unsubstituted C₁-C₄ alkyl;
R³ is selected from H, C₆-C₁₀ aryl, and 5-10 membered heteroaryl, wherein each heteroaryl and aryl is unsubstituted or substituted with one, two, or three substituents selected from C₁-C₄ alkyl, C₃-C₆ heterocycloalkyl, aralkyl, C₁-C₄ alkylsulfonyl, carboxy, amino, substituted amino, C₃-C₇ cycloalkyl, phenyl, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl; and R^{a} is H or substituted or unsubstituted C₁-C₆ alkyl; or R³ and R^{a} together with the nitrogen atom to which R³ and R^{a} are bound form 5-10 membered heterocyclyl that is unsubstituted or substituted with one or two substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, or C₁-C₄ alkoxy, each of which is substituted or unsubstituted, and halo, cyano, and hydroxyl,
or a pharmaceutically acceptable salt thereof.

In some embodiments, R is selected from H, substituted or unsubstituted C₁-C₄ alkyl, C₃-C₇ cycloalkyl, phenyl, 5-10 membered heteroaryl, 4-10 membered heterocyclyl, C₅-C₁₀ bicycloalkyl. C₅-C₁₀ spirocycloalkyl, 5-10 membered bicycloheterocyclyl, and 7-12 membered spiroheterocyclyl;
wherein each of said cycloalkyl, bicycloalkyl and spirocycloalkyl is independently unsubstituted or substituted with one, two, or three substituents selected from C₁-C₄ alkyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl;
wherein each of said heterocyclyl, bicycloheterocyclyl and spiroheterocyclyl is independently unsubstituted or substituted with one, two, or three substituents selected from C₁-C₄ alkyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl;
wherein said heteroaryl is unsubstituted or substituted with one, two, or three substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl; and
wherein said phenyl is unsubstituted or substituted with one, two, or three substituents selected from C₁-C₄ alkyl, C₁-C₄ alkylsulfonyl, carboxy, aminocarbonyl C₁-C₄ alkylaminocarbonyl, C₁-C₄ alkoxycarbonyl, and C₁-C₄ alkoxy, each of which is substituted or unsubstituted, and halo, cyano, and hydroxyl.

In some embodiments, R is selected from H, methyl, ethyl, fluoroethyl, isopropyl, 1-cyano-1-methylethyl, CH₃OCH₂CH₂OCH₂—, CH₃O(C═O)CH₂—, carboxymethyl, 3-fluorophenylmethyl, 4-fluorophenylmethyl, 3-pyridylmethyl, and 4-pyridylmethyl.

In some embodiments, R is selected from 3-pyrazolyl, 4-pyrazolyl, 1H-1,2,3-triazol-4-yl 3-pyridinyl, 4-pyridinyl, 2-pyridinyl, 3-thienyl, phenyl, 5-indolyl, 6-indolyl, 5-indazolyl, 5-benzimidazolyl, 1H-pyrrolo[2,3-b]pyridin-5-yl, 1,2,3,4-tetrahydroquinolin-7-yl, 5,6,7,8-tetrahydroquinolin-6-yl, 1,2,3,4-tetrahydroquinolin-6-yl, 6-isoquinolinyl, and quinolin-6-yl, each of which is independently unsubstituted or substituted with one, two, or three substituents selected from methyl, methoxy, ethoxy, isopropoxy, carboxy, (2-methoxyeth-1-yl), methoxycarbonyl, methylaminocarbonyl, aminocarbonyl, methylsulfonyl, hydroxy, chloro, fluoro, and cyano.

In some embodiments, R is bicyclo[1.1.1]pentan-1-yl that is unsubstituted or substituted with one, two, or three substituents selected from methyl, methoxy, carboxy, methoxycarbonyl, methylaminocarbonyl, aminocarbonyl hydroxy, fluoro, and cyano.

In some embodiments, R is selected from bicyclo[1.1.1]pentan-1-yl, 3-methyl-bicyclo[1.1.1]pentan-1-yl, 3-methoxycarbonyl-bicyclo[1.1.1]pentan-1-yl, 3-carboxyl-bicyclo[1.1.1]pentan-1-yl, 3-aminocarbonyl-bicyclo[1.1.1]pentan-1-yl, 3-methylaminocarbonyl-bicyclo[1.1.1]pentan-L-yl, 3-hydroxy-bicyclo[1.1.1]pentan-1-yl, 3-fluoro-bicyclo[1.1.1]pentan-1-yl, 3-cyano-bicyclo[1.1.1]pentan-1-yl, and bicyclo[2.2.2]octan-1-yl.

In some embodiments, R is spiro[3.3]heptan-2-yl or 2-oxaspiro[3.3]heptan-6-yl.

In some embodiments, R is selected from 1,1-dioxo-1λ⁶-thian-4-yl, 2-oxabicyclo[2.1.1]hexan-4-yl and 8-oxabicyclo[3.2.1]octan-3-yl.

In some embodiments, R is selected from cyclopropyl, 1-cyanocyclopropyl, 1-trifluoromethyl-cyclopropyl, 2-fluorocyclopropyl, 2,2-difluorocyclopropyl, cyclobutyl, 3-fluorocyclobutyl, 2-methylcyclobutyl 3-methylcyclobutyl, 3,3-difluorocyclobutyl, 3-methoxycarbonylcyclobutyl, 3-carboxy-cyclobutyl, 3,3-difluorocyclopentyl, 3-cyanocyclobutyl, 3-hydroxycyclopentyl, cyclopentyl, 3-fluorocyclopentyl, and 3-cyanocyclopentyl.

In some embodiments, R is piperidin-4-yl or 1-methylpiperidin-4-yl.

In some embodiments, R is selected from 1-methyl-3-pyrazolyl, 3-pyrazolyl, 4-pyrazolyl, 1-methyl-4-pyrazolyl, 1-(2-methoxyeth-1-yl)-4-pyrazolyl, 1-ethyl-4-pyrazolyl, 1-fluoroethyl-4-pyrazolyl, 1-trifluoroethyl-4-pyrazolyl, 1-dimethylaminoethyl-4-pyrazolyl, 1-tert-butyl-4-pyrazolyl, 1-(1-methyl-4-piperidinyl)-4-pyrazolyl, 1-oxan-4-yl-4-pyrazolyl, 1-oxetan-3-yl-4-pyrazolyl, 1H-1,2,3-triazol-4-yl, 2-methylpyridin-4-yl, 2,6-dimethylpyridin-4-yl, 2-methylpyridin-5-yl, 2-methylpyridin-6-yl, 2-hydroxy-4-pyridinyl, 2-methoxy-4-pyridinyl, 2-chloro-4-pyridinyl, 2-aminocarbonyl-4-pyrolidyl, 2-methoxy-5-pyridinyl, 2-aminocarbonyl-5-pyridinyl, 4-amino-2-pyridinyl, 3-thienyl, 4-cyanophenyl, 3-cyanophenyl, 3-methylsulfonylphenyl, 4-methylsulfonylphenyl, 3,4-difluorophenyl, 4-fluorophenyl, 3-chlorophenyl, 4-chlorophenyl, 3-methoxyphenyl, 3-ethoxyphenyl, 3-isopropoxyphenyl, 4-methoxymethoxyphenyl, 4-fluorophenyl, 4-fluoro-3-methoxyphenyl, 5-indolyl, 1-methyl-5-Indolyl, 6-indolyl, 1-methyl-indol-6-yl, 4-chloro-6-indolyl, 7-fluoro-5-indolyl, 3-cyano-5-indolyl, 1-(2-methoxyeth-1-yl)-5-indolyl, 5-indazolyl, 5-benzimidazolyl, 2-methyl-5-benzimidazolyl, 1H-pyrrolo[2,3-b]pyridin-5-yl, 1-methyl-1,2,3,4-tetrahydroquinolin-7-yl, 5,6,7,8-tetrahydroquinolin-6-yl, 1-methyl-1,2,3,4-tetrahydroquinolin-6-yl, 1-(2-methoxyeth-1-yl)-1,2,3,4-tetrahydroquinolin-6-yl, 6-isoquinolinyl, 3-cyano-quinolin-6-yl, 3-fluoroquinolin-6-yl, 3-methoxyquinolin-6-yl, and 8-methoxyquinolin-6-yl.

In some embodiments, R² is selected from methyl, ethyl, benzyl, trifluoromethyl, 4-fluorobenzyl, methoxymethyl, 1-methyl-4-piperidinylmethyl, 4-pyridinylmethyl, 1-pyrazolylmethyl, and 1-methyl-4-pyrazolylmethyl and R^{2a} is H.

In some embodiments, R² is methyl; and R^{2a} is H.

In some embodiments, R² is methyl; and R^{a} is methyl.

In some embodiments, R³ is pyrazolo[1,5-a]pyrimidin-7-yl, phenyl or pyridyl, each of which is independently unsubstituted or substituted with one, two, or three substituents selected from chloro, fluoro, bromo, cyano, (C₁₋₃ alkyl, hydroxymethyl, C₁₋₃ haloalkyl, cyclopropyl, aminocarbonyl, di[C₁₋₃ alkyl]aminocarbonyl, di[C₁₋₃ alkyl]aminocarbonyl, methoxycarbonyl, carboxy, hydroxyl, C₁₋₃ alkoxy, C₁₋₃ alkoxyalkyl, pyridyl, and phenyl that is unsubstituted or substituted with one or two substituents selected from chloro, fluoro, bromo, cyano, methyl, ethyl, hydroxydiethyl, trifluoromethyl, methoxymethyl, methoxypropyl, cyanopropyl, cyclopropyl, carboxy, methoxycarbonyl, aminocarbonyl, methylaminocarbonyl, dimethylaminocarbonyl, hydroxyl, and methoxy.

In some embodiments, R³ is 3-pyridyl that is unsubstituted or substituted with one or two substituents selected from chloro, fluoro, bromo, cyano, methyl, ethyl, hydroxymethyl, 2-hydroxy-2-methylethyl, trifluoromethyl, methoxymethyl, methoxypropyl, 2-cyano-2-methylethyl, cyclopropyl, carboxy, methoxycarbonyl, aminocarbonyl, methylaminocarbonyl, dimethylaminocarbonyl, hydroxyl, methoxy, pyridyl and phenyl that is unsubstituted or substituted with one or two substituents selected from chloro, fluoro, and methoxy.

In some embodiments, R¹ is tert-butyl, 1-methylpropyl, 2-methylbutyl, 2,2-dimethylbutyl, cyclohexyl, bicyclo[2.2.2]octan-1-yl-CH₂—, bicyclo[3.1.0]hexan-2-yl-CH₂— or bicyclo[1.1.1]pentan-1-yl-CH₂—.

In some embodiments, R¹ is cyclopentyl-CH₂— or cyclohexyl-CH₂—.

In some embodiments, R³ and R^{a} together with the nitrogen atom to which R³ and R^{a} are bound form dihydroindol-1-yl, 4-methyl-7-chloro-tetrahydroquinoxalin-1-yl, 5-chloro-1,2,3,4-tetrahydroquinolin-2-yl or 7-chloro-1,2,3,1-tetrahydroquinolin-1-yl, wherein the dihydroindol-1-yl or tetrahydroquinoline-2-yl each are unsubstituted or substituted with one or two substituents selected from halo and methoxy.

In some embodiments, R^{a} is H.

In some embodiments, R³ and R^{a} together with the nitrogen atom to which R³ and R^{a} are bound form a 9 or 10 membered heterocyclyl that is a fused ring system of an aromatic ring fused to a non-aromatic ring, wherein said heterocyclyl is unsubstituted or substituted with one or two substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, hydroxyl, and cyano.

In some embodiments, R³ and R^{a} together with the nitrogen atom to which R³ and R^{a} are bound form a 5, 9, or 10 membered heteroaryl, wherein said heteroaryl is unsubstituted or substituted with one or two substituents selected from C₁-C₄ alkyl, C₁-C₄ alkylsulfonyl, carboxy, aminocarbonyl, C₁-C₄ alkylaminocarbonyl, C₁-C₄ alkoxycarbonyl and C₁-C₄ alkoxy, each of which is substituted or unsubstituted, and halo, hydroxyl, and cyano.

In some embodiments, R³ and R^{a} together with the nitrogen atom to which R³ and R^{a} are bound form 1,2,3,4-tetrahydroquinoline-1-yl that is unsubstituted or substituted with one or two substituents selected from C₁-C₄ alkyl, C₁-C₄ alkylsulfonyl, carboxy, aminocarbonyl, C₁-C₄ alkylaminocarbonyl. C₁-C₄ alkoxycarbonyl, and C₁-C₄ alkoxy, each of which is substituted or unsubstituted, and halo, cyano, and hydroxy.

In some embodiments, the present disclosure provides a compound of Formula 3

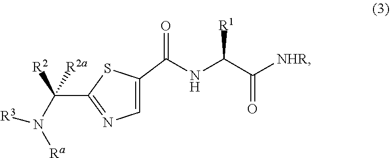

wherein
R is selected from H, unsubstituted or substituted C₁-C₄ alkoxy, unsubstituted or substituted C₁-C₄ alkyl, C₃-C₇cycloalkyl, C₅-C₁₀ bicycloalkyl, C₅-C₁₀ spirocycloalkyl, C₆-C₁₀ aryl, 5-10 membered heteroaryl, 4-10 membered heterocyclyl, 5-10 membered bicycloheterocyclyl, and 7-12 membered spiroheterocyclyl;
   wherein each of said cycloalkyl, bicycloalkyl and spirocycloalkyl is independently unsubstituted or substituted with one, two, or three substituents selected from heterocyclyl, C₁-C₄ alkyl, carboxy, aminocarbonyl, C₁-C₄ alkylaminocarbonyl, C₁-C₄ dialkylaminocarbonyl, C₁-C₄ alkoxycarbonyl, hydroxyl, amino, and C₁-C₄ alkoxy, each of which is substituted or unsubstituted, and halo, cyano, and hydroxyl;
   wherein each of said heterocyclyl, bicycloheterocyclyl, and spiroheterocyclyl is independently unsubstituted or substituted with one, two, or three substituents selected from heterocyclyl, C₁-C₄ alkyl, carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, cyano, and hydroxyl;
   wherein said heteroaryl is unsubstituted or substituted with one, two, or three substituents selected from heterocyclyl, C₁-C₄ alkyl, C₁-C₄ alkylsulfonyl, carboxy, aminocarbonyl, C₁-C₄ alkylaminocarbonyl C₁-C₄ dialkylaminocarbonyl, C₁-C₄ alkoxycarbonyl, amino, and C₁-C₄ alkoxy, each of which is substituted or unsubstituted, and halo, cyano, and hydroxyl; and
   wherein said aryl is unsubstituted or substituted with one, two, or three substituents selected from heterocyclyl, C₁-C₁ alkyl, C₁-C₄ alkylsulfonyl, carboxy, aminocarbonyl, C₁-C₄ alkylaminocarbonyl, C₁-C₄ dialkylaminocarbonyl C₁-C₄ alkoxycarbonyl, amino, and C₁-C₄ alkoxy, each of which is substituted or unsubstituted, and halo, cyano, and hydroxyl;
R¹ is selected from C₃-C₉ alkyl, cyclohexyl, cyclopentyl-C₁₋₂-alkyl, cyclohexyl-C₁₋₂-alkyl, bicyclo[2.2.2]octan-1-yl-C₁₋₂-alkyl, bicyclo[3.1.0]hexan-2-yl-C₁₋₂-alkyl and bicyclo[1.1.1]pentan-1-yl-C₁₋₂-alkyl, each of which is unsubstituted;
R² is selected from H, substituted or unsubstituted C₁-C₄ alkyl, C₁-C₂ haloalkyl, substituted or unsubstituted C₁-C₄ alkoxy-C₁-C₄ alkyl, substituted or unsubstituted 5-6 membered heterocyclyl-C₁-C₄ alkyl, substituted or unsubstituted aryl-C₁-C₄ alkyl, and substituted or unsubstituted 5-6 membered heteroaryl-C₁₋₂ alkyl;
R^{2a} is H or substituted or unsubstituted C₁₋₂ alkyl;
R³ is selected from H, C₆-C₁₀ aryl, and 5-10 membered heteroaryl, wherein each heteroaryl and aryl is unsubstituted or substituted with one, two, or three substituents selected from C₁-C₄ alkyl, C₃-C₆ heterocycloalkyl, aralkyl, C₁-C₄ alkylsulfonyl, carboxy, amino, substituted amino, C₃-C₇ cycloalkyl, phenyl, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl; and R^{a} is H or substituted or unsubstituted C₁-C₆ alkyl; or R³ and R^{a} together with the nitrogen atom to which R³ and R^{a} are bound form 5-10 membered heterocyclyl that is unsubstituted or substituted with one or two substituents selected from C₁-C₄ alkyl, C₁-C₄alkylsulfonyl, carboxy, aminocarbonyl, C₁-C₄ alkylaminocarbonyl, C₁-C₄ alkoxycarbonyl, or C₁-C₄ alkoxy, each of which is substituted or unsubstituted, and halo, cyano, and hydroxyl,
or a pharmaceutically acceptable salt thereof.

In some embodiments, R is selected from H, substituted or unsubstituted C₁-C₄ alkyl, C₃-C₇ cycloalkyl, phenyl, 5-10 membered heteroaryl, 4-10 membered heterocyclyl, C₅-C₁₀ bicycloalkyl, C₅-C₁₀ spirocycloalkyl 5-10 membered bicycloheterocyclyl, and 7-12 membered spiroheterocyclyl;
wherein each of said cycloalkyl, bicycloalkyl and spirocycloalkyl is independently unsubstituted or substituted with one, two, or three substituents selected from C₁-C₄ alkyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl;
wherein each of said heterocyclyl, bicycloheterocyclyl and spiroheterocyclyl is independently unsubstituted or substituted with one, two, or three substituents selected from C₁-C₄ alkyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl; wherein said heteroaryl is unsubstituted or substituted with one, two, or three substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl; and
wherein said phenyl is unsubstituted or substituted with one, two, or three substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl.

In some embodiments, R is selected from H, methyl, ethyl, fluoroethyl, isopropyl, 1-cyano-1-methylethyl, CH₃OCH₂CH₂OCH₂—, CH₃O(C═O)CH₂—, carboxymethyl, 3-fluorophenylmethyl, 4-fluorophenylmethyl, 3-pyridylmethyl, and 4-pyridylmethyl.

In some embodiments, R is selected from 3-pyrazolyl, 4-pyrazolyl, 1H-1,2,3-triazol-4-yl, 3-pyridinyl, 4-pyridinyl, 2-pyridinyl, 3-thienyl, phenyl, 5-indolyl, 6-indolyl, 5-indazolyl, 5-benzimidazolyl, 1H-pyrrolo[2,3-b]pyridin-5-yl, 1,2,3,4-tetrahydroquinolin-7-yl, 5,6,7,8-tetrahydroquinolin-6-yl, 1,2,3,4-tetrahydroquinolin-6-yl, 6-isoquinolinyl, and quinolin-6-yl, each of which is independently unsubstituted or substituted with one, two, or three substituents selected from methyl, methoxy, ethoxy, isopropoxy, carboxy, (2-methoxyeth-1-yl), methoxycarbonyl, methylaminocarbonyl, aminocarbonyl, methylsulfonyl, hydroxy, chloro, fluoro, and cyano.

In some embodiments, R is bicyclo[1.1.1]pentan-1-yl that is unsubstituted or substituted with one, two, or three substituents selected from methyl, methoxy, carboxy, methoxycarbonyl, methylaminocarbonyl, aminocarbonyl hydroxy, fluoro, and cyano.

In some embodiments, R is selected from bicyclo[1.1.1]pentan-1-yl, 3-methyl-bicyclo[1.1.1]pentan-1-yl, 3-methoxycarbonyl-bicyclo[1.1.1]pentan-1-yl, 3-carboxyl-bicyclo[1.1.1]pentan-1-yl, 3-aminocarbonyl-bicyclo[1.1.1]pentan-1-yl, 3-methylaminocarbonyl-bicyclo[1.1.1]pentan-1-yl, 3-hydroxy-bicyclo[1.1.1]pentan-1-yl, 3-fluoro-bicyclo[1.1.1]pentan-1-yl, 3-cyano-bicyclo[1.1.1]pentan-1-yl, and bicyclo[2.2.2]octan-1-yl.

In some embodiments, R is spiro[3.3]heptan-2-yl or 2-oxaspiro[3.3]heptan-6-yl.

In some embodiments, R is selected from 1,1-dioxo-1λ⁶-thian-4-yl, 2-oxabicyclo[2.1.1]hexan-4-yl and 8-oxabicyclo[3.2.1]octan-3-yl.

In some embodiments, R is selected from cyclopropyl, 1-cyanocyclopropyl, 1-trifluoromethyl-cyclopropyl, 2-fluorocyclopropyl, 2,2-difluorocyclopropyl, cyclobutyl, 3-fluorocyclobutyl, 2-methylcyclobutyl 3-methylcyclobutyl, 3,3-difluorocyclobutyl, 3-methoxycarbonylcyclobutyl, 3-carboxy-cyclobutyl, 3,3-difluorocyclopentyl, 3-cyanocyclobutyl, 3-hydroxycyclopentyl, cyclopentyl, 3-fluorocyclopentyl, and 3-cyanocyclopentyl.

In some embodiments, R is piperidin-4-yl or 1-methylpiperidin-4-yl.

In some embodiments, R is selected from 1-methyl-3-pyrazolyl, 3-pyrazolyl, 4-pyrazolyl, 1-methyl-4-pyrazolyl, 1-(2-methoxyeth-1-yl)-4-pyrazolyl, 1-ethyl-4-pyrazolyl, 1-fluoroethyl-4-pyrazolyl, 1-trifluoroethyl-4-pyrazolyl, 1-dimethylaminoethyl-4-pyrazoyl, 1-tert-butyl-4-pyrazolyl, 1-(1-methyl-4-piperidinyl)-4-pyrazolyl, 1-oxan-4-yl-4-pyrazolyl, 1-oxetan-3-yl-4-pyrazolyl, 1H-1,2,3-triazol-4-yl, 2-methylpyridin-4-yl, 2,6-dimethylpyridin-4-yl, 2-methylpyridin-5-yl, 2-methylpyridin-6-yl, 2-hydroxy-4-pyridinyl, 2-methoxy-4-pyridinyl, 2-chloro-4-pyridinyl, 2-aminocarbonyl-4-pyridinyl, 2-methoxy-5-pyridinyl, 2-aminocarbonyl-5-pyridinyl, 4-amino-2-pyridinyl, 3-thienyl, 4-cyanophenyl, 3-cyanophenyl, 3-methylsulfonylphenyl 4-methylsulfonylphenyl, 3,4-difluorophenyl, 4-fluorophenyl, 3-chlorophenyl, 4-chlorophenyl, 3-methoxyphenyl, 3-ethoxyphenyl, 3-isopropoxyphenyl, 4-methoxyethoxyphenyl, 4-fluorophenyl, 4-fluoro-3-methoxyphenyl, 5˜indolyl, 1-methyl-5-indolyl, 6-indolyl, 1-methyl-indol-6-yl, 4-chloro-6-indolyl, 7-fluoro-5-indolyl, 3-cyano-5-indolyl, 1-(2-methoxyeth-1-yl)-5-indolyl, 5-indazolyl, 5-benzimidazolyl 2-methyl-5-benzimidazolyl, 1H-pyrrolo[2,3-b]pyridin-5-yl, 1-methyl-1,2,3,4-tetrahydroquinolin-7-yl, 5,6,7,8-tetrahydroquinolin-6-yl, 1-methyl-1,2,3,4-tetrahydroquinolin-6-yl, 1-(2-methoxyeth-1-yl)-1,2,3,4-tetrahydroquinolin-6-yl, 6-isoquinolinyl, 3-cyano-quinolin-6-yl, 3-fluoroquinolin-6-yl, and 3-methoxyquinolin-6-yl.

In some embodiments, R² is selected from methyl, ethyl, benzyl, trifluoromethyl, 4-fluorobenzyl, methoxymethyl, 1-methyl-4-piperidinylmethyl, 4-pyridinyl ethyl, 1-pyrazolylmethyl, and 1-methyl-4-pyrazolylmethyl; and R^{2a} is H.

In some embodiments, R² is methyl; and R^{2a} is H.

In some embodiments, R² is methyl; and R^{2a} is methyl.

In some embodiments, R² is H and R^{2a} is H.

In some embodiments, R² is pyrazolo[1,5-a]pyrimidin-7-yl, phenyl or pyridyl, each of which is unsubstituted or substituted with one, two, or three substituents selected from chloro, fluoro, bromo, cyano, C₁₋₃ alkyl, hydroxymethyl, C₁₋₃ haloalkyl, cyclopropyl aminocarbonyl, di[C₁₋₃ alkyl]aminocarbonyl, di[C₁₋₃ alkyl]aminocarbonyl, methoxycarbonyl, carboxy, hydroxyl, C₁₋₃ alkoxy, C₁₋₃ alkoxyalkyl, pyridyl, and phenyl that is unsubstituted or substituted with one or two substituents selected from chloro, fluoro, bromo, cyano, methyl, ethyl, hydroxymethyl, trifluoromethyl, methoxymethyl, methoxypropyl, cyanopropyl, cyclopropyl, carboxy, methoxycarbonyl, aminocarbonyl, methylaminocarbonyl, dimethylaminocarbonyl, hydroxyl, and methoxy.

In some embodiments, R³ is 3-pyridyl that is unsubstituted or substituted with one or two substituents selected from chloro, fluoro, bromo, cyano, methyl, ethyl, hydroxymethyl, 2-hydroxy-2-methylethyl, trifluoromethyl, methoxymethyl, methoxypropyl, 2-cyano-2-methylethyl, cyclopropyl, carboxy, methoxycarbonyl, aminocarbonyl, methylaminocarbonyl, dimethylaminocarbonyl, hydroxyl, methoxy, pyridyl and phenyl that is unsubstituted or substituted with one or two substituents selected from chloro, fluoro, and methoxy.

In some embodiments, R¹ is selected from tert-butyl, 1-methylpropyl, 2-methylbutyl, 2,2-dimethylbutyl, cyclohexyl, bicyclo[2.2.2]octan-1-yl-CH₂—, bicyclo[3.1.0]hexan-2-yl-CH₂—, and bicyclo[1.1.1]pentan-1-yl-CH₂—.

In some embodiments, R¹ is cyclopentyl, cyclopentyl-CH₂—, or cyclohexyl-CH₂—.

In some embodiments, R³ and R^{a} together with the nitrogen atom to which R and W are bound form a 9 or 10 membered heterocyclyl that is a fused ring system of an aromatic ring fused to a non-aromatic ring, wherein said heterocyclyl is unsubstituted or substituted with one or two substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, hydroxyl, and cyano.

In some embodiments, R³ and R^{a} together with the nitrogen atom to which R³ and R^{a} are bound form a 5, 9, or 10 membered heteroaryl, wherein said heteroaryl is unsubstituted or substituted with one or two substituents selected from C₁-C₄ alkyl, C₁-C₄ alkylsulfonyl, carboxy, aminocarbonyl, C₁-C₄ alkylaminocarbonyl, C₁-C₁ alkoxycarbonyl, and C₁-C₄ alkoxy, each of which is substituted or unsubstituted, and halo, hydroxyl, and cyano.

In some embodiments, R³ and R^{a} together with the nitrogen atom to which R³ and R^{a} are bound form dihydroindol-1-yl, 4-methyl-7-chloro-tetrahydroquinoxalin-1-yl, 5-chloro-1,2,3,4-tetrahydroquinolin-2-yl or 7-chloro-1,2,3,4-tetrahydroquinolin-1-yl, wherein the dihydroindol-1-yl or tetrahydroquinoline-1-yl each are independently unsubstituted or substituted with one or two substituents selected from halo and methoxy.

In some embodiments, the present disclosure provides a compound of Formula 4.

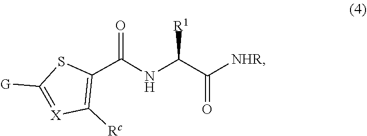

wherein G is

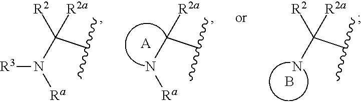

X is CR^{b} or N;
R is selected from H unsubstituted or substituted C₁-C₄ alkoxy, unsubstituted or substituted C₁-C₄ alkyl, C₃-C₇ cycloalkyl, C₅-C₁₀ bicycloalkyl, C₅-C₁₀ spirocycloalkyl, C₆-C₁₀ aryl, 5-10 membered heteroaryl, 4-10 membered heterocyclyl, 5-10 membered bicycloheterocyclyl, and 7-12 membered spiroheterocyclyl;
   wherein each of said cycloalkyl, bicycloalkyl and spirocycloalkyl is independently unsubstituted or substituted with one, two, or three substituents selected from heterocyclyl, C₁-C₄ alkyl, carboxy, aminocarbonyl, C₁-C₄ alkylaminocarbonyl, C₁-C₄ dialkylaminocarbonyl, C₁-C₄ alkoxycarbonyl, hydroxyl, amino, and C₁-C₄ alkoxy, each of which is substituted or unsubstituted, and halo, hydroxyl, and cyano;
   wherein each of said heterocyclyl, bicycloheterocyclyl, and spiroheterocyclyl is independently unsubstituted or substituted with one, two, or three substituents selected from heterocyclyl, C₁-C₄ alkyl, carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, hydroxyl, and cyano;
   wherein said heteroaryl is unsubstituted or substituted with one, two, or three substituents selected from heterocyclyl, C₁-C₄ alkyl, C₁-C₄ alkylsulfonyl, carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, hydroxyl, and cyano; and
   wherein said aryl is unsubstituted or substituted with one, two, or three substituents selected from heterocyclyl, C₁-C₄ alkyl, C₁-C₄ alkylsulfonyl, carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, hydroxyl and cyano;
R¹ is selected from C₃-C₈ alkyl, cyclohexyl, cyclopentyl-C₁₋₂-alkyl, cyclohexyl-C₁₋₂-alkyl, bicyclo[2.2.2]octan-1-yl-C₁₋₂-alkyl, bicyclo[3.1.0]hexan-2-yl-C₁₋₂-alkyl and bicyclo[1.1.1]pentan-1-yl-C₁₋₂-alkyl, each of which is unsubstituted;
R² is selected from H, substituted or unsubstituted C₁-C₄ alkyl, C₁-C₂ haloalkyl, substituted or unsubstituted C₁-C₄ alkoxy-C₁-C₄ alkyl, substituted or unsubstituted 5-6 membered heterocyclyl-C₁-C₄ alkyl, substituted or unsubstituted aryl-C₁-C₄ alkyl, and substituted or unsubstituted 5-6 membered heteroaryl-C₁₋₂ alkyl;
R^{2a} is H or substituted or unsubstituted C₁-C₄ alkyl;
R³ is selected from H, C₆-C₁₀ aryl, and 5-10 membered heteroaryl, wherein each heteroaryl and aryl is unsubstituted or substituted with one, two, or three substituents selected from C₁-C₄ alkyl, C₃-C₆ heterocycloalkyl, aralkyl, C₁-C₄ alkylsulfonyl, carboxy, amino, substituted amino, C₃-C₇ cycloalkyl, phenyl, aminocarbonyl, C₁-C₄ alkylaminocarbonyl, C₁-C₄ alkoxycarbonyl, and CiC_{G} alkoxy, each of which is substituted or unsubstituted, and halo, hydroxyl, and cyano; and
R^{a} is H or C₁-C₆ alkyl;
ring A is a 9-10 membered heterocyclyl that is unsubstituted or substituted with one or two substituents selected from C₁-C₄ alkyl C₁-C₄ alkylsulfonyl, carboxy, aminocarbonyl, C₁-C₄ alkylaminocarbonyl, C₁-C₄ alkoxycarbonyl, hydroxyl, and C₁-C₄ alkoxy, each of which is substituted or unsubstituted, and halo, hydroxyl, and cyano;
ring B is a 5-10 membered heterocyclyl that is unsubstituted or substituted with one or two substituents selected from C₃-C₇ alkyl, C₁-C₄ alkylsulfonyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, hydroxyl, and cyano; and
R^{b} and R^{c} are independently selected from C₁-C₄ alkyl, C₃-C₆ heterocycloalkyl, aralkyl, C₁-C₄ alkylsulfonyl, carboxy, amino, C₃-C₇ cycloalkyl, aminocarbonyl, C₁-C₄ alkylaminocarbonyl, C₁-C₄ alkoxycarbonyl, and C₁-C₄ alkoxy, each of which is substituted or unsubstituted, and H, halo, hydroxyl, and cyano,
provided R² and R^{2a} are not both H when:
(i) X is CH, R^{c} is H, R¹ is cyclopentylmethyl or cyclohexylmethyl, R^{a} is H or ethyl, R³ is 3-chloropyridin-5-yl, 3-chloro-6-methyl-pyridin-5-yl, 4-chloropyridin-2-yl, or 3-chlorophenyl, and R is H, methyl, ethyl, methoxy, hydroxyethyl, hydroxypropyl, methoxyethyl, cyclopropyl, 3-hydroxycyclobutyl, 3-(1,5-diaminopent-1-ylcarbonyloxy)cyclobutyl, 3-(N²,N⁶-bis(tert-butoxycarbonyl)lysyl)oxycyclobutyl, 3-methylcarbonyloxycyclobutyl, or oxetan-3-yl, or
(ii) X is N, R^{c} is H, R¹ is cyclohexyl or cyclohexylethyl, R^{a} is H, R³ is H or 2-fluoro-4-(3-(4-methylpiperazin-1-yl)-3-oxo-2-propionamidopropyl)phenyl, and R is H or methyl,
or a pharmaceutically-acceptable salt thereof.

In some embodiments, R is selected from H, substituted or unsubstituted C₁-C₄ alkyl, C₃-C₇ cycloalkyl, phenyl, 5-10 membered heteroaryl, 4-10 membered heterocyclyl, C₅-C₁₀ bicycloalkyl, C₅-C₁₀ spirocycloalkyl 5-10 membered bicycloheterocyclyl, and 7-12 membered spiroheterocyclyl;
wherein each of said cycloalkyl, bicycloalkyl and spirocycloalkyl is independently unsubstituted or substituted with one, two, or three substituents selected from C₁-C₄ alkyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl;
wherein each of said heterocyclyl, bicycloheterocyclyl and spiroheterocyclyl is independently unsubstituted or substituted with one, two, or three substituents selected from C₁-C₄ alkyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl;
wherein said heteroaryl is unsubstituted or substituted with one, two, or three substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl; and
wherein said phenyl is unsubstituted or substituted with one, two, or three substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl.

In some embodiments, R is selected from H, methyl, ethyl, fluoroethyl, isopropyl, 1-cyano-1-methylethyl, CH₃OCH₂CHC₂—, CH₃O(C═O)CH₂, carboxymethyl, 3-fluorophenylmethyl, 4-fluorophenylmethyl, 3-pyridylmethyl, and 4-pyridylmethyl.

In some embodiments, R is selected from 3-pyrazolyl, 4-pyrazolyl, 1H1,2,3-triazol-4-yl, 3-pyridinyl, 4-pyridinyl, 2-pyridinyl, 3-thienyl, phenyl, 5-indolyl, 6-indolyl, 5-indazolyl, 5-benzimidazolyl, 1H-pyrrolo[2,3-b]pyridin-5-yl, 1,2,3,4 tetrahydroquinolin-7-yl, 5,6,7,8-tetrahydroquinolin-6-yl, 1,2,3,4-tetrahydroquinolin-6-yl-6-isoquinolinyl, and quinolin-6-yl; each of which is unsubstituted or substituted with one, two, or three substituents selected from methyl, methoxy, ethoxy, isopropoxy, carboxy, (2-methoxyeth-1-yl), methoxycarbonyl, methylaminocarbonyl, aminocarbonyl, methylsulfonyl, hydroxy, chloro, fluoro, and cyano.

In some embodiments, R is bicyclo[1.1.1]pentan-1-yl that is unsubstituted or substituted with one, two, or three substituents selected from methyl, methoxy, carboxy, methoxycarbonyl, methylaminocarbonyl, aminocarbonyl hydroxy, fluoro, and cyano.

In some embodiments, R is selected from bicyclo[1.1.1]pentan-1-yl, 3-methyl-bicyclo[1.1.1]pentan-1-yl, 3-methoxycarbonyl-bicyclo[1.1.1]pentan-1-yl, 3-carboxyl-bicyclo[1.1.1]pentan-1-yl, 3-aminocarbonyl-bicyclo[1.1.1]pentan-1-yl, 3-methylaminocarbonyl-bicyclo[1.1.1]pentan-1-yl, 3-hydroxy-bicyclo[1.1.1]pentan-1-yl, 3-fluoro-bicyclo[1.1.1]pentan-1-yl, 3-cyano-bicyclo[1.1.1]pentan-1-yl, and bicyclo[2.2.2]octan-1-yl.

In some embodiments, R is spiro[3.3]heptan-2-yl or 2-oxaspiro[3.3]heptan-6-yl.

In some embodiments, R is selected from 1,1-dioxo-1λ⁶-thian-4-yl, 2-oxabicyclo[2.1.1]hexan-4-yl, and 8-oxabicyclo[3.2.1]octan-3-yl.

In some embodiments, R is selected from cyclopropyl, 1-cyanocyclopropyl, 1-trifluoromethyl-cyclopropyl, 2-fluorocyclopropyl, 2,2-difluorocyclopropyl, cyclobutyl, 3-fluorocyclobutyl, 2-methylcyclobutyl, 3-methylcyclobutyl, 3,3-difluorocyclobutyl, 3-methoxycarbonylcyclobutyl, 3-carboxy-cyclobutyl, 3,3-difluorocyclopentyl, 3-cyanocyclobutyl, 3-hydroxycyclopentyl, cyclopentyl, 3-fluorocyclopentyl, and 3-cyanocyclopentyl.

In some embodiments, R is selected from piperidin-4-yl, 1-methylpiperidin-4-yl, and 3-oxetanyl.

In some embodiments, R is selected from 1-methyl-3-pyrazolyl, 3-pyrazolyl, 4-pyrazolyl, 1-methyl-4-pyrazolyl, 1-(2-methoxyeth-1-yl)-4-pyrazolyl, 1-ethyl-4-pyrazolyl, 1-fluoroethyl-4-pyrazolyl, 1-trifluoroethyl-4l-pyrazolyl, 1-dimethylaminoethyl-4-pyrazoyl, 1-tert-butyl-4-pyrazolyl, 1-(1-methyl-4-piperidinyl)-4-pyrazolyl, 1-oxan-4-yl-4-pyrazolyl, 1-oxetan-3-yl-4-pyrazolyl, 1H-1,2,3-triazol-4-yl, 2-methylpyridin-4-yl, 2,6-dimethylpyridin-4-yl, 2-methylpyridin-5-yl, 2-methylpyridin-6-yl, 2-hydroxy-4-pyridinyl, 2-methoxy-4-pyridinyl, 2-chloro-4-pyridinyl, 2-aminocarbonyl-4-pyridinyl, 2-methoxy-5-pyridinyl, 2-aminocarbonyl-5-pyridinyl, 4-amino-2-pyridinyl, 3-thienyl, 4-cyanophenyl, 3-cyanophenyl, 3-methylsulfonylphenyl, 4-methylsulfonylphenyl, 3,4-difluorophenyl, 4-fluorophenyl, 3-chlorophenyl, 4-chlorophenyl, 3-methoxyphenyl, 3-ethoxyphenyl, 3-isopropoxyphenyl, 4-methoxymethoxyphenyl, 4-fluorophenyl, 4-fluoro-3-methoxyphenyl, 5-indolyl, 1-methyl-5-indolyl, 6-indolyl, 1-methyl-Indol-6-JI, 4-chloro-6-indolyl, 7-fluoro-5-indolyl, 3-cyano-5-indolyl, 1-(2-methoxyeth-1-yl)-5-indolyl, 5-indazolyl, 5-benzimidazolyl, 2-methyl-5-benzimidazolyl, 1H-pyrrolo[2,3-b]pyridin-5-yl, 1-methyl-1,2,3,4-tetrahydroquinolin-7-yl, 5,6,7,8-tetrahydroquinolin-6-yl, 1-methyl-1,2,3,4-tetrahydroquinolin-6-yl, 1-(2-methoxyeth-1-yl)-1,2,3,4-tetrahydroquinolin-6-yl, 6-isoquinolinyl, 3-cyano-quinolin-6-yl, 3-fluoroquinolin-6-yl, and 3-methoxyquinolin-6-yl.

In some embodiments, R² is selected from methyl, ethyl, benzyl, trifluoromethyl, 4-fluorobenzyl, methoxymethyl, 1-methyl-4-piperidinylmethyl, 4-pyridinylmethyl, 1-pyrazolylmethyl, and 1-methyl-4-pyrazolylmethyl; and R is H.

In some embodiments, R² is methyl; and R^{2a} is H.

In some embodiments, R² is methyl; and R^{2a} is methyl.

In some embodiments, R² is H; and R^{2a} is H.

In some embodiments, R³ is pyrazolo[1,5-a]pyrimidin-7-yl, phenyl or pyridyl, each of which is unsubstituted or substituted with one, two, or three selected from chloro, fluoro, bromo, cyano, C₁₋₃ alkyl, hydroxymethyl, C₁₋₃ haloalkyl, cyclopropyl, aminocarbonyl, di[C₁₋₃ alkyl]aminocarbonyl, di[C₁₋₃ alkyl]aminocarbony, methoxycarbonyl, carboxy, hydroxyl, C₁₋₃ alkoxy, C₁₋₃ alkoxyalkyl, pyridyl, and phenyl that is unsubstituted or substituted with one or two substituents selected from chloro, fluoro, bromo, cyano, methyl, ethyl, hydroxymethyl, trifluoromethyl, methoxymethyl, methoxypropyl, cyanopropyl, cyclopropyl, carboxy, methoxycarbonyl, aminocarbonyl, methylaminocarbonyl, dimethylaminocarbonyl, hydroxyl, and methoxy.

In some embodiments, R³ is 3-pyridyl that is unsubstituted or substituted with one or two substituents selected from chloro, fluoro, bromo, cyano, methyl, ethyl, hydroxymethyl, 2-hydroxy-2-methylethyl, trifluoromethyl, methoxymethyl, methoxypropyl, 2-cyano-2-methylethyl, cyclopropyl, carboxy, methoxycarbonyl, aminocarbonyl, methylaminocarbonyl, dimethylaminocarbonyl, hydroxyl, methoxy, pyridyl and phenyl that is unsubstituted or substituted with one or two substituents selected from chloro, fluoro, and methoxy.

In some embodiments, R¹ is selected from tert-butyl, 1-methylpropyl, 2-methyl butyl, 2,2-dimethylbutyl, cyclohexyl, bicyclo[2.2.2]octan-1-yl-CH₂—, bicyclo[3.1.0]hexan-2-yl-CH₂— and bicyclo[1.1.1]pentan-1-yl-CH₂—.

In some embodiments, R¹ is cyclopentyl-CH₂— or cyclohexyl-CH₂—.

In some embodiments, ring A is dihydroindol-1-yl, 4-methyl-7-chloro-tetrahydroquinoxalin-1-yl, 5-chloro-1,2,3,4-tetrahydroquinolin-2-yl or 7-chloro-1,2,3,4-tetrahydroquinolin-1-yl wherein the dihydroindol-1-yl or tetrahydroquinolin-1-yl each are independently unsubstituted of substituted with one or two substituents selected from halo or methoxy.

In some embodiments, R^{a} is H.

In some embodiments, R⁴ is H.

In some embodiments, ring A is a 9 or 10 membered heterocyclyl that is a fused ring system of an aromatic ring fused to a non-aromatic ring, wherein said heterocyclyl is unsubstituted or substituted with one or two substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, hydroxyl, and C₁-C₄ alkoxy, each of which is substituted or unsubstituted, and halo, hydroxyl, and cyano.

In some embodiments, ring B is a 9 or 10 membered heterocyclyl that is a fused ring system of an aromatic ring fused to a non-aromatic ring, wherein said heterocyclyl is unsubstituted or substituted with one or two substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, hydroxyl, and cyano.

In some embodiments, ring B is a 5, 9, or 10 membered heteroaryl, wherein said heteroaryl is unsubstituted or substituted with one or two substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, hydroxyl, and cyano.

In some embodiments, ring B is 1,2,3,4-tetrahydroquinolin-1-yl that is unsubstituted or substituted with one or two substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl.

In some embodiments, ring A is 1,2,3,4-tetrahydroquinolin-1,2-diyl that is unsubstituted or substituted with one or two substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl. In some embodiments, the present disclosure provides a compound of formula 5:

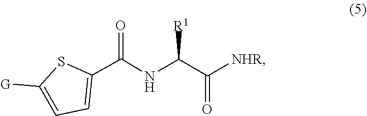

wherein
wherein G is

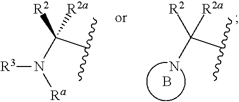

R is selected from H, unsubstituted or substituted C₁-C₄ alkoxy, unsubstituted or substituted C₁-C₄alkyl, C₃-C₇ cycloalkyl, C₅-C₁₀ bicycloalkyl, C₅-C₁₀ spirocycloalkyl, C₆-C₁₀ aryl, 5-10 membered heteroaryl, 4-10 membered heterocyclyl, 5-10 membered bicycloheterocyclyl, and 7-12 membered spiroheterocyclyl;
   wherein each of said cycloalkyl, bicycloalkyl and spirocycloalkyl is independently unsubstituted or substituted with one, two, or three substituents selected from heterocyclyl, C₁-C₄ alkyl, carboxy, aminocarbonyl, C₁-C₄ alkylaminocarbonyl, C₁-C₄ dialkylaminocarbonyl, C₁-C₄ alkoxycarbonyl, hydroxyl, amino, and C₁-C₄ alkoxy, each of which is substituted or unsubstituted, and halo, cyano, and hydroxyl;
   wherein each of said heterocyclyl, bicycloheterocyclyl, and spiroheterocyclyl is independently unsubstituted or substituted with one, two, or three substituents selected from heterocyclyl, C₁-C₄ alkyl, carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, cyano, and hydroxyl;
   wherein said heteroaryl is unsubstituted or substituted with one, two, or three substituents selected from heterocyclyl, C₁-C₄ alkyl, C₁-C₄ alkylsulfonyl, carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, cyano, and hydroxyl; and
   wherein said aryl is unsubstituted or substituted with one, two, or three substituents selected from heterocyclyl, C₁-C₄ alkyl, C₁-C₄ alkylsulfonyl, carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, cyano, and hydroxyl;
R¹ is selected from C₁-C₄ alkyl, cyclohexyl, cyclopentyl-C₁₋₂-alkyl, cyclohexyl-C₁₋₂-alkyl, bicyclo[2.2.2]octan-1-yl-C₁₋₂-alkyl, bicyclo[3.1.0]hexan-2-yl-C₁₋₂-alkyl and bicyclo[1.1.1]pentan-1-yl-C₁₋₂-alkyl, each of which is unsubstituted;
   R² is selected from substituted or unsubstituted C₁-C₄ alkyl, C₁-C₂ haloalkyl, substituted or unsubstituted C₁-C₄ alkoxy-C₁-C₄ alkyl, substituted or unsubstituted 5-6 membered heterocyclyl-C₁-C₄ alkyl, substituted or unsubstituted aryl-C₁-C₄ alkyl, and substituted or unsubstituted 5-6 membered heteroaryl-C₁₋₂ alkyl;
R^{2a} is H or substituted or unsubstituted C₁-C₄ alkyl;
R³ is selected from H, C₆-C₁₀ aryl, and 5-10 membered heteroaryl, wherein each heteroaryl and aryl is unsubstituted or substituted with one, two, or three substituents selected from C₁-C₄ alkyl, C₃-C₆ heterocycloalkyl, aralkyl, C₁-C₄ alkylsulfonyl, carboxy, amino, substituted amino, C₃-C₇ cycloalkyl, phenyl, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl; and
R^{a} is H or substituted or unsubstituted C₁-C₆ alkyl;
ring B is a 5-10 membered heterocyclyl that is unsubstituted or substituted with one or two substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, or C₁-C₄ alkoxy, each of which is substituted or unsubstituted, and halo, cyano, and hydroxyl,
or a pharmaceutically acceptable salt thereof.

In some embodiments, R is selected from H, substituted or unsubstituted C₁-C₄ alkyl, C₃-C₇ cycloalkyl, phenyl, 5-10 membered heteroaryl, 4-10 membered heterocyclyl, C₅-C₁₀ bicycloalkyl, C₅-C₁₀ spirocycloalkyl, 5-10 membered bicycloheterocyclyl, and 7-12 membered spiroheterocyclyl; wherein each of said cycloalkyl, bicycloalkyl and spirocycloalkyl is independently unsubstituted or substituted with one, two, or three substituents selected from C₁-C₄ alkyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl;
wherein each of said heterocyclyl, bicycloheterocyclyl and spiroheterocyclyl is independently unsubstituted or substituted with one, two, or three substituents selected from C₁-C₄ alkyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl;
   wherein said heteroaryl is unsubstituted or substituted with one, two, or three substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl; and
   wherein said phenyl is unsubstituted or substituted with one, two, or three substituents selected from C₁-C₄ alkyl, C₁-C₄ alkylsulfonyl, carboxy, aminocarbonyl, C₁-C₁ alkylaminocarbonyl, C₁-C₄ alkoxycarbonyl, and C₁-C₄ alkoxy, each of which is substituted or unsubstituted, and halo, cyano, and hydroxyl.

In some embodiments, R is selected from H, methyl, ethyl, fluoroethyl, isopropyl, 1-cyano-1-methylethyl, CHOCH₂CH₂OCH₂—, CH₃O(C═O)CH₂—, carboxymethyl, 3-fluorophenylmethyl, 4-fluorophenylmethyl, 3-pyridylmethyl, and 4-pyridylmethyl.

In some embodiments, R is selected from 3-pyrazolyl, 4-pyrazolyl, 1H-1,2,3-triazol-4-yl, 3-pyridinyl, 4-pyridinyl, 2-pyridinyl, 3-thienyl, phenyl, 5-indolyl, 6-indolyl, 5-indazolyl, 5-benzimidazolyl, 1H-pyrrolo[2,3-b]pyridin-5-yl, 1,2,3,4-tetrahydroquinolin-1-yl, 5,6,7,8-tetrahydroquinolin-6-yl, 1,2,3,4-tetrahydroquinolin-6-yl, 6-isoquinolinyl, and quinolin-6-yl, each of which is independently unsubstituted or substituted with one, two, or three substituents selected from methyl, methoxy, ethoxy, isopropoxy, carboxy, (2-methoxyeth-1-yl), methoxycarbonyl, methylaminocarbonyl, aminocarbonyl, methylsulfonyl, hydroxy, chloro, fluoro, and cyano.

In some embodiments, R is bicyclo[1.1.1]pentan-1-yl that is unsubstituted or substituted with one, two, or three substituents selected from methyl, methoxy, carboxy, methoxycarbonyl, methylaminocarbonyl, aminocarbonyl hydroxy, fluoro, and cyano.

In some embodiments, R is selected from bicyclo[1.1.1]pentan-1-yl, 3-methyl-bicyclo[1.1.1]pentan-1-yl, 3-methoxycarbonyl-bicyclo[1.1.1]pentan-1-yl, 3-carboxyl-bicyclo[1.1.1]pentan-1-yl, 3-aminocarbonyl-bicyclo[1.1.1]pentan-1-yl, 3-methylaminocarbonyl-bicyclo[1.1.1]pentan-1-yl, 3-hydroxy-bicyclo[1.1.1]pentan-1-yl, 3-fluoro-bicyclo[1.1.1]pentan-1-yl, 3-cyano-bicyclo[1.1.1]pentan-1-yl, and bicyclo[2.2.2]octan-1-yl.

In some embodiments, R is spiro[3.3]heptan-2-yl or 2-oxaspiro[3.3]heptan-6-yl.

In some embodiments, R is selected from 1,1-dioxo-1λ⁶-thian-4-yl, 2-oxabicyclo[2.1.1]hexan-4-yl and 8-oxabicyclo[3.2.1]octan-3-yl.

In some embodiments, R is selected from cyclopropyl, 1-cyanocyclopropyl, 1-trifluoromethyl-cyclopropyl, 2-fluorocyclopropyl, 2,2-difluorocyclopropyl, cyclobutyl, 3-fluorocyclobutyl, 2-methylcyclobutyl 3-methylcyclobutyl, 3,3-difluorocyclobutyl, 3-methoxycarbonylcyclobutyl, 3-carboxy-cyclobutyl, 3,3-difluorocyclopentyl, 3-cyanocyclobutyl, 3-hydroxycyclopentyl, cyclopentyl, 3-fluorocyclopentyl, and 3-cyanocyclopentyl.

In some embodiments, R is piperidin-4-yl or 1-methylpiperidin-4-yl.

In some embodiments, R is selected from 1-methyl-3-pyrazolyl, 3-pyrazolyl, 4-pyrazolyl, 1-methyl-4-pyrazoyl, 1-(2-methoxyeth-1-yl)-4-pyrazolyl, 1-ethyl-4-pyrazolyl, 1-fluoroethyl-pyrazolyl, 1-trifluoroethyl-4-pyrazolyl, 1-dimethylaminoethyl-4-pyrazolyl, 1-tert-butyl-4-pyrazolyl, 1-(1-methyl-4-piperidinyl)-4-pyrazolyl, 1-oxan-4-yl-4-pyrazolyl, 1-oxetan-3-yl-4-pyrazolyl, 1H-1,2,3-triazol-4-yl, 2-methylpyridin-4-yl, 2,6-dimethylpyridin-4-yl, 2-methylpyridin-5-yl, 2-methylpyridin-6-yl, 2-hydroxy-4-pyridinyl, 2-methoxy-4-pyridinyl, 2-chloro-4-pyridinyl, 2-aminocarbonyl-4-pyridinyl, 2-methoxy-5-pyridinyl, 2-aminocarbonyl-5-pyridinyl, 4-amino-2-pyridinyl, 3-thienyl, 4-cyanophenyl, 3-cyanophenyl, 3-methylsulfonylphenyl, 4-methylsulfonylphenyl, 3,4-difluorophenyl, 4-fluorophenyl, 3-chlorophenyl, 4-chlorophenyl, 3-methoxyphenyl, 3-ethoxyphenyl, 3-isopropoxyphenyl, 4-methoxymethoxyphenyl, 4-fluorophenyl, 4-fluoro-3-methoxyphenyl, 5-indolyl, 1-methyl-5-indolyl, 6-indolyl, 1-methyl-indol-6-yl, 4-chloro-6-indolyl, 7-fluoro-5-indolyl, 3-cyano-5-indolyl, 1-(2-methoxyeth-1-yl)-5-indolyl, 5-indazolyl, 5-benzimidazolyl, 2-methyl-5-benzimidazolyl, 1H-pyrrolo[2,3-b]pyridin-5-yl, 1-methyl-1,2,3,4-tetrahydroquinolin-7-yl, 5,6,7,8-tetrahydroquinolin-6-yl, 1-methyl-1,2,3,4-tetrahydroquinolin-6-yl, 1-(2-methoxyeth-1-yl)-1,2,3,4-tetrahydroquinolin-6-yl, 6-isoquinolinyl, 3-cyano-quinolin-6-yl, 3-fluoroquinolin-6-yl, 3-methoxyquinolin-6-yl, and 8-methoxyquinolin-6-yl.

In some embodiments, R² is selected from methyl, ethyl, benzyl, trifluoromethyl, 4-fluorobenzyl, methoxymethyl, 1-methyl-4-piperidinylmethyl, 4-pyridinylmethyl, 1-pyrazolylmethyl, and 1-methyl-4-pyrazolylmethyl; and R^{2a} is H.

In some embodiments, R² is methyl; and R^{2a} is H.

In some embodiments, R² is methyl; and R^{2a} is methyl.

In some embodiments, R³ is pyrazolo[1,5-a]pyrimidin-7-yl, phenyl or pyridyl, each of which is independently unsubstituted or substituted with one, two, or three substituents selected from chloro, fluoro, bromo, cyano, C₁₋₃ alkyl, hydroxymethyl, C₁₋₃ haloalkyl, cyclopropyl, aminocarbonyl, di[C₁₋₃ alkyl]aminocarbonyl, di[C₁₋₃ alkyl]aminocarbonyl, methoxycarbonyl, carboxy, hydroxyl, C₁₋₃ alkoxy, C₁₋₃ alkoxyalkyl, pyridyl, and phenyl that is unsubstituted or substituted with one or two substituents selected from chloro, fluoro, bromo, cyano, methyl, ethyl, hydroxymethyl, trifluoromethyl, methoxymethyl, m ethoxypropyl, cyanopropyl, cyclopropyl, carboxy, methoxycarbonyl, aminocarbonyl, methylaminocarbonyl, dimethylaminocarbonyl, hydroxyl, and methoxy.

In some embodiments, R³ is 3-pyridyl that is unsubstituted or substituted with one or two substituents selected from chloro, fluoro, bromo, cyano, methyl, ethyl, hydroxymethyl, 2-hydroxy-2-methylethyl, trifluoromethyl, methoxymethyl, methoxypropyl, 2-cyano-2-methylethyl, cyclopropyl, carboxy, methoxycarbonyl, aminocarbonyl, methylaminocarbonyl, dimethylaminocarbonyl, hydroxyl, methoxy, pyridyl and phenyl that is unsubstituted or substituted with one or two substituents selected from chloro, fluoro, and methoxy.

In some embodiments, R¹ is tert-butyl, 1-methylpropyl, 2-methylbutyl, 2,2-dimethylbutyl, cyclohexyl, bicyclo[2.2.2]octan-1-yl-CH₂—, bicyclo[3.1.0]hexan-2-yl-CH₂— or bicyclo[1.1.1]pentan-1-yl-CH₂—.

In some embodiments, R¹ is cyclopentyl-CH₂— or cyclohexyl-CH₂—.

In some embodiments, ring B is dihydroindol-1-yl, 4-methyl-7-chloro-tetrahydroquinoxalin-1-yl 5-chloro-1,3,4-tetrahydroquinolin-2-yl or 7-chloro-1,2,3,4-tetrahydroquinolin-1-yl, wherein the dihydroindol-1-yl or tetrahydroquinoline-1-yl each are unsubstituted or substituted with one or two substituents selected from halo and methoxy.

In some embodiments, R¹ is H.

In some embodiments, ring B is a 9 or 10 membered heterocyclyl that is a fused ring system of an aromatic ring fused to a non-aromatic ring, wherein said heterocyclyl is unsubstituted or substituted with one or two substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, hydroxyl, and cyano.

In some embodiments, ring B is a 5, 9, or 10 membered heteroaryl, wherein said heteroaryl is unsubstituted or substituted with one or two substituents selected from C₁-C₁ alkyl, C₁-C₄ alkylsulfonyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, hydroxyl, and cyano.

In some embodiments, ring B is 1,2,3,4-tetrahydroquinoline-1-yl that is unsubstituted or substituted with one or two substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxy.

In some embodiments, the present disclosure provides a compound of formula 6:

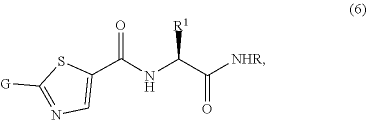

wherein
wherein G is

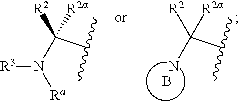

R is selected from H, unsubstituted or substituted C₁-C₄ alkoxy, unsubstituted or substituted C₁-C₄ alkyl, C₃-C₇ cycloalkyl, C₅-C₁₀ bicycloalkyl, C₅-C₁₀ spirocycloalkyl, C₆-C₁₀ aryl, 5-10 membered heteroaryl, 4-10 membered heterocyclyl, 5-10 membered bicycloheterocyclyl, and 7-12 membered spiroheterocyclyl;
   wherein each of said cycloalkyl, bicycloalkyl and spirocycloalkyl is independently unsubstituted or substituted with one, two, or three substituents selected from heterocyclyl, C₁-C₄ alkyl, carboxy, aminocarbonyl, C₁-C₄ alkylaminocarbonyl C₁-C₄ dialkylaminocarbonyl, C₁-C₄ alkoxycarbonyl, hydroxyl, amino, and C₁-C₄ alkoxy, each of which is substituted or unsubstituted, and halo, cyano, and hydroxyl;
   wherein each of said heterocyclyl, bicycloheterocyclyl, and spiroheterocyclyl is independently unsubstituted or substituted with one, two, or three substituents selected from heterocyclyl, C₁-C₄ alkyl, carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, cyano, and hydroxyl;
   wherein said heteroaryl is unsubstituted or substituted with one, two, or three substituents selected from heterocyclyl, C₁-C₄ alkyl, C₁-C₄ alkylsulfonyl, carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, cyano, and hydroxyl, and
   wherein said aryl is unsubstituted or substituted with one, two, or three substituents selected from heterocyclyl, C₁-C₄ alkyl, C₁-C₄ alkylsulfonyl carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, cyano, and hydroxyl;
R¹ is selected from C₃-C₈ alkyl, cyclohexyl, cyclopentyl-C₁₋₂-alkyl, cyclohexyl-C₁₋₂-alkyl, bicyclo[2.2.2]octan-1-yl-C₁₋₂-alkyl, bicyclo[3.1.0]hexan-2-yl-C₁₋₂-alkyl, and bicyclo[1.1.1]pentan-1-yl-C₁₋₂-alkyl, each of which is unsubstituted;
R² is selected from H, substituted or unsubstituted C₁-C₄ alkyl, C₁-C₂ haloalkyl, substituted or unsubstituted C₁-C₄ alkoxy-C₁-C₄ alkyl, substituted or unsubstituted 5-6 membered heterocyclyl-C₁-C₄ alkyl, substituted or unsubstituted aryl-C₁-C₄ alkyl, and substituted or unsubstituted 5-6 membered heteroaryl-C₁₋₂ alkyl;
R^{2a} is H or substituted or unsubstituted C₁-C₄ alkyl;
R³ is selected from H, C₆-C₁₀ aryl, and 5-10 membered heteroaryl, wherein each heteroaryl and aryl is unsubstituted or substituted with one, two, or three substituents selected from C₁-C₄ alkyl, C₃-C₆ heterocycloalkyl, aralkyl, C₁-C₄ alkylsulfonyl, carboxy, amino, substituted amino, C₃-C₇ cycloalkyl, phenyl, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl; and
R^{a} is H or substituted or unsubstituted C₁-C₆ alkyl;
ring B is a 5-10 membered heterocyclyl that is unsubstituted or substituted with one or two substituents selected from C₁-C₄ alkyl, C₁-C₄ alkylsulfonyl carboxy, aminocarbonyl, C₁-C₄ alkylaminocarbonyl, C₁-C₄ alkoxycarbonyl, or C₁-C₄ alkoxy, each of which is substituted or unsubstituted, and halo, cyano, and hydroxyl,
   or a pharmaceutically acceptable salt thereof.

In some embodiments, R is selected from H, substituted or unsubstituted C₁-C₄ alkyl, C₃-C₇ cycloalkyl, phenyl, 5-10 membered heteroaryl, 4-10 membered heterocyclyl, C₅-C₁₀ bicycloalkyl, C₅-C₁₀ spirocycloalkyl, 5-10 membered bicycloheterocyclyl, and 7-12 membered spiroheterocyclyl;
wherein each of said cycloalkyl, bicycloalkyl and spirocycloalkyl is independently unsubstituted or substituted with one, two, or three substituents selected from C₁-C₄ alkyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl;
wherein each of said heterocyclyl, bicycloheterocyclyl and spiroheterocyclyl is independently unsubstituted or substituted with one, two, or three substituents selected from C₁-C₄ alkyl, carboxy, aminocarbonyl, C₁-C₁ alkylaminocarbonyl, C₁-C₄ alkoxycarbonyl, and C₁-C₄ alkoxy, each of which is substituted or unsubstituted, and halo, cyano, and hydroxyl;
wherein said heteroaryl is unsubstituted or substituted with one, two, or three substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl; and
wherein said phenyl is unsubstituted or substituted with one, two, or three substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl.

In some embodiments, R is selected from H, methyl, ethyl, fluoroethyl, isopropyl, 1-cyano-1-methylethyl, CH—OCH₂CH₂OCH₂—, CH₃O(C═O)CH₂—, carboxymethyl, 3-fluorophenylmethyl, 4-fluorophenylmethyl, 3-pyridylmethyl, and 4-pyridylmethy.

In some embodiments, R is selected from 3-pyrazolyl, 4-pyrazolyl, 1H-1,2,3-triazol-4-yl, 3-pyridinyl, 4-pyridinyl, 2-pyridinyl, 3-thienyl, phenyl, 5-indolyl, 6-indolyl, 5-indazolyl, 5-benzimidazolyl, 1H-pyrrolo[2,3-b]pyridin-5-yl, 1,2,3,4-tetrahydroquinolin-7-yl, 5,6,7,8-tetrahydroquinolin-6-yl, 1,2,3,4-tetrahydroquinolin-6-yl, 6-isoquinolinyl, and quinolin-6-yl, each of which is independently unsubstituted or substituted with one, two, or three substituents selected from methyl, methoxy, ethoxy, isopropoxy, carboxy, (2-methoxyeth-1-yl), methoxycarbonyl, methylaminocarbonyl, aminocarbonyl, methylsulfonyl, hydroxy, chloro, fluoro, and cyano.

In some embodiments, R is bicyclo[1.1.1]pentan-1-yl that is unsubstituted or substituted with one, two, or three substituents selected from methyl, methoxy, carboxy, methoxycarbonyl, methylaminocarbonyl, aminocarbonyl hydroxy, fluoro, and cyano.

In some embodiments, R is selected from bicyclo[1.1.1]pentan-1-yl, 3-methyl-bicyclo[1.1.1]pentan-1-yl, 3-methoxy carbonyl-bicyclo[1.1.1]pentan-1-yl, 3-carboxyl-bicyclo[1.1.1]pentan-1-yl, 3-aminocarbonyl-bicyclo[1.1.1]pentan-1-yl, 3-methylaminocarbonyl-bicyclo[1.1.1]pentan-1-yl, 3-hydroxy-bicyclo[1.1.1]pentan-1-yl, 3-fluoro-bicyclo[1.1.1]pentan-1-yl, 3-cyano-bicyclo[1.1.1]pentan-1-yl, and bicyclo[2.2.2]octan-1-yl.

In some embodiments, R is spiro[3.3]heptan-2-yl or 2-oxaspiro[3.3]heptan-6-y.

In some embodiments, R is selected from 1,1-dioxo-1λ⁶-thian-4-yl, 2-oxabicyclo[2.1.1]hexan-4-yl and 8-oxabicyclo[3.2,]octan-3-yl.

In some embodiments, R is selected from cyclopropyl, 1-cyanocyclopropyl, 1-trifluoromethyl-cyclopropyl, 2-fluorocyclopropyl, 2,2-difluorocyclopropyl, cyclobutyl, 3-fluorocyclobutyl, 2-methylcyclobutyl 3-methylcyclobutyl, 3,3-difluorocyclobutyl, 3-methoxycarbonylcyclobutyl, 3-carboxy-cyclobutyl, 3,3-difluorocyclopentyl, 3-cyanocyclobutyl, 3-hydroxycyclopentyl, cyclopentyl, 3-fluorocyclopentyl, and 3-cyanocyclopentyl.

In some embodiments, R is piperidin-4-yl or 1-methylpiperidin-4-yl.

In some embodiments, R is selected from 1-methyl-3-pyrazolyl, 3-pyrazolyl, 4-pyrazolyl, 1-methyl-4-pyrazolyl, 1-(2-methoxyeth-1-yl)-4-pyrazolyl, 1-ethyl-4-pyrazolyl, 1-fluoroethyl-4-pyrazolyl, 1-trifluoroethyl-4-pyrazolyl, 1-dimethylaminoethyl-4-pyrazolyl, 1-tert-butyl-4-pyrazolyl, 1-(1-methyl-4-piperidinyl)˜4-pyrazolyl, 1-oxan-4-yl-4-pyrazolyl, 1-oxetan-3-yl-4-pyrazolyl, 1H-1,2,3-triazol-4-yl, 2-methylpyridin-4-yl, 2,6-dimethylpyridin-4-yl, 2-methylpyridin-5-yl, 2-methylpyridin-6-yl, 2-hydroxy-4-pyridinyl, 2-methoxy-4-pyridinyl, 2-chloro-4-pyridinyl, 2-aminocarbonyl-4-pyridinyl, 2-methoxy-5-pyridinyl, 2-aminocarbonyl-5-pyridinyl, 4-amino-2-pyridinyl, 3-thienyl, 4-cyanophenyl, 3-cyanophenyl, 3-methylsulfonylphenyl, 4-methylsulfonylphenyl, 3,4-difluorophenyl, 4-fluorophenyl, 3-chlorophenyl, 4-chlorophenyl, 3-methoxyphenyl, 3-ethoxyphenyl, 3-isopropoxyphenyl, 4-methoxyethoxyphenyl, 4-fluorophenyl, 4-fluoro-3-methoxyphenyl, 5-indolyl, 1-methyl-5-indolyl, 6-indolyl, 1-methyl-indol-6-yl, 4-chloro-6-indolyl, 7-fluoro-5-indolyl, 3-cyano-5-indolyl, 1-(2-methoxyeth-1-yl)-5-indolyl, 5-indazolyl, 5-benzimidazolyl, 2-methyl-5-benzimidazolyl, 1H-pyrrolo[2,3-b]pyridin-5-yl, 1-methyl-1,2,3,4˜tetrahydroquinolin-7-yl, 5,6,7,8-tetrahydroquinolin-6-yl, 1-methyl-1,2,3,4-tetrahydroquinolin-6-yl, 1(2-methoxyeth-1-yl)-1,2,3,4-tetrahydroquinolin-6-yl 6-isoquinolinyl, 3-cyano-quinolin-6-yl, 3-fluoroquinolin-6-yl, and 3-methoxyquinolin-6-y.

In some embodiments, R² is selected from methyl, ethyl, benzyl, trifluoromethyl, 4-fluorobenzyl, methoxymethyl, 1-methyl-4-piperidinylmethyl, 4-pyridinylmethyl, 1-pyrazolylmethyl, and 1-methyl-4-pyrazolylmethyl; and R^{2a} is H.

In some embodiments, R² is methyl; and R^{2a} is H.

In some embodiments, R² is methyl; and R^{2a} is methyl.

In some embodiments, R² is 1-1 and R² is H.

In some embodiments, R³ is pyrazolo[1,5-a]pyrimidin-7-yl, phenyl or pyridyl, each of which is unsubstituted or substituted with one, two, or three substituents selected from chloro, fluoro, bromo, cyano, C₁₋₃ alkyl, hydroxymethyl, C₁₋₃ haloalkyl, cyclopropyl, aminocarbonyl, di[C₁₋₃ alkyl]aminocarbonyl, di[C₁₋₃ alkyl]aminocarbonyl, methoxycarbonyl, carboxy, hydroxyl, C₁₋₃ alkoxy, C₁₋₃ alkoxyalkyl, pyridyl, and phenyl that is unsubstituted or substituted with one or two substituents selected from chloro, fluoro, bromo, cyano, methyl, ethyl, hydroxymethyl, trifluoromethyl, methoxymethyl, methoxypropyl, cyanopropyl, cyclopropyl, carboxy, methoxycarbonyl, aminocarbonyl, methylaminocarbonyl, dimethylaminocarbonyl, hydroxyl, and methoxy.

In some embodiments, R³ is 3-pyridyl that is unsubstituted or substituted with one or two substituents selected from chloro, fluoro, bromo, cyano, methyl, ethyl, hydroxymethyl, 2-hydroxy-2-methylethyl, trifluoromethyl, methoxymethyl, methoxypropyl, 2-cyano-2-methylethyl, cyclopropyl, carboxy, methoxycarbonyl, aminocarbonyl, methylaminocarbonyl, dimethylaminocarbonyl, hydroxyl, methoxy, pyridyl and phenyl that is unsubstituted or substituted with one or two substituents selected from chloro, fluoro, and methoxy.

In some embodiments, R¹ is selected from tert-butyl, 1-methylpropyl, 2-methylbutyl, 2,2-dimethylbutyl, cyclohexyl, bicyclo[2.2.2]octan-1-yl-CH₂—, bicyclo[3.1.0]hexan-2-yl-CH₂—, and bicyclo[1.1.1]pentan-1-yl-CH₂—.

In some embodiments, R¹ is cyclopentyl, cyclopentyl-CH₂—, or cyclohexyl-CH₂—.

In some embodiments, ring B is dihydroindol-1-yl, 4-methyl-7-chloro-tetrahydroquinoxalin-1-yl, 5-chloro-1,2,3,4-tetrahydroquinolin-2-yl or 7-chloro-1,2,3,4-tetrahydroquinolin-1-yl, wherein the dihydroindol-1-yl or tetrahydroquinoline-1-yl each are independently unsubstituted or substituted with one or two substituents selected from halo and methoxy.

In some embodiments, ring B is a 9 or 10 membered heterocyclyl that is a fused ring system of an aromatic ring fused to a non-aromatic ring, wherein said heterocyclyl is unsubstituted or substituted with one or two substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, hydroxyl, and cyano.

In some embodiments, ring B is a 5, 9, or 10 membered heteroaryl, wherein said heteroaryl is unsubstituted or substituted with one or two substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, and C₁-C₄alkoxy, each of which is substituted or unsubstituted, and halo, hydroxyl, and cyano.

In some embodiments, the compound is selected from Tables A-L, or a pharmaceutically acceptable salt thereof.

In some embodiments, the present disclosure provides a pharmaceutical composition comprising a compound as described herein or a pharmaceutically acceptable salt thereof, and a pharmaceutically acceptable carrier.

In some embodiments, the present disclosure provides a method of treating cancer, the method comprising administering to a human in need thereof an effective amount of a compound described herein.

In some embodiments, the cancer is selected from the group consisting of brain, glioblastomas, leukemias, lymphomas, Bannayan-Zonana syndrome, Cowden disease, Lhermitte-Duclos disease, breast, inflammatory breast cancer, Wilm's tumor. Ewing's sarcoma, Rhabdomyosarcoma, ependymoma, medulloblastoma, neuroblastoma, small cell lung, endometrium, cervix, esophagus, colon, gastric, bladder, head and neck, kidney, lung, liver, melanoma, ovarian, pancreatic, prostate, mesothelioma, sarcoma, osteosarcoma, giant cell tumor of bone, and thyroid.

Non-limiting examples of compounds of the current disclosure include the following in Table A:

TABLE A

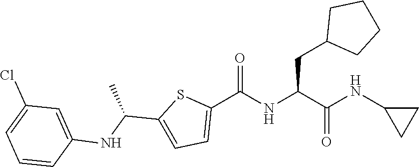 [SMILES: C[C@@H](Nc1cccc(Cl)c1)c1ccc(C(=O)N[C@@H](CC2CCCC2)C(=O)NC2CC2)s1]

(2S)-2-({5-[(1R)-1-[(3-
chlorophenyl)amino]ethyl]thiophen-2-
yl}formamido)-3-cyclopentyl-N-
cyclopropylpropanamide

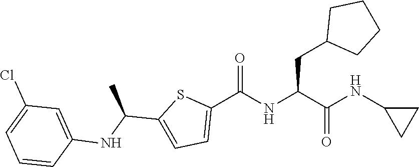 [SMILES: C[C@H](Nc1cccc(Cl)c1)c1ccc(C(=O)N[C@@H](CC2CCCC2)C(=O)NC2CC2)s1]

(2S)-2-({5-[(1S)-1-[(3-
chlorophenyl)amino]ethyl]thiophen-2-
yl}formamido)-3-cyclopentyl-N-
cyclopropylpropanamide

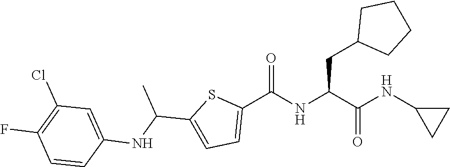 [SMILES: CC(Nc1ccc(F)c(Cl)c1)c1ccc(C(=O)N[C@@H](CC2CCCC2)C(=O)NC2CC2)s1]

(2S)-2-[(5-{1-[(3-chloro-4-
fluorophenyl)amino]ethyl}thiophen-2-
yl)formamido]-3-cyclopentyl-N-
cyclopropylpropanamide

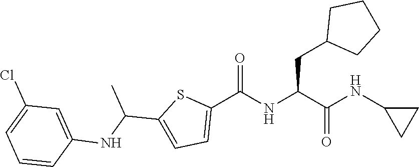 [SMILES: CC(Nc1cccc(Cl)c1)c1ccc(C(=O)N[C@@H](CC2CCCC2)C(=O)NC2CC2)s1]

(2S)-2-[(5-{1-[(3-
chlorophenyl)amino]ethyl}thiophen-2-
yl)formamido]-3-cyclopentyl-N-
cyclopropylpropanamide

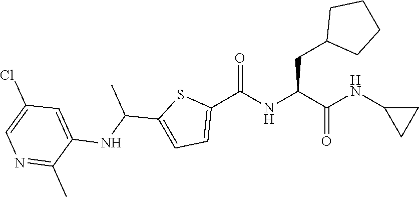 [SMILES: Cc1ncc(Cl)cc1NC(C)c1ccc(C(=O)N[C@@H](CC2CCCC2)C(=O)NC2CC2)s1]

(2S)-2-[(5-{1-[(5-chloro-2-methylpyridin-3-
yl)amino]ethyl}thiophen-2-yl)formamido]-3-
cyclopentyl-N-cyclopropylpropanamide

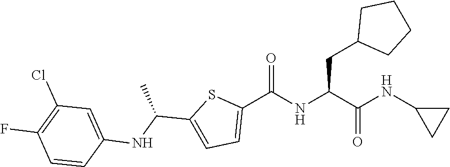 [SMILES: C[C@@H](Nc1ccc(F)c(Cl)c1)c1ccc(C(=O)N[C@@H](CC2CCCC2)C(=O)NC2CC2)s1]

(2S)-2-({5-[(1R)-1-[(3-chloro-4-
fluorophenyl)amino]ethyl]thiophen-2-
yl}formamido)-3-cyclopentyl-N-
cyclopropylpropanamide

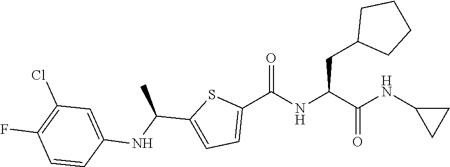 [SMILES: C[C@H](Nc1ccc(F)c(Cl)c1)c1ccc(C(=O)N[C@@H](CC2CCCC2)C(=O)NC2CC2)s1]

(2S)-2-({5-[(1S)-1-[(3-chloro-4-
fluorophenyl)amino]ethyl]thiophen-2-
yl}formamido)-3-cyclopentyl-N-
cyclopropylpropanamide

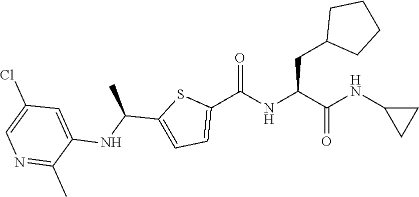 [SMILES: Cc1ncc(Cl)cc1N[C@@H](C)c1ccc(C(=O)N[C@@H](CC2CCCC2)C(=O)NC2CC2)s1]

(2S)-2-({5-[(1S)-1-[(5-chloro-2-methylpyridin-
3-yl)amino]ethyl]thiophen-2-yl}formamido)-3-
cyclopentyl-N-cyclopropylpropanamide

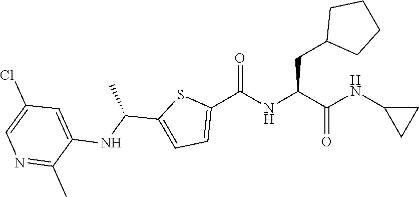 [SMILES: Cc1ncc(Cl)cc1N[C@H](C)c1ccc(C(=O)N[C@@H](CC2CCCC2)C(=O)NC2CC2)s1]

(2S)-2-({5-[(1R)-1-[(5-chloro-2-methylpyridin-
3-yl)amino]ethyl]thiophen-2-yl}formamido)-3-
cyclopentyl-N-cyclopropylpropanamide

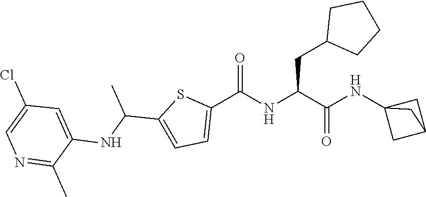 [SMILES: Cc1ncc(Cl)cc1NC(C)c1ccc(C(=O)N[C@@H](CC2CCCC2)C(=O)NC23CC(C2)C3)s1]

(2S)-N-{bicyclo[1.1.1]pentan-1-yl}-2-[(5-{1-
[(5-chloro-2-methylpyridin-3-
yl)amino]ethyl}thiophen-2-yl)formamido]-3-
cyclopentylpropanamide

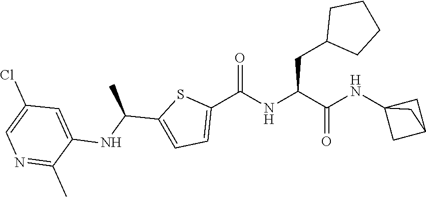 [SMILES: Cc1ncc(Cl)cc1N[C@@H](C)c1ccc(C(=O)N[C@@H](CC2CCCC2)C(=O)NC23CC(C2)C3)s1]

(2S)-N-{bicyclo[1.1.1]pentan-1-yl}-2-({5-
[(1S)-1-[(5-chloro-2-methylpyridin-3-
yl)amino]ethyl]thiophen-2-yl}formamido)-3-
cyclopentylpropanamide

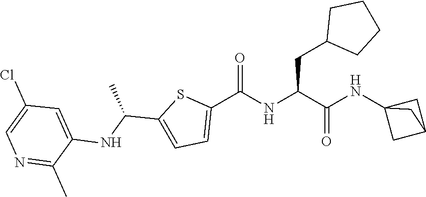 [SMILES: Cc1ncc(Cl)cc1N[C@H](C)c1ccc(C(=O)N[C@@H](CC2CCCC2)C(=O)NC23CC(C2)C3)s1]

(2S)-N-{bicyclo[1.1.1]pentan-1-yl}-2-({5-
[(1R)-1-[(5-chloro-2-methylpyridin-3-
yl)amino]ethyl]thiophen-2-yl}formamido)-3-
cyclopentylpropanamide

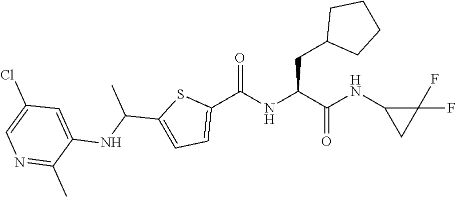 [SMILES: Cc1ncc(Cl)cc1NC(C)c1ccc(C(=O)N[C@@H](CC2CCCC2)C(=O)NC2CC2(F)F)s1]

(2S)-2-[(5-{1-[(5-chloro-2-methylpyridin-3-
yl)amino]ethyl}thiophen-2-yl)formamido]-3-
cyclopentyl-N-(2,2-
difluorocyclopropyl)propanamide

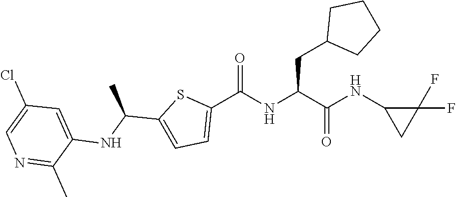 [SMILES: Cc1ncc(Cl)cc1N[C@@H](C)c1ccc(C(=O)N[C@@H](CC2CCCC2)C(=O)NC2CC2(F)F)s1]

(2S)-2-({5-[(1S)-1-[(5-chloro-2-methylpyridin-
3-yl)amino]ethyl]thiophen-2-yl}formamido)-3-
cyclopentyl-N-(2,2-
difluorocyclopropyl)propanamide

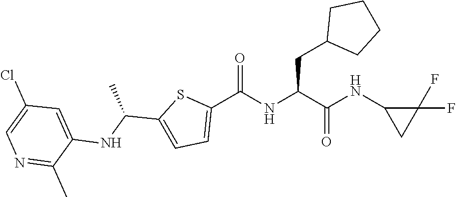 [SMILES: Cc1ncc(Cl)cc1N[C@H](C)c1ccc(C(=O)N[C@@H](CC2CCCC2)C(=O)NC2CC2(F)F)s1]

(2S)-2-({5-[(1R)-1-[(5-chloro-2-methylpyridin-
3-yl)amino]ethyl]thiophen-2-yl}formamido)-3-
cyclopentyl-N-(2,2-
difluorocyclopropyl)propanamide

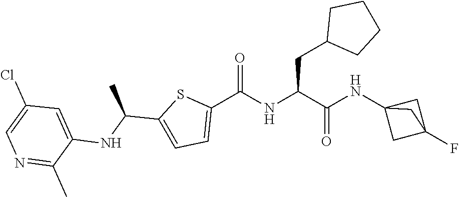 [SMILES: Cc1ncc(Cl)cc1N[C@@H](C)c1ccc(C(=O)N[C@@H](CC2CCCC2)C(=O)NC23CC(F)(C2)C3)s1]

(2S)-2-({5-[(1S)-1-[(5-chloro-2-methylpyridin-
3-yl)amino]ethyl]thiophen-2-yl}formamido)-3-
cyclopentyl-N-{3-fluorobicyclo[1.1.1]pentan-
1-yl}propanamide

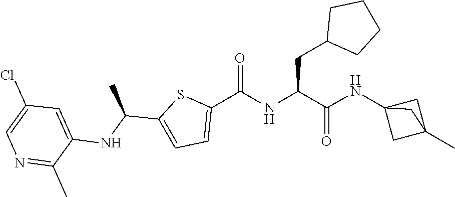 [SMILES: Cc1ncc(Cl)cc1N[C@@H](C)c1ccc(C(=O)N[C@@H](CC2CCCC2)C(=O)NC23CC(C)(C2)C3)s1]

(2S)-2-({5-[(1S)-1-[(5-chloro-2-methylpyridin-
3-yl)amino]ethyl]thiophen-2-yl}formamido)-3-
cyclopentyl-N-{3-methylbicyclo[1.1.1]pentan-
1-yl}propanamide

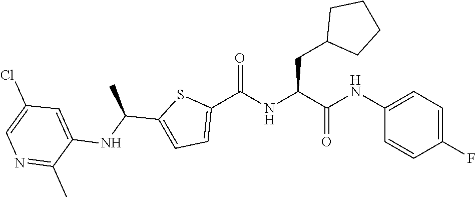 [SMILES: Cc1ncc(Cl)cc1N[C@@H](C)c1ccc(C(=O)N[C@@H](CC2CCCC2)C(=O)Nc2ccc(F)cc2)s1]

(2S)-2-({5-[(1S)-1-[(5-chloro-2-methylpyridin-
3-yl)amino]ethyl]thiophen-2-yl}formamido)-3-
cyclopentyl-N-(4-fluorophenyl)propanamide

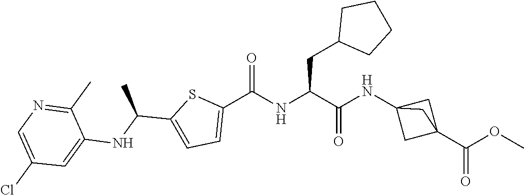 [SMILES: COC(=O)C12CC(NC(=O)[C@H](CC3CCCC3)NC(=O)c3ccc([C@H](C)Nc4cc(Cl)cnc4C)s3)(C1)C2]

methyl 3-[(2S)-2-({5-[(1S)-1-[(5-chloro-2-
methylpyridin-3-yl)amino]ethyl]thiophen-2-
yl}formamido)-3-
cyclopentylpropanamido]bicyclo[1.1.1]pentane-
1-carboxylate

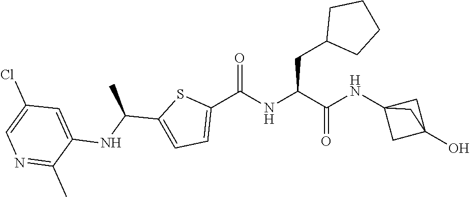 [SMILES: Cc1ncc(Cl)cc1N[C@@H](C)c1ccc(C(=O)N[C@@H](CC2CCCC2)C(=O)NC23CC(O)(C2)C3)s1]

(2S)-2-({5-[(1S)-1-[(5-chloro-2-methylpyridin-
3-yl)amino]ethyl]thiophen-2-yl}formamido)-3-
cyclopentyl-N-{3-
hydroxybicyclo[1.1.1]pentan-1-
yl}propanamide

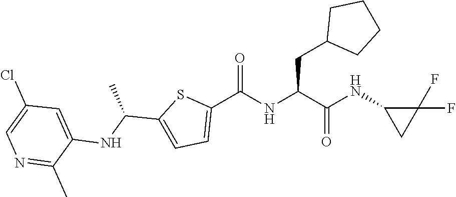 [SMILES: Cc1ncc(Cl)cc1N[C@H](C)c1ccc(C(=O)N[C@@H](CC2CCCC2)C(=O)N[C@H]2CC2(F)F)s1]

(2S)-2-({5-[(1R)-1-[(5-chloro-2-methylpyridin-
3-yl)amino]ethyl]thiophen-2-yl}formamido)-3-
cyclopentyl-N-[(1S)-2,2-
difluorocyclopropyl]propanamide

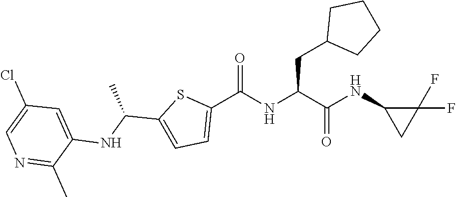 [SMILES: Cc1ncc(Cl)cc1N[C@H](C)c1ccc(C(=O)N[C@@H](CC2CCCC2)C(=O)N[C@@H]2CC2(F)F)s1]

(2S)-2-({5-[(1R)-1-[(5-chloro-2-methylpyridin-
3-yl)amino]ethyl]thiophen-2-yl}formamido)-3-
cyclopentyl-N-[(1R)-2,2-
difluorocyclopropyl]propanamide

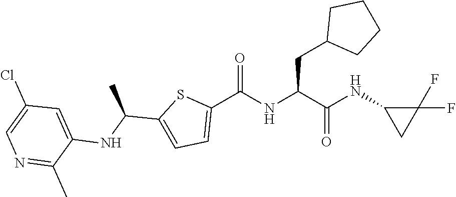 [SMILES: Cc1ncc(Cl)cc1N[C@@H](C)c1ccc(C(=O)N[C@@H](CC2CCCC2)C(=O)N[C@H]2CC2(F)F)s1]

(2S)-2-({5-[(1S)-1-[(5-chloro-2-methylpyridin-
3-yl)amino]ethyl]thiophen-2-yl}formamido)-3-
cyclopentyl-N-[(1S)-2,2-
difluorocyclopropyl]propanamide

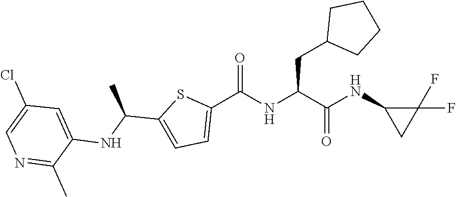 [SMILES: Cc1ncc(Cl)cc1N[C@@H](C)c1ccc(C(=O)N[C@@H](CC2CCCC2)C(=O)N[C@@H]2CC2(F)F)s1]

(2S)-2-({5-[(1S)-1-[(5-chloro-2-methylpyridin-
3-yl)amino]ethyl]thiophen-2-yl}formamido)-3-
cyclopentyl-N-[(1R)-2,2-
difluorocyclopropyl]propanamide

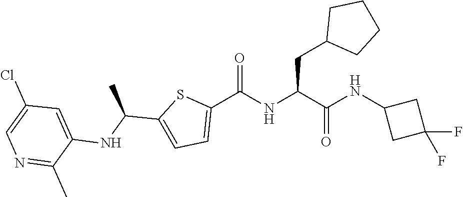 [SMILES: Cc1ncc(Cl)cc1N[C@@H](C)c1ccc(C(=O)N[C@@H](CC2CCCC2)C(=O)NC2CC(F)(F)C2)s1]

(2S)-2-({5-[(1S)-1-[(5-chloro-2-methylpyridin-
3-yl)amino]ethyl]thiophen-2-yl}formamido)-3-
cyclopentyl-N-(3,3-
difluorocyclobutyl)propanamide

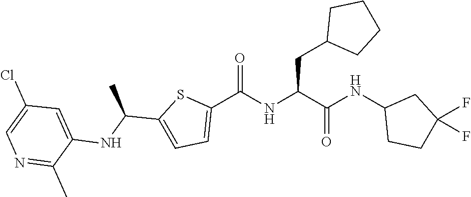 [SMILES: Cc1ncc(Cl)cc1N[C@@H](C)c1ccc(C(=O)N[C@@H](CC2CCCC2)C(=O)NC2CCC(F)(F)C2)s1]

(2S)-2-({5-[(1S)-1-[(5-chloro-2-methylpyridin-
3-yl)amino]ethyl]thiophen-2-yl}formamido)-3-
cyclopentyl-N-(3,3-
difluorocyclopentyl)propanamide

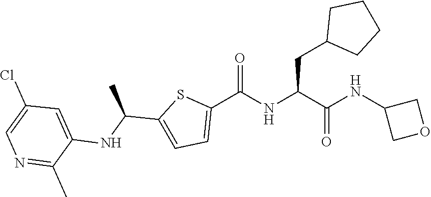 [SMILES: Cc1ncc(Cl)cc1N[C@@H](C)c1ccc(C(=O)N[C@@H](CC2CCCC2)C(=O)NC2COC2)s1]

(2S)-2-({5-[(1S)-1-[(5-chloro-2-methylpyridin-
3-yl)amino]ethyl]thiophen-2-yl}formamido)-3-
cyclopentyl-N-(oxetan-3-yl)propanamide

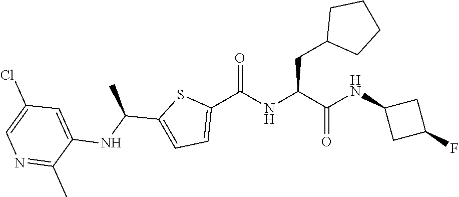 [SMILES: Cc1ncc(Cl)cc1N[C@@H](C)c1ccc(C(=O)N[C@@H](CC2CCCC2)C(=O)N[C@H]2C[C@@H](F)C2)s1]

(2S)-2-({5-[(1S)-1-[(5-chloro-2-methylpyridin-
3-yl)amino]ethyl]thiophen-2-yl}formamido)-3-
cyclopentyl-N-[(1s,3s)-3-
fluorocyclobutyl]propanamide

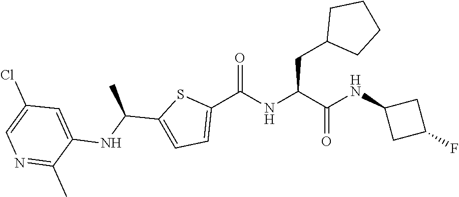 [SMILES: Cc1ncc(Cl)cc1N[C@@H](C)c1ccc(C(=O)N[C@@H](CC2CCCC2)C(=O)N[C@H]2C[C@H](F)C2)s1]

(2S)-2-({5-[(1S)-1-[(5-chloro-2-methylpyridin-
3-yl)amino]ethyl]thiophen-2-yl}formamido)-3-
cyclopentyl-N-[(1r,3r)-3-
fluorocyclobutyl]propanamide

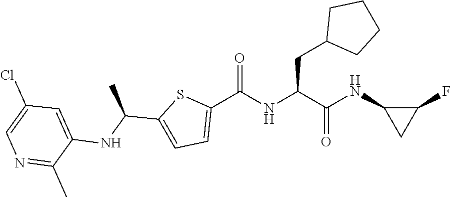 [SMILES: Cc1ncc(Cl)cc1N[C@@H](C)c1ccc(C(=O)N[C@@H](CC2CCCC2)C(=O)N[C@@H]2C[C@@H]2F)s1]

(2S)-2-({5-[(1S)-1-[(5-chloro-2-methylpyridin-
3-yl)amino]ethyl]thiophen-2-yl}formamido)-3-
cyclopentyl-N-[(1R,2S)-2-
fluorocyclopropyl]propanamide

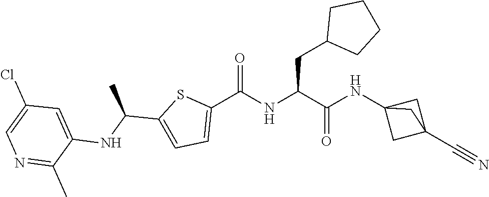 [SMILES: Cc1ncc(Cl)cc1N[C@@H](C)c1ccc(C(=O)N[C@@H](CC2CCCC2)C(=O)NC23CC(C#N)(C2)C3)s1]

(2S)-2-({5-[(1S)-1-[(5-chloro-2-methylpyridin-
3-yl)amino]ethyl]thiophen-2-yl}formamido)-
N-{3-cyanobicyclo[1.1.1]pentan-1-yl}-3-
cyclopentylpropanamide

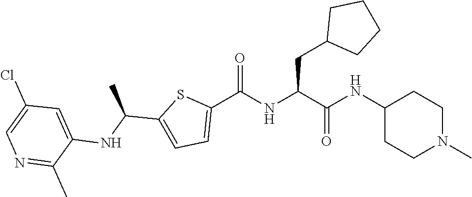 [SMILES: Cc1ncc(Cl)cc1N[C@@H](C)c1ccc(C(=O)N[C@@H](CC2CCCC2)C(=O)NC2CCN(C)CC2)s1]

(2S)-2-({5-[(1S)-1-[(5-chloro-2-methylpyridin-
3-yl)amino]ethyl]thiophen-2-yl}formamido)-3-
cyclopentyl-N-(1-methylpiperidin-4-
yl)propanamide

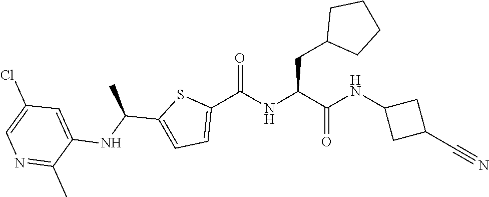 [SMILES: Cc1ncc(Cl)cc1N[C@@H](C)c1ccc(C(=O)N[C@@H](CC2CCCC2)C(=O)NC2CC(C#N)C2)s1]

(2S)-2-({5-[(1S)-1-[(5-chloro-2-methylpyridin-
3-yl)amino]ethyl]thiophen-2-yl}formamido)-
N-(3-cyanocyclobutyl)-3-
cyclopentylpropanamide

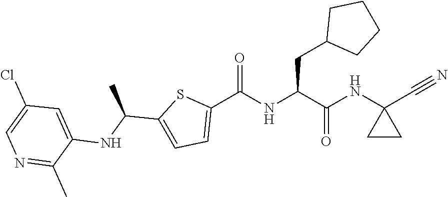 [SMILES: Cc1ncc(Cl)cc1N[C@@H](C)c1ccc(C(=O)N[C@@H](CC2CCCC2)C(=O)NC2(C#N)CC2)s1]

(2S)-2-({5-[(1S)-1-[(5-chloro-2-methylpyridin-
3-yl)amino]ethyl]thiophen-2-yl}formamido)-
N-(1-cyanocyclopropyl)-3-
cyclopentylpropanamide

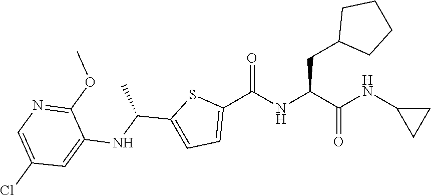 [SMILES: COc1ncc(Cl)cc1N[C@H](C)c1ccc(C(=O)N[C@@H](CC2CCCC2)C(=O)NC2CC2)s1]

(2S)-2-({5-[(1R)-1-[(5-chloro-2-
methoxypyridin-3-yl)amino]ethyl]thiophen-2-
yl}formamido)-3-cyclopentyl-N-
cyclopropylpropanamide

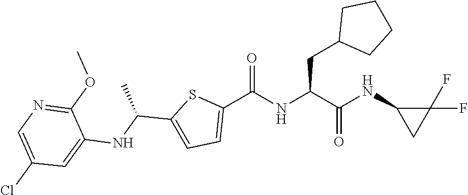 [SMILES: COc1ncc(Cl)cc1N[C@H](C)c1ccc(C(=O)N[C@@H](CC2CCCC2)C(=O)N[C@@H]2CC2(F)F)s1]

(2S)-2-({5-[(1R)-1-[(5-chloro-2-
methoxypyridin-3-yl)amino]ethyl]thiophen-2-
yl}formamido)-3-cyclopentyl-N-[(1R)-2,2-
difluorocyclopropyl]propanamide

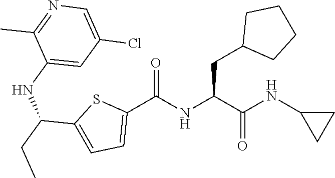 [SMILES: CC[C@H](Nc1cc(Cl)cnc1C)c1ccc(C(=O)N[C@@H](CC2CCCC2)C(=O)NC2CC2)s1]

(2S)-2-({5-[(1S)-1-[(5-chloro-2-methylpyridin-
3-yl)amino]propyl]thiophen-2-yl}formamido)-
3-cyclopentyl-N-cyclopropylpropanamide

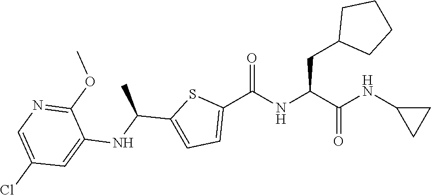 [SMILES: COc1ncc(Cl)cc1N[C@@H](C)c1ccc(C(=O)N[C@@H](CC2CCCC2)C(=O)NC2CC2)s1]

(2S)-2-({5-[(1S)-1-[(5-chloro-2-
methoxypyridin-3-yl)amino]ethyl]thiophen-2-
yl}formamido)-3-cyclopentyl-N-
cyclopropylpropanamide

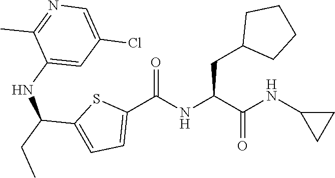 [SMILES: CC[C@@H](Nc1cc(Cl)cnc1C)c1ccc(C(=O)N[C@@H](CC2CCCC2)C(=O)NC2CC2)s1]

(2S)-2-({5-[(1R)-1-[(5-chloro-2-methylpyridin-
3-yl)amino]propyl]thiophen-2-yl}formamido)-
3-cyclopentyl-N-cyclopropylpropanamide

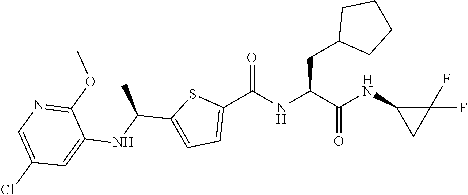 [SMILES: COc1ncc(Cl)cc1N[C@@H](C)c1ccc(C(=O)N[C@@H](CC2CCCC2)C(=O)N[C@@H]2CC2(F)F)s1]

(2S)-2-({5-[(1S)-1-[(5-chloro-2-
methoxypyridin-3-yl)amino]ethyl]thiophen-2-
yl}formamido)-3-cyclopentyl-N-[(1R)-2,2-
difluorocyclopropyl]propanamide

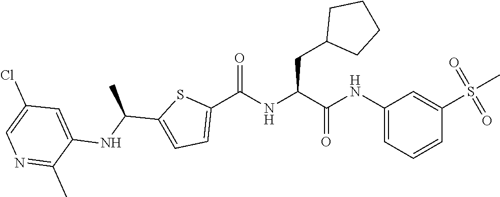 [SMILES: Cc1ncc(Cl)cc1N[C@@H](C)c1ccc(C(=O)N[C@@H](CC2CCCC2)C(=O)Nc2cccc(S(C)(=O)=O)c2)s1]

(2S)-2-({5-[(1S)-1-[(5-chloro-2-methylpyridin-
3-yl)amino]ethyl]thiophen-2-yl}formamido)-3-
cyclopentyl-N-(3-
methanesulfonylphenyl)propanamide

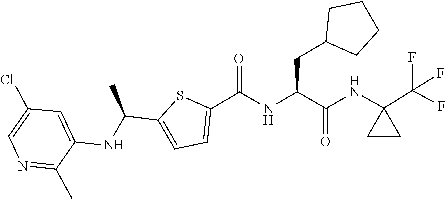 [SMILES: Cc1ncc(Cl)cc1N[C@@H](C)c1ccc(C(=O)N[C@@H](CC2CCCC2)C(=O)NC2(C(F)(F)F)CC2)s1]

(2S)-2-({5-[(1S)-1-[(5-chloro-2-methylpyridin-
3-yl)amino]ethyl]thiophen-2-yl}formamido)-3-
cyclopentyl-N-[1-
(trifluoromethyl)cyclopropyl]propanamide

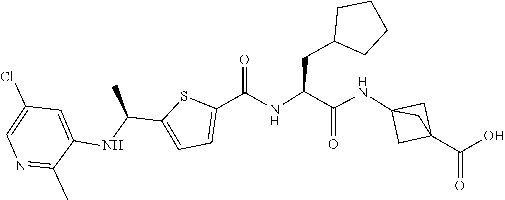 [SMILES: Cc1ncc(Cl)cc1N[C@@H](C)c1ccc(C(=O)N[C@@H](CC2CCCC2)C(=O)NC23CC(C(=O)O)(C2)C3)s1]

3-[(2S)-2-({5-[(1S)-1-[(5-chloro-2-
methylpyridin-3-yl)amino]ethyl]thiophen-2-
yl}formamido)-3-
cyclopentylpropanamido]bicyclo[1.1.1]pentane-
1-carboxylic acid

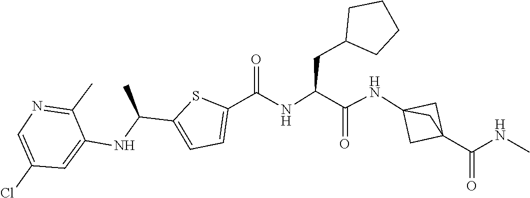 [SMILES: CNC(=O)C12CC(NC(=O)[C@H](CC3CCCC3)NC(=O)c3ccc([C@H](C)Nc4cc(Cl)cnc4C)s3)(C1)C2]

3-[(2S)-2-({5-[(1S)-1-[(5-chloro-2-
methylpyridin-3-yl)amino]ethyl]thiophen-2-
yl}formamido)-3-cyclopentylpropanamido]-N-
methylbicyclo[1.1.1]pentane-1-carboxamide

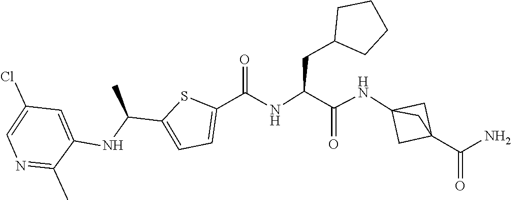 [SMILES: Cc1ncc(Cl)cc1N[C@@H](C)c1ccc(C(=O)N[C@@H](CC2CCCC2)C(=O)NC23CC(C(N)=O)(C2)C3)s1]

3-[(2S)-2-({5-[(1S)-1-[(5-chloro-2-
methylpyridin-3-yl)amino]ethyl]thiophen-2-
yl}formamido)-3-
cyclopentylpropanamido]bicyclo[1.1.1]pentane-
1-carboxamide

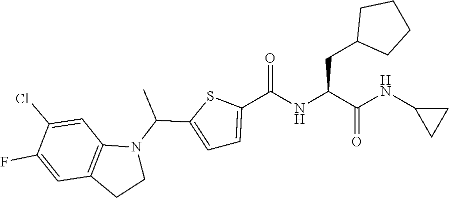 [SMILES: CC(c1ccc(C(=O)N[C@@H](CC2CCCC2)C(=O)NC2CC2)s1)N1CCc2cc(F)c(Cl)cc21]

(2S)-2-({5-[1-(6-chloro-5-fluoro-2,3-dihydro-
1H-indol-1-yl)ethyl]thiophen-2-
yl}formamido)-3-cyclopentyl-N-
cyclopropylpropanamide

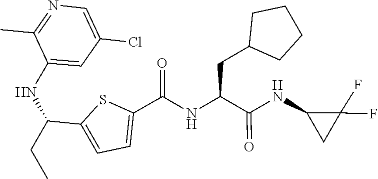 [SMILES: CC[C@H](Nc1cc(Cl)cnc1C)c1ccc(C(=O)N[C@@H](CC2CCCC2)C(=O)N[C@@H]2CC2(F)F)s1]

(2S)-2-({5-[(1S)-1-[(5-chloro-2-methylpyridin-
3-yl)amino]propyl]thiophen-2-yl}formamido)-
3-cyclopentyl-N-[(1R)-2,2-
difluorocyclopropyl]propanamide

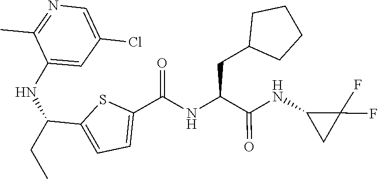 [SMILES: CC[C@H](Nc1cc(Cl)cnc1C)c1ccc(C(=O)N[C@@H](CC2CCCC2)C(=O)N[C@H]2CC2(F)F)s1]

(2S)-2-({5-[(1S)-1-[(5-chloro-2-methylpyridin-
3-yl)amino]propyl]thiophen-2-yl}formamido)-
3-cyclopentyl-N-[(1S)-2,2-
difluorocyclopropyl]propanamide

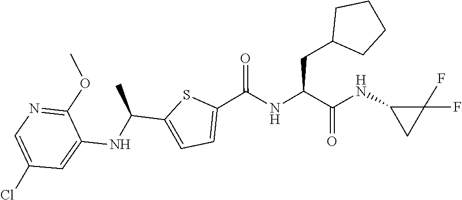 [SMILES: COc1ncc(Cl)cc1N[C@@H](C)c1ccc(C(=O)N[C@@H](CC2CCCC2)C(=O)N[C@H]2CC2(F)F)s1]

(2S)-2-({5-[(1S)-1-[(5-chloro-2-
methoxypyridin-3-yl)amino]ethyl]thiophen-2-
yl}formamido)-3-cyclopentyl-N-[(1S)-2,2-
difluorocyclopropyl]propanamide

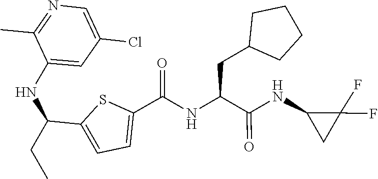 [SMILES: CC[C@@H](Nc1cc(Cl)cnc1C)c1ccc(C(=O)N[C@@H](CC2CCCC2)C(=O)N[C@@H]2CC2(F)F)s1]

(2S)-2-({5-[(1R)-1-[(5-chloro-2-methylpyridin-
3-yl)amino]propyl]thiophen-2-yl}formamido)-
3-cyclopentyl-N-[(1R)-2,2-
difluorocyclopropyl]propanamide

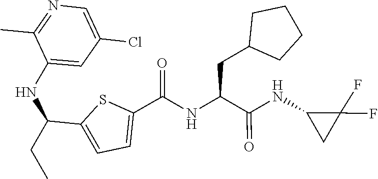 [SMILES: CC[C@@H](Nc1cc(Cl)cnc1C)c1ccc(C(=O)N[C@@H](CC2CCCC2)C(=O)N[C@H]2CC2(F)F)s1]

(2S)-2-({5-[(1R)-1-[(5-chloro-2-methylpyridin-
3-yl)amino]propyl]thiophen-2-yl}formamido)-
3-cyclopentyl-N-[(1S)-2,2-
difluorocyclopropyl]propanamide

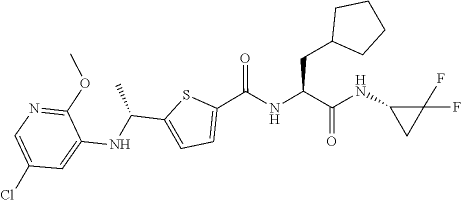 [SMILES: COc1ncc(Cl)cc1N[C@H](C)c1ccc(C(=O)N[C@@H](CC2CCCC2)C(=O)N[C@H]2CC2(F)F)s1]

(2S)-2-({5-[(1R)-1-[(5-chloro-2-
methoxypyridin-3-yl)amino]ethyl]thiophen-2-
yl}formamido)-3-cyclopentyl-N-[(1S)-2,2-
difluorocyclopropyl]propanamide

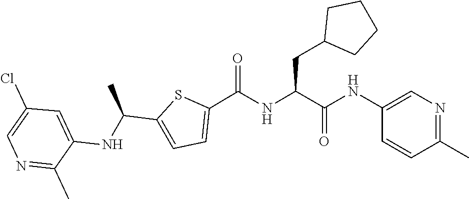 [SMILES: Cc1ccc(NC(=O)[C@H](CC2CCCC2)NC(=O)c2ccc([C@H](C)Nc3cc(Cl)cnc3C)s2)cn1]

(2S)-2-({5-[(1S)-1-[(5-chloro-2-methylpyridin-
3-yl)amino]ethyl]thiophen-2-yl}formamido)-3-
cyclopentyl-N-(6-methylpyridin-3-
yl)propanamide

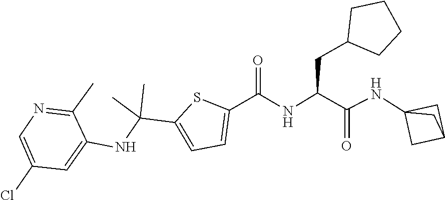 [SMILES: Cc1ncc(Cl)cc1NC(C)(C)c1ccc(C(=O)N[C@@H](CC2CCCC2)C(=O)NC23CC(C2)C3)s1]

(2S)-N-{bicyclo[1.1.1]pentan-1-yl}-2-[(5-{2-
[(5-chloro-2-methylpyridin-3-yl)amino]propan-
2-yl}thiophen-2-yl)formamido]-3-
cyclopentylpropanamide

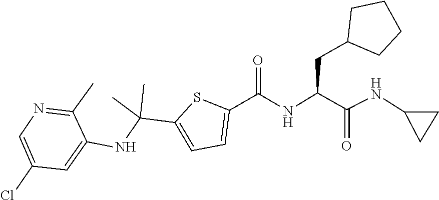 [SMILES: Cc1ncc(Cl)cc1NC(C)(C)c1ccc(C(=O)N[C@@H](CC2CCCC2)C(=O)NC2CC2)s1]

(2S)-2-[(5-{2-[(5-chloro-2-methylpyridin-3-
yl)amino]propan-2-yl}thiophen-2-
yl)formamido]-3-cyclopentyl-N-
cyclopropylpropanamide

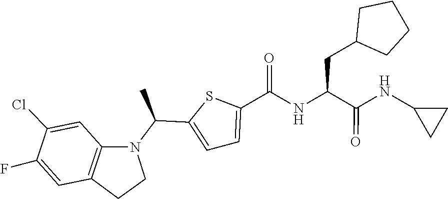 [SMILES: C[C@@H](c1ccc(C(=O)N[C@@H](CC2CCCC2)C(=O)NC2CC2)s1)N1CCc2cc(F)c(Cl)cc21]

(2S)-2-({5-[(1S)-1-(6-chloro-5-fluoro-2,3-
dihydro-1H-indol-1-yl)ethyl]thiophen-2-
yl}formamido)-3-cyclopentyl-N-
cyclopropylpropanamide

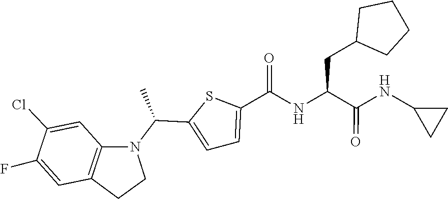 [SMILES: C[C@H](c1ccc(C(=O)N[C@@H](CC2CCCC2)C(=O)NC2CC2)s1)N1CCc2cc(F)c(Cl)cc21]

(2S)-2-({5-[(1R)-1-(6-chloro-5-fluoro-2,3-
dihydro-1H-indol-1-yl)ethyl]thiophen-2-
yl}formamido)-3-cyclopentyl-N-
cyclopropylpropanamide

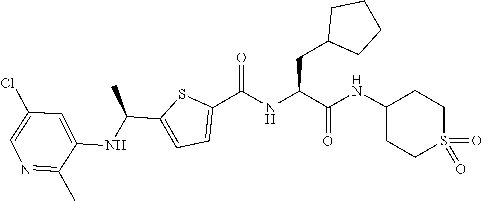 [SMILES: Cc1ncc(Cl)cc1N[C@@H](C)c1ccc(C(=O)N[C@@H](CC2CCCC2)C(=O)NC2CCS(=O)(=O)CC2)s1]

(2S)-2-({5-[(1S)-1-[(5-chloro-2-methylpyridin-
3-yl)amino]ethyl]thiophen-2-yl}formamido)-3-
cyclopentyl-N-(1,1-dioxo-1λ⁶-thian-4-
yl)propanamide

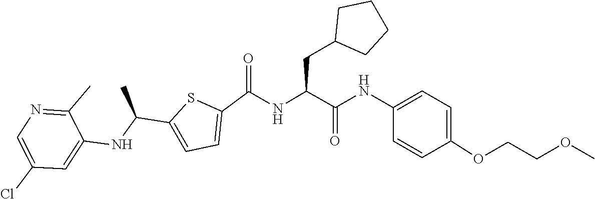 [SMILES: COCCOc1ccc(NC(=O)[C@H](CC2CCCC2)NC(=O)c2ccc([C@H](C)Nc3cc(Cl)cnc3C)s2)cc1]

(2S)-2-({5-[(1S)-1-[(5-chloro-2-methylpyridin-
3-yl)amino]ethyl]thiophen-2-yl}formamido)-3-
cyclopentyl-N-[4-(2-
methoxyethoxy)phenyl]propanamide

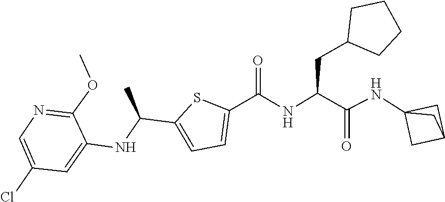 [SMILES: COc1ncc(Cl)cc1N[C@@H](C)c1ccc(C(=O)N[C@@H](CC2CCCC2)C(=O)NC23CC(C2)C3)s1]

(2S)-N-{bicyclo[1.1.1]pentan-1-yl}-2-({5-
[(1S)-1-[(5-chloro-2-methoxypyridin-3-
yl)amino]ethyl]thiophen-2-yl}formamido)-3-
cyclopentylpropanamide

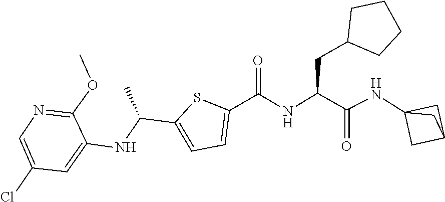 [SMILES: COc1ncc(Cl)cc1N[C@H](C)c1ccc(C(=O)N[C@@H](CC2CCCC2)C(=O)NC23CC(C2)C3)s1]

(2S)-N-{bicyclo[1.1.1]pentan-1-yl}-2-({5-
[(1R)-1-[(5-chloro-2-methoxypyridin-3-
yl)amino]ethyl]thiophen-2-yl}formamido)-3-
cyclopentylpropanamide

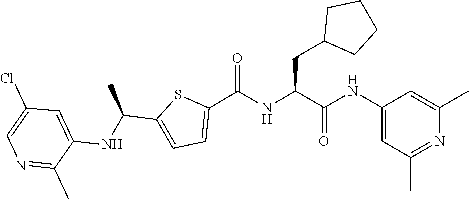 [SMILES: Cc1cc(NC(=O)[C@H](CC2CCCC2)NC(=O)c2ccc([C@H](C)Nc3cc(Cl)cnc3C)s2)cc(C)n1]

(2S)-2-({5-[(1S)-1-[(5-chloro-2-methylpyridin-
3-yl)amino]ethyl]thiophen-2-yl}formamido)-3-
cyclopentyl-N-(2,6-dimethylpyridin-4-
yl)propanamide

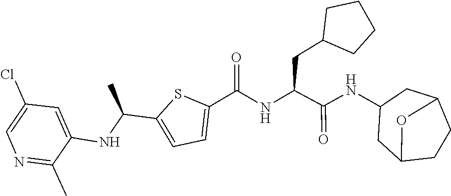 [SMILES: Cc1ncc(Cl)cc1N[C@@H](C)c1ccc(C(=O)N[C@@H](CC2CCCC2)C(=O)NC2CC3CCC(C2)O3)s1]

(2S)-2-({5-[(1S)-1-[(5-chloro-2-methylpyridin-
3-yl)amino]ethyl]thiophen-2-yl}formamido)-3-
cyclopentyl-N-{8-oxabicyclo[3.2.1]octan-3-
yl}propanamide

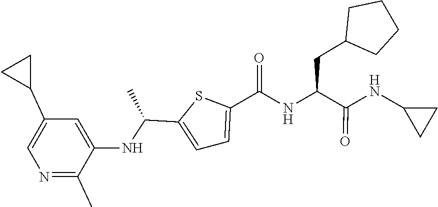 [SMILES: Cc1ncc(C2CC2)cc1N[C@H](C)c1ccc(C(=O)N[C@@H](CC2CCCC2)C(=O)NC2CC2)s1]

(2S)-3-cyclopentyl-N-cyclopropyl-2-({5-[(1R)-
1-[(5-cyclopropyl-2-methylpyridin-3-
yl)amino]ethyl]thiophen-2-
yl}formamido)propanamide

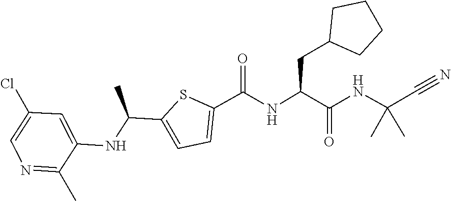 [SMILES: Cc1ncc(Cl)cc1N[C@@H](C)c1ccc(C(=O)N[C@@H](CC2CCCC2)C(=O)NC(C)(C)C#N)s1]

(2S)-2-({5-[(1S)-1-[(5-chloro-2-methylpyridin-
3-yl)amino]ethyl]thiophen-2-yl}formamido)-
N-(1-cyano-1-methylethyl)-3-
cyclopentylpropanamide

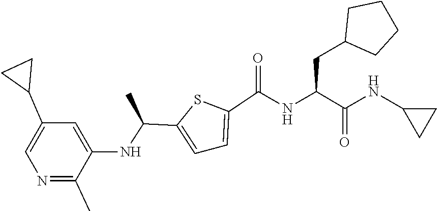 [SMILES: Cc1ncc(C2CC2)cc1N[C@@H](C)c1ccc(C(=O)N[C@@H](CC2CCCC2)C(=O)NC2CC2)s1]

(2S)-3-cyclopentyl-N-cyclopropyl-2-({5-[(1S)-
1-[(5-cyclopropyl-2-methylpyridin-3-
yl)amino]ethyl]thiophen-2-
yl}formamido)propanamide

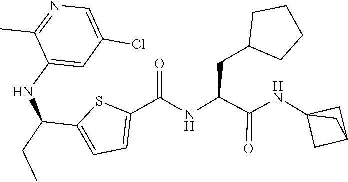 [SMILES: CC[C@@H](Nc1cc(Cl)cnc1C)c1ccc(C(=O)N[C@@H](CC2CCCC2)C(=O)NC23CC(C2)C3)s1]

(2S)-N-{bicyclo[1.1.1]pentan-1-yl}-2-({5-
[(1R)-1-[(5-chloro-2-methylpyridin-3-
yl)amino]propyl]thiophen-2-yl}formamido)-3-
cyclopentylpropanamide

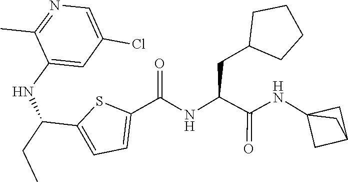 [SMILES: CC[C@H](Nc1cc(Cl)cnc1C)c1ccc(C(=O)N[C@@H](CC2CCCC2)C(=O)NC23CC(C2)C3)s1]

(2S)-N-{bicyclo[1.1.1]pentan-1-yl}-2-({5-
[(1S)-1-[(5-chloro-2-methylpyridin-3-
yl)amino]propyl]thiophen-2-yl}formamido)-3-
cyclopentylpropanamide

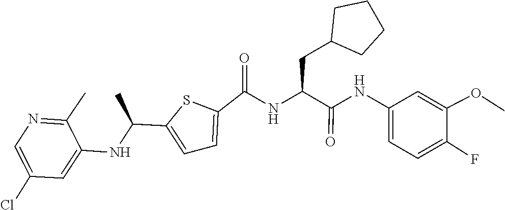 [SMILES: COc1cc(NC(=O)[C@H](CC2CCCC2)NC(=O)c2ccc([C@H](C)Nc3cc(Cl)cnc3C)s2)ccc1F]

(2S)-2-({5-[(1S)-1-[(5-chloro-2-methylpyridin-
3-yl)amino]ethyl]thiophen-2-yl}formamido)-3-
cyclopentyl-N-(4-fluoro-3-
methoxyphenyl)propanamide

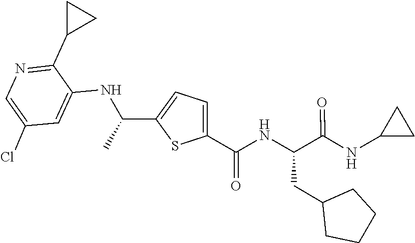 [SMILES: C[C@H](Nc1cc(Cl)cnc1C1CC1)c1ccc(C(=O)N[C@@H](CC2CCCC2)C(=O)NC2CC2)s1]

(2S)-2-({5-[(1S)-1-[(5-chloro-2-
cyclopropylpyridin-3-yl)amino]ethyl]thiophen-
2-yl}formamido)-3-cyclopentyl-N-
cyclopropylpropanamide

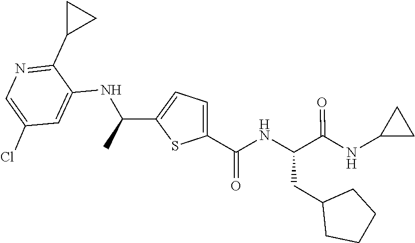 [SMILES: C[C@@H](Nc1cc(Cl)cnc1C1CC1)c1ccc(C(=O)N[C@@H](CC2CCCC2)C(=O)NC2CC2)s1]

(2S)-2-({5-[(1R)-1-[(5-chloro-2-
cyclopropylpyridin-3-yl)amino]ethyl]thiophen-
2-yl}formamido)-3-cyclopentyl-N-
cyclopropylpropanamide

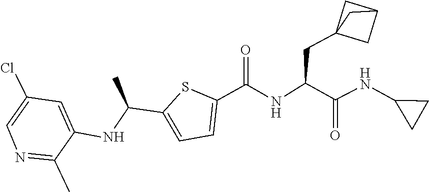 [SMILES: Cc1ncc(Cl)cc1N[C@@H](C)c1ccc(C(=O)N[C@@H](CC23CC(C2)C3)C(=O)NC2CC2)s1]

(2S)-3-{bicyclo[1.1.1]pentan-1-yl}-2-({5-
[(1S)-1-[(5-chloro-2-methylpyridin-3-
yl)amino]ethyl]thiophen-2-yl}formamido)-N-
cyclopropylpropanamide

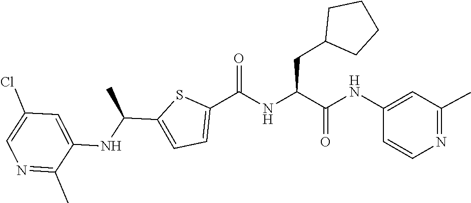 [SMILES: Cc1cc(NC(=O)[C@H](CC2CCCC2)NC(=O)c2ccc([C@H](C)Nc3cc(Cl)cnc3C)s2)ccn1]

(2S)-2-({5-[(1S)-1-[(5-chloro-2-methylpyridin-
3-yl)amino]ethyl]thiophen-2-yl}formamido)-3-
cyclopentyl-N-(2-methylpyridin-4-
yl)propanamide

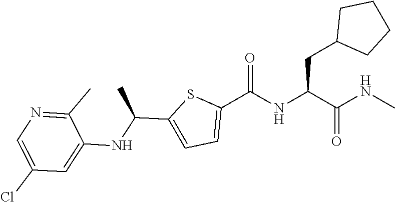 [SMILES: CNC(=O)[C@H](CC1CCCC1)NC(=O)c1ccc([C@H](C)Nc2cc(Cl)cnc2C)s1]

(2S)-2-({5-[(1S)-1-[(5-chloro-2-methylpyridin-
3-yl)amino]ethyl]thiophen-2-yl}formamido)-3-
cyclopentyl-N-methylpropanamide

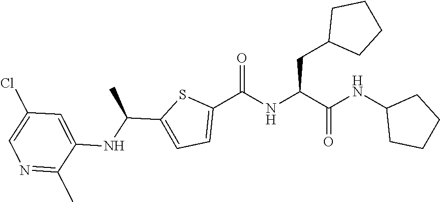 [SMILES: Cc1ncc(Cl)cc1N[C@@H](C)c1ccc(C(=O)N[C@@H](CC2CCCC2)C(=O)NC2CCCC2)s1]

(2S)-2-({5-[(1S)-1-[(5-chloro-2-methylpyridin-
3-yl)amino]ethyl]thiophen-2-yl}formamido)-
N,3-dicyclopentylpropanamide

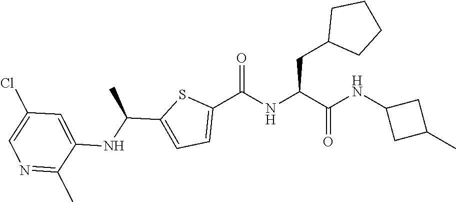 [SMILES: Cc1ncc(Cl)cc1N[C@@H](C)c1ccc(C(=O)N[C@@H](CC2CCCC2)C(=O)NC2CC(C)C2)s1]

(2S)-2-({5-[(1S)-1-[(5-chloro-2-methylpyridin-
3-yl)amino]ethyl]thiophen-2-yl}formamido)-3-
cyclopentyl-N-(3-
methylcyclobutyl)propanamide

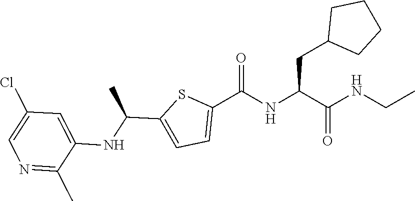 [SMILES: CCNC(=O)[C@H](CC1CCCC1)NC(=O)c1ccc([C@H](C)Nc2cc(Cl)cnc2C)s1]

(2S)-2-({5-[(1S)-1-[(5-chloro-2-methylpyridin-
3-yl)amino]ethyl]thiophen-2-yl}formamido)-3-
cyclopentyl-N-ethylpropanamide

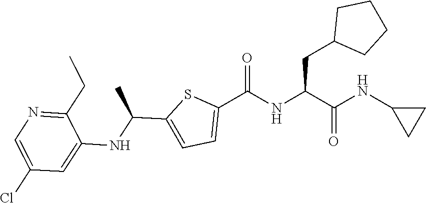 [SMILES: CCc1ncc(Cl)cc1N[C@@H](C)c1ccc(C(=O)N[C@@H](CC2CCCC2)C(=O)NC2CC2)s1]

(2S)-2-({5-[(1S)-1-[(5-chloro-2-ethylpyridin-3-
yl)amino]ethyl]thiophen-2-yl}formamido)-3-
cyclopentyl-N-cyclopropylpropanamide

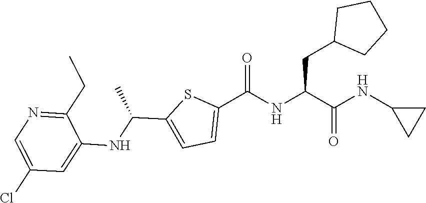 [SMILES: CCc1ncc(Cl)cc1N[C@H](C)c1ccc(C(=O)N[C@@H](CC2CCCC2)C(=O)NC2CC2)s1]

(2S)-2-({5-[(1R)-1-[(5-chloro-2-ethylpyridin-3-
yl)amino]ethyl]thiophen-2-yl}formamido)-3-
cyclopentyl-N-cyclopropylpropanamide

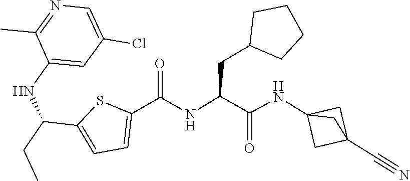 [SMILES: CC[C@H](Nc1cc(Cl)cnc1C)c1ccc(C(=O)N[C@@H](CC2CCCC2)C(=O)NC23CC(C#N)(C2)C3)s1]

(2S)-2-({5-[(1S)-1-[(5-chloro-2-methylpyridin-
3-yl)amino]propyl]thiophen-2-yl}formamido)-
N-{3-cyanobicyclo[1.1.1]pentan-1-yl}-3-
cyclopentylpropanamide

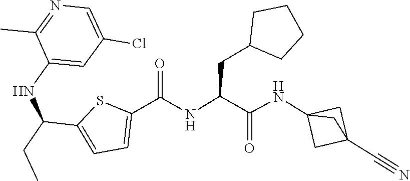 [SMILES: CC[C@@H](Nc1cc(Cl)cnc1C)c1ccc(C(=O)N[C@@H](CC2CCCC2)C(=O)NC23CC(C#N)(C2)C3)s1]

(2S)-2-({5-[(1R)-1-[(5-chloro-2-methylpyridin-
3-yl)amino]propyl]thiophen-2-yl}formamido)-
N-{3-cyanobicyclo[1.1.1]pentan-1-yl}-3-
cyclopentylpropanamide

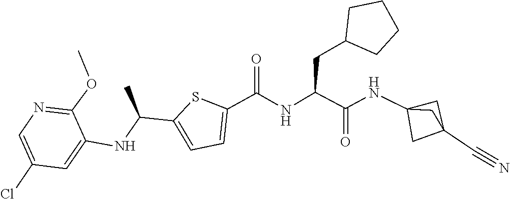 [SMILES: COc1ncc(Cl)cc1N[C@@H](C)c1ccc(C(=O)N[C@@H](CC2CCCC2)C(=O)NC23CC(C#N)(C2)C3)s1]

(2S)-2-({5-[(1S)-1-[(5-chloro-2-
methoxypyridin-3-yl)amino]ethyl]thiophen-2-
yl}formamido)-N-{3-
cyanobicyclo[1.1.1]pentan-1-yl}-3-
cyclopentylpropanamide

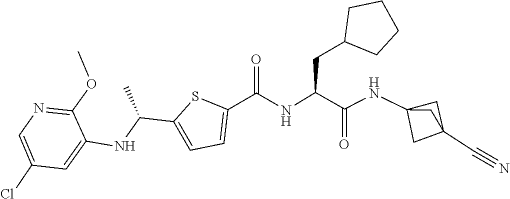 [SMILES: COc1ncc(Cl)cc1N[C@H](C)c1ccc(C(=O)N[C@@H](CC2CCCC2)C(=O)NC23CC(C#N)(C2)C3)s1]

(2S)-2-({5-[(1R)-1-[(5-chloro-2-
methoxypyridin-3-yl)amino]ethyl]thiophen-2-
yl}formamido)-N-{3-
cyanobicyclo[1.1.1]pentan-1-yl}-3-
cyclopentylpropanamide

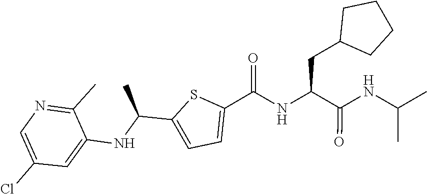 [SMILES: Cc1ncc(Cl)cc1N[C@@H](C)c1ccc(C(=O)N[C@@H](CC2CCCC2)C(=O)NC(C)C)s1]

(2S)-2-({5-[(1S)-1-[(5-chloro-2-methylpyridin-
3-yl)amino]ethyl]thiophen-2-yl}formamido)-3-
cyclopentyl-N-(propan-2-yl)propanamide

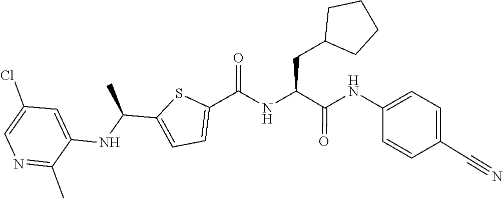 [SMILES: Cc1ncc(Cl)cc1N[C@@H](C)c1ccc(C(=O)N[C@@H](CC2CCCC2)C(=O)Nc2ccc(C#N)cc2)s1]

(2S)-2-({5-[(1S)-1-[(5-chloro-2-methylpyridin-
3-yl)amino]ethyl]thiophen-2-yl}formamido)-
N-(4-cyanophenyl)-3-cyclopentylpropanamide

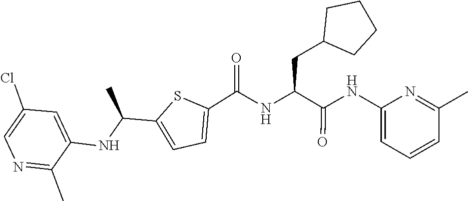 [SMILES: Cc1cccc(NC(=O)[C@H](CC2CCCC2)NC(=O)c2ccc([C@H](C)Nc3cc(Cl)cnc3C)s2)n1]

(2S)-2-({5-[(1S)-1-[(5-chloro-2-methylpyridin-
3-yl)amino]ethyl]thiophen-2-yl}formamido)-3-
cyclopentyl-N-(6-methylpyridin-2-
yl)propanamide

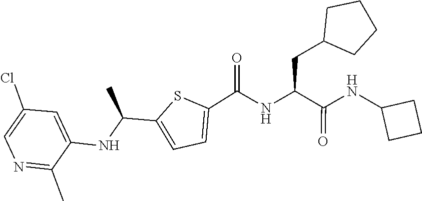 [SMILES: Cc1ncc(Cl)cc1N[C@@H](C)c1ccc(C(=O)N[C@@H](CC2CCCC2)C(=O)NC2CCC2)s1]

(2S)-2-({5-[(1S)-1-[(5-chloro-2-methylpyridin-
3-yl)amino]ethyl]thiophen-2-yl}formamido)-
N-cyclobutyl-3-cyclopentylpropanamide

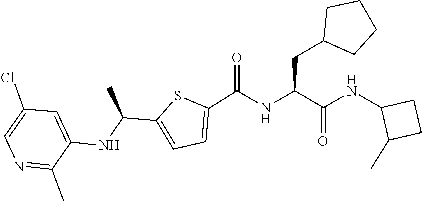 [SMILES: Cc1ncc(Cl)cc1N[C@@H](C)c1ccc(C(=O)N[C@@H](CC2CCCC2)C(=O)NC2CCC2C)s1]

(2S)-2-({5-[(1S)-1-[(5-chloro-2-methylpyridin-
3-yl)amino]ethyl]thiophen-2-yl}formamido)-3-
cyclopentyl-N-(2-
methylcyclobutyl)propanamide

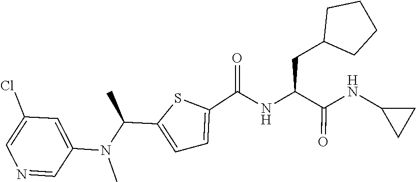 [SMILES: C[C@@H](c1ccc(C(=O)N[C@@H](CC2CCCC2)C(=O)NC2CC2)s1)N(C)c1cncc(Cl)c1]

(2S)-2-({5-[(1S)-1-[(5-chloropyridin-3-
yl)(methyl)amino]ethyl]thiophen-2-
yl}formamido)-3-cyclopentyl-N-
cyclopropylpropanamide

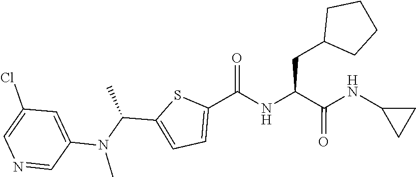 [SMILES: C[C@H](c1ccc(C(=O)N[C@@H](CC2CCCC2)C(=O)NC2CC2)s1)N(C)c1cncc(Cl)c1]

(2S)-2-({5-[(1R)-1-[(5-chloropyridin-3-
yl)(methyl)amino]ethyl]thiophen-2-
yl}formamido)-3-cyclopentyl-N-
cyclopropylpropanamide

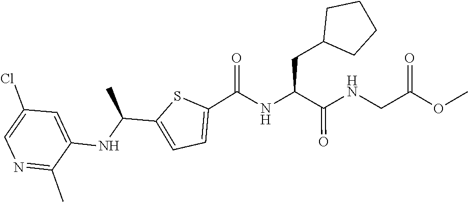 [SMILES: COC(=O)CNC(=O)[C@H](CC1CCCC1)NC(=O)c1ccc([C@H](C)Nc2cc(Cl)cnc2C)s1]

methyl 2-[(2S)-2-({5-[(1S)-1-[(5-chloro-2-
methylpyridin-3-yl)amino]ethyl]thiophen-2-
yl}formamido)-3-
cyclopentylpropanamido]acetate

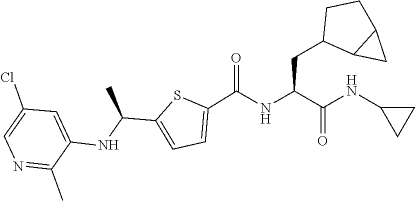 [SMILES: Cc1ncc(Cl)cc1N[C@@H](C)c1ccc(C(=O)N[C@@H](CC2CCC3CC32)C(=O)NC2CC2)s1]

(2S)-3-{bicyclo[3.1.0]hexan-2-yl}-2-({5-[(1S)-
1-[(5-chloro-2-methylpyridin-3-
yl)amino]ethyl]thiophen-2-yl}formamido)-N-
cyclopropylpropanamide

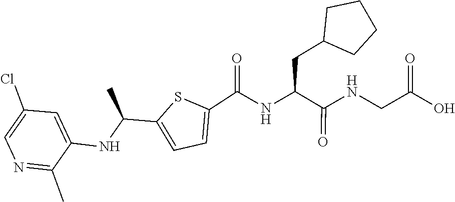 [SMILES: Cc1ncc(Cl)cc1N[C@@H](C)c1ccc(C(=O)N[C@@H](CC2CCCC2)C(=O)NCC(=O)O)s1]

2-[(2S)-2-({5-[(1S)-1-[(5-chloro-2-
methylpyridin-3-yl)amino]ethyl]thiophen-2-
yl}formamido)-3-
cyclopentylpropanamido]acetic acid

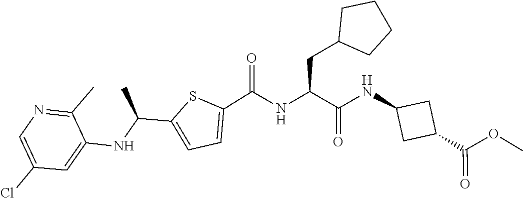 [SMILES: COC(=O)[C@H]1C[C@H](NC(=O)[C@H](CC2CCCC2)NC(=O)c2ccc([C@H](C)Nc3cc(Cl)cnc3C)s2)C1]

methyl (1r,3r)-3-[(2S)-2-({5-[(1S)-1-[(5-
chloro-2-methylpyridin-3-
yl)amino]ethyl]thiophen-2-yl}formamido)-3-
cyclopentylpropanamido]cyclobutane-1-
carboxylate

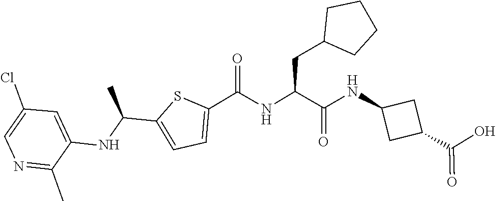 [SMILES: Cc1ncc(Cl)cc1N[C@@H](C)c1ccc(C(=O)N[C@@H](CC2CCCC2)C(=O)N[C@H]2C[C@H](C(=O)O)C2)s1]

(1r,3r)-3-[(2S)-2-({5-[(1S)-1-[(5-chloro-2-
methylpyridin-3-yl)amino]ethyl]thiophen-2-
yl}formamido)-3-
cyclopentylpropanamido]cyclobutane-1-
carboxylic acid

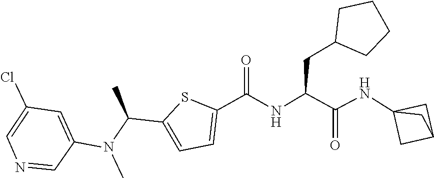 [SMILES: C[C@@H](c1ccc(C(=O)N[C@@H](CC2CCCC2)C(=O)NC23CC(C2)C3)s1)N(C)c1cncc(Cl)c1]

(2S)-N-{bicyclo[1.1.1]pentan-1-yl}-2-({5-
[(1S)-1-[(5-chloropyridin-3-
yl)(methyl)amino]ethyl]thiophen-2-
yl}formamido)-3-cyclopentylpropanamide

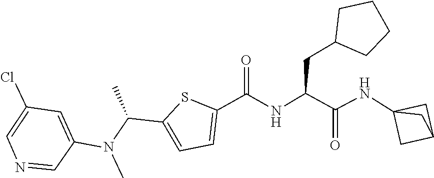 [SMILES: C[C@H](c1ccc(C(=O)N[C@@H](CC2CCCC2)C(=O)NC23CC(C2)C3)s1)N(C)c1cncc(Cl)c1]

(2S)-N-{bicyclo[1.1.1]pentan-1-yl}-2-({5-
[(1R)-1-[(5-chloropyridin-3-
yl)(methyl)amino]ethyl]thiophen-2-
yl}formamido)-3-cyclopentylpropanamide

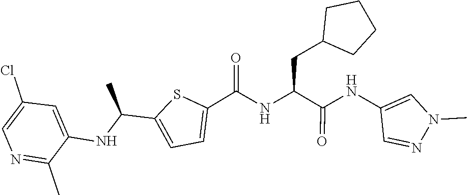 [SMILES: Cc1ncc(Cl)cc1N[C@@H](C)c1ccc(C(=O)N[C@@H](CC2CCCC2)C(=O)Nc2cnn(C)c2)s1]

(2S)-2-({5-[(1S)-1-[(5-chloro-2-methylpyridin-
3-yl)amino]ethyl]thiophen-2-yl}formamido)-3-
cyclopentyl-N-(1-methyl-1H-pyrazol-4-
yl)propanamide

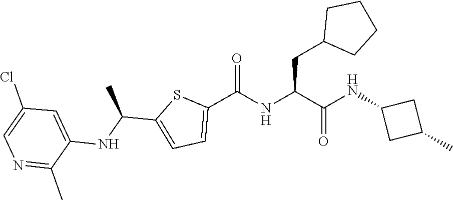 [SMILES: Cc1ncc(Cl)cc1N[C@@H](C)c1ccc(C(=O)N[C@@H](CC2CCCC2)C(=O)N[C@H]2C[C@@H](C)C2)s1]

(2S)-2-({5-[(1S)-1-[(5-chloro-2-methylpyridin-
3-yl)amino]ethyl]thiophen-2-yl}formamido)-3-
cyclopentyl-N-[(1r,3s)-3-
methylcyclobutyl]propanamide

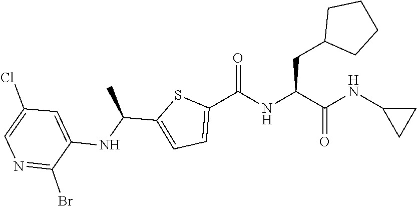 [SMILES: C[C@H](Nc1cc(Cl)cnc1Br)c1ccc(C(=O)N[C@@H](CC2CCCC2)C(=O)NC2CC2)s1]

(2S)-2-({5-[(1S)-1-[(2-bromo-5-chloropyridin-
3-yl)amino]ethyl]thiophen-2-yl}formamido)-3-
cyclopentyl-N-cyclopropylpropanamide

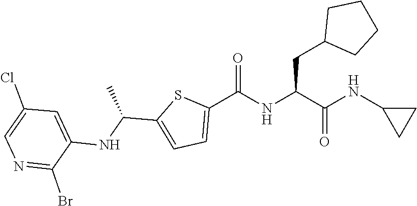 [SMILES: C[C@@H](Nc1cc(Cl)cnc1Br)c1ccc(C(=O)N[C@@H](CC2CCCC2)C(=O)NC2CC2)s1]

(2S)-2-({5-[(1R)-1-[(2-bromo-5-chloropyridin-
3-yl)amino]ethyl]thiophen-2-yl}formamido)-3-
cyclopentyl-N-cyclopropylpropanamide

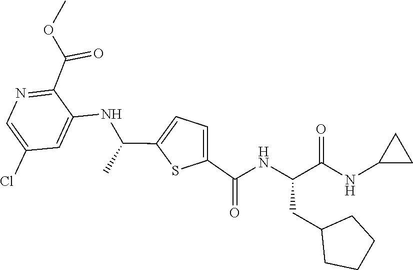 [SMILES: COC(=O)c1ncc(Cl)cc1N[C@@H](C)c1ccc(C(=O)N[C@@H](CC2CCCC2)C(=O)NC2CC2)s1]

methyl 5-chloro-3-{[(1S)-1-(5-{[(1S)-2-
cyclopentyl-1-(cyclopropylcarbamoyl)
ethyl]carbamoyl}thiophen-
2-yl)ethyl]amino}pyridine-2-carboxylate

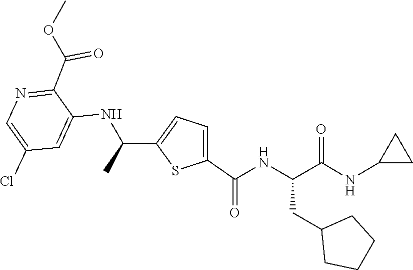 [SMILES: COC(=O)c1ncc(Cl)cc1N[C@H](C)c1ccc(C(=O)N[C@@H](CC2CCCC2)C(=O)NC2CC2)s1]

methyl 5-chloro-3-{[(1R)-1-(5-{[(1S)-2-
cyclopentyl-1-(cyclopropylcarbamoyl)
ethyl]carbamoyl}thiophen-
2-yl)ethyl]amino}pyridine-2-carboxylate

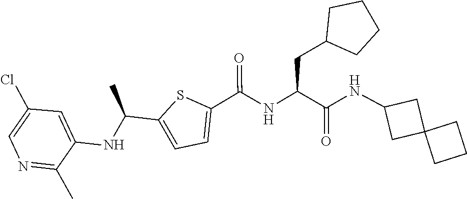 [SMILES: Cc1ncc(Cl)cc1N[C@@H](C)c1ccc(C(=O)N[C@@H](CC2CCCC2)C(=O)NC2CC3(CCC3)C2)s1]

(2S)-2-({5-[(1S)-1-[(5-chloro-2-methylpyridin-
3-yl)amino]ethyl]thiophen-2-yl}formamido)-3-
cyclopentyl-N-{spiro[3.3]heptan-2-
yl}propanamide

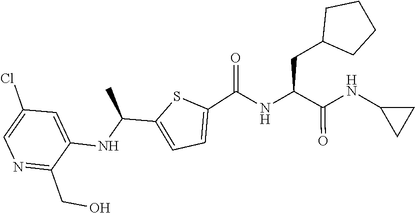 [SMILES: C[C@H](Nc1cc(Cl)cnc1CO)c1ccc(C(=O)N[C@@H](CC2CCCC2)C(=O)NC2CC2)s1]

(2S)-2-({5-[(1S)-1-{[5-chloro-2-
(hydroxymethyl)pyridin-3-
yl]amino}ethyl]thiophen-2-yl}formamido)-3-
cyclopentyl-N-cyclopropylpropanamide

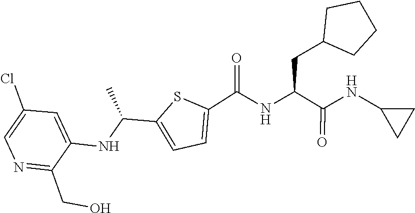 [SMILES: C[C@@H](Nc1cc(Cl)cnc1CO)c1ccc(C(=O)N[C@@H](CC2CCCC2)C(=O)NC2CC2)s1]

(2S)-2-({5-[(1R)-1-{[5-chloro-2-
(hydroxymethyl)pyridin-3-
yl]amino}ethyl]thiophen-2-yl}formamido)-3-
cyclopentyl-N-cyclopropylpropanamide

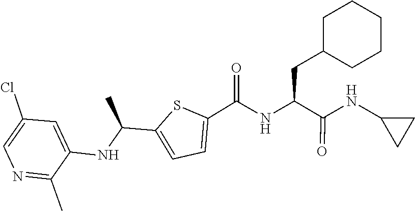 [SMILES: Cc1ncc(Cl)cc1N[C@@H](C)c1ccc(C(=O)N[C@@H](CC2CCCCC2)C(=O)NC2CC2)s1]

(2S)-2-({5-[(1S)-1-[(5-chloro-2-methylpyridin-
3-yl)amino]ethyl]thiophen-2-yl}formamido)-3-
cyclohexyl-N-cyclopropylpropanamide

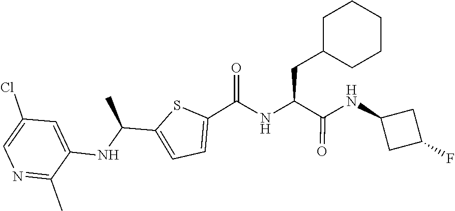 [SMILES: Cc1ncc(Cl)cc1N[C@@H](C)c1ccc(C(=O)N[C@@H](CC2CCCCC2)C(=O)N[C@H]2C[C@H](F)C2)s1]

(2S)-2-({5-[(1S)-1-[(5-chloro-2-methylpyridin-
3-yl)amino]ethyl]thiophen-2-yl}formamido)-3-
cyclohexyl-N-[(1r,3r)-3-
fluorocyclobutyl]propanamide

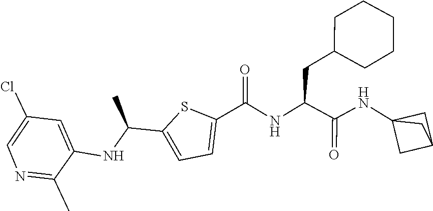 [SMILES: Cc1ncc(Cl)cc1N[C@@H](C)c1ccc(C(=O)N[C@@H](CC2CCCCC2)C(=O)NC23CC(C2)C3)s1]

(2S)-N-{bicyclo[1.1.1]pentan-1-yl}-2-({5-
[(1S)-1-[(5-chloro-2-methylpyridin-3-
yl)amino]ethyl]thiophen-2-yl}formamido)-3-
cyclohexylpropanamide

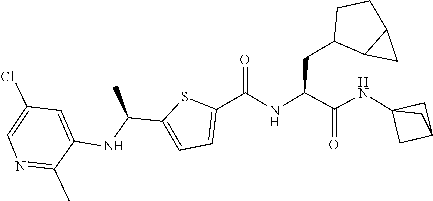 [SMILES: Cc1ncc(Cl)cc1N[C@@H](C)c1ccc(C(=O)N[C@@H](CC2CCC3CC32)C(=O)NC23CC(C2)C3)s1]

(2S)-N-{bicyclo[1.1.1]pentan-1-yl}-3-
{bicyclo[3.1.0]hexan-2-yl}-2-({5-[(1S)-1-[(5-
chloro-2-methylpyridin-3-
yl)amino]ethyl]thiophen-2-
yl}formamido)propanamide

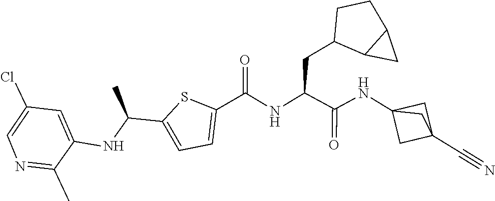 [SMILES: Cc1ncc(Cl)cc1N[C@@H](C)c1ccc(C(=O)N[C@@H](CC2CCC3CC32)C(=O)NC23CC(C#N)(C2)C3)s1]

(2S)-3-{bicyclo[3.1.0]hexan-2-yl}-2-({5-[(1S)-
1-[(5-chloro-2-methylpyridin-3-
yl)amino]ethyl]thiophen-2-yl}formamido)-N-
{3-cyanobicyclo[1.1.1]pentan-1-
yl}propanamide

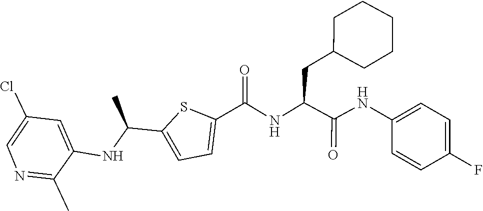 [SMILES: Cc1ncc(Cl)cc1N[C@@H](C)c1ccc(C(=O)N[C@@H](CC2CCCCC2)C(=O)Nc2ccc(F)cc2)s1]

(2S)-2-({5-[(1S)-1-[(5-chloro-2-methylpyridin-
3-yl)amino]ethyl]thiophen-2-yl}formamido)-3-
cyclohexyl-N-(4-fluorophenyl)propanamide

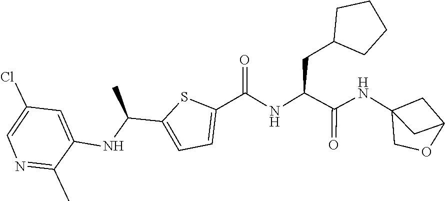 [SMILES: Cc1ncc(Cl)cc1N[C@@H](C)c1ccc(C(=O)N[C@@H](CC2CCCC2)C(=O)NC23COC(C2)C3)s1]

(2S)-2-({5-[(1S)-1-[(5-chloro-2-methylpyridin-
3-yl)amino]ethyl]thiophen-2-yl}formamido)-3-
cyclopentyl-N-{2-oxabicyclo[2.1.1]hexan-4-
yl}propanamide

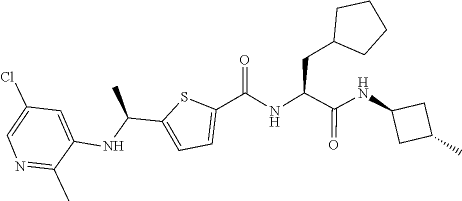 [SMILES: Cc1ncc(Cl)cc1N[C@@H](C)c1ccc(C(=O)N[C@@H](CC2CCCC2)C(=O)N[C@H]2C[C@H](C)C2)s1]

(2S)-2-({5-[(1S)-1-[(5-chloro-2-methylpyridin-3-
yl)amino]ethyl]thiophen-2-yl}formamido)-3-
cyclopentyl-N-[(1s,3r)-3-
methylcyclobutyl]propanamide

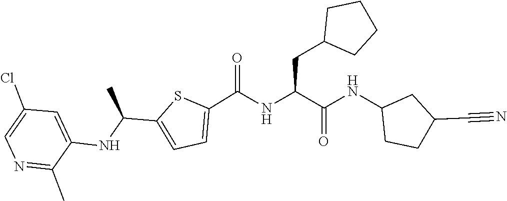 [SMILES: Cc1ncc(Cl)cc1N[C@@H](C)c1ccc(C(=O)N[C@@H](CC2CCCC2)C(=O)NC2CCC(C#N)C2)s1]

(2S)-2-({5-[(1S)-1-[(5-chloro-2-methylpyridin-
3-yl)amino]ethyl]thiophen-2-yl}formamido)-
N-(3-cyanocyclopentyl)-3-
cyclopentylpropanamide

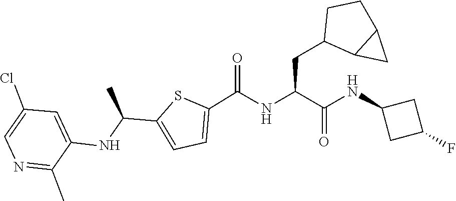 [SMILES: Cc1ncc(Cl)cc1N[C@@H](C)c1ccc(C(=O)N[C@@H](CC2CCC3CC32)C(=O)N[C@H]2C[C@H](F)C2)s1]

(2S)-3-{bicyclo[3.1.0]hexan-2-yl}-2-({5-[(1S)-
1-[(5-chloro-2-methylpyridin-3-
yl)amino]ethyl]thiophen-2-yl}formamido)-N-
[(1r,3r)-3-fluorocyclobutyl]propanamide

In cellular studies, overexpression of WIP1 in E1A transduced rat embryonic fibroblasts was sufficient to induce transformation foci (Nannenga et al, Molecular Carcinogenesis 45, 594-604 (2006)). Conversely, murine embryonic fibroblasts homozygous for WIP1 deletion showed significantly reduced tumor growth when co-transfected with the transforming oncogenes 1-RAS and E1A, HRAS and ErbB2, or HRAS and Myc (Bulavin et. al., Nature Genetics 36, 343-350 (2004)). WIP1 −/− mice also demonstrated tumor-resistance to both induced tumor models, using infection with MTV driven ErbB2 or HRAS, as well as a decreased incidence of spontaneous leukemias and sarcomas. Similarly, WIP1 −/− mice were relatively resistant to Eμ-Myc induced lymphomas (Shreeram et al., Journal of Experimental Medicine 203, 2793-2799 (2006).

Overexpression of WIP1 is reported to result in delay or suppression of base-excision repair by inhibition of UNG2 and P53 Similarly, its overexpression suppresses DNA double-strand-break repair by inhibition of the ATM response, inhibiting repair by both homologous recombination and non-homologous end joining. Conversely, silencing or knockout of Wip1 has been reported to elevate the activity of multiple stress response pathways, resulting in elevated levels of phospho-p38, phospho-p53(S15), p53 response genes p21/Waf, p 1 6INK4A, and ARF (Bulavin et al., Nature Genetics 36, 343-350 (2004)), and elevated γH2AX and DNA damage-associated nuclear foci (Moon et al., Journal of Biological Chemistry 285, 12935-12947 (2010)).

Amplified or overexpressed Wip1 has also been proposed to promote tumorigenesis in multiple cancers by suppressing the regulatory activity of a host of its tumor suppressor substrates. Amplification of the WIP1/PPM1D gene locus on 17q23 has been reported in breast cancer (Li et al., Nature Genetics 31, 133-134. (2002)), ovarian clear cell carcinoma (Hirasawa et al. Clinical Cancer Research 9, 1995-2004 (2003)), neuroblastoma (Saito-Ohara et al., Journal of Experimental Medicine 203, 2793-2709 (2003)), and pancreatic adenocarcinoma (Loukopoulos et al., Cancer Science 98, 392-400 (2007)), Moreover, elevated expression of WIP1 is reported in mediilloblastomas (Castellino et al., Journal of Neuro-Oncology 86, 245-256 (2008)) and gastric carcinomas (Fuku et al., Pathology International 57, 566-571 (2007)). In several of these tumors, gene amplification has been confirmed to correlate with elevated protein expression.

Inhibition of WIP1 by small molecule inhibitors or by RNA interference has been shown to have antiproliferative effects in tumor cells. In cell culture studies, silencing of WIP1 by RINA interference results in elevated phosphorylation and activation of multiple tumor suppressors including p53, Chk2 and γH2AX, and results in tumor cell growth inhibition. (Fujimoto et al., Cell Death and Differentiation 13, 1170-1180 (2006); Lu et al., Cancer Cell 12, 342-351 (2007)). Several groups have also reportedly identified WIP1 inhibitors, describing antiproliferative activity in WIP1 amplified or overexpressing cell lines (Belova et al., Cancer Biology and Therapy 4, 1154-1158. (2005); Rayter et al., Oncogene 27, 1036-1044 (2008)).

This invention also relates to compounds exemplified in the Experimental section.

The compound of Formula (I) or a salt thereof may exist in stereoisomeric forms (e.g., it contains one or more asymmetric carbon atoms). The individual stereoisomers (enantiomers and diastereomers) and mixtures of these are included within the scope of the present invention. The invention also covers the individual isomers of the compound or salt represented by Formula (I) as mixtures with isomers thereof in which one or more chiral centers are inverted. Likewise, it is understood that a compound or salt of Formula (I) may exist in tautomeric forms other than that shown in the formula and these are also included within the scope of the present invention. It is to be understood that the present invention includes all combinations and subsets of the particular groups defined hereinabove. The scope of the present invention includes mixtures of stereoisomers as well as purified enantiomers or enantiomerically/diastereomerically enriched mixtures. Also included within the scope of the invention are any wholly or partially deuterated isotopes of the compounds of Formula (1). Also included within the scope of the invention are individual isomers of the compound represented by Formula (I), as well as any wholly or partially equilibrated mixtures thereof. The present invention also includes the individual isomers of the compound or salt represented by the Formula (I) as well as mixtures with isomers thereof in which one or more chiral centers are inverted.

It will be appreciated by those skilled in the art that certain protected derivatives of compounds of formula (1), which may be made prior to a final deprotection stage, may not possess pharmacological activity as such, but may, in certain instances, be administered orally or parenterally and thereafter metabolized in the body to form compounds of the invention which are pharmacologically active. Such derivatives may therefore be described as “prodrugs”. Further, certain compounds of the invention may act as prodrugs of other compounds of the invention. All protected derivatives and prodrugs of compounds of the invention are included within the scope of the invention. It will further be appreciated by those skilled in the art, that certain moieties, known to those skilled in the art as “pro-moieties” may be placed on appropriate functionalities when such functionalities are present within compounds of the invention. Preferred prodrugs for compounds of the invention include: esters, carbonate esters, hemi-esters, phosphate esters, nitro esters, sulfate esters, sulfoxides, amides, carbamates, azo-compounds, phosphamides, glycosides, ethers, acetals and ketals. The scope of the present invention also includes prodrugs of the present compounds.

### Definitions

Terms are used within their accepted meanings. The following definitions are meant to clarify, but not limit, the terms defined.

As used herein, the term “alkyl” (or “alkylene”) refers to a straight or branched chain alkyl, preferably having from one to twelve carbon atoms. Other preferred alkyl is “lower alkyl” having one to six carbon atoms. Examples of “alkyl” as used herein include methyl, ethyl, propyl, isopropyl, isobutyl, n-butyl, t-butyl, isopentyl, n-pentyl, and the like, as well as substituted versions thereof.

As used herein, the term “substituted alkyl” (or “alkylene”) refers to a straight or branched chain alkyl, preferably having from one to twelve carbon atoms. Suitable substituents are selected from the group consisting of halogen, amino, substituted amino, urea, cyano, hydroxyl, alkoxy, alkylthio, alkylsulfonyl, amidosulfonyl, carboxylic acid, ester, carboxamide, and aminocarbonyl.

As used herein, the term “cycloalkyl” refers to mono- or polycyclic non-aromatic saturated ring, which can be unsubstituted or substituted with one or more substituents unless specified otherwise. Exemplary “cycloalkyl” groups include, but are not limited to, cyclopropyl, cyclobutyl, cyclopentyl, cyclohexyl, cycloheptyl, and the like, as well as and substituted versions thereof unless specified otherwise.

As used herein, the term “alkoxy” refers to the group —ORₐ, where Rₐ is C₁-C₁₀ alkyl or C₃-C₇cycloalkyl as defined above. As used herein, the term “substituted amino” is meant —NR′R″ wherein each R′ and R″ is independently selected from a group including hydrogen, unsubstituted C₁-C₁₀alkyl, acyl, unsubstituted C₃-C₇cycloalkyl, wherein at least one of R′ and R″ is not hydrogen. Examples of substituted amino includes, but are not limited to alkylamino, dialkylamino, acylamino, and cycloalkylamino.

As used herein, the term “heterocycle” or “heterocyclyl” or “heterocycloalkyl” refers to unsubstituted mono- or polycyclic ring, system containing one or more heteroatoms, as well as substituted versions thereof unless specified otherwise. Preferred heteroatoms include N, O, and S, including N-oxides, sulfur oxides, and dioxides. Heterocyclic rings can include polycyclic rings, which can contain, for example, fused, spiro, or bridged ring junction.

Examples of “heterocyclic” groups include, but are not limited to oxetanyl, tetrahydrofuranyl, pyranyl, 1,4-dioxanyl, 1,3-dioxanyl, piperidinyl, pyrrolidinyl, morpholinyl, azetidinyl, piperazinyl, pyrrolidinonyl, piperazinonyl, pyrazolidinyl, 3-pyrazolyl, 4-pyrazolyl, 11H-1,2,3-triazol-4-yl, 3-pyridinyl, 4-pyridinyl, 2-pyridinyl, 3-thienyl, phenyl, 5-indolyl, 6-indolyl, 5-indazolyl, 5-benzimidazolyl, 1H-pyrrolo[2,3-b]pyridin-5-yl, 1,2,3,4-tetrahydroquinolin-1-yl, 5,6,7,8-tetrahydroquinolin-6-yl, 1,2,3,4-tetrahydroquinolin-6-yl, 6-isoquinolinyl, and quinolin-6-yl, and their various tautomers, as well as unsubstituted and substituted versions thereof unless specified otherwise. Such tautomers include 2-hydroxypyridine and 2-pyridone.

As used herein, the term “aryl” unless otherwise defined, is meant aromatic, hydrocarbon, ring system. The ring system may be monocyclic or fused polycyclic (e.g., bicyclic, tricyclic, etc.). In various embodiments, the monocyclic aryl ring is C₅-C₁₀, or C₅-C₇, or C₅-C₆, where these carbon numbers refer to the number of carbon atoms that form the ring system. A C₆ ring system, i.e. a phenyl ring, is a suitable aryl group. In various embodiments, the polycyclic ring is a bicyclic aryl group, where suitable bicyclic aryl groups are C₈-C₁₂, or C₉-C₁₀. A naphthyl ring, which has 10 carbon atoms, is a suitable polycyclic aryl group. Suitable substituents for aryl include, but are not limited to the example substituents described in the definition of “substituted”.

As used herein, the term “heteroaryl”, unless otherwise defined, is meant an aromatic ring system containing carbon(s) and at least one heteroatom, and substituted variants thereof unless specified otherwise. Heteroaryl mar be monocyclic or polycyclic, substituted or unsubstituted. A monocyclic heteroaryl group may have 1 to 4 heteroatoms in the ring, while a polycyclic heteroaryl may contain 1 to 10 hetero atoms. A polycyclic heteroaryl ring may contain fused ring junctions, for example, bicyclic heteroaryl is a polycyclic heteroaryl. Bicyclic heteroaryl rings may contain from S to 12 member atoms. Monocyclic heteroaryl rings may contain from 5 to 8 member atoms (carbons and heteroatoms). Example heteroaryl groups include: benzofuran, benzothiophene, furan, imidazole, indole, isothiazole, oxazole, pyrazine, pyrazole, pyridazine, pyridine, pyrimidine, pyrrole, quinoline, quinazoline, quinoxaline, thiazole, and thiophene. Suitable substituents for heteroaryl include, but are not limited to the example substituents described in the definition of “substituted”.

As used herein, the term “cyano” refers to the group —CN.

As used herein, the term “acyl” refers to the group —C(O)R^{b}, where R^{b} is alkyl, cycloalkyl, or heterocyclyl, as each is defined herein.

As used herein, the term “optionally” means that the subsequently described event(s) may or may not occur, and includes both event(s) that occur and event(s) that do not occur.

As used herein, unless otherwise defined, the phrase “substituted” or variations thereof denote a substitution, including multiple degrees of substitution, with one or more substituents, preferably one, two or three. The phrase should not be interpreted as duplicative of the substitutions herein described and depicted. Exemplary substituents groups include acyl, alkyl, substituted alkyl, alkylsulfonyl, alkoxy, alkoxycarbonyl, cyano, halogen, haloalkyl, hydroxyl, oxo, amide, sulfamide, urea, amino, substituted amino, acylamino, phenylcarbonyl, dialkylaminosulfonamide, morpholino, sulfonamide, thiourea, carboxylic acid, ester, and nitro.

The invention further provides a pharmaceutical composition (also referred to as pharmaceutical formulation) comprising a compound of Formulas (1-6) or pharmaceutically acceptable salt, thereof and one or more excipients (also referred to as carriers and/or diluents in the pharmaceutical arts). The excipients are acceptable in the sense of being compatible with the other ingredients of the formulation and not deleterious to the recipient thereof (i.e., the patient). In accordance with another aspect of the invention there is provided a process for the preparation of a pharmaceutical composition comprising mixing (or admixing) a compound of Formulas (1-6) or salt thereof with at least one excipient.

### Combinations

When a compound of Formulas (1-6) is administered for the treatment of cancer, the term “co-administering” and derivatives thereof as used herein is meant either simultaneous administration or any manner of separate sequential administration of a WIP1 inhibiting compound, as described herein, and a further active ingredient or ingredients, known to be useful in the treatment of cancer, including chemotherapy and radiation treatment. The term further active ingredient or ingredients, as used herein, includes any compound or therapeutic agent known to or that demonstrates advantageous properties when administered to a patient in need of treatment for cancer. Preferably, if the administration is not simultaneous, the compounds are administered in a close time proximity to each other. Furthermore, it does not matter if the compounds are administered in the same dosage form, e.g. one compound may be administered topically and another compound may be administered orally.

Typically, any anti-neoplastic-agent that has activity versus a susceptible tumor being treated may be co-administered in the treatment of cancer in the present invention. Examples of such agents can be found in Cancer Principles and Practice f Oncology by V. T. Devita and S. Hellman (editors) 6^{th} edition (Feb. 15, 2001), Lippincott Williams & Wilkins Publishers A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase 11 inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; and cell cycle signaling inhibitors. Examples of a further active ingredient or ingredients for use in combination or co-administered with the present WIP1 inhibiting compounds are chemotherapeutic agents. Anti-microtubule or anti-mitotic agents are phase specific agents active against the microtubules of tumor cells during M or the mitosis phase of the cell cycle Examples of anti-microtubule agents include, but are not limited to, diterpenoids and vinca alkaloids.

In one embodiment, the cancer treatment method of the claimed invention includes the co-administration a compound of Formulas (1-6) and/or a pharmaceutically acceptable salt, hydrate, solvate or pro-drug thereof and at least one anti-neoplastic agent, such as one selected from the group consisting of anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, FAS inhibitors, HDAC inhibitors, and cell cycle signalling inhibitors.

In some embodiments, disclosed herein is a method of treating a cancer, the method comprising administering to a subject in need thereof a therapeutically-effective amount of a compound of the disclosure. A compound of the invention can, for example, slow the proliferation of cancer cell lines, or kill cancer cells. Non-limiting examples of cancer that can be treated by a compound of the invention include: acute lymphoblastic leukemia, acute myeloid leukemia, adrenocortical carcinoma, AIDS-related cancers, AIDS-related lymphoma, anal cancer, appendix cancer, astrocytomas, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenström macroglobulinemia, and Wilms tumor.

### Pharmaceutically-Acceptable Salts.

The invention provides the use of pharmaceutically-acceptable salts of any therapeutic compound described herein. Pharmaceutically-acceptable salts include, for example, acid-addition salts and base-addition sails. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the invention. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, ceriman magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the present disclosure. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrazole, imidazole, or pyrazine.

In some embodiments, an ammonium salt is a triethyl amine salt, a trimethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrazole salt, a pyridazine salt, a pyrimidine salt, an imidazole salt, or a pyrazine salt.

Acid addition salts can arise from the addition of an acid to a compound of the present disclosure. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisic acid, gluconic acid, glucuronic acid, saccharic acid, formic acid, benzoic acid, glutamic acid, pantothenic acid, acetic acid, trifluoroacetic acid, mandelic acid, cinnamic acid, aspartic acid, stearic acid, palmitic acid, glycolic acid, propionic acid, butyric acid, furanic acid, succinic acid, methanesulfonic acid, ethanesulfonic acid, benzenesulfonic acid, p-toluenesulfonic acid, citric acid, oxalic acid, or maleic acid. In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisate salt, a gluconate salt, a glucuronate salt, a saccharate salt, a formate salt, a benzoate salt, a glutamate salt, a pantothenate salt, an acetate salt, a trifluoroacetate salt, a mandelate salt, a cinnamate salt, an aspartate salt, a stearate salt, a palmitate salt, a glycolate salt, a propionate salt, a butyrate salt, a furanoate salt, a succinate salt, a methanesulfonate salt, an ethanesulfonate salt, a benzenesulfonate salt, a p-toluenesulfonate salt, a citrate salt, an oxalate salt, or a maleate salt.

Pharmaceutical Compositions of the Invention.

A pharmaceutical composition of the invention can be used, for example, before, during, or after treatment of a subject with, for example, another pharmaceutical agent. Subjects can be, for example, elderly adults, adults, adolescents, pre-adolescents, children, toddlers, infants, neonates, and non-human animals. In some embodiments, a subject is a patient. A pharmaceutical composition of the invention can be a combination of any pharmaceutical compounds described herein with other chemical components, such as carriers, stabilizers, diluents, dispersing agents, suspending agents, thickening agents, and/or excipients. The pharmaceutical composition facilitates administration of the compound to an organism. Pharmaceutical compositions can be administered in therapeutically effective amounts as pharmaceutical compostions by various forms and routes including, for example, intravenous, subcutaneous, intramuscular, oral, parenteral, ophthalmic, subcutaneous, transdermal, nasal, vaginal, and topical administration. A pharmaceutical composition can be administered in a local manner, for example, via injection of the compound directly into an organ, optionally in a depot or sustained release formulation or implant. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

For oral administration, pharmaceutical compositions can be formulated by combining the active compounds with pharmaceutically-acceptable carriers or excipients. Such carriers can be used to formulate liquids, gels, syrups, elixirs, slurries, or suspensions, for oral ingestion by a subject. Non-limiting examples of solvents used in an oral dissolvable formulation can include water, ethanol, isopropanol, saline, physiological saline, DMSO, dimethylformamide, potassium phosphate buffer, phosphate buffer saline (PBS), sodium phosphate buffer, 4-2-hydroxyethyl-1-piperazineethanesulfonic acid buffer (HEPES), 3-(N-morpholino)propanesulfonic acid buffer (MOPS), piperazine-N,N¢-bis(2-ethanesulfonic acid) buffer (PIPES), and saline sodium citrate buffer (SSC). Non-limiting examples of co-solvents used in an oral dissolvable formulation can include sucrose, urea, cremophor, DMSO, and potassium phosphate buffer.

Pharmaceutical preparations can be formulated for intravenous administration. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

The active compounds can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

The compounds of the invention can be applied topically to the skin, or a body cavity, for example, oral, vaginal, bladder, cranial, spinal, thoracic, or pelvic cavity of a subject. The compounds of the invention can be applied to an accessible body cavity.

The compounds can also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, and PEG. In suppository forms of the compositions, a low-melting wax such as a mixture of fatty acid glycerides, optionally in combination with cocoa butter, can be melted.

In practicing the methods of treatment or use provided herein, therapeutically-effective amounts of the compounds described herein are administered in pharmaceutical compositions to a subject having a disease or condition to be treated. In some embodiments, the subject is a mammal such as a human. A therapeutically-effective amount can vary widely depending on the severity of die disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the active compounds into preparations that can be used pharmaceutically. Formulations can be modified depending upon the route of administration chosen. Pharmaceutical compositions comprising a compound described herein can be manufactured, for example, by mixing, dissolving, emulsifying, encapsulating, entrapping, or compression processes.

The pharmaceutical compositions can include at least one pharmaceutically-acceptable carrier, diluent, or excipient and compounds described herein as free-base or pharmaceutically-acceptable salt form. Pharmaceutical compositions can contain solubilizers, stabilizers, tonicity enhancing agents, buffers and preservatives.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the invention include liquid, powder, gel, nanosuspension, nanoparticle, microgel, aqueous or oily suspensions, emulsion and any combination thereof. Non-limiting examples of pharmaceutically-acceptable excipients suitable for use in the invention include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof.

A composition of the invention can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that release rates and release profiles of the active agent can be matched to physiological and Chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses. In some, a controlled release formulation is a delayed release form A delayed release form can be formulated to delay a compound's action for an extended period of time. A delayed release form can be formulated to delay the release of an effective dose of one or more compounds, for example, for about 4, about 8, about 12, about 16, or about 24 h. A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16 or about 24 h. Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

Multiple therapeutic agents can be administered in any order or simultaneously. In some embodiments, a compound of the invention is administered in combination with, before, or after treatment with another therapeutic agent. If simultaneously, the multiple therapeutic agents can be provided in a single, unified form, or in multiple forms, for example, as multiple separate pills. The agents can be packed together or separately, in a single package or in a plurality of packages. One or all of the therapeutic agents can be given in multiple doses. If not simultaneous, the timing between the multiple doses can vary to as much as about a month Therapeutic agents described herein can be administered before, during, or after the occurrence of a disease or condition, and the timing of administering the composition containing a therapeutic agent can vary. For example, the compositions can be used as a prophylactic and can be administered continuously to subjects with a propensity to conditions or diseases in order to lessen a likelihood of the occurrence of the disease or condition. The compositions can be administered to a subject during or as soon as possible after the onset of the symptoms. The administration of the therapeutic agents can be initiated within the first 48 h of the onset of the symptoms, within the first 24 h of the onset of the symptoms, within the first 6 h of the onset of the symptoms, or within 3 h of the onset of the symptoms. The initial administration can be via any route practical, such as by any route described herein using any formulation described herein.

A compound can be administered as soon as is practical after the onset of a disease or condition is detected or suspected, and for a length of time necessary for the treatment of the disease, such as, for example, from about 1 month to about 3 months. In some embodiments, the length of time a compound can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 21 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, or about 10 years. The length of treatment can vary for each subject.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a presentative, Formulations for injection can be presented in unit dosage form, for example, in ampoules, or in multi-dose containers with a preservative. Pharmaceutical compositions provided herein, can be administered in conjunction with other therapies, for example, chemotherapy, radiation, surgery, anti-inflammatory agents, and selected vitamins. The other agents can be administered prior to, after, or concomitantly with the pharmaceutical compositions. Depending on the intended mode of administration, the pharmaceutical compositions can be in the form of solid, semi-solid or liquid dosage forms, such as, for example, tablets, suppositories, pills, capsules, powders, liquids, suspensions, lotions, creams, or gels, for example, in unit dosage form suitable for single administration of a precise dosage. For solid compositions, nontoxic solid carriers include, for example, pharmaceutical grades of mannitol, lactose, starch, magnesium stearate, sodium saccharin, tale, cellulose, glucose, sucrose, and magnesium carbonate. Non-limiting examples of pharmaceutically active agents suitable for combination with compositions of the disclosure include anti-infectives, i.e., aminoglycosides, antiviral agents, antimicrobials, anticholinergics/antispasmodics, antidiabetic agents, antihypertensive agents, antineoplastics, cardiovascular agents, central nervous system agents, coagulation modifiers, hormones, immunologic agents, immunosuppressive agents, and ophthalmic preparations.

Compounds can be delivered via liposomal technology. The use of liposomes as drug carriers can increase the therapeutic index of the compounds. Liposomes are composed of natural phospholipids, and can contain mixed lipid chains with surfactant properties (e.g., egg phosphatidylethanolamine). A liposome design can employ surface ligands for attaching to unhealthy tissue. Non-limiting examples of liposomes include the multilamellar vesicle (MLV), the small unilamellar vesicle (SUV), and the large unilamellar vesicle (LUV). Liposomal physicochemical properties can be modulated to optimize penetration through biological barriers and retention at the site of administration, and to reduce a likelihood of developing premature degradation and toxicity to non-target tissues. Optimal liposomal properties depend on the administration route: large-sized liposomes show good retention upon local injection, small-sized liposomes are better suited to achieve passive targeting PEGylation reduces the uptake of the liposomes by the liver and spleen, and increases the circulation time, resulting in increased localization at the inflamed site due to the enhanced permeability and retention (EPR) effect. Additionally, liposomal surfaces can be modified to achieve selective delivery of the encapsulated drug to specific target cells. Non-limiting examples of targeting ligands include monoclonal antibodies, vitamins, peptides, and polysaccharides specific for receptors concentrated on the surface of cells associated with the disease.

Non-limiting examples of dosage forms suitable for use in the disclosure include liquid, elixir, nanosuspension, aqueous or oily suspensions, drops, syrups, and any combination thereof. Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, plant cellulosic material and spheronization agents, and any combination thereof.

Compositions of the invention can be packaged as a kit. In some embodiments, a kit includes written instructions on the administration/use of the composition. The written material can be, for example, a label. The written material can suggest conditions methods of administration. The instructions provide the subject and the supervising physician with the best guidance for achieving the optimal clinical outcome from the administration of the therapy. The written material can be a label. In some embodiments, the label can be approved by a regulatory agency, for example the U.S. Food and Drug Administration (FDA), the European Medicines Agency (EMA), or other regulatory agencies.

### Dosing.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are liquids in vials or ampoules. Aqueous suspension compositions can be packaged in single-dose non-reclosable containers. Multiple-dose reclosable containers can be used, for example, in combination with a preservative. Formulations for parenteral injection can be presented in unit dosage form, for example, in ampoules, or in multi-dose containers with a preservative.

A dose can be expressed in terms of an amount of the drug divided by the mass of the subject, for example, milligrams of drug per kilograms of subject body mass. A compound described herein can be present in a composition in a range of from about 1 tug to about 2000 mg; from about 100 mag to about 2000 mg; from about 10 mg to about 2000 mg; from about 5 mg to about 1000 mg, from about 10 mg to about 500 mg, from about 50 mg to about 250 tug, from about 100 mg to about 200 mg, from about 1 mg to about 50 mag, from about 50 mg to about 100 mg, from about 100 mg to about 150 mug, from about 150 tug to about 200 mg, from about 200 mg to about 250 mg, from about 250 m; to about 300 mg, from about 300 mg to about 350 mag, from about 350 mag to about 400 mg, from about 400 mg to about 450 mg, from about 450 mg to about 500 mg, from about 500 mg to about 550 mg, from about 550 mg to about 600 mg, from about 600 tug to about 650 mg, from about 650 mg to about 700 mg, from about 700 mg to about 750 mg, from about 750 mg to about 800 tug, from about 800 mg to about 850 mg, from about 850 mg to about 900 mg, from about 900 mag to about 950 mg, or from about 950 mg to about 1000 mg.

In some embodiments, a compound is administered in an amount ranging from about 5 mg/kg to about 50 mg/kg, 250 mg/kg to about 2000 mg/kg, about 10 mg/kg to about 800 mg/kg, about 50 mg/kg to about 400 mg/kg, about 100 mg/kg to about 300 mg/kg, or about 150 mg/kg to about 200 mg/kg. In some embodiments, a compound described herein can be present in a composition in a range of from about 20 mg/kg to about 400 mg/kg. In some embodiments, a compound described herein can be present in a composition in a range of from about 20 mg/kg to about 240 mg/kg. In some embodiments, a compound described herein can be present in a composition in a range of from about 75 mg/kg to about 150 tug/kg. In some embodiments, a compound described herein can be present in a composition in a range of from about 75 mg/kg to about 150 mg/kg. In some embodiments, a compound described herein can be present in a composition in a range of from about 100 mg/kg to about 150 mg/kg.

In some embodiments, a compound described herein can be present in a composition in an amount of about 75 mg/kg. In some embodiments, a compound described herein can be present in a composition in an amount of about 100 mg/kg. In some embodiments, a compound described herein can be present in a composition in an amount of about 150 mg/kg. In some embodiments, a compound described herein can be present in a composition in an amount of about 200 mg/kg. In some embodiments, a compound described herein can be present in a composition in an amount of about 250 ng/kg. In some embodiments, a compound described herein can be present in a composition in an amount of about 400 mg/kg.

A compound described herein can be present in a composition in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 ng, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 ng, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 ng, about 1100 mg, about 1150 mg, about 1200 mg, about 1250 mag, about 1300 mg, about 1350 mg, about 1100 mg, about 1450 mg, about 1500 mg, about 1550 mg, about, 1600 mg, about 1650 mg, about 1700 mg, about 1750 mg, about 1800 mg, about 1850 ng, about 1900 mg, about 1950 mg, or about 2000 mg, about 2050 mg, about 2100 mg, about 2150 mg, about 2200 mg, about 2250 ng, about 2300 mg, about 2350 mg, about 2400 mg, about 2450 mg, about 2500 mg, about 2550 ng, about 2600 mg, about 2650 mg, about 2700 mg, about 2750 mag, about 2800 mg, about 2850 mg, about 2900 mg, about 2950 mg, or about 3000 mg.

In some embodiments, a compound described herein can be present in a composition in an amount of about 100 mg, about 120 mg, about 140 mg, about 160 mg, about 180 mg, about 200 mg, about 220 mg, about 240 mug, about 260 mg, about 280 mug, or about 300 mg. In some embodiments, a compound described herein can be present in a composition in an amount of about 150 mg. In some embodiments, a compound described herein can be present in a composition in an amount of about 170 mg. In some embodiments, a compound described herein can be present in a composition in an amount of about 280 mg. In some embodiments, a compound described herein can be present in a composition in an amount of about 300 mg.

### General Synthetic Schemes

The compounds of this invention can be synthesized according to the procedures described in WO2012149102. In addition, the compounds of this invention can be synthesized according to the following procedure of Schemes I-VIII, wherein the substituents are as defined for Formulas 1-6 above, except where further noted.

The following abbreviations are used:
RT room temperature
DCM, CH₂Cl₂ dichloromethane
TEA triethylamine
DMF dimethylformamide
DMSO dimethyl sulfoxide
K₂CO₃ potassium carbonate
ACN acetonitrile
TFA trifluoroacetic acid
FA formic acid
HCl hydrochloric acid
HOAc, AcOH acetic acid
THF tetrahydrofuran
CDCl₃ Deuterated chloroform
DMSO-d6 Deuterated dimethylsulfoxide
EtOAc, EA ethyl acetate
Na₂SO₄ sodium sulfate
mg milligram
g gran
ml, mL milliliter
h hour
min minutes
PE petroleum ether
MgSO₄ magnesium sulfate
H₂O water
Na₂CO₃ sodium carbonate
MeOH methanol
MeOH-d4 deuterated methanol
Boc tert-butyloxycarbonyl
EtOH ethanol
PE petroleum ether
CaCO₃ potassium phosphate
KOH potassium hydroxide
LiOH lithium hydroxide
TMSCl Trimethylsilyl chloride
BH₃·THF borane tetrahydrofuran
MeMgBr methyl magnesium bromide
TBAI tetra-n-butylammonium iodide
T3P propanephosphonic acid anhydride
SiO2 silica
N2 nitrogen
Eq equivalents
PBr₃ phosphorous tribromide
NH₃ ammonia

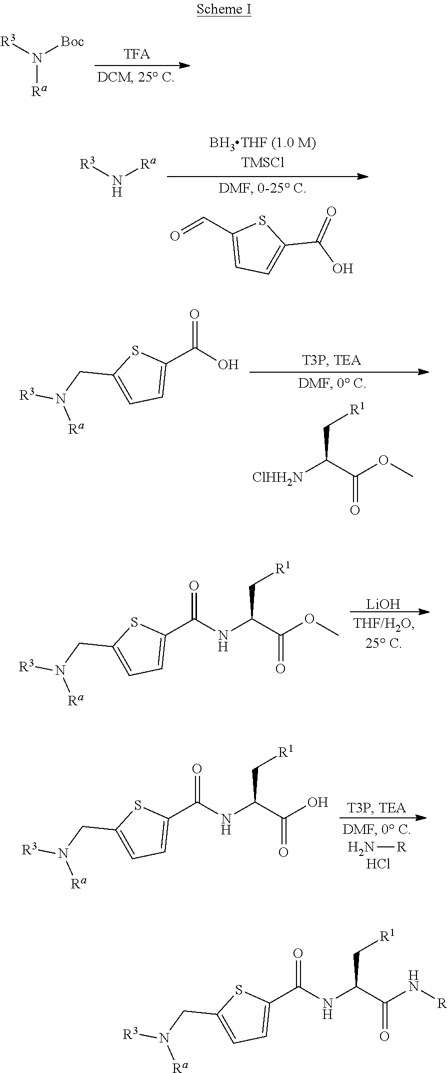

The compounds of the invention can be synthesized according to Scheme I. Boc protected secondary amines (e.g. where R^{a} is methyl) in DCM are deprotected, such as with TFA to afford the free amine. TMSCl and BH₃·THF are added to a mixture of the amine and 5-formylthiophene-2-carboxylic acid in a solvent such as DMF, at a temperature, e.g. 0° C., to afford 5-aminomethylthiophene-2-carboxylic acid. To a mixture of 5-aminomethylthiophene-2-carboxylic acid and a substituted methyl 2-aminopropanoate hydrochloride in a solvent such as DMF is added TEA and T₃P at a temperature, e.g. 0° C., to afford methyl 2-[[5-[substituted amino]methyl]thiophene-2-carbonyl]amino]-propanoate. Hydrolysis of the methyl 2-[[5-[substituted amino]methyl]thiophene-2-carbonyl]amino]-propan oate with LiOH affords the free acid. Coupling of the acid with a substituted amine, such as in the presence of base (e.g. TEA) and T₃P in a solvent such as DMF affords die compounds of the invention

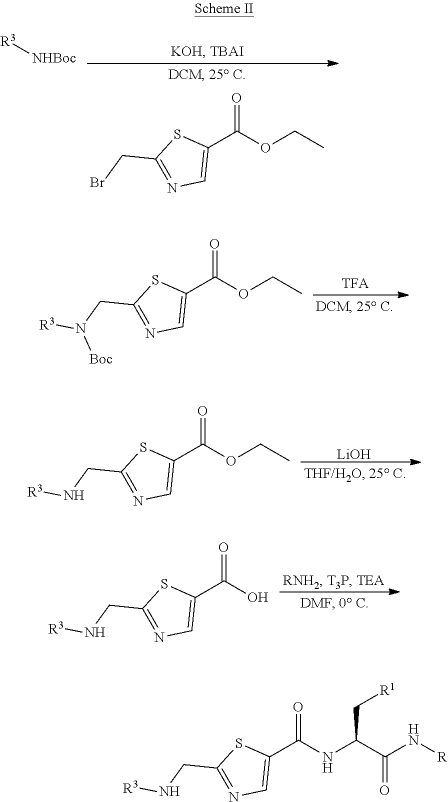

The thiazoles of the invention can be synthesized according to Scheme II. Boc protected primary amines (e.g. where R^{a} is H) in DCM are coupled with 2-bromomethylthiazolyl-2-alkyl ester, such as with KOH and TPAI to provide the Boc-aminomethylthiazolyl-2-alkyl ester. The protected amino is deprotected, such as with TFA to afford the free amine. Hydrolysis of the alkyl ester, such as with LiOH affords the free acid. Coupling of the acid with a substituted amine, such as in the presence of base (e.g. TEA) and T₃P in a solvent such as DMF affords the compounds of the invention.

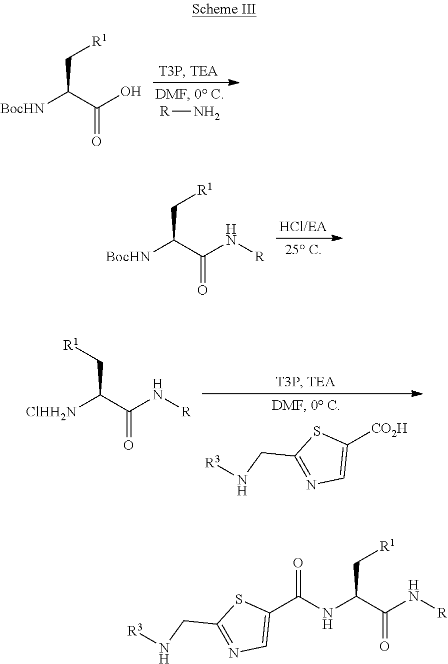

The thiazoles of the invention can be synthesized according to Scheme III, Boc protected primary amine acids (e.g. where R^{a} is H) are coupled with amines, such as in the presence of base (e.g. TEA) and T₃P in a solvent such as DMF affords the R-substituted amides. The protected compounds are deprotected, such as with HCl to afford the free amine. Coupling of a thiazolyl acid with the amine, such as in the presence of base (e.g. TEA) and T₃P in a solvent such as DMF affords the compounds of the invention.

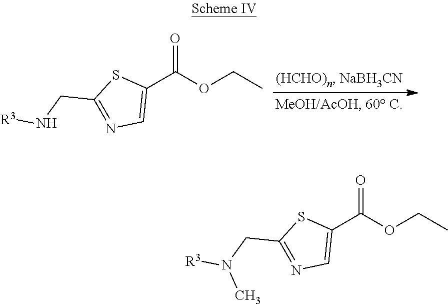

The thiazole intermediate of the invention can be synthesized according to Scheme IV. Secondary amines are alkylated, such as with formaldehyde in the presence of sodium cyano borohydride, in a solvent such as MeOH and AcOH.

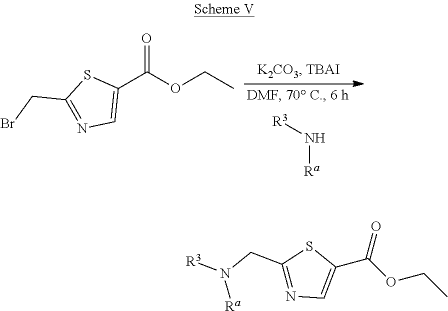

The thiazole ester intermediates of the invention can be synthesized according to Scheme V. Where R³ and R^{a} forms a nitrogen containing heterocycles or heteroaryls) in DMF are coupled with 2-bromomethylthiazolyl-2-alkyl ester, such as with K₂CO₃ and TPAI to provide the substituted aminomethylthiazolyl-2-alkyl ester.

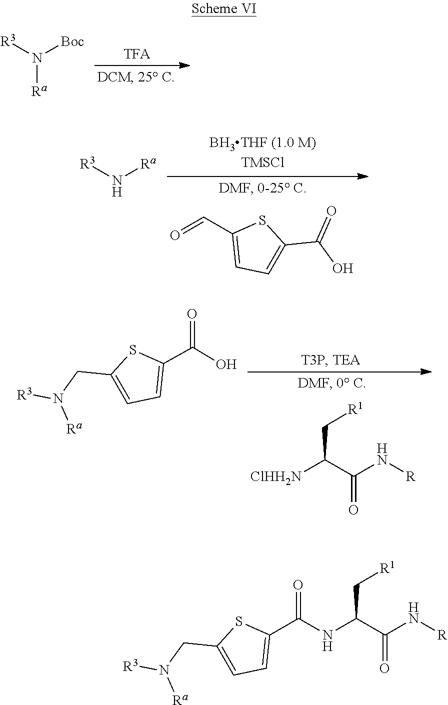

The compounds of the invention can be synthesized according to Scheme I. Boc protected secondary amines (e.g. where R^{a} is methyl) in DCM are deprotected, such as with TFA to afford the free amine. TMSCl and BH₃. THF are added to a mixture of the amine and 5-formylthiophene-2-carboxylic acid in a solvent such as DMF, at a temperature, e.g. 0° C., to afford 5-aminomethylthiophene-2-carboxylic acid. To a mixture of 5-aminomethylthiophene-2-carboxylic acid and a substituted methyl 2-aminopropanoate hydrochloride in a solvent such as DMF is added TEA and T₃P at a temperature, e.g. 0° C., to afford the compounds of the invention.

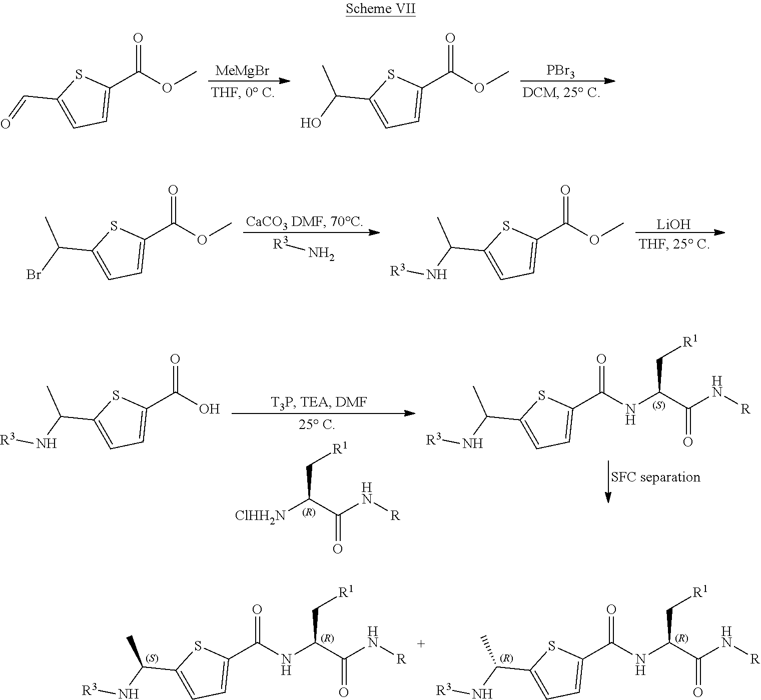

The compounds of the invention can be synthesized according to Scheme VII. Conversion of methyl 5-formylthiophene-2-carboxylate to the hydroxymethyl derivative results from treatment of the MeMgBr Grignard reagent in a solvent, such as THF at a temperature below RT, such as at 0° C. The methyl 5-(1-hydroxyethyl) thiophene-2-carboxylate is halogenated such as with PBr₃ in a solvent such as DCM to afford the methyl 5-(1-bromoethyl) thiophene-2-carboxylate. Treatment of the bromoethyl-thiophene with a substituted-amine and a base, such as CaCO₃ at a temperature above RT, such as at about 70° C., affords the substituted amine thienyl carboxylate Hydrolysis of the ester to the corresponding acid is achieved with LiOH. The thiophene-2-carboxylic acid is coupled with the substituted-2-amino-propanamide, such as with T₃P and TEA, at a temperature below RT, such as at about 0° C., afford the desired compound.

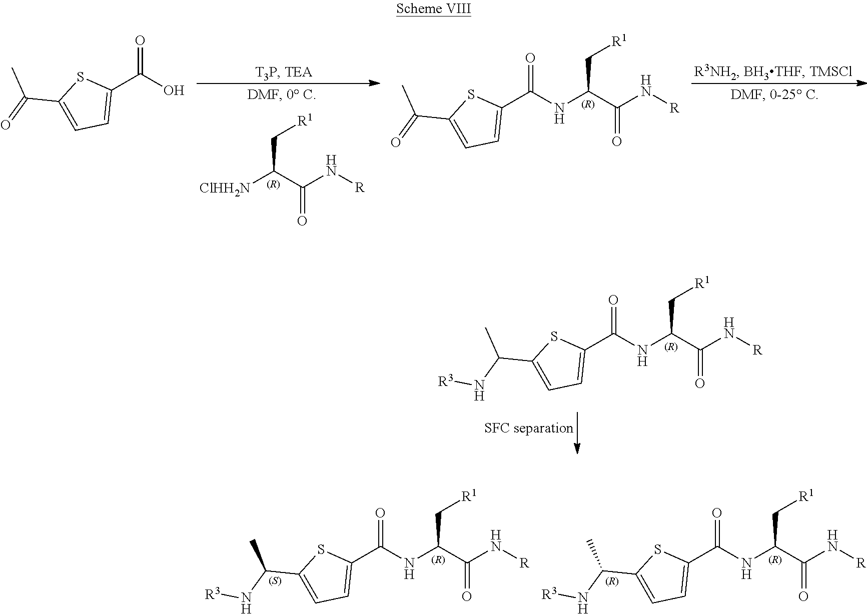

The compounds of the present disclosure can be synthesized according to Scheme VIII. A substituted aminopropanamide is coupled with 5-acetylthiophene-2-carboxylic acid, such as with TEA and T₃P, in an organic solvent such as DMF, at a temperature below RT, such as 0° C. to afford 5-acetyl-2-oxo-ethyl]thiophene-2-carboxamide. The 5-acetyl-2-oxo-ethyl]thiophene-2-carboxamide is coupled with a substituted anine, such as with borane and TMSCl, in an organic solvent such as DTF, at a temperature below RT, such as 0° C. to afford the compounds of the invention. Racemic mixtures can be separated by chiral chromatography, to afford the title individual enantiomers.

The following examples contain detailed descriptions of the methods of preparation of compounds of Formulas 1-6. These detailed descriptions fall within the scope, and serve to exemplify, the above described General Synthetic Procedures which form part of the invention. These detailed descriptions are presented for illustrative purposes only and are not intended as a restriction on the scope of the invention. All parts are by weight and temperatures are in Degrees centigrade unless otherwise indicated.

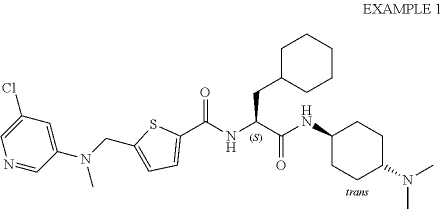

### 5-[[(5-chloro-3-pyridyl)-methyl-amino]methyl]-N-[(1S)-1-(cyclohexyl methyl)-2-[[4-(dimethylamino)cyclohexyl]amino]-2-oxo-ethyl]thiophene-2-carboxamide

### Step 1—5-chloro-N-methyl-pyridin-3-amine

To a solution of tert-butyl N-(5-chloro-3-pyridyl)-N-methylcarbamate (2.00 g, 8.24 mmol, 1.0 eq) in DCM (16 mL) was added TFA (12.32 g 108.05 mmol, 8 mL, 13.11 eq), then the mixture was stirred at 25° C. for 1 h under N₂ atmosphere. The reaction mixture was poured into saturated Na₂CO₃ (100 ml), and extracted with DCM (3×50 mL), then the combined organic layers were washed with saturated brine (1×50 mL), dried with anhydrous Na₂SO₄, filtered and concentrated under reduced pressure to afford 5-chloro-N-methyl-pyridin-3-amine (1.00 g, 7.01 mmol, 85.11% yield) as a light yellow solid LC-MS (ES+, m/z): 142.1 [(M+H)+].

### Step 2—5-[[(5-chloro-3-pyridyl)-methyl-amino]methyl]thiophene-2-carboxylic acid

To a mixture of 5-chloro-N-methyl-pyridin-3-anine (Step 1, 103 g 7.22 mmol, 1.0 eq) and 5-formylthiophene-2-carboxylic acid (3.38 g, 21.67 mmol, 3.0 eq) in DMF (5 mL) was added TMSCl (3.92 g, 36.12 mmol, 4.58 mL, 5.0 eq) and BH₃·THF (1 M, 14.45 mL, 2.0 eq) at 0° C., then the mixture was stirred at 25° C. for 16 h tinder N₂ atmosphere. The reaction mixture was poured into saturated Na₂CO₃ (80 mL), aq. HCl (1M) was added to a to pH=6.0, and the aqueous layer was extracted with EtOAc (3×40 mL), then the combined organic layers were washed with saturated brine (40 mL), dried with anhydrous Na₂SO₄, filtered and concentrated under reduced pressure to give a residue. The residue was purified by prep-HPLC (column: Phenomenex luna C₁₈ 250*50 mm*10 μm; mobile phase: [water (0.04% HCl)-ACN]; B %: 20%-50%, 10 imin) to afford 5-[[(5-chloro-3-pyridyl)-methyl-amino]methyl]thiophene-2-carboxylic acid (600.0 mg, 212 mmol, 29.38% yield) as a light yellow solid. LC-MS (ES+, m/z): 282.1 [(M+H)+].

### Step 3—methyl (2S)-2-[[5-[[(5-chloro-3-pyridyl)-methyl-amino]methyl]thiophene-2-carbonyl]amino]-3-cyclohexyl-propanoate

To a mixture of 5-[[(5-chloro-3-pyridyl)-methyl-amino]methyl]thiophene-2-carboxylic acid (Step 2, 600.0 mg, 2.12 mmol, 1.0 eq) and methyl (2S)-2-amino-3-cyclohexyl-propanoate hydrochloride (564.6 mg, 2.55 mmol, 1.2 eq) in DMF (5 mL) was added TEA (1.07 g, 10.61 mmol, 1.48 mL, 5.0 eq) and T₃P (2.70 g, 4.24 mmol, 2.52 mL, 50% purity, 2.0 eq) at 0° C. The mixture was stirred at 0° C. for 0.5 h under N₂ atmosphere. The reaction mixture was poured into H₂O (50 mL), and then extracted with EtOAc (3×30 mL). The combined organic layers were washed with saturated brine (30 mL), dried with anhydrous Na₂SO₄, filtered, and concentrated under reduced pressure to give a residue. The residue was purified by prep-TLC (SiO₂, PE:EA=1:1, Rf=0.6) to afford methyl (2S)-2-[[5-[[(5-chloro-3-pyridyl)-methyl-amino]methyl]thiophene-2-carbonyl]amino]-3-cycohexyl-propanoate (700.0 mg, 1.56 mmol, 73.31% yield) as a white solid, LC-MS (EST, m/z): 149.1 [(M+H)+].

### Step 4—(2S)-2-[[5-[[(5-chloro-3-pyridyl)-methyl-amino]methyl]thiophene-2-carbonyl]amino]-3-cyclohexyl-propanoic acid

To a solution of methyl (2S)-2-[[5-[[(5-chloro-3-pyridyl)-methyl-amino]methyl]thiophene-2-carbonyl]amino]-3-cyclohexyl-propanoate (Step 3, 700.0 mg, 1.56 mmol, 1.0 eq) in THF (3 mL) was added LiOH (3 M, 1.56 mL, 3.0 eq), then the mixture was stirred at 25° C. for 2 h under N₂ atmosphere. The reaction mixture was adjusted to pH=˜4 with 3N aq. HCl (10 mL), then extracted with EtOAc (3×30 mL). The combined organic layers were washed with saturated brine (30 mL, dried over anhydrous Na₂SO₄, filtered and concentrated tinder reduced pressure to afford (2S)-2-[[5-[[(5-chloro-3-pyridyl)-methyl-amino]methyl]thiophene-2-carbonyl]amino]-3-cyclohexyl-propanoic acid (538.0 mg, crude) as white solid. LC-MS (ES+, m/z): 435.1 [(M+H)+].

### Step 5—5-[[(5-chloro-3-pyridyl)-methyl-amino]methyl]-N-[(1S)-1-(cyclohexyl methyl)-2-[[4-(dimethylamino)cyclohexyl]amino]-2-oxo-ethyl]thiophene-2-carboxamide (Compound 298)

To a mixture of (2S)-2-[[5-[[(5-chloro-3-pyridyl)-methyl-amino]methyl]thiophene-2-carbonyl]amino]-3-cyclohexyl-propanoic acid (Step 4, 160.0 mg, 367.00 mol, 1.0 eq) and N⁴,N⁴-dimethylcyclohexane-1,4-diamine hydrochloride (78.7 mg 440.40 μmol, 1.2 eq) in DMF (2 mL) was added TEA (185.6 mg, 1.84 mmol 255.41 μL, 5.0 eq) and T₃P (467.0 mg, 734.00 μmol, 436.53 μL, 50% purity, 2.0 eq) at 0° C. The mixture was stirred at 0° C. for 0.5 b under N₂ atmosphere. The reaction mixture was poured into H₂O (50 mL), the aqueous phase was extracted with EtOAc (3×30 mL). The combined organic layers were washed with saturated brine (30 mL), dried with anhydrous Na₂SO₄, filtered and concentrated under reduced pressure to give a residue. The residue was purified by prep-HPLC (column: Phenomenex Luna C₁₈ 150*30 mm*5 μm; mobile phase. [water (0.2% FA)-ACN]; B %: 15%-50%, 8 min) to afford 5-[[(5-chloro-3-pyridyl)-methyl-amino]methyl]-N-[(1S)-1-(cyclohexylmethyl)-2-[[4-(dimethylamino) cyclohexyl]amino]-2-oxo-ethyl]thiophene-2-carboxamide (28.0 mg, 49.98 μmol, 13.62% yield, 100% purity) as a white solid. LC-MS (ES+, m/z): 559.1 [(M+H)+]. ¹H NMR (400 MHz, DMSO-d₆) δ ppm 8.38 (br d, J=7.95 Hz, 1H) 8.24 (s, 1H) 8.13 (d, J=2.45 Hz, 1H) 7.87 (br d, J=1.71 Hz, 2H) 7.76 (br d, J=1.96 Hz, 1H) 7.25 (d, J=1.96 Hz, 1H) 7.02 (br d, J=3.06 Hz, 1H) 4.83 (s, 2H) 1.34-4.42 (m, 1H) 3.43 (br s, 1H) 3.02 (s, 3H) 2.22 (br s, 6H) 1.71-1.84 (m, 4H) 1.52-1.70 (m, 8H) 1.43-1.50 (m, 2H) 1.05-1.26 (m, 8H) 0.80-0.92 (n, 3H) ¹H NMR (400 MHz, METHANOL-d₆) δ ppm 8.55 (s, 1H) 8.05 (d, J=2.45 Hz, 1H) 7.84 (d, J=1.59 Hz, 1H) 7.65 (d, J=3.67 Hz, 1H) 7.27 (t, J=2.14 Hz, 1H) 7.02 (d, J=3.55 Hz, 1H) 4.50 (dd, J=9.54, 5.75 Hz, 1H) 3.62 (br t, J=10.45 Hz, 1H) 3.11 (s, 3H) 2.72-2.83 (m, 1H) 2.59 (br s, 6H) 2.02 (br s, 4H) 1.59-1.79 (m, 8H) 1.43-1.52 (m, 2H) 1.35 (br s, 3H) 1.16-1.26 (m, 3H) 0.86-1.03 (m, 2H).

### Example 2

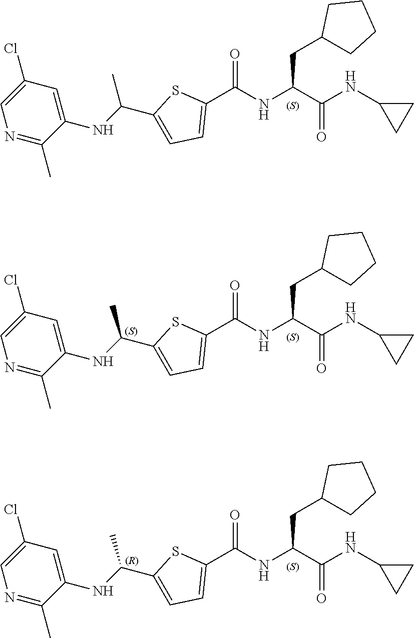

### 5-[1-[(5-chloro-2-methyl-3-pyridyl) amino]ethyl]-N-[(1S)-1-(cyclopentylmethyl)-2-(cyclopropylamino)-2-oxo-ethyl]thiophene-2-carboxamide

### 5-[(1R)-1-[(5-chloro-2-methyl-3-pyridyl)amino]ethyl]-N-[(1S)-1-(cyclopentylmethyl)-2-(cyclopropylamino)-2-oxo-ethyl]thiophene-2-carboxamide

### Step 1—methyl 5-(1-hydroxyethyl) thiophene-2-carboxylate

To a solution of methyl 5-formylthiophene-2-carboxylate (3.00 g, 17.63 mmol, 1.00 eq) in THF (30 mL) was added MeMgBr (3 M, 7.64 mL, 1.30 eq) at 0° C., the reaction was stirred at 0° C. for 30 min. Water (100 mL) was added to the reaction and the aqueous phase was extracted with EtOAc (3×100 mL). The combined organic phase was washed with brine (100 mL), dried with anhydrous Na₂SO₄ filtered and concenrated in vacuum. The residue was purified by column chromatography (SiO₂, PE:EtOAc=100:1 to 5:1, TM/R_{f}=0.5) to afford methyl 5-(1-hydroxyethyl) thiophene-2-carboxylate (2.00 g, 10.74 mmol, 60.92% yield) as a white solid. LC-MS (ES⁺, m/z):187.1 [(M+H)⁺]

### Step 2—methyl 5-(1-bromoethyl) thiophene-2-carboxylate

To a mixture of methyl 5-(1-hydroxyethyl) thiophene-2-carboxylate (Step 1, 1.40 g, 7.52 mmol, 1.00 eq) in DCM (14 mL) was added PBr₃ (6.10 g, 22.55 mmol, 3.00 eq) at 25° C., and the reaction was stirred at 25° C. for 2 hr. DCM (35 mL) was added to the reaction. The mixture was purified by column chromatography (SiO₂, PE:EtOAc=100:1) to afford methyl 5-(1-bromoethyl) thiophene-2-carboxylate (2.44 g, crude) as a yellow solid.

### Step 3—methyl 5-[1-[(5-chloro-2-methyl-3-pyridyl) amino]ethyl]thiophene-2-carboxylate

To a mixture of methyl 5-(1-bromoethyl)thiophene-2-carboxylate (Step 2, 2.44 g, 9.81 mmol, 100 eq) and 5-chloro-2-methyl-pyridin-3-amine (1.68 g, 11.77 mmol, 1.20 eq) in DMF (24 mL) were added CaCO₃ (4.91 g, 49.03 mmol, 5.00 eq) at 25° C., and the reaction was stirred at 70° C. for 4 hr. To the reaction was added water (25 mL), and the aqueous phase was extracted with EtOAc (3×25 mL). The combined organic phase was dried with anhydrous Na₂SO₄, filtered and concentrated in vacuum. The residue was purified by prep-TLC (SiO₂, PE:EtOAc=1:1, TM/R_{f}=0.60) to afford methyl 5-[1-[(5-chloro-2-methyl-3-pyridyl)amino]ethyl]thiophene-2-carboxylate (490.0 mg, 1.58 mmol, 16.08% yield) as a yellow solid. LC-MS (ES⁺, m/z):311.0 [(M+H)⁺]

### Step 4—5-[1-[(5-chloro-2-methyl-3-pyridyl) amino]ethyl]thiophene-2-carboxylic acid

To a mixture of methyl 5-[1-[(5-chloro-2-methyl-3-pyridyl)amino]ethyl]thiophene-2-carboxylate (Step 3, 326.00 mg, 1.05 mmol, 1.00 eq) in THF (1 mL), H₂O (0.5 mL) was added LiOH·H₂O (88.00 mg, 2.10 mmol, 2.00 eq) at 25° C., and the reaction was stirred at 25° C. for 6 hr. To the reaction was added water (10 mL), and the pH value was adjust to pH=3 with HC (2N). The aqueous phase was extracted with EtOAc (3×10 mL). The combined organic phase was dried with anhydrous Na₂SO₄, filtered and concentrated in vacuum to afford 5-[1-[(5-chloro-2-methyl-3-pyridyl)amino]ethyl]thiophene-2-carboxylic acid (209.0 mg, crude) as a yellow solid. LC-MS (ES⁺, m/z):297.0 [(M+H)⁺].

### Step 5—5-[1-[(5-chloro-2-methyl-3-pyridyl) amino]ethyl]-N-[(1S)-1-(cyclopentylmethyl)-2-(cyclopropylamino)-2-oxo-ethyl]thiophene-2-carboxamide (Compound 5)

To a mixture of 5-[1-[(5-chloro-2-methyl-3-pyridyl)amino]ethyl]thiophene-2-carboxylic acid (129.00 mg, 434.68 mmol, 1.00 eq) and (2S)-2-amino-3-cyclopentyl-N-cyclopropyl-propanamide; hydrochloride (12140 mg, 52.61 mmol, 1.20 eq) in DMF (1.3 mL) were added TEA (351.90 mg, 3.48 mmol, 484.02 μL, 8.00 eq) and T₃P (829.80 mg, 130 mmol, 775.55 μL, 50% purity, 3.00 eq) at 0° C. The reaction was stirred at 25° C. for 6 hr. To the reaction was added water (10 mL), and the aqueous phase was extracted with EtOAc (3×10 mL). The combined organic phase was dried with anhydrous Na₂SO₄, filtered and concentrated in vacuum. The residue was purified by prep-HPLC (column: Waters Xbridge BEH C18 100×30 mm×10 um; mobile phase: [water (NH₄HCO₃)-ACN]; B %: 40%-70%, 10 min) to afford 5-[1-[(5-chloro-2-methyl-3-pyridyl)amino]ethyl]-N-[(1S)-1-(cyclopentylmethyl)-2-(cyclopropylamino)-2-oxo-ethyl]thiophene-2-carboxamide (13.0 mg, 27.37 μmol, 6.30% yield, 100% purity) as a white solid.

### Step 6—Isolation of 5-[(1S)-1-[(5-chloro-2-methyl-3-pyridyl)amino]ethyl]-N-[(1S)-1-(cyclopentylmethyl)-2-(cyclopropylamino)-2-oxo-ethyl]thiophene-2-carboxamide and 5-[(1R)-1-[(5-chloro-2-methyl-3-pyridyl)amino]ethyl]-N-[(1S)-1-(cyclopentylmethyl)-2-(cyclopropylamino)-2-oxo-ethyl]thiophene-2-carboxamide (Compounds 8-9)

5-[1-[(5-chloro-2-methyl-3-pyridyl)amino]ethyl]-N-[(1S)-1-(cyclopentylmethyl)-2-(cyclopropylamino)-2-oxo-ethyl]thiophene-2-carboxamide (100 mg) was further separated by prep-SFC (column: DAICEL CHIRALPAK AD (250 mM*30 mm, 10 um); mobile phase: [0.1% NH₃ H₂O ETOH]; B %: 60%-60%, 15 min) to afford the title compound (23.4 mg 49.26 μmol, 26.00% yield) as a white solid.

5-[1-[(5-Chloro-2-methyl-3-pyridyl)amino]ethyl]-N-[(1S)-1-cyclopentylmethyl)-2-(cyclopropylamino)-2-oxo-ethyl]thiophene-2-carboxamide 100 mg) was further separated by prep-SFC (column: DAICEL CHIRALPAK AD (250 mm*30 mm, 10 um); mobile phase: [0.1% NH₃ H₂O ETOH]; B %: 60%3-0%, 15 min) to afford the title compound (23.4 mg, 49.26 μmol, 26.00% yield) as a white solid.

### Example 3

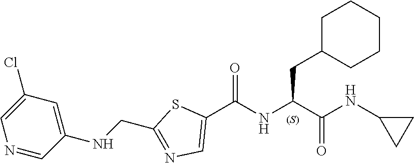

### 2-[[(5-chloro-3-pyridyl)amino]methyl]-N-[(1S)-1-(cyclohexylmethyl)-2-(cyclopropylamino)-2-oxo-ethyl]thiazole-5-carboxamide

### Step 1—ethyl 2-[[tert-butoxycarbonyl-(5-chloro-3-pyridyl)amino]methyl]thiazole-5-carboxylate

To a mixture of tert-butyl N-(5-chloro-3-pyridyl)carbamate (850.0 mg, 3.72 mmol, 1.0 eq) and ethyl 2-(bromomethyl)thiazole-5-carboxylate (929.6 mg, 3.72 mmol, 1.0 eq) in DCM (6 mL) was added KOH (625.6 mg, 11.15 mmol 3.0 eq) and TBAI (686.4 mg, 1.86 mmol, 0.5 eq), then the mixture was stirred at 25° C. for 1 h tinder N₂ atmosphere. The reaction mixture was poured into H₂O (50 mL), then extracted with DCM (30 mL). The combined organic layers were washed with saturated brine (30 ml), dried with anhydrous Na₂SO₄, filtered and concentrated under reduced pressure to give a residue. The residue was purified by prep-TLC (SiO₂, PE:EA=21, Rf=0.3) to afford ethyl 2-[[tert-butoxycarbonyl-(5-chloro-3-pyridyl)amino]methyl]thiazole-5-carboxylate (1.00 g, 2.51 mmol, 67.62% yield) as a yellow oil. LC-MS (ES+, m/z): 397.1 [(M+H)+].

### Step 2—ethyl 2-[[(5-chloro-3-pyridyl)amino]methyl]thiazole-5-carboxylate

To a solution of ethyl 2-[[tert-butoxycarbonyl-(5-chloro-3-pyridyl)amino]methyl]thiazole-5-carboxylate (Step 1, 700.0 mg, 1.76 mmol, 1.0 eq) in DCM (8 mL) was added TFA (6.16 g, 54.03 mmol, 4 mL, 30.71 eq), then the mixture was stirred at 25° C. for 2 h under N₂ atmosphere. The reaction mixture was poured into saturated Na₂CO₃ (30 ml), and extracted with DCM (3×20 mL). The combined organic layers were washed with saturated brine (20 mL), dried with anhydrous Na₂SO₄, filtered and concentrated under reduced pressure to give a residue. The residue was purified by prep-TLC (SiO₂, PE:EA=2:1, Rf=0.5) to afford ethyl 2[[(5-chloro-3-pyridyl)amino]-methyl]thiazole-5-carboxylate (400.0 mg, 1.48 mmol, 84.30% yield) as a yellow oil. LC-MS (ES+, m/z): 297.1 [(M+H)+].

### Step 3—2-[[(5-chloro-3-pyridyl)amino]methyl]thiazole-5-carboxylic acid

To a solution of ethyl 2-[[(5-chloro-3-pyridyl)amino]methyl]thiazole-5-carboxylate (Step 2, 500.0 mg, 1.68 mmol, 1.0 eq) in THE (3 mL) was added LiOH (3 M, 1.68 mL, 3.0 eq), then the mixture was stirred at 25° C. for 2 h under N₂ atmosphere. The reaction mixture was adjusted to pH=˜4 with 3N aq. HCl (10 mL), then extracted with EA (3×20 mL). The combined organic layers were washed with saturated brine (20 m), dried over anhydrous Na₂SO₄, filtered and concentrated under reduced pressure to afford 2-[[(5-chloro-3-pyridyl amino]methyl]thiazole-5-carboxylic acid (400.0 mg, crude) as a yellow solid. LC-MS (ES+, m/z): 269.0 [(M+H)+].

### Step 4—2-[[(5-chloro-3-pyridyl)amino]methyl]-N-[(1S)-1-(cyclohexylmethyl)-2-(cyclopropylamino)-2-oxo-ethyl]thiazole-5-carboxamide (Compound 299)

To a mixture of 2-[[(5-chloro-3-pyridyl)amino]methyl]thiazole-5-carboxylic acid (Step 3, 160.0 mg, 593.24 μmol, 1.0 eq) and (2S)-2-amino-3-cyclohexyl-N-cyclopropyl-propanamide hydrochloride (175.6 mg, 711.89 μmol, 1.2 eq.) in DMF (3 mL) was added TEA (300.1 mg, 2.97 mmol, 412.85 μL, 5.0 eq) and T₃P (755.0 mg, 1.19 mmol, 705.63 μL, 50% purity, 2.0 eq) at 0° C., The mixture was stirred at 0° C. for 0.5 h under N₂ atmosphere. The reaction mixture was poured into H₂O (50 mL), then extracted with eA (3×30 mL), then the combined organic layers were washed with saturated brine (30 mL), dried with anhydrous Na₂SO₄, filtered and concentrated under reduced pressure to give a residue. The residue was purified by prep-HP-LC (column: Phenomenex C₁₈ 75*30 mm*3 um, mobile phase: [water (NH₃H₂O+NH₄HCO₃)-ACN]; B %: 30%-65%, 8 min) to afford 2-[[(5-chloro-3-pyridyl)amino]methyl]-N-[(1S)-1-(cyclohexylmethyl)-2-(cyclopropylamino)-2-oxo-ethyl]thiazole-5-carboxamide (25.4 mg, 54.59 μmol, 9.20% yield, 99.3% purity) as a white solid. LC-MS (ES+, m/z) 461.1 [(M+H)⁺]

The preparation of the following compound in Table 3 follows the procedure of that described above:

TABLE B

| Cpd | | | LC-MS | Yield & |
|---|---|---|---|---|
| # | Structure | IUPAC | (ES+, m/z) | status |
| 294 | 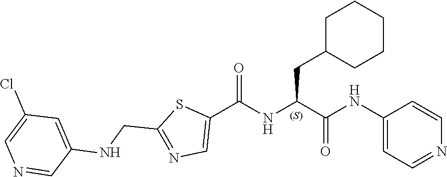 | 2-[[(5-chloro-3- pyridyl)amino]methyl]- N-[(1S)-1- (cyclohexylmethyl)-2- oxo-2-(4- pyridylamino)ethyl] thiazole-5-carboxamide | 498.1 [(M + H)⁺] | 29.9 mg, 13.2% white solid |

### Example 4

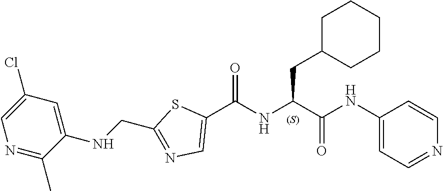

### 2-[[(5-chloro-2-methyl-3-pyridyl)amino]methyl]-N-[(1S)-1-(cyclohexylmethyl)-2-oxo-2-(4-pyridylamino)ethyl]thiazole-5-carboxamide

### Step 1—tert-butyl N-[(1S)-1-(cyclohexylmethyl)-2-oxo-2-(4-pyridylamino)ethyl]carbamate

To a solution of (2S)-2-(tert-butoxycarbonylamino)-3-cyclohexyl-propanoic acid (5.00 g, 18.43 mmol, 1.0 eq) and pyridine-4-amine (1.73 g, 18.43 mmol, 3.10 mL, 1.0 eq) in DMF (100 mL) was added TEA (9.32 g, 92.13 mmol, 12.82 mL, 5.0 eq) and T₃P (11.73 g, 36.85 mmol, 10.96 mL, 2.0 eq) at 0° C. The mixture was stirred at 0° C. for 0.5 h under N₂ atmosphere. The reaction mixture was poured into water (100 mL), then extracted with EA (3×50 mL). The combined organic layers were washed with saturated brine (50 mL), dried over anhydrous Na₂SO₄, filtered and concentrated under reduced pressure to give a residue. The residue was purified by column chromatography (SiO₂, PE/EA=20/1 to 0/1) to afford tert-butyl N-[(1S)-1-(cyclohexylmethyl)-2-oxo-2-(4-pyridylamino)ethyl]carbamate (3.50 g, 10.07 mmol, 54.67% yield) as a white solid. LC-MS (ES⁺, m/z):348.2 [(M+H)⁺]

### Step 2—(2S)-2-amino-3-cyclohexyl-N-(4-pyridyl)propanamide hydrochloride

To a solution of tert-butyl N-[(1S)-1-(cyclohexylmethylamino) ethyl]carbamate (Step 1, 2.50 g, 7.20 mmol, 1.0 eq) in EtOAc was added HCl/EtOAc (4 M, 10 mL) at 0° C. The reaction mixture was stirred at 25° C. for 4 hr under N₂ atmosphere. The reaction mixture was concentrated under reduced pressure to give a residue. The crude (S)-2-amino-3-cyclohexyl-N-(pyridin-4-yl)propanamide hydrochloride (1.60 g, 5.64 mmol, 78.35% yield) was used in the next step without further purification. LC-MS (ES⁺, m/z):248.1 [(M+H)⁺]

### Step 3—N-[(1S)-1-(cyclohexylmethyl)-2-(oxetan-3-ylamino)-2-oxo-ethyl]-5-[[3-(trifluoromethyl)anilino]methyl]thiophene-2-carboxamide (Compound 300)

To a solution of 2-[[(5-chloro-2-methyl-3-pyridyl)amino]methyl]thiazole-5-carboxylic acid (Step 2, 80.0 Mg, 281.95 μmol, 1.0 eq) and (2S)-2-amino-3-cyclohexyl-N-(4-pyridyl)propanamide hydrochloride (80.0 mg, 281.95 μmol, 10 eq) in DMF (200 mL) was added TEA (142.6 mg, 1.41 mmol, 196.22 μL, 5.0 eq) and T₃P (179.4 mg, 563.91 μmol, 167.69 μL, 2.0 eq) at 0° C., The reaction mixture was stirred at 0° C. for 0.5 h under N₂ atmosphere. The reaction mixture was poured into water (30 mL), then extracted with EtOAc (3×10 mL). The combined organic layers were washed with saturated brine (10 mL), dried over anhydrous Na₂SO₄, filtered and concentrated under reduced pressure to give a residue. The residue was purified by prep-HPLC (column: Waters Xbridge Prep OBD C₁₈ 150*40 mm*10 μm; mobile phase: [water (0.05% NH₃ H₂O+10 mM NH₄HCO₃)-ACN]; B %: 20%-55%, 8 min) to afford 2-[[(5-chloro-2-methyl-3-pyridyl)amino]methyl]-N-[(1S)-1-(cyclohexylmethyl)-2-oxo-2-(4-pyridylamino)ethyl]thiazole-5-carboxamide (27.3 mg, 53.21 μmol, 18.87% yield, 100% purity) as a white solid. LC-MS (ES+, m/z)

The preparation of the following compound in Table C follows the above procedure:

TABLE C

| Cpd | | | LC-MS | Yield & |
|---|---|---|---|---|
| # | Structure | IUPAC | (ES+, m/z) | status |
| 287 | 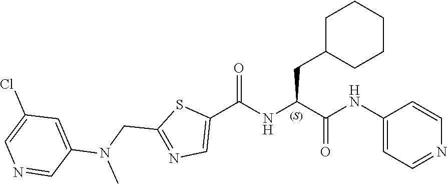 | 2-[[(5-chloro-3- pyridyl)-methyl-amino] methyl]-N-[(1S)-1- (cyclohexylmethyl)-2- oxo-2-(4- pyridylamino)ethyl] thiazole-5-carboxamide | 513.0 [(M + H)⁺] | 24.3 mg, 26.8% as white solid |

### Example 5

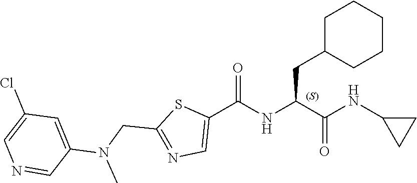

### 2-[[(5-Chloro-3-pyridyl)-methyl-amino]methyl]-N-[(1S)-1-(cyclohexylmethyl)-2-oxo-2-(4-pyridylamino)ethyl]thiazole-5-carboxamide (Compound 301)

To a solution of 2-[[(5-chloro-3-pyridyl)-methyl-amino]methyl]thiazole-5-carboxylic acid (180.0 mg, 634.40 umol, 1.0 eq) and (2S)-2-amino-3-cyclohexyl-N-cyclopropyl-propanamide hydrochloride (156.5 mg, 634.40 μmol, 1.0 eq) in DMF (2 mL) was added TEA (320.9 mg, 3.17 mmol, 441.50 L, 5.0 eq) and T₃P (807.4 mg, 1.27 mmol, 754.59 L, 50% purity, 2.0 eq) at 0° C. The mixture was stirred at 0° C. for 0.5 hr under N atmosphere. The reaction mixture was poured into H₂O (30 mL), then extracted with EA (3×10 mL). The combined organic phase was washed with saturated brine (10 mL), dried with anhydrous Na₂SO₄, filtered and concentrated in vacuum to give a residue. The residue was purified by prep-HPLC (column: Waters Xbridge Prep OBD Cis 150*40 mm*10 μm; mobile phase:water (NH₃H₂O+NH₄HCO₃)-ACN]; B %:40%-75%, 8 min) to afford 2-[[(5-chloro-3-pyridyl)-methyl-amino]methyl]N-[(1S)-1-(cyclohexylmethyl)-2-(cyclopropylamino)-2-oxo-ethyl]thiazole-5-carboxamide (26.8 mg, 54.78 umol, 8.63% yield, 97.3% purity) as a white solid. LC-MS (ES+, m/z) 513.0.

The preparation of the following compounds in Table D follows the above procedure

TABLE D

| Cpd | | | LC-MS | Yield & |
|---|---|---|---|---|
| # | Structure | IUPAC | (ES+, m/z) | status |
| 288 | 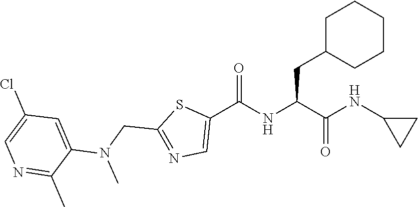 [SMILES: Cc1ncc(Cl)cc1N(C)Cc1ncc(C(=O)N[C@@H](CC2CCCCC2)C(=O)NC2CC2)s1] | (S)-2-(((5-chloro-2- methylpyridin-3- yl)(methyl)amino)methyl)- N-(3-cyclohexyl-1- (cyclopropylamino)-1- oxopropan-2-yl)thiazole-5- carboxamide | 490.0 [(M + H)⁺] | 28.6 mg, 16.7% as white solid |
| 289 | 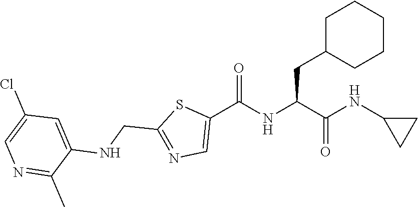 [SMILES: Cc1ncc(Cl)cc1NCc1ncc(C(=O)N[C@@H](CC2CCCCC2)C(=O)NC2CC2)s1] | 2-[[(5-chloro-2-methyl-3- pyridyl)amino]methyl]-N- [(1S)-1-(cyclohexylmethyl)- 2-(cyclopropylamino)-2- oxo-ethyl]thiazole-5- carboxamide | 475.0 [(M + H)⁺] | 17.8 mg, 10.6% as white solid |

### Example 6

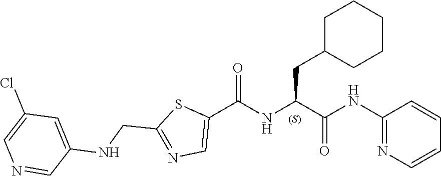

### 2-[[(5-chloro-3-pyridyl)amino]methyl]-N-1(1S)-1-(cyclohexylmethyl)-2-oxo-2-(2-pyridylamino)ethyl]thiazole-5-carboxamide

### Step 1—tert-butyl N-[(1S)-1-(cyclohexylmethyl)-2-oxo-2-(2 pyridylamino)ethyl]carbamate

To a mixture of (2S)-2-(tert-butoxycarbonylamino)-3-cyclohexyl-propanoic acid (5.00 g; 18.43 mmol, 1.0 eq) and 93yridine-2-amine (1.73 g, 18.43 mmol, 1.0 eq) in DMF (50 mL) was added TEA (9.32 g, 92.13 mmol, 12.82 mL, 5.0 eq) and T₃P (23.45 g, 36.85 mmol, 21.92 mL, 50% purity, 2.0 eq) at 0° C., and the mixture was stirred at 0° C. for 0.5 h under N₂ atmosphere. The reaction mixture was poured into water (1.00 mL), then extracted with EA (3×50 mL). The combined organic layers were washed with saturated brine (50 mL), dried over anhydrous Na₂SO₄, filtered and concentrated under reduced pressure to give a residue. The residue was purified by prep-HPLC (basic condition:column:Xtimate C18 10 u 250 mm*80 mm; mobile phase: [water (NH₃H₂O+NH₄HCO₃)-ACN]; B %: 40%-70%, 34 min) to afford tert-butyl N-[(1S)-1-(cyclohexylmethyl)-2-oxo-2-(2-pyridylamino)ethyl]carbamate (2.00 g, 5.76 mmol, 31.24% yield) as a white solid. LC-MS (ES⁺, m/z): 348.5 [(M+H)⁺]

### Step 2—(S)-2-amino-3-cyclohexyl-N-(pyridin-2-yl)propanamide

To a solution of tert-butyl N-[(1S)-1-(cyclohexylmethyl)-2-oxo-2-(2-pyridylamino) ethyl]carbamate (Step 1, 2.50 g, 7.20 mmol, 1.0 eq) in EtOAc (20 mL) was added HCl/EtOAc (4 M, 1.80 mL, 1.0 eq) The mixture was stirred at 25° C. for 4 hr under N₂ atmosphere. The reaction was concentrated in vacuum to give a residue to afford (S)-2-amino-3-cyclohexyl-N-(pyridin-2-yl)propanamide hydrochloride (1.80 g) which was used as is in the next step. LC-MS (ES⁺, m/z): 248.2 [(M+H)⁺]

### Step 3—(2S)-2-[[3-[[(5-chloro-2-methyl-3-pyridyl)amino]methyl]benzoyl]amino]-3-cyclopentyl-propanoic acid (Compound 302)

To a mixture of (S)-2-amino-3-cyclohexyl-N-(pyridin-2-yl)propanamide (136.8 mg, 482.00 μmol, 1.0 eq) and 2-[[(5-chloro-3-pyridyl)amino]methyl]thiazol-5-carboxylic acid (130.0 mg, 482.00 μmol, 1.0 eq) in DMF (2 mL) was added TEA (243.9 mg, 2.41 mmol, 335.45 μL, 5.0 eq) and T₃P (613.5 mg, 964.01 μmol, 573.32 μL, 50% purity, 2.0 eq) at 0° C. The mixture was stirred at 0° C. for 0.5 hr under N₂ atmosphere. The reaction mixture was poured into water (30 mL), then extracted with EA (3×10 mL). The combined organic layers were washed with saturated brine (10 mL), dried over anhydrous Na₂SO₄, filtered and concentrated under reduced pressure to give a residue. The residue was purified by prep-HPLC (column: Waters Xbridge Prep OBD C18 150*40 mm*10 um; mobile phase. [water (NH₃H₂O+NH₄HCO₃)-ACN]; B3%: 30%-60%, 8 min (50.0 mg, yield 20%, purity 92.4%) as a light yellow solid. The material was further separated by prep-HPLC (column: Phenomenex Luna C18.75*303 mm*3 um; mobile phase: [water (FA)-ACN]; B %: 35%-75%, 8 min) to afford 2-[[(5-chloro-3-pyridyl)amino]methyl]-N-[(1S)-1-(cyclohexylmethyl)-2-oxo-2-(2-pyridylamino)ethyl]thiazole-5-carboxamide (26.6 mg, 53.30 μmol, 11.06% yield N/A purity) as a light yellow solid, LC-MS (ES+, m/z) 499.1 [(M+H)⁺]

The preparation of the following compound in Table E follows the above procedure

TABLE E

| Cpd | | | LC-MS | Yield & |
|---|---|---|---|---|
| # | Structure | IUPAC | (ES+, m/z) | status |
| 290 | 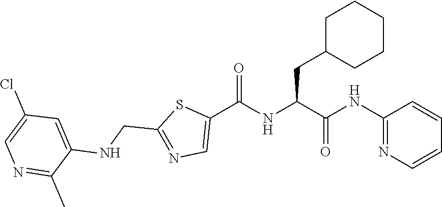 [SMILES: Cc1ncc(Cl)cc1NCc1ncc(C(=O)N[C@@H](CC2CCCCC2)C(=O)Nc2ccccn2)s1] | 5-[2-[[(5-chloro-2-methyl- 3-pyridyl)amino]methyl]- N-[(1S)-1- (cyclohexylmethyl)-2-oxo- 2-(2- pyridylamino)ethyl]thiazole- 5-carboxamide | 513.0 [(M + H)⁺] | 26.3 mg, 10.8% as white solid |
| 291 | 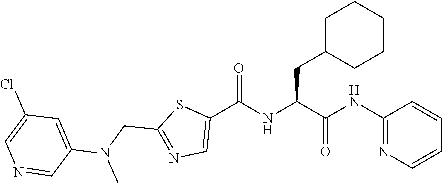 [SMILES: CN(Cc1ncc(C(=O)N[C@@H](CC2CCCCC2)C(=O)Nc2ccccn2)s1)c1cncc(Cl)c1] | 2-[[(5-chloro-3-pyridyl)- methyl-amino]methyl]-N- [(1S)-1- (cyclohexylmethyl)-2-oxo- 2-(2- pyridylamino)ethyl]thiazole- 5-carboxamide | 513.0 [(M + H)⁺] | 23.9 mg, 7.2% as white solid |

### Example 7

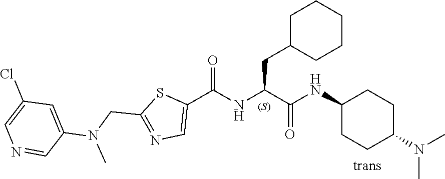

### 2-[[(5-chloro-3-pyridyl)-methyl-amino]methyl]-N-1(1S)-1-(cyclohexylmethyl)-2-[[4-(dimethylamino)cyclohexyl]amino]-2-oxo-ethyl]thiazole-5-carboxamide

### Step 1—ethyl 2-[[(5-chloro-3-pyridyl)-methyl-amino]methyl]thiazole-5-carboxylate

To a solution of ethyl 2[[(5-chloro-3-pyridyl)amino]methyl]thiazole-5-carboxylate (500.0 rag, 1.68 mmol, 1.0 eq) in in MeOH (5 mL) and AcOH (2.5 mL), was added formaldehyde (60.5 mg, 2.02 mmol, 55.51 μL, 1.2 eq) The mixture was stirred at 60° C. for 16 h wider N₂ atmosphere. NaBH₃CN (527.6 mg, 8.40 mmol, 5.0 eq) was added and the mixture was stirred at 60° C. for 0.5 h under N₂ atmosphere. The reaction mixture was adjusted to pH=˜7 with saturated Na₂CO₃, then extracted with EtOAc (3×15 mL). The combined organic phase was washed with saturated brine (15 mL), dried with anhydrous Na₂SO₄, filtered and concentrated under vacuum. The residue was purified by prep-TLC (SiO₂, DCM:MeOH=10:1, Rf=0.65) to afford ethyl 2-[[(5-chloro-3-pyridyl)-methyl-amino]methyl]thiazole-5-carboxylate (180.0 mg, 577.32 μmol, 34.38% yield) as a yellow liquid. LC-MS (ES⁺, m/z): 312.0 [(M+H)⁺]

### Step 2—2-[[(5-chloro-3-pyridyl)-methyl-amino]methyl]thiazole-5-carboxylic acid

To a solution of ethyl 2-[[(5-chloro-3-pyridyl)-methyl-amino]methyl]thiazole-5-carboxylate (Step 1, 180.0 mg, 577.32 μmol, 1.0 eq) in THE (2 mL) was added LiOH (3 M, 577.32 μL, 3.0 eq). The mixture was stirred at 25° C. for 2 hr under N₂ atmosphere. The reaction mixture was adjusted to pH=−˜4 with 3N aq. HCl, then extracted with EA (3×10 mL). The combined organic phase was washed with saturated brine (2×10 mL), dried with anhydrous Na₂SO₄ filtered and concentrated in vacuum to afford 2-[[(5-chloro-3-pyridyl)-methyl-amino]methyl]thiazole-5-carboxylic acid (180.0 mg) as a yellow solid. LC-MS (ES⁺, m/z): 284.0 [(M+H)⁺]

### Step 3—methyl (2S)-2-[[2-[[(5-chloro-3-pyridyl)-methyl-amino]methyl]thiazole-5-carbonyl]amino]-3-cyclohexyl-propanoate

To a solution of 2-[[(5-chloro-3-pyridyl)-methyl-amino]methyl]thiazole-5-carboxylic acid (Step 2, 180.0 mg, 634.40 μmol, 1.0 eq) and methyl (2S)-2-amino-3-cyclohexyl-propanoate hydrochloride (140.6 mg, 634.40 μmol, 1.0 eq) in DMF (2 mL) was added TEA (320.9 mug, 3.17 mmol, 441.50 μL, 5.0 eq) and T₃P (807.4 mg, 1.27 mmol, 754.59 μL, 50% purity, 2.0 eq) at 0° C. The mixture was stirred at 0° C. for 0.5 h under N₂ atmosphere. The reaction mixture was poured into H₂O (50 mL), then extracted with EA (3×30 mL). The combined organic phase was washed with saturated brine (30 mL), dried with anhydrous Na₂SO₄, filtered and concentrated in vacuum to give a residue to afford methyl (2S)-2-[[2-[[(5-chloro-3-pyridyl)-methyl-amino]methyl]thiazole-5-carbonyl]amino]-3-cyclohexyl-propanoate (190.0 mg) as a yellow solid. LC-MS (ES⁺, m/z): 451.2 [(M+H)⁺]

### Step 4—(2S)-2-[[2-[[(5-chloro-3-pyridyl)-methyl-amino]methyl]thiazole-5-carbonyl]amino]-3-cyclohexyl-propanoic acid

To a solution of methyl (2S)-2-[[2-[[(5-chloro-3-pyridyl)-methyl-amino]methyl]thiazole-5 carbonyl]amino]-3-cyclohexyl-propanoate (Step 3, 190.0 mg, 421.30 μmol, 1.0 eq) in THE (2 mL) was added LiOH (3 M, 42130 μL, 30 eq). The mixture was stirred at 25° C. for 2 hr under N₂ atmosphere. The reaction mixture was adjusted to pH=˜4 with 3N aq. HCl, then extracted with EA (3×5 mL). The combined organic phase was washed with saturated brine (2×5 mL), dried with anhydrous Na₂SO₄, filtered and concentrated in vacuum to afford (2S)-2-[2-[[(5-chloro-3-pyridyl)-methyl-amino]methyl]thiazole-5-carbonyl]amino]-3-cyclohexyl-propanoic acid (110.0 mg) as a yellow solid. LC-MS (ES⁺, m/z): 437.1 [(M+H)⁺]

### Step 5—2-[[(5-chloro-3-pyridyl)-methyl-amino]methyl]-N-[(1S)-1-(cyclohexylmethyl)-2-[[4-(dimethylamino)cyclohexyl]amino]-2-oxo-ethyl]thiazole-5-carboxamide (Compound 303)

To a solution of (2S)-2-[[2-[[(5-chloro-3-pyridyl)-methyl-amino]methyl]thiazole-5-carbonyl]amino]-3-cyclohexyl-propanoic acid (90.0 mg, 205.97 μmol, 1.0 eq) and N⁴,N⁴-dimethylcyclohexane-1,4-diamine hydrochloride (36.8 mg, 205.97 μmol, 1.0 eq) in DMF (2 mL) was added TEA (104.2 mg, 1.03 mmol, 143.34 μL, 5.0 eq) and T₃P (262.1 ng, 411.94 μmol, 244.99 μL, 50% purity, 2.0 eq) at 0° C. The mixture was stirred at 0° C. for 0.5 hr under N₂ atmosphere. The reaction mixture was poured into H₂O (10 mL), then extracted with EA (3×5 mL). The combined organic phase was washed with saturated brine (2×5 mL), dried with anhydrous Na₂SO₄, filtered and concentrated in vacuum to give a residue. The residue was purified by prep-HPLC (FA condition, column: Phenomenex Luna C18 75′30 mm*3 um, mobile phase: [water (FA)-ACN], B %: 25%-55%, 8 min) to afford the title compound 2-[[(5-chloro-3-pyridyl)-methyl-amino]methyl]-N-[(1S)-1-(cyclohexylmethyl)-2-[[4-(dimethylamino)cyclohexyl]amino]-2-oxo-ethyl]thiazole-5-carboxamide (15.2 mg, 26.16 μmol, 12.70% yield, 96.6% purity) as a white solid. LC-MS (ES⁺, m/z): 561.2 [(M)⁺]¹H NMR (400 MHz, DMSO-d₆) δ ppm 0.82-0.82 (m, 1H) 0.83-0.88 (m, 1H) 1.08-1.25 (m, 8H) 1.47-1.68 (m, 8H) 1.75-1.80 (m, 3H) 2.22 (s, 7H) 3.10 (s, 3H) 4.36-4.14 (m, 1H) 4.95-4.98 (m, 2H) 7.28 (t, J=2.26 Hz, 1H) 7.87-7.92 (m, 2H) 8.11-8.14 (m, 1H) 8.20-8.24 (m, 1H) 8.41 (s, 1H) 8.60-8.63 (m, 1H)

### Example 8

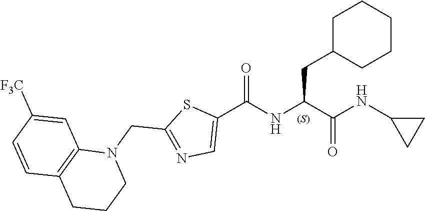

### (S)—N-(3-cyclohexyl-1-(cyclopropylamino)-1-oxopropan-2-yl)-2-((7-(trifluoromethyl)-3,4-dihydroquinolin-1(2H)-yl)methyl)thiazole-5-carboxamide

### Step 1—ethyl 2-((7-(trifluoromethyl)-3,4-dihydroquinolin-1(2H)-yl)methyl)thiazole-5-carboxylate

To a solution of 7-(trifluoromethyl)-1,2,34-tetrahydroquinoline (301.6 mg, 1.50 mmol, 48.41 μL, 1.0 eq) and ethyl 2-(bromomethyl)thiazole-5-carboxylate (450.0 mg, 1.80 mmol, 1.2 eq) in DCM (5 mL) was added TBAI (553.8 mg, 1.50 mmol, 1.0 eq) and K₂CO₃ (621.6 mg, 4.50 mmol, 3.0 eq). The mixture was stirred at 25° C. for 12 hr under N₂ atmosphere. The reaction mixture was poured into water (50 mL), extracted with EA (3×30 mL). The combined organic layers were washed with 30 ml, of brine, dried over Na₂SO₄, filtered and concentrated under reduced pressure to give a residue. The residue was purified by prep-HPLC (column: Waters Xbridge BEH C18 250*50 mm*10 um; mobile phase: [water(NH₄HCO₃)-ACN]; B %: 55%-85%, 10 min) to afford ethyl 2-((7-(trifluoromethyl)-3,4-dihydroquinolin-1(2H)-yl)methyl)thiazole-5-carboxylate (280.0 mg, 755.96 μmol, 50.42% yield) as a light yellow solid. LC-MS (ES⁺, m/z) 371.2 [(M+H)⁺]

### Step 2—2-((7-(trifluoromethyl)-3,4-dihydroquinolin-1(2H)-yl)methyl)thiazole-5-carboxylic acid

To a mixture of ethyl 2-[[7-(trifluoromethyl)-3,4-dihydro-2H-quinolin-1-yl]methyl]thiazole-5-carboxylate (Step 1, 150.0 mg, 404.98 μmol, 1.0 eq) in THF (1.2 mL) was added LiOH (1 M, 1.21 mL, 3.0 eq). The mixture was stirred at 25° C. for 2 h under N₂ atmosphere. The reaction mixture was adjusted to pH=3 by aqueous HCl (1 M), and extracted with EA (3×30 mL) The combined organic layers were washed with 30 mL of brine, dried over Na₂SO₄, filtered and concentrated under reduced pressure to afford 2-((7-(trifluoromethyl)-3,4-dihydroquinolin-1(2H)-yl)methyl)thiazole-5-carboxylic acid (140.0 mg, crude) as a yellow solid. The material was used as is in the next step. LC-MS (ES⁺, m/z): 343.1 [(M+H)⁺]

### Step 3—(S)—N-(3-cyclohexyl-1-(cyclopropylamino)-1-oxopropan-2-yl)-2-((7-(trifluoromethyl)-3,4-dihydroquinolin-1(2H)-yl)methyl)thiazole-5-carboxamide (Compound 304)

To a solution of 2-[7-(trifluoromethyl)-3,4-dihydro-2H-quinolin-yl]methyl]thiazole-5-carboxylic acid (Step 2, 140.0 mg, 408.96 μmol, 1.0 eq) and (2S-2-amino-3-cyclohexyl-N-cyclopropyl-propanamide (129.0 mg, 613.43 μmol, 1.5 eq) in DMF (2 mL) was added TEA (206.9 mg, 2.04 mmol, 284.61 μL, 5.0 eq) and T₃P (390.4 mg, 613.43 μmol, 364.83 μL, 50% purity, 0.5 eq) at 0° C. The mixture was stirred at 0° C. for 0.5 h under N₂ atmosphere. The reaction mixture was diluted with 30 mL of water and extracted with EA (3×10 mL). The combined organic layers were washed with 10 mL of saturated brine, dried over Na₂SO₄, filtered and concentrated under reduced pressure to give a residue. The residue was purified by prep-HPLC (column: Waters Xbridge BEH C18 100*30 mm*10 um; mobile phase: [water(NH₄HCO₃)-ACN]; B %: 40%-70%, 8 min) to afford (S)—N-(3-cyclohexyl-1-(cyclopropylamino)-1-oxopropan-2-yl)-2-((7-(trifluoromethyl)-3,4-dihydroquinolin-1(2H)-yl)methyl)thiazole-5-carboxamide (28.0 mg, 52.37 μmol, 12.81% yield, 100.0% purity) as a white solid. LC-MS (ES⁺, m/z): 5353 [(M+H)⁺]. ¹H NMR (400 MHz, DMSO-d₆) δ=8.62 (d, J=8.2 Hz, 1H), 8.45-8.39 (m, 1H), 8.08 (d, J=4.3 Hz, 1H), 7.13 (d, J=7.5 Hz, 1H), 6.87-6.79 (m, 2H), 4.85 (s, 2H), 4.39-4.31 (m, 1H), 3.51-3.45 (m, 2H), 2.78 (t, J=6.0 Hz, 2H), 2.60 (qt, J=3.9, 7.4 Hz, 1H), 1.94 (quin, J=5.9 Hz, 2H), 1.70-1.59 (m, 4H), 1.58-1.52 (m, 2H), 1.51-1.43 (m, 1H), 1.32-1.01 (m, 4H), 0.95-0.77 (m, 2H), 0.63-0.54 (m, 2H), 0.43-0.32 (m, 2H)

### Example 9

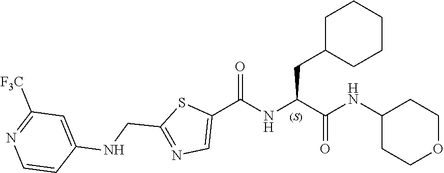

### N-[(1S)-1-(cyclohexylmethyl)-2-oxo-2-(tetrahydropyran-4-ylamino)ethyl]-2-[[[2-(trifluoromethyl)-4-pyridyl]amino]methyl]thiazole-5-carboxamide

### Step 1—methyl (2S)-3-cyclohexyl-2-[[2-[[[2-(trifluoromethyl)-4-pyridyl]amino]methyl]thiazole-5-carbonyl]amino]propanoate

To a solution of 2-[[[2-(trifluoromethyl)-4-pyridyl]amino]methyl]thiazole-5-carboxylic acid (300.0 mg, 989.25 μmol, 10 eq) and methyl (2S)-2-amino-3-cyclohexyl-propanoate hydrochloride (263.2 mg, 1.19 mmol, 1.2 eq) in DMF (5 mL) was added TEA (500.5 mg, 4.95 mmol, 688.46 μL, 5.0 eq) and T₃P (1.26 g, 1.98 mmol, 1.18 mL 50% purity, 2.0 eq) at 0° C., then the mixture was stirred at 0° C. for 0.5 h under N₂ atmosphere. The reaction mixture was poured into water (30 mL), then extracted with EA (3×10 mL), The combined organic layers were washed with saturated brine (10 mL), dried over anhydrous Na₂SO₄, filtered and concentrated under reduced pressure to give a residue. The residue was purified by prep-TLC (SiO₂, PE:EA=0:1, Rf=0.54) to afford methyl (2S)-3-cyclohexyl-2-[[2-[[[2-(trifluoromethyl)-4-pyridyl]amino]methyl]thiazole-5-carbonyl]amino]propanoate (300.0 mg, 637.61 μmol, 64.45% yield) as a yellow solid. LC-MS (ES⁺, M/z):471.1 [(M+H)⁺]

### Step 2—(2S)-3-cyclohexyl-2-[[2-[[[2-(trifluoromethyl)-4-pyridyl]amino]methyl]thiazole-5-carbonyl]amino]propanoic acid

To a mixture of methyl (2S)-3-cyclohexyl-2-[[2-[[[2-(trifluoromethyl)-4-pyridyl]amino]methyl]-thiazole-5-carbonyl]amino]propanoate (Step 1, 300.0 mg, 637.61 mmol, 1.0 eq) in THF (5 mL) was added LiOH (3 M, 637.61 μL, 3.0 eq), then the mixture was stirred at 25° C. for 2 b under N₂ atmosphere. The reaction mixture was adjusted to pH=4 with 3 N aq. HCl, then extracted with EtOAc (3×20 mL). The combined organic layers were washed with saturated brine (20 mL), dried over anhydrous Na₂SO₄, filtered and concentrated under reduced pressure to afford (2S)-3-cyclohexyl-2-[[2-[[[2-(trifluoromethyl)-4-pyridyl]amino]methyl]-thiazole-5-carbonyl]amino]-propanoic acid (260.0 mg, crude) as a yellow solid. LC-MS (ES⁺, m/z):457.1 [(M+H)⁺]

### Step 3—N-[(1S)-1-(cyclohexylmethyl)-2-oxo-2-(tetrahydropyran-4-ylamino)ethyl]-2-[[[2-(trifluoromethyl)-4-pyridyl]amino]methyl]thiazole-5-carboxamide (Compound 305)

To a mixture of (2S)-3-cyclohexyl-2-[[2-[[[2-(trifluoromethyl)-4-pyridyl]amino]methyl]thiazole-5-carbonyl]amino]propanoic acid (Step 2, 260.0 mg, 569.57 μmol, 1.0 eq) and tetrahydropyran-4-amine (69.1 mg, 683.49 μmol, 1.2 eq) in DMF (3 mL) was added TEA (288.2 mg, 2.85 mmol, 396.39 μL, 5.0 eq) and T₃P (724.9 mg, 1.14 mmol, 677.49 μL, 50% purity, 2.0 eq) at 0° C., then the mixture was stirred at 0° C. for 0.5 h under N₂ atmosphere. The reaction mixture was poured into water (30 mL), then extracted with EtOAc (3×10 mL). The combined organic layers were washed with saturated brine (10 mL), dried over anhydrous Na₂SO₄, filtered and concentrated under reduced pressure to give a residue. The residue was purified by prep-HPLC (basic condition, column: Waters Xbridge Prep OBD C18 150*40 mm*10 um; mobile phase: [water (0.05% NH₃H₂O+10 mM NH₄HCO₃)-ACN]; B %: 20%-60%, 8 min) to afford N-[(1S)-1-(cyclohexylmethyl)-2-oxo-2-(tetrahydropyran-4-ylamino)ethyl]-2-[[[2-(trifluoromethyl)-4-pyridyl]amino]methyl]thiazole-5-carboxamide (30.2 mg, 55.97 μmol, 9.83% yield, 100.0% purity) as a yellow solid. LC-MS (ES⁺, m/z):540.1 [(M+H)⁺], ¹H NMR (400 MHz, DMSO-d₆) δ=8.63 (d, J=8.3 Hz, 1H), 8.43 (s, 1H), 8.20 (d, J=5.7 Hz, 1H), 8.03 (d, J=7.7 Hz, 1H), 7.95 (t, J=6.2 Hz, 1H), 7.04 (d, J=1.7 Hz, 1H), 6.81-6.74 (m, 1H), 4.77 (d, J=6.2 Hz, 2H), 4.47-4.39 (m, 1H), 3.83-3.77 (m, 2H), 3.76-3.67 (m, 1H), 3.34 (br s, 1H), 3.31-3.27 (m, 1H), 1.70-1.52 (m, 9H), 1.46-1.35 (m, 2H), 1.31-1.23 (m, 1H), 1.18-1.05 (m, 3H), 0.95-0.83 (m, 2H)

### Example 10

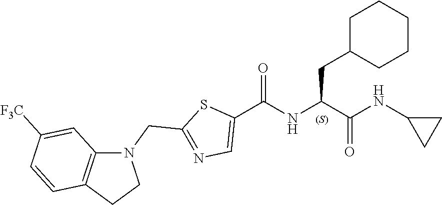

### N-[(1S)-1-(cyclohexylmethyl)-2-(cyclopropylamino)-2-oxo-ethyl]-2-[[6-(trifluoromethyl)indolin-1-yl]methyl]thiazole-5-carboxamide

### Step 1—ethyl 2-[[6-(trifluoromethyl)indolin-1-yl]methyl]thiazole-5-carboxylate

To a mixture of 6-(trifluoromethyl)indoline (600.0 mg, 3.21 mmol, 1.0 eq) and ethyl 2-(bromomethyl)thiazole-5-carboxylate (801.8 ng, 3.21 mmol, 1.0 eq) in DMF (1 mL) was added CaCO₃ (962.5 ng, 9.62 mmol, 3.0 eq). The mixture was stirred at 50° C. for 2 h under N₂ atmosphere. The reaction mixture was poured into water (30 mL), then extracted with DCM (3×20 mL). The combined organic layers were washed with saturated brine (20 mL), dried over anhydrous Na₂SO₄, filtered and concentrated under reduced pressure to give a residue. The residue was purified by column chromatography (SiO₂, PE/EA=20/1 to 5/1) to afford ethyl 2-[[6-(trifluoromethyl)indolin-1-yl]methyl]thiazole-5-carboxylate (1.00 g, 2.81 mmol, 87.53% yield) as a yellow solid. LC-MS (ES⁺, m/z):357.1 [(M+H)⁺]

### Step 2—2-[[6-(trifluoromethyl)indolin-1-yl]methyl]thiazole-5-carboxylic acid

To a mixture of ethyl 2-[[6-(trifluoroethyl)indolin-1-yl]methyl]thiazole-5-carboxylate (Step 1, 1.00 g, 2.81 mmol, 1.0 eq) in THF (10 mL) was added LiOH (3 M, 2.81 mL, 3.0 eq) at 25° C. The mixture was stirred at 25° C. for 2 h under N₂ atmosphere. The reaction mixture was adjusted to pH=6 with 3N aq. HCl, then extracted with EtOAc (3×30 mL). The combined organic layers were washed with saturated brine (30 mL), dried over anhydrous Na₂SO₄, filtered and concentrated under reduced pressure to afford 2-[[6-(trifluoromethyl)indolin-1-yl]methyl]thiazole-5-carboxylic acid (900.0 mg, crude) as a yellow solid. The material was used as is in the next step without further purification. LC-MS (ES⁺, m/z):329.1 [(M+H)⁺]

### Step 3—N-[(1S)-1-(cyclohexylmethyl)-2-(cyclopropylamino)-2-oxo-ethyl]-2-[[6-(trifluoromethyl)indolin-1-yl]methyl]thiazole-5-carboxamide (Compound 306)

To a mixture of 2-[[6-(trifluoromethyl)indolin-1-yl]methyl]thiazole-5-carboxylic acid (Step 2, 180.0 mg, 548.26 μmol, 1.0 eq) and (2S)-2-amino-3-cyclohexyl-N-cyclopropyl-propanamide hydrochloride (162.4 mg, 657.92 μmol, 1.2 eq) in DMF (1 mL) was added TEA (277.3 mg, 274 mmol, 381.56 μL, 5.0 eq) and T₃P (697.8 mg, 1.10 mmol 652.14 μL, 50% purity, 2.0 eq) at 0° C. The mixture was stirred at 0° C. for 0.5 h under N atmosphere. The reaction mixture as poured into water (30 mL), then extracted with EtOAc (3×20 mL). The combined organic layers were washed with saturated brine (20 mL), dried over anhydrous Na₂SO₄, filtered and concentrated under reduced pressure to give a residue. The residue was purified by prep-HPLC (column: Waters Xbridge Prep OBD C18 150*40 mm*10 um; mobile phase: [water (NH₃H₂O+NH₄HCO₃)-ACN]; B %: 35%-65%, 8 min) to afford N-[(1S)-1-(cyclohexylmethyl)-2-(cyclopropylamino)-2-oxo-ethyl]-2-[[6-(trifluoromethyl)indolin-1-yl]methyl]thiazole-5-carboxamide (23.9 mg, 45.82 mol, 8.360 yield, 99.8% purity) was obtained as a white solid. LC-MS (ES+, m/z) 521.0 [(M+H)⁺]

The preparation of the following compounds in Table F follows the above procedure:

TABLE F

| Cpd | | | LC-MS |
|---|---|---|---|
| # | Structure | IUPAC | (ES+, m/z) |
| 310 | 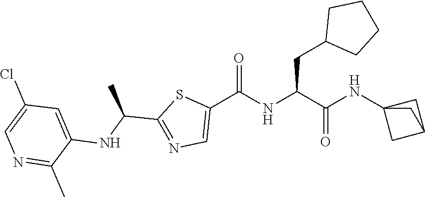 [SMILES: Cc1ncc(Cl)cc1N[C@@H](C)c1ncc(C(=O)N[C@@H](CC2CCCC2)C(=O)NC23CC(C2)C3)s1] | (2S)-N- {bicyclo[1.1.1]pentan-1- yl}-2-({2-[(1S)-1-[(5- chloro-2-methylpyridin-3- yl)amino]ethyl]-1,3-thiazol- 5-yl}formamido)-3- cyclopentylpropanamide | 502.07 |
| 311 | 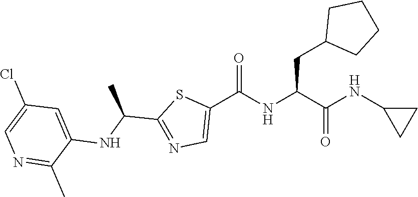 [SMILES: Cc1ncc(Cl)cc1N[C@@H](C)c1ncc(C(=O)N[C@@H](CC2CCCC2)C(=O)NC2CC2)s1] | (2S)-2-({2-[(1S)-1-[(5- chloro-2-methylpyridin-3- yl)amino]ethyl]-1,3-thiazol- 5-yl}formamido)-3- cyclopentyl-N- cyclopropylpropanamide | 476.04 |
| 118 | 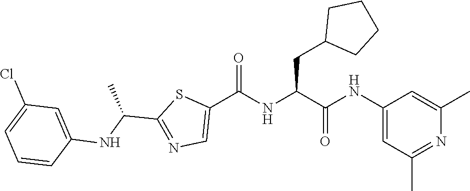 [SMILES: Cc1cc(NC(=O)[C@H](CC2CCCC2)NC(=O)c2cnc([C@@H](C)Nc3cccc(Cl)c3)s2)cc(C)n1] | (2S)-2-({2-[(1R)-1-[(3- chlorophenyl)amino]ethyl]- 1,3-thiazol-5- yl}formamido)-3- cyclopentyl-N-(2,6- dimethylpyridin-4- yl)propanamide | 526.1 |
| 119 | 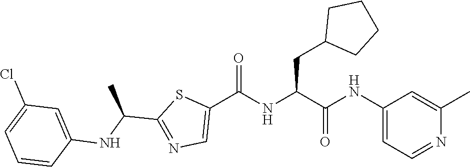 [SMILES: Cc1cc(NC(=O)[C@H](CC2CCCC2)NC(=O)c2cnc([C@H](C)Nc3cccc(Cl)c3)s2)ccn1] | (2S)-2-({2-[(1S)-1-[(3- chlorophenyl)amino]ethyl]- 1,3-thiazol-5- yl}formamido)-3- cyclopentyl-N-(2- methylpyridin-4- yl)propanamide | 512.07 |
| 120 | 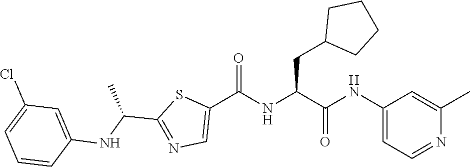 [SMILES: Cc1cc(NC(=O)[C@H](CC2CCCC2)NC(=O)c2cnc([C@@H](C)Nc3cccc(Cl)c3)s2)ccn1] | (2S)-2-({2-[(1R)-1-[(3- chlorophenyl)amino]ethyl]- 1,3-thiazol-5- yl}formamido)-3- cyclopentyl-N-(2- methylpyridin-4- yl)propanamide | 512.07 |
| 121 | 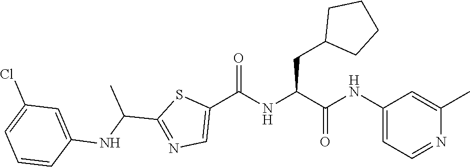 [SMILES: Cc1cc(NC(=O)[C@H](CC2CCCC2)NC(=O)c2cnc(C(C)Nc3cccc(Cl)c3)s2)ccn1] | (2S)-2-[(2-{1-[(3- chlorophenyl)amino]ethyl}- 1,3-thiazol-5- yl)formamido]-3- cyclopentyl-N-(2- methylpyridin-4- yl)propanamide | 512.07 |
| 122 | 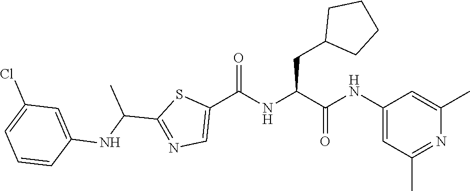 [SMILES: Cc1cc(NC(=O)[C@H](CC2CCCC2)NC(=O)c2cnc(C(C)Nc3cccc(Cl)c3)s2)cc(C)n1] | (2S)-2-[(2-{1-[(3- chlorophenyl)amino]ethyl}- 1,3-thiazol-5- yl)formamido]-3- cyclopentyl-N-(2,6- dimethylpyridin-4- yl)propanamide | 526.1 |
| 123 | 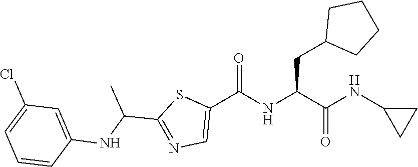 [SMILES: CC(Nc1cccc(Cl)c1)c1ncc(C(=O)N[C@@H](CC2CCCC2)C(=O)NC2CC2)s1] | (2S)-2-[(2-{1-[(3- chlorophenyl)amino]ethyl}- 1,3-thiazol-5- yl)formamido]-3- cyclopentyl-N- cyclopropylpropanamide | 461.02 |
| 124 | 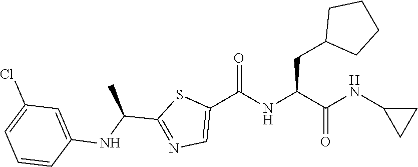 [SMILES: C[C@H](Nc1cccc(Cl)c1)c1ncc(C(=O)N[C@@H](CC2CCCC2)C(=O)NC2CC2)s1] | (2S)-2-({2-[(1S)-1-[(3- chlorophenyl)amino]ethyl]- 1,3-thiazol-5- yl}formamido)-3- cyclopentyl-N- cyclopropylpropanamide | 461.02 |
| 125 | 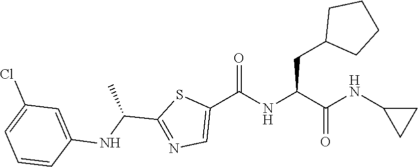 [SMILES: C[C@@H](Nc1cccc(Cl)c1)c1ncc(C(=O)N[C@@H](CC2CCCC2)C(=O)NC2CC2)s1] | (2S)-2-({2-[(1R)-1-[(3- chlorophenyl)amino]ethyl]- 1,3-thiazol-5- yl}formamido)-3- cyclopentyl-N- cyclopropylpropanamide | 461.02 |
| 126 | 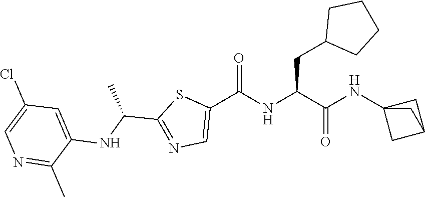 [SMILES: Cc1ncc(Cl)cc1N[C@H](C)c1ncc(C(=O)N[C@@H](CC2CCCC2)C(=O)NC23CC(C2)C3)s1] | (2S)-N- {bicyclo[1.1.1]pentan-1- yl}-2-({2-[(1R)-1-[(5- chloro-2-methylpyridin-3- yl)amino]ethyl]-1,3-thiazol- 5-yl}formamido)-3- cyclopentylpropanamide | 502.07 |
| 127 | 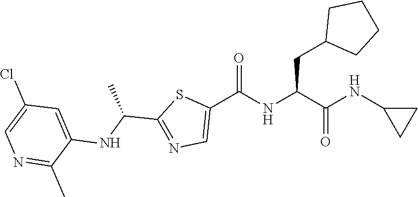 [SMILES: Cc1ncc(Cl)cc1N[C@H](C)c1ncc(C(=O)N[C@@H](CC2CCCC2)C(=O)NC2CC2)s1] | (2S)-2-({2-[(1R)-1-[(5- chloro-2-methylpyridin-3- yl)amino]ethyl]-1,3-thiazol- 5-yl}formamido)-3- cyclopentyl-N- cyclopropylpropanamide | 476.04 |
| 128 | 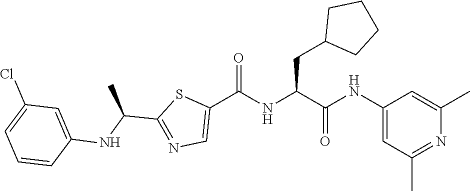 [SMILES: Cc1cc(NC(=O)[C@H](CC2CCCC2)NC(=O)c2cnc([C@H](C)Nc3cccc(Cl)c3)s2)cc(C)n1] | (2S)-2-({2-[(1S)-1-[(3- chlorophenyl)amino]ethyl]- 1,3-thiazol-5- yl}formamido)-3- cyclopentyl-N-(2,6- dimethylpyridin-4- yl)propanamide | 526.1 |

### Example 11

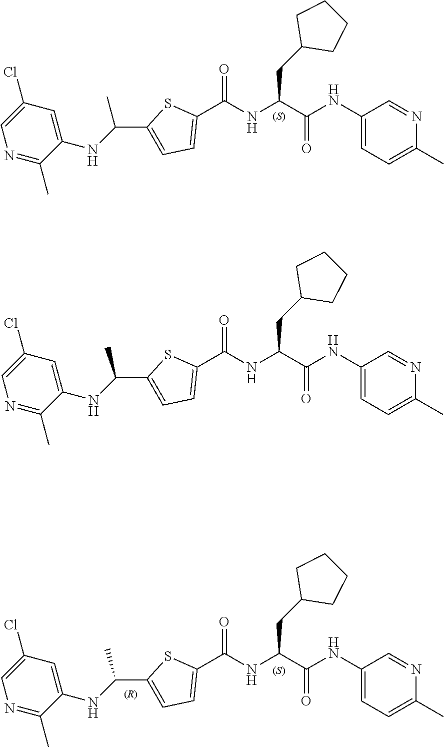

### (2S)-2-({5-[(1S)-1-[(5-chloro-2-methylpyridin-3-yl)amino]ethyl]thiophen-2-yl}formamido)-3-cyclopentyl-N-(6-methylpyridin-3-yl)propanamide

### Step 1. Preparation of 1,1-dimethylethyl N-[(1S)-1-(cyclopentylmethyl)-2-[(6-methyl-3-pyridyl) amino]-2-oxo-ethyl]carbamate

To a mixture of (2S)-3-cyclopentyl-2-(1,1-dimethylethoxycarbonylamino)propanoic acid (200 mg, 777.23 μmol), 6-methylpyridin-3-amine (126.07 mg, 1.17 mmol) and TEA (235.50 mg, 2.33 mmol) in DMF (5 mL) was added T₃P (1.17 mmiol, 1.43 mL). The resulting mixture was stirred at RT for 1h, then partitioned between EtOAc and water. The aqueous phase was extracted with EtOAc (3×). The combined organic phase washed with H₂O and brine, dried over MgSO₄₄, filtered and concentrated under reduced pressure. The residue was purified by chromatography on silica gel eluting with 0-40% EtOAc/Hexane to afford 1,1-dimethylethyl N-[(1S)-1-(cyclopentylmethyl)-2-(6-methyl-3-pyridyl) amino]-2-oxo-ethyl]carbamate (181.3 mg, Yield 67% 0).

### Step 2) Preparation of (2S)-2-azanyl-3-cyclopentyl-N-(6-methyl-3-pyridyl)propanamide Hydrochloride

To a solution of 1,1-dimethylethyl N-[(1S)-cyclopentylmethyl)-2[(6-methyl-3-pyridyl)amino]-2-oxo-ethyl]carbamate (Step 1, 125 mg, 359.76 μol) in THF (3 ML) was added 4M HCl in dioxane (359.76 μmol, 1 mL). The resulting mixture was stirred at RT for 18h. The solution concentrated under reduced pressure. The residue (100 mg) was used in the next step without further purification.

### Step 3 Preparation of (2S)-2-({5-[(1S)-1-[(5-chloro-2-methylpyridin-3-yl)amino]ethyl]thiophen-2-yl}formamido)-3-cyclopentyl-N-(6-methylpyridin-3-yl)propanamide (Compound 53)

To a mixture of crude 5-[(1S)-1-[(5-chloro-2-methyl-3-pyridyl)amino]ethyl]thiophene-2-carboxylic acid (Step 2, 60 mg, 202.18 μmol), (2S)-2-amino-3-cyclopentyl-N-(6-methyl-3-pyridyl)propanamide hydrochloride (55.03 mg, 193.92 μmol) and TEA (102.10 mg, 1.01 mmol) in DMF (3 mL) was added T₃P (303.26 μmol, 240.00 μL). The resulting mixture was stirred at RT for 1h and then partitioned between EtOAc and water. The aqueous phase was extracted with EtOAc (3×). The combined organic phase washed with water, dried over MgSO₄₄, filtered and concentrated under vacuum. The residue was purified by reverse phase HPLC using a gradient of water 0.1% FA/ACN 0.1% FA to afford the title compound (26 mg, Yield 21%). LC-MS. [M+H]⁺ 526.2.

The compounds in Table G were prepared in an analogous manner as described in the method.

TABLE G

| Cpd | | | LC-MS |
|---|---|---|---|
| No. | Structure | IUPAC | (ES+, m/z) |
| 73 | 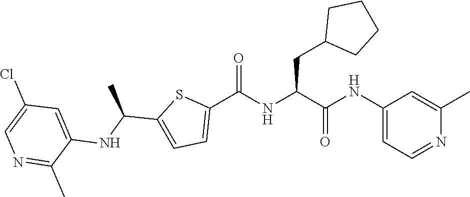 [SMILES: Cc1cc(NC(=O)[C@H](CC2CCCC2)NC(=O)c2ccc([C@H](C)Nc3cc(Cl)cnc3C)s2)ccn1] | (2S)-2-({5-[(1S)-1-[(5-chloro-2- methylpyridin-3-yl)amino]ethyl]thiophen- 2-yl}formamido)-3-cyclopentyl-N-(2- methylpyridin-4-yl)propanamide | 526.2 |
| 86 | 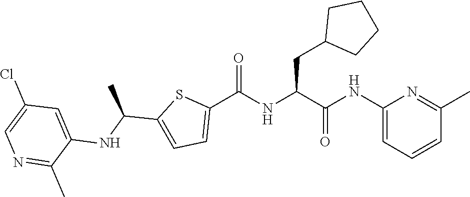 [SMILES: Cc1cccc(NC(=O)[C@H](CC2CCCC2)NC(=O)c2ccc([C@H](C)Nc3cc(Cl)cnc3C)s2)n1] | 2S)-2-({5-[(1S)-1-[(5-chloro-2- methylpyridin-3-yl)amino]ethyl]thiophen- 2-yl}formamido)-3-cyclopentyl-N-(2- methylpyridin-6-yl)propanamide | 526.2 |
| 98 | 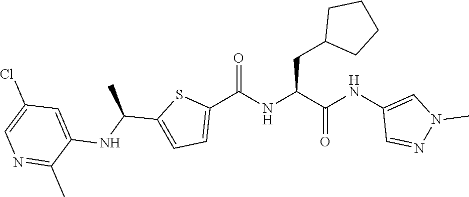 [SMILES: Cc1ncc(Cl)cc1N[C@@H](C)c1ccc(C(=O)N[C@@H](CC2CCCC2)C(=O)Nc2cnn(C)c2)s1] | (2S)-2-({5-[(1S)-1-[(5-chloro-2- methylpyridin-3-yl)amino]ethyl]thiophen- 2-yl}formamido)-3-cyclopentyl-N-(1- methyl-1H-pyrazol-4-yl)propanamide | 515.2 |

### Example 12

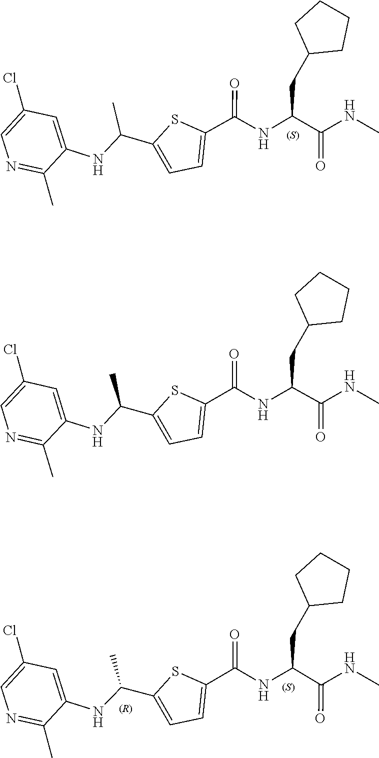

### (2S)-2-({5-[(1S)-1-[(5-chloro-2-methylpyridin-3-yl)amino]ethyl]thiophen-2-yl}formamido)-3-cyclopentyl-N-methylpropanamide (Compound 74)

To a mixture of (2S)-2-[[5-[(1S)-1-[(5-chloro-2-methylpyridin-3-yl)amino]ethyl]thiophen-2-carbonyl]amino]-3-cyclopentyl-propanoic acid (60 mg, 137.63 μmol), methanamine hydrochlroide (27.88 mg, 412.88 μmol, 31.01 μL) and TEA (111.20 mg, 1.10 mmol) in DMF (3 mL) was added T₃P (206.44 μmol, 0.17 mL). The resulting mixture was stirred at RT for 1h, then partitioned between EtOAc and water. The aqueous phase was extracted with EtOAc (3×). The combined organic phase washed with water and brine, dried over MgSO₄₄ filtered and concentrated under vacuum. The residue was purified by reverse phase HPLC using a gradient of water 0.1% FA/ACN 0.1% FA to afford the title compound (30 mg, Yield 48%) LC-MS: [M+H]⁺ 449.2.

The compounds in table H were prepared in an analogous manner as described in the method.

TABLE H

| Cpd | | | LC-MS |
|---|---|---|---|
| No. | Structure | IUPAC | (ES+, m/z) |
| 75 | 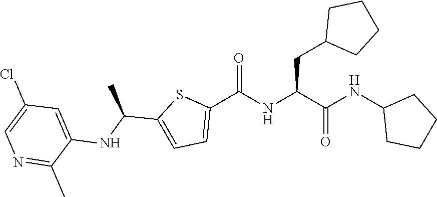 [SMILES: Cc1ncc(Cl)cc1N[C@@H](C)c1ccc(C(=O)N[C@@H](CC2CCCC2)C(=O)NC2CCCC2)s1] | (2S)-2-({5-[(1S)-1-[(5-chloro-2- methylpyridin-3-yl)amino]ethyl]thiophen- 2-yl}formamido)-N,3- dicyclopentylpropanamide | 449.20 |
| 76 | 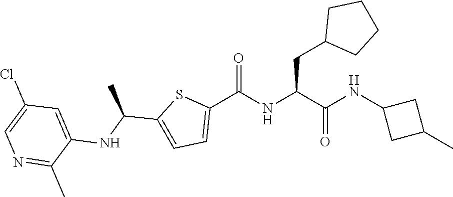 [SMILES: Cc1ncc(Cl)cc1N[C@@H](C)c1ccc(C(=O)N[C@@H](CC2CCCC2)C(=O)NC2CC(C)C2)s1] | (2S)-2-({5-[(1S)-1-[(5-chloro-2- methylpyridin-3-yl)amino]ethyl]thiophen- 2-yl}formamido)-3-cyclopentyl-N-(3- methylcyclobutyl)propanamide | 503.2 |
| 87 | 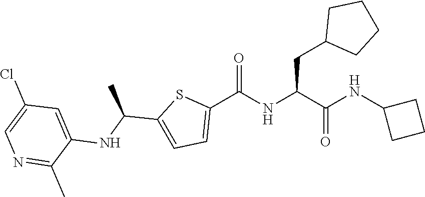 [SMILES: Cc1ncc(Cl)cc1N[C@@H](C)c1ccc(C(=O)N[C@@H](CC2CCCC2)C(=O)NC2CCC2)s1] | (2S)-2-({5-[(1S)-1-[(5-chloro-2- methylpyridin-3-yl)amino]ethyl]thiophen- 2-yl}formamido)-N-cyclobutyl-3- cyclopentylpropanamide | 489.2 |
| 88 | 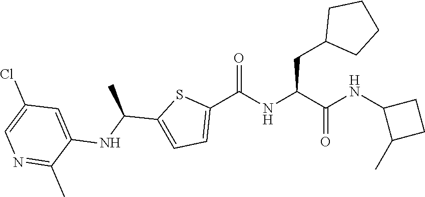 [SMILES: Cc1ncc(Cl)cc1N[C@@H](C)c1ccc(C(=O)N[C@@H](CC2CCCC2)C(=O)NC2CCC2C)s1] | (2S)-2-({5-[(1S)-1-[(5-chloro-2- methylpyridin-3-yl)amino]ethyl]thiophen- 2-yl}formamido)-3-cyclopentyl-N-(2- methylcyclobutyl)propanamide | 503.2 |
| 94 | 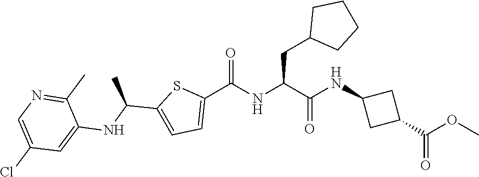 [SMILES: COC(=O)[C@H]1C[C@H](NC(=O)[C@H](CC2CCCC2)NC(=O)c2ccc([C@H](C)Nc3cc(Cl)cnc3C)s2)C1] | methyl (1r,3r)-3-[(2S)-2-({5-[(1S)-1-[(5- chloro-2-methylpyridin-3- yl)amino]ethyl]thiophen-2- yl}formamido)-3- cyclopentylpropanamido]cyclobutane-1- carboxylate | 547.2 |
| 95 | 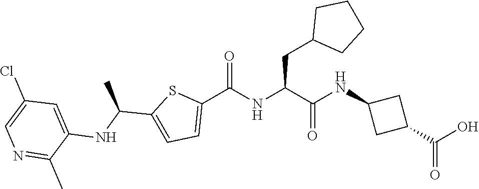 [SMILES: Cc1ncc(Cl)cc1N[C@@H](C)c1ccc(C(=O)N[C@@H](CC2CCCC2)C(=O)N[C@H]2C[C@H](C(=O)O)C2)s1] | (1r,3r)-3-[(2S)-2-({5-[(1S)-1-[(5-chloro-2- methylpyridin-3-yl)amino]ethyl]thiophen- 2-yl}formamido)-3- cyclopentylpropanamido]cyclobutane-1- carboxylic acid | 533.2 |
| 99 | 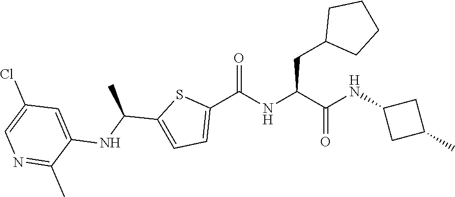 [SMILES: Cc1ncc(Cl)cc1N[C@@H](C)c1ccc(C(=O)N[C@@H](CC2CCCC2)C(=O)N[C@H]2C[C@@H](C)C2)s1] | (2S)-2-({5-[(1S)-1-[(5-chloro-2- methylpyridin-3-yl)amino]ethyl]thiophen- 2-yl}formamido)-3-cyclopentyl-N- [(1r,3s)-3-methylcyclobutyl]propanamide | 503.2 |
| 113 | 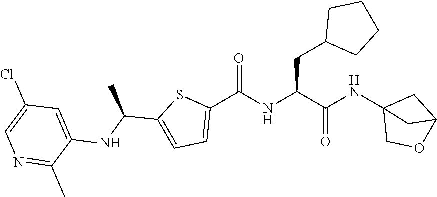 [SMILES: Cc1ncc(Cl)cc1N[C@@H](C)c1ccc(C(=O)N[C@@H](CC2CCCC2)C(=O)NC23COC(C2)C3)s1] | (2S)-2-({5-[(1S)-1-[(5-chloro-2- methylpyridin-3-yl)amino]ethyl]thiophen- 2-yl}formamido)-3-cyclopentyl-N-{2- oxabicyclo[2.1.1]hexan-4-ylpropanamide | 517.2 |
| 114 | 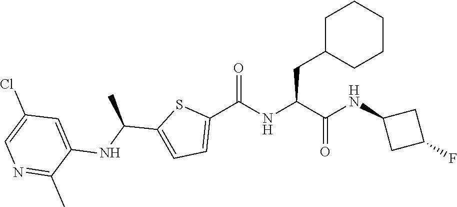 [SMILES: Cc1ncc(Cl)cc1N[C@@H](C)c1ccc(C(=O)N[C@@H](CC2CCCCC2)C(=O)N[C@H]2C[C@H](F)C2)s1] | (2S)-2-({5-[(1S)-1-[(5-chloro-2- methylpyridin-3-yl)amino]ethyl]thiophen- 2-yl}formamido)-3-cyclopentyl-N- [(1s,3r)-3-methylcyclobutyl]propanamide | 503.2 |
| 115 | 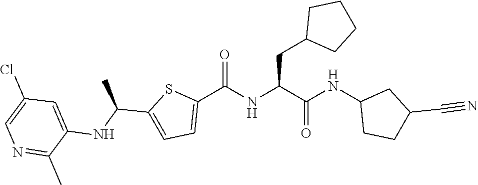 [SMILES: Cc1ncc(Cl)cc1N[C@@H](C)c1ccc(C(=O)N[C@@H](CC2CCCC2)C(=O)NC2CCC(C#N)C2)s1] | (2S)-2-({5-[(1S)-1-[(5-chloro-2- methylpyridin-3-yl)amino]ethyl]thiophen- 2-yl}formamido)-N-(3- cyanocyclopentyl)-3- cyclopentylpropanamide | 528.2 |
| 297 | 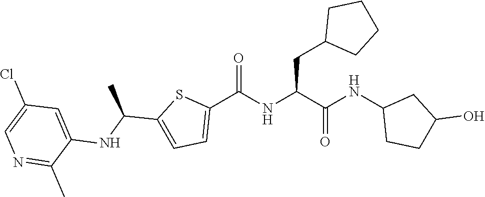 [SMILES: Cc1ncc(Cl)cc1N[C@@H](C)c1ccc(C(=O)N[C@@H](CC2CCCC2)C(=O)NC2CCC(O)C2)s1] | (2S)-2-({5-[(1S)-1-[(5-chloro-2- methylpyridin-3-yl)amino]ethyl]thiophen- 2-yl}formamido)-3-cyclopentyl-N-(3- hydroxycyclopentyl)propanamide | 519.2 |

### Example 13

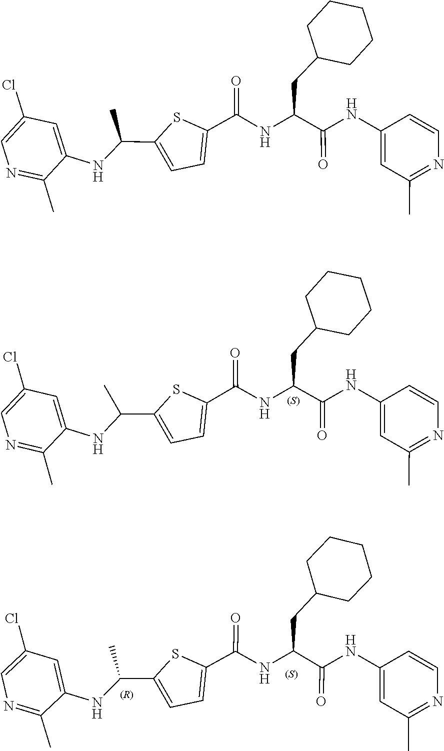

### (2S)-2-({5-[(1S)-1-[(5-chloro-2-methylpyridin-3-yl)amino]ethyl]thiophen-2-yl}formamido)-3-cyclohexyl-N-(2-methylpyridin-4-yl)propanamide (Compound 307)

To a mixture of 5-[(1 S)-1-[(5-chloro-2-methyl-3-pyridyl)amino]ethyl]thiophene-2-carboxylic acid (50 mg, 1.68 μmol), (2S)-2-amino-3-cyclohexyl-N-(2-methyl-4-pyridyl) 110yridine 110 de hydrochloride (60.0 mg, 202 μmol) and TEA (84.8 mg, 0.84 mmol) in DMF (3 mL) was added T₃P (252 μmol 200.00 μL). The resulting mixture was stirred at RT for 1h, then partitioned between EtOAc and water. The aqueous phase was extracted with EtOAc (3×). The combined organic phase washed with water, dried over MgSO₄₄, filtered and concentrated under reduced pressure. The residue was purified by reverse phase HPLC using a gradient of water 0.1% FA/ACN 0.1% FA to afford the title compound (58 mg, Yield 64%). LC-MS. [M+H]⁺ 540.1.

### Example 14

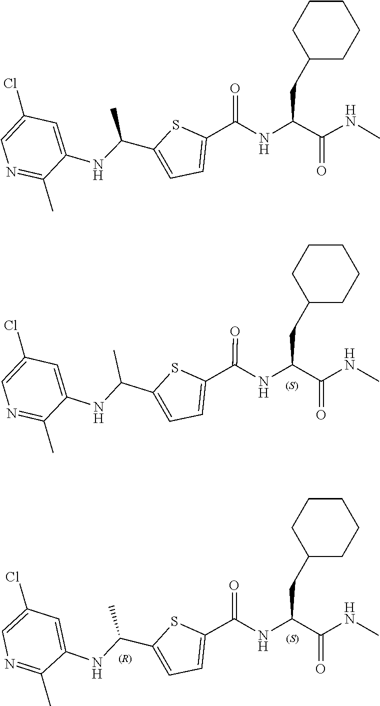

### (2S)-2-({5-[(1S)-1-[(5-chloro-2-methylpyridin-3-yl)amino]ethyl]thiophen-2-yl}formamido)-3-cyclohexyl-N-methylpropanamide

### Step 1) Preparation of methyl (2S)-2-[[5-[(1S)-1-[(5-chloro-2-methyl-3-pyridyl)amino]ethyl]thiophene-2-carbonyl]amino]-3-cyclohexyl-propanoate

To a mixture of 5-[(1S)-1-[(5-chloro-2-methyl-3-pyridyl)amino]ethyl]thiophene-2-carboxylic acid (300 mg, 1.01 mmol), methyl (2S)-2-azanyl-3-cyclohexyl-propanoate hydrochloride (291.3 mg, 1.314 mmol) and TEA (510.49 mg, 5.05 mmol) in DMF (5 mL) was added T₃P (1.52 mmol, 1.2 mL). The resulting mixture was stirred at RT for 1h, then partitioned between EtOAc and water. The aqueous phase was extracted with EtOAc (3×). The combined organic phase washed with water and brine, dried over MgSO₄), filtered and concentrated under reduced pressure. The residue was purified by chromatography on silica gel eluting with 20-100% EtOAc/Hexane to afford methyl (2S)-2-[[5-[(1S)-1-[(5-chloro-2-methyl-3-pyridyl)amino]ethyl]thiophene-2-carbonyl]amino]-3-cyclohexyl-propanoate (334 mg, Yield 71%).

### Step 2) Preparation of (2S)-2-[[5-[(S)-1-[(5-chloro-2-methyl-3-pyridyl)amino]ethyl]thiophene-2-carbonyl]amino]-3-cyclohexyl-propanoic acid

To a stirred solution of methyl (2S)-2-[[5-[(1S)-1(5-chloro-2-methyl-3-pyridyl)amino]ethyl]thiophene-2-carbonyl]amino]-3-cyclohexyl-propanoate (Step 1, 334 mg, 720 μmol) in THE (5.0 mL) was added LiOH (2 M, 2 mL). The reaction mixture was stirred at RT for 1 d. The solvent was removed in vacuo and the aqueous layer was acidified with conc. HCl. The precipitate was filtered, washed with water and dried to afford (2S)-2-[[5-[(1)-1-[(5-chloro-2-methyl-3-pyridyl)amino]ethyl]thiophene-2-carbonyl]amino]-3-cyclohexyl-propanoic acid (171 mg, Yield 53%).

### Step 3) Preparation of (2S)-2-({5-[(1S)-1-[(5-chloro-2-methylpyridin-3-yl)amino]ethyl]thiophen-2-yl}formamido)-3-cyclohexyl-N-methylpropanamide (Compound 308)

To a mixture of (2S)-2-[[5-[(1)-1-[(5-chloro-2-methyl-3-pyridyl)amino]ethyl]thiophene-2-carbonyl]amino]-3-cyclohexyl-propanoic acid (Step 2, 60 mg, 133 μmol), methanamine hydrochloride (45 mg, 667 μmol) and TEA (94 mg, 0.931 mmol) in DMF (3 mL) was added T₃P (200 μmol, 0.16 mL) The resulting mixture was stirred at RT for 1h, then partitioned between EtOAc and water. The aqueous phase was extracted with EtOAc (3×). The combined organic phase washed with water then brine, dried over MgSO₄₄, filtered and concentrated under vacuum. The residue was purified by reverse phase HPLC using a gradient of water 0.1% FA/ACN 0.1%. FA to afford (2S)-2-({5-[(1S)-1-[(5-chloro-2-methylpyridin-3-yl)amino]ethyl]thiophen-2-yl}formamido)-3-cyclohexyl-N-methylpropanamide (21 mg, Yield 48%). LC-MS. [M+H]⁺ 463.2.

The compound in table I were prepared in an analogous manner as described in the method.

TABLE I

| | | | LC-MS |
|---|---|---|---|
| | | | (ES+, |
| No. | Structure | IUPAC | m/z) |
| 309 | 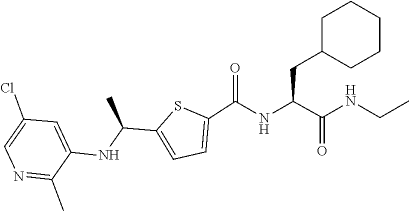 [SMILES: CCNC(=O)[C@H](CC1CCCCC1)NC(=O)c1ccc([C@H](C)Nc2cc(Cl)cnc2C)s1] | (2S)-2-({5-[(1S)-1-[(5-chloro-2- methylpyridin-3-yl)amino]ethyl]thiophen- 2-yl}formamido)-3-cyclohexyl-N- ethylpropanamide | 477.2 |

### Example 15

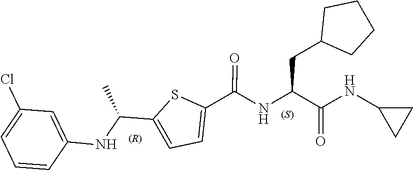

### 5-[1-(3-chloroanilino) ethyl]-N-[(1S)-1-(cyclopentylmethyl)-2-(cyclopropylamino)-2-oxo-ethyl]thiophene-2-carboxamide

### Step 1—5-acetyl-N-[(1S)-1-(cyclopentylmethyl)-2-(cyclopropylamino)-2-oxo-ethyl]thiophene-2-carboxamide

To a solution of (2S)-2-amino-3-cyclopentyl-N-cyclopropyl-propanamide hydrochloride (1.50 g, 6.46 mmol, 1.10 eq) in DMF (10 mL) were added 5-acetylthiophene-2-carboxylic acid (1.00 g, 5.88 mmol, 1.00 eq), TEA (1.78 g 17.63 mmol, 2.45 mL, 3.00 eq) and T₃P (5.61 g, 8.81 mmol, 5.24 mL, 50% purity, 1.50 eq) at 0° C. for 0.5 hr. The reaction mixture was added to water (100 ml), and the aqueous phase was extracted with EtOAc (3×50 mL). The combined organic phase was washed with brine (100 mL), dried with anhydrous Na₂SO₄, filtered and concentrated in vacuum. The crude product was triturated with EtOAc (10 mL) at 25° C. for 5 mi to afford 5-acetyl-N-[(1S)-1-(cyclopentylmethyl)-2-(cyclopropylamino)-2-oxo-ethyl]thiophene-2-carboxamide (1.60 g, 4.59 mmol, 78.14% yield) as a white solid. LC-MS (ES⁺, m/z): 347.1 [(M−H)⁻].

### Step 2—5-[1-(3-chloroanilino) ethyl]-N-[(1S)-1-(cyclopentylmethyl)-2-(cyclopropylamino)-2-oxo-ethyl]thiophene-2-carboxamide (Compound 1)

To a solution of 3-chloroaniline (219.70 mg, 1.72 mmol, 183.05 μL, 1.00 eq) and 5-acetyl-N-[(1S)-1-(cyclopentylmethyl)-2-(cyclopropylamino)-2-oxo-ethyl]thiophene-2-carboxamide (Step 1, 600.00 mg, 1.72 mmol, 1.00 eq) in DMF (6 mL) were added TMSCl (467.7 mg, 4.30 mmol, 546.34 L, 2.5 eq) and BH₃·THF (1 M, 6.37 mL, 3.7 eq) at 0° C. under N₂. The reaction was stirred at 25° C. for 14 h. The reaction mixture was poured into water (20 mL), then extracted with EtOAc (3×20 m). The combined organic layers were washed with saturated brine (20 mL), dried over anhydrous Na₂SO₄, filtered and concenrated in vacuum. The residue was purified by prep-HPLC (column: Waters Xbridge BEH C18 250*50 mm*10 um; mobile phase: [water (NH₄HCO₃)-ACN]; B %:45%-85%, 10 min) to afford 5-[1-(3-chloroanilino)ethyl]-N-[(1S)-1-(cyclopentylmethyl)-2-(cyclopropylamino)-2-oxo-ethyl]thiophene-2-carboxamide (150.0 mug, 326.07 μmol, 18.94% yield, 100% purity) as a white solid. The racemic mixture was (90.0 mg, 195.64 μmol, 1.0 eq) was further separated by prep-SFC (column: DAICEL CHIRALPAK AD (250 mm*30 mm 10 um); mobile phase: [0.1% NH₃H₂O ETOH]; B %: 55%-55%, 15 min) to afford the enantiomers 5-[(1R)-1-3-chloroanilino)ethyl]-N-[(1S)-1-(cycopentylmethyl)-2-(cyclopropylamino)-2-oxo-ethyl]thiophene-2-carboxamide (24.5 mg, 53.26 μmol, 27.22% yield) as a white solid and 5-[(1R)-1-(3-chloroanilino)ethyl]-N-[(1S)-1-(cyclopentylmethyl)-2-(cyclopropylamino)-2-oxo-ethyl]thiophene-2-carboxamide (22.8 mg, 49.56 μmol, 25.33% yield) as a white solid.

The compounds in Table J and Table K were prepared in an analogous manner as described in the method.

TABLE J

| | | | LC-MS | |
|---|---|---|---|---|
| Cpd | | | (ES⁺, | |
| ID | Structure | IUPAC | m/z) | Yield & status |
| 4 | 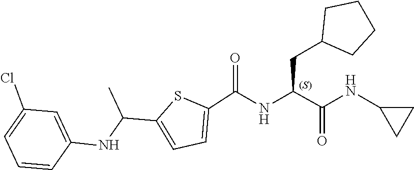 | 5-[1-(3- chloroanilino)ethyl]-N- [(1S)-1- (cyclopentylmethyl)-2- (cyclopropylamino)-2-oxo- ethyl]thiophene-2- carboxamide | 460.1 [(M + H)⁺] | 150 mg, 18.9% as white solid |
| 2 | 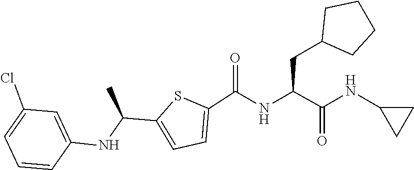 [SMILES: C[C@H](Nc1cccc(Cl)c1)c1ccc(C(=O)N[C@@H](CC2CCCC2)C(=O)NC2CC2)s1] | 5-[(1S)-1-(3- chloroanilino)ethyl]-N- [(1S)-1- (cyclopentylmethyl)-2- (cyclopropylamino)-2-oxo- ethy l]thiophene-2- carboxamide | 460.1 [(M + H)⁺] | 24.5 mg, 27.2% as white solid |
| 3 | 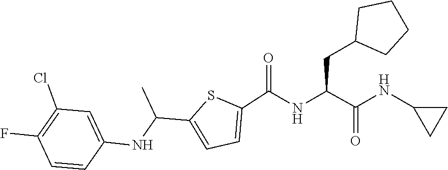 [SMILES: CC(Nc1ccc(F)c(Cl)c1)c1ccc(C(=O)N[C@@H](CC2CCCC2)C(=O)NC2CC2)s1] | 5-[1-(3-chloro-4-fluoro- anilino)ethyl]-N-[(1S)-1- (cyclopentylmethyl)-2- (cyclopropylamino)-2-oxo- ethyl]thiophene-2- carboxamide | 478.1 [(M + H)⁺] | 160 mg, 19.4% as white solid |
| 6 | 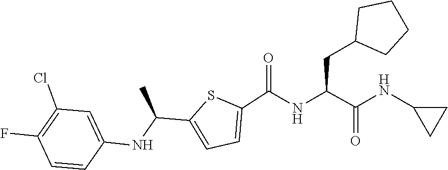 [SMILES: C[C@H](Nc1ccc(F)c(Cl)c1)c1ccc(C(=O)N[C@@H](CC2CCCC2)C(=O)NC2CC2)s1] | 5-[(1S)-1-(3-chloro-4- fluoro-anilino)ethyl]-N- [(1S)-1- (cyclopentylmethyl)-2- (cyclopropylamino)-2-oxo- ethyl]thiophene-2- carboxamide | 478.2 [(M + H)⁺] | 27.1 mg, 30.1% as white solid |
| 7 | 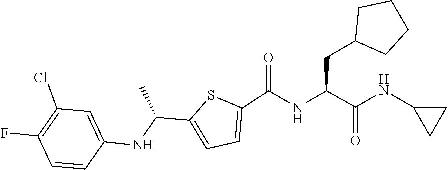 [SMILES: C[C@@H](Nc1ccc(F)c(Cl)c1)c1ccc(C(=O)N[C@@H](CC2CCCC2)C(=O)NC2CC2)s1] | 5-[(1R)-1-(3-chloro-4- fluoro-anilino)ethyl]-N- [(1S)-1- (cyclopentylmethyl)-2- (cyclopropylamino)-2-oxo- ethyl]thiophene-2- carboxamide | 478.2 [(M + H)⁺] | 24.8 mg, 27.6% as white solid |

TABLE K

| | | | LC-MS |
|---|---|---|---|
| # | Structure | IUPAC | (ES⁺, m/z) |
| 11 | 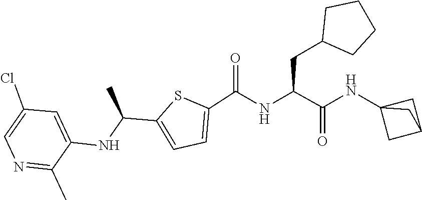 [SMILES: Cc1ncc(Cl)cc1N[C@@H](C)c1ccc(C(=O)N[C@@H](CC2CCCC2)C(=O)NC23CC(C2)C3)s1] | (2S)-N-{bicyclo[1.1.1]pentan-1-yl}- 2-({5-[(1S)-1-[(5-chloro-2- methylpyridin-3- yl)amino]ethyl]thiophen-2- yl}formamido)-3- cyclopentylpropanamide | 501.09 |
| 12 | 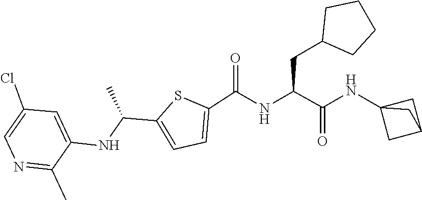 [SMILES: Cc1ncc(Cl)cc1N[C@H](C)c1ccc(C(=O)N[C@@H](CC2CCCC2)C(=O)NC23CC(C2)C3)s1] | (2S)-N-{bicyclo[1.1.1]pentan-1-yl}- 2-({5-[(1R)-1-[(5-chloro-2- methylpyridin-3- yl)amino]ethyl]thiophen-2- yl}formamido)-3- cyclopentylpropanamide | 501.09 |
| 13 | 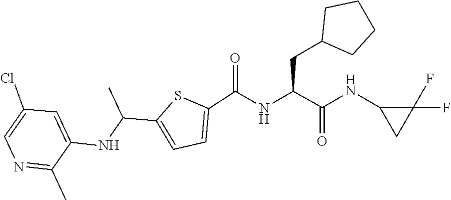 [SMILES: Cc1ncc(Cl)cc1NC(C)c1ccc(C(=O)N[C@@H](CC2CCCC2)C(=O)NC2CC2(F)F)s1] | (2S)-2-[(5-{1-[(5-chloro-2- methylpyridin-3- yl)amino]ethyl}thiophen-2- yl)formamido]-3-cyclopentyl-N-(2,2- difluorocyclopropyl)propanamide | 511.03 |
| 14 | 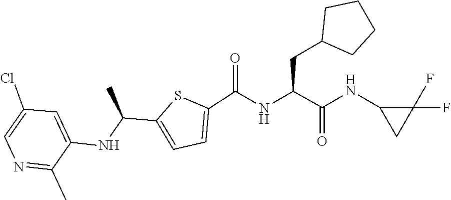 [SMILES: Cc1ncc(Cl)cc1N[C@@H](C)c1ccc(C(=O)N[C@@H](CC2CCCC2)C(=O)NC2CC2(F)F)s1] | (2S)-2-({5-[(1S)-1-[(5-chloro-2- methylpyridin-3- yl)amino]ethyl]thiophen-2- yl}formamido)-3-cyclopentyl-N-(2,2- difluorocyclopropyl)propanamide | 511.03 |
| 15 | 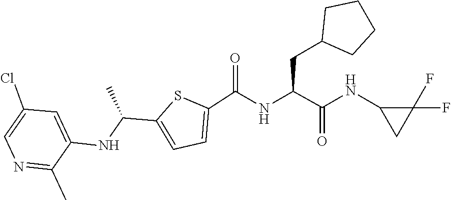 [SMILES: Cc1ncc(Cl)cc1N[C@H](C)c1ccc(C(=O)N[C@@H](CC2CCCC2)C(=O)NC2CC2(F)F)s1] | (2S)-2-({5-[(1R)-1-[(5-chloro-2- methylpyridin-3- yl)amino]ethyl]thiophen-2- yl}formamido)-3-cyclopentyl-N-(2,2- difluorocyclopropyl)propanamide | 511.03 |
| 16 | 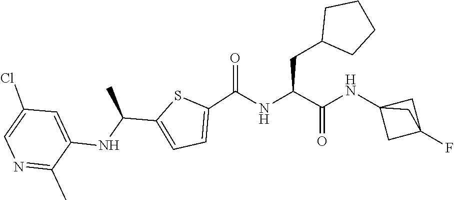 [SMILES: Cc1ncc(Cl)cc1N[C@@H](C)c1ccc(C(=O)N[C@@H](CC2CCCC2)C(=O)NC23CC(F)(C2)C3)s1] | (2S)-2-({5-[(1S)-1-[(5-chloro-2- methylpyridin-3- yl)amino]ethyl]thiophen-2- yl}formamido)-3-cyclopentyl-N-{3- fluorobicyclo[1.1.1]pentan-1- yl}propanamide | 519.08 |
| 17 | 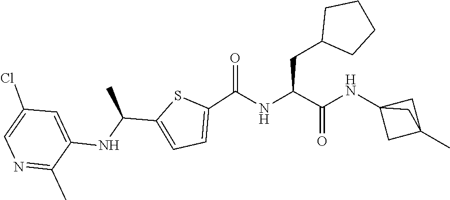 [SMILES: Cc1ncc(Cl)cc1N[C@@H](C)c1ccc(C(=O)N[C@@H](CC2CCCC2)C(=O)NC23CC(C)(C2)C3)s1] | (2S)-2-({5-[(1S)-1-[(5-chloro-2- methylpyridin-3- yl)amino]ethyl]thiophen-2- yl}formamido)-3-cyclopentyl-N-{3- methylbicyclo[1.1.1]pentan-1- yl}propanamide | 515.11 |
| 18 | 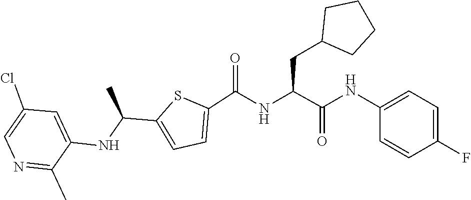 [SMILES: Cc1ncc(Cl)cc1N[C@@H](C)c1ccc(C(=O)N[C@@H](CC2CCCC2)C(=O)Nc2ccc(F)cc2)s1] | (2S)-2-({5-[(1S)-1-[(5-chloro-2- methylpyridin-3- yl)amino]ethyl]thiophen-2- yl}formamido)-3-cyclopentyl-N-(4- fluorophenyl)propanamide | 529.07 |
| 19 | 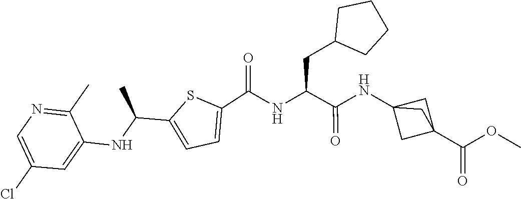 [SMILES: COC(=O)C12CC(NC(=O)[C@H](CC3CCCC3)NC(=O)c3ccc([C@H](C)Nc4cc(Cl)cnc4C)s3)(C1)C2] | methyl 3-[(2S)-2-({5-[(1S)-1-[(5- chloro-2-methylpyridin-3- yl)amino]ethyl]thiophen-2- yl}formamido)-3- cyclopentylpropanamido]bicyclo[1.1. 1]pentane-1-carboxylate | 559.12 |
| 20 | 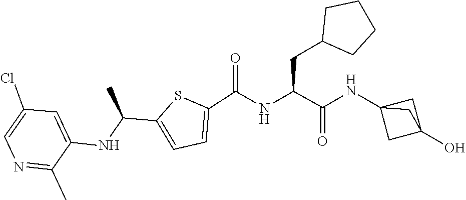 [SMILES: Cc1ncc(Cl)cc1N[C@@H](C)c1ccc(C(=O)N[C@@H](CC2CCCC2)C(=O)NC23CC(O)(C2)C3)s1] | (2S)-2-({5-[(1S)-1-[(5-chloro-2- methylpyridin-3- yl)amino]ethyl]thiophen-2- yl}formamido)-3-cyclopentyl-N-{3- hydroxybicyclo[1.1.1]pentan-1- yl}propanamide | 517.09 |
| 21 | 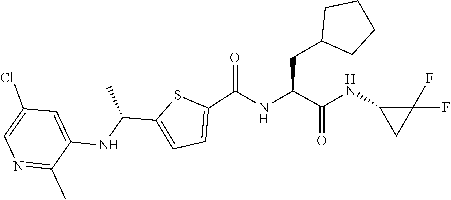 [SMILES: Cc1ncc(Cl)cc1N[C@H](C)c1ccc(C(=O)N[C@@H](CC2CCCC2)C(=O)N[C@H]2CC2(F)F)s1] | (2S)-2-({5-[(1R)-1-[(5-chloro-2- methylpyridin-3- yl)amino]ethyl]thiophen-2- yl}formamido)-3-cyclopentyl-N- [(1S)-2,2- difluorocyclopropyl]propanamide | 511.03 |
| 22 | 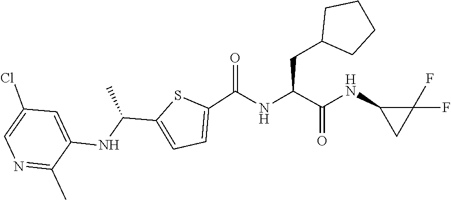 [SMILES: Cc1ncc(Cl)cc1N[C@H](C)c1ccc(C(=O)N[C@@H](CC2CCCC2)C(=O)N[C@@H]2CC2(F)F)s1] | (2S)-2-({5-[(1R)-1-[(5-chloro-2- methylpyridin-3- yl)amino]ethyl]thiophen-2- yl}formamido)-3-cyclopentyl-N- [(1R)-2,2- difluorocyclopropyl]propanamide | 511.03 |
| 23 | 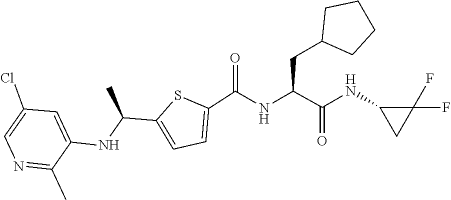 [SMILES: Cc1ncc(Cl)cc1N[C@@H](C)c1ccc(C(=O)N[C@@H](CC2CCCC2)C(=O)N[C@H]2CC2(F)F)s1] | (2S)-2-({5-[(1S)-1-[(5-chloro-2- methylpyridin-3- yl)amino]ethyl]thiophen-2- yl}formamido)-3-cyclopentyl-N- [(1S)-2,2- difluorocyclopropyl]propanamide | 511.03 |
| 24 | 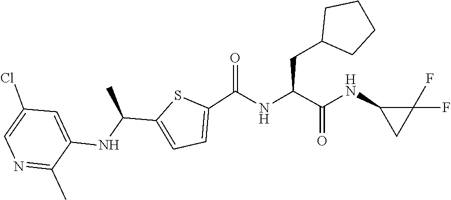 [SMILES: Cc1ncc(Cl)cc1N[C@@H](C)c1ccc(C(=O)N[C@@H](CC2CCCC2)C(=O)N[C@@H]2CC2(F)F)s1] | (2S)-2-({5-[(1S)-1-[(5-chloro-2- methylpyridin-3- yl)amino]ethyl]thiophen-2- yl}formamido)-3-cyclopentyl-N- [(1R)-2,2- difluorocyclopropyl]propanamide | 511.03 |
| 25 | 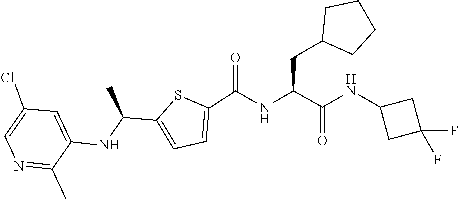 [SMILES: Cc1ncc(Cl)cc1N[C@@H](C)c1ccc(C(=O)N[C@@H](CC2CCCC2)C(=O)NC2CC(F)(F)C2)s1] | (2S)-2-({5-[(1S)-1-[(5-chloro-2- methylpyridin-3- yl)amino]ethyl]thiophen-2- yl}formamido)-3-cyclopentyl-N-(3,3- difluorocyclobutyl)propanamide | 525.06 |
| 26 | 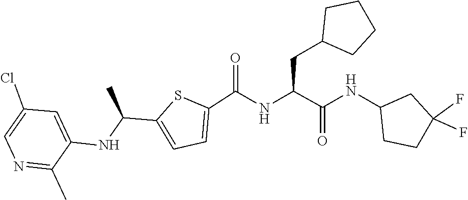 [SMILES: Cc1ncc(Cl)cc1N[C@@H](C)c1ccc(C(=O)N[C@@H](CC2CCCC2)C(=O)NC2CCC(F)(F)C2)s1] | (2S)-2-({5-[(1S)-1-[(5-chloro-2- methylpyridin-3- yl)amino]ethyl]thiophen-2- yl}formamido)-3-cyclopentyl-N-(3,3- difluorocyclopentyl)propanamide | 539.08 |
| 27 | 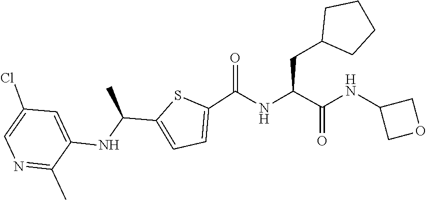 [SMILES: Cc1ncc(Cl)cc1N[C@@H](C)c1ccc(C(=O)N[C@@H](CC2CCCC2)C(=O)NC2COC2)s1] | (2S)-2-({5-[(1S)-1-[(5-chloro-2- methylpyridin-3- yl)amino]ethyl]thiophen-2- yl}formamido)-3-cyclopentyl-N- (oxetan-3-yl)propanamide | 491.05 |
| 28 | 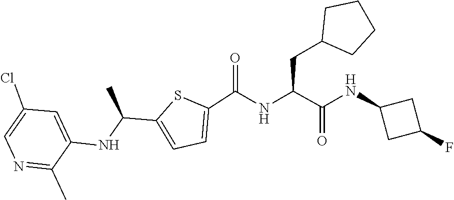 [SMILES: Cc1ncc(Cl)cc1N[C@@H](C)c1ccc(C(=O)N[C@@H](CC2CCCC2)C(=O)N[C@H]2C[C@@H](F)C2)s1] | (2S)-2-({5-[(1S)-1-[(5-chloro-2- methylpyridin-3- yl)amino]ethyl]thiophen-2- yl}formamido)-3-cyclopentyl-N- [(1s,3s)-3- fluorocyclobutyl]propanamide | 507.07 |
| 29 | 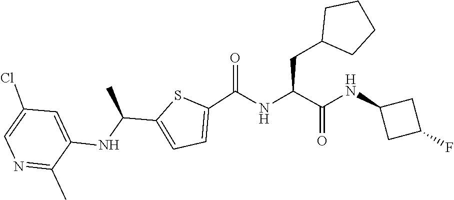 [SMILES: Cc1ncc(Cl)cc1N[C@@H](C)c1ccc(C(=O)N[C@@H](CC2CCCC2)C(=O)N[C@H]2C[C@H](F)C2)s1] | (2S)-2-({5-[(1S)-1-[(5-chloro-2- methylpyridin-3- yl)amino]ethyl]thiophen-2- yl}formamido)-3-cyclopentyl-N- [(1r,3r)-3- fluorocyclobutyl]propanamide | 507.07 |
| 30 | 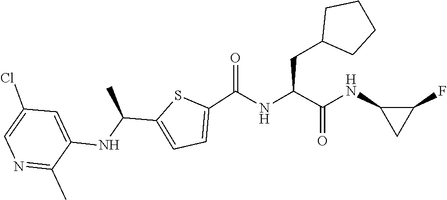 [SMILES: Cc1ncc(Cl)cc1N[C@@H](C)c1ccc(C(=O)N[C@@H](CC2CCCC2)C(=O)N[C@@H]2C[C@@H]2F)s1] | (2S)-2-({5-[(1S)-1-[(5-chloro-2- methylpyridin-3- yl)amino]ethyl]thiophen-2- yl}formamido)-3-cyclopentyl-N- [(1R,2S)-2- fluorocyclopropyl]propanamide | 493.04 |
| 31 | 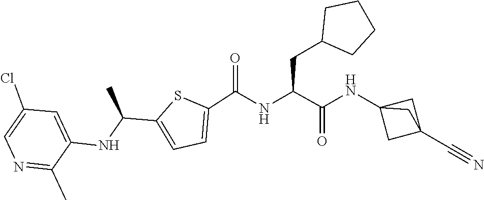 [SMILES: Cc1ncc(Cl)cc1N[C@@H](C)c1ccc(C(=O)N[C@@H](CC2CCCC2)C(=O)NC23CC(C#N)(C2)C3)s1] | (2S)-2-({5-[(1S)-1-[(5-chloro-2- methylpyridin-3- yl)amino]ethyl]thiophen-2- yl}formamido)-N-{3- cyanobicyclo[1.1.1]pentan-1-yl}-3- cyclopentylpropanamide | 526.1 |
| 32 | 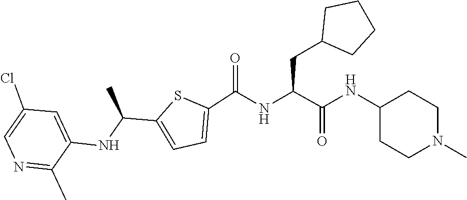 [SMILES: Cc1ncc(Cl)cc1N[C@@H](C)c1ccc(C(=O)N[C@@H](CC2CCCC2)C(=O)NC2CCN(C)CC2)s1] | (2S)-2-({5-[(1S)-1-[(5-chloro-2- methylpyridin-3- yl)amino]ethyl]thiophen-2- yl}formamido)-3-cyclopentyl-N-(1- methylpiperidin-4-yl)propanamide | 532.14 |
| 33 | 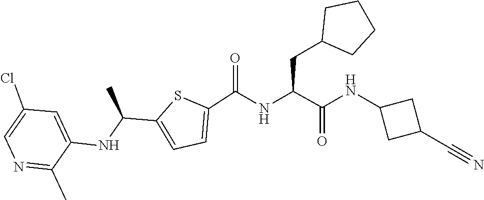 [SMILES: Cc1ncc(Cl)cc1N[C@@H](C)c1ccc(C(=O)N[C@@H](CC2CCCC2)C(=O)NC2CC(C#N)C2)s1] | (2S)-2-({5-[(1S)-1-[(5-chloro-2- methylpyridin-3- yl)amino]ethyl]thiophen-2- yl}formamido)-N-(3- cyanocyclobutyl)-3- cyclopentylpropanamide | 514.09 |
| 34 | 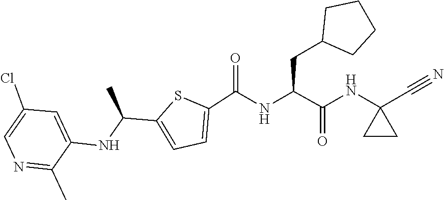 [SMILES: Cc1ncc(Cl)cc1N[C@@H](C)c1ccc(C(=O)N[C@@H](CC2CCCC2)C(=O)NC2(C#N)CC2)s1] | (2S)-2-({5-[(1S)-1-[(5-chloro-2- methylpyridin-3- yl)amino]ethyl]thiophen-2- yl}formamido)-N-(1- cyanocyclopropyl)-3- cyclopentylpropanamide | 500.06 |
| 35 | 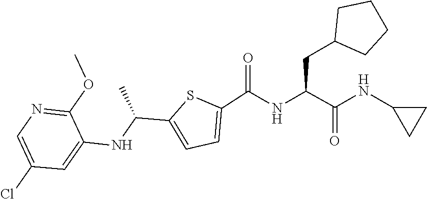 [SMILES: COc1ncc(Cl)cc1N[C@H](C)c1ccc(C(=O)N[C@@H](CC2CCCC2)C(=O)NC2CC2)s1] | (2S)-2-({5-[(1R)-1-[(5-chloro-2- methoxypyridin-3- yl)amino]ethyl]thiophen-2- yl}formamido)-3-cyclopentyl-N- cyclopropylpropanamide | 491.05 |
| 36 | 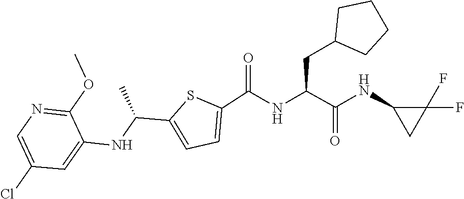 [SMILES: COc1ncc(Cl)cc1N[C@H](C)c1ccc(C(=O)N[C@@H](CC2CCCC2)C(=O)N[C@@H]2CC2(F)F)s1] | (2S)-2-({5-[(1R)-1-[(5-chloro-2- methoxypyridin-3- yl)amino]ethyl]thiophen-2- yl}formamido)-3-cyclopentyl-N- [(1R)-2,2- difluorocyclopropyl]propanamide | 527.03 |
| 37 | 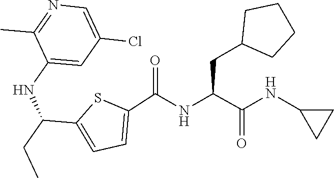 [SMILES: CC[C@H](Nc1cc(Cl)cnc1C)c1ccc(C(=O)N[C@@H](CC2CCCC2)C(=O)NC2CC2)s1] | (2S)-2-({5-[(1S)-1-[(5-chloro-2- methylpyridin-3- yl)amino]propyl]thiophen-2- yl}formamido)-3-cyclopentyl-N- cyclopropylpropanamide | 489.08 |
| 38 | 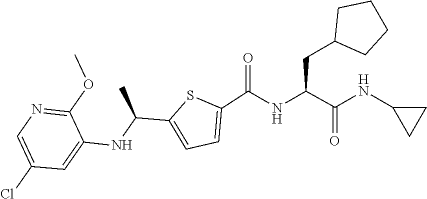 [SMILES: COc1ncc(Cl)cc1N[C@@H](C)c1ccc(C(=O)N[C@@H](CC2CCCC2)C(=O)NC2CC2)s1] | (2S)-2-({5-[(1S)-1-[(5-chloro-2- methoxypyridin-3- yl)amino]ethyl]thiophen-2- yl}formamido)-3-cyclopentyl-N- cyclopropylpropanamide | 491.05 |
| 39 | 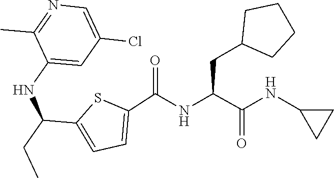 [SMILES: CC[C@@H](Nc1cc(Cl)cnc1C)c1ccc(C(=O)N[C@@H](CC2CCCC2)C(=O)NC2CC2)s1] | (2S)-2-({5-[(1R)-1-[(5-chloro-2- methylpyridin-3- yl)amino]propyl]thiophen-2- yl}formamido)-3-cyclopentyl-N- cyclopropylpropanamide | 489.08 |
| 40 | 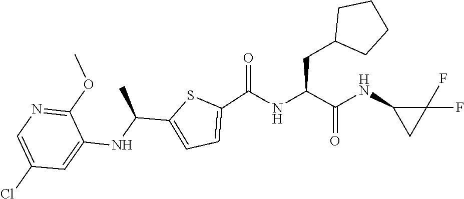 [SMILES: COc1ncc(Cl)cc1N[C@@H](C)c1ccc(C(=O)N[C@@H](CC2CCCC2)C(=O)N[C@@H]2CC2(F)F)s1] | (2S)-2-({5-[(1S)-1-[(5-chloro-2- methoxypyridin-3- yl)amino]ethyl]thiophen-2- yl}formamido)-3-cyclopentyl-N- [(1R)-2,2- difluorocyclopropyl]propanamide | 527.03 |
| 41 | 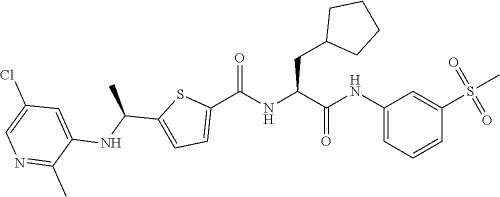 [SMILES: Cc1ncc(Cl)cc1N[C@@H](C)c1ccc(C(=O)N[C@@H](CC2CCCC2)C(=O)Nc2cccc(S(C)(=O)=O)c2)s1] | (2S)-2-({5-[(1S)-1-[(5-chloro-2- methylpyridin-3- yl)amino]ethyl]thiophen-2- yl}formamido)-3-cyclopentyl-N-(3- methanesulfonylphenyl)propanamide | 589.17 |
| 42 | 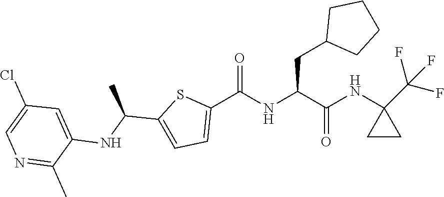 [SMILES: Cc1ncc(Cl)cc1N[C@@H](C)c1ccc(C(=O)N[C@@H](CC2CCCC2)C(=O)NC2(C(F)(F)F)CC2)s1] | (2S)-2-({5-[(1S)-1-[(5-chloro-2- methylpyridin-3- yl)amino]ethyl]thiophen-2- yl}formamido)-3-cyclopentyl-N-[1- (trifluoromethyl)cyclopropyl]propan- amide | 543.05 |
| 43 | 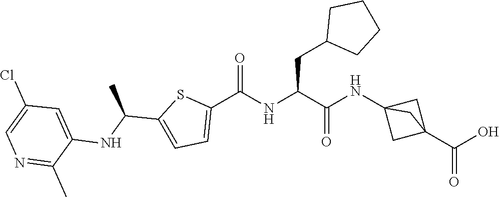 [SMILES: Cc1ncc(Cl)cc1N[C@@H](C)c1ccc(C(=O)N[C@@H](CC2CCCC2)C(=O)NC23CC(C(=O)O)(C2)C3)s1] | 3-[(2S)-2-({5-[(1S)-1-[(5-chloro-2- methylpyridin-3- yl)amino]ethyl]thiophen-2- yl}formamido)-3- cyclopentylpropanamido]bicyclo[1.1. 1]pentane-1-carboxylic acid | 545.1 |
| 44 | 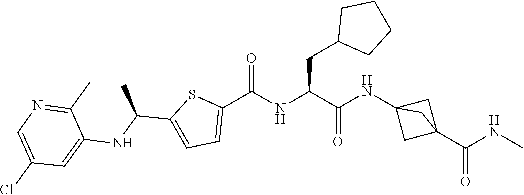 [SMILES: CNC(=O)C12CC(NC(=O)[C@H](CC3CCCC3)NC(=O)c3ccc([C@H](C)Nc4cc(Cl)cnc4C)s3)(C1)C2] | 3-[(2S)-2-({5-[(1S)-1-[(5-chloro-2- methylpyridin-3- yl)amino]ethyl]thiophen-2- yl}formamido)-3- cyclopentylpropanamido]-N- methylbicyclo[1.1.1]pentane-1- carboxamide | 558.14 |
| 45 | 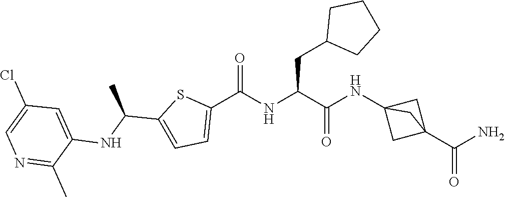 [SMILES: Cc1ncc(Cl)cc1N[C@@H](C)c1ccc(C(=O)N[C@@H](CC2CCCC2)C(=O)NC23CC(C(N)=O)(C2)C3)s1] | 3-[(2S)-2-({5-[(1S)-1-[(5-chloro-2- methylpyridin-3- yl)amino]ethyl]thiophen-2- yl}formamido)-3- cyclopentylpropanamido]bicyclo[1.1. 1]pentane-1-carboxamide | 544.11 |
| 46 | 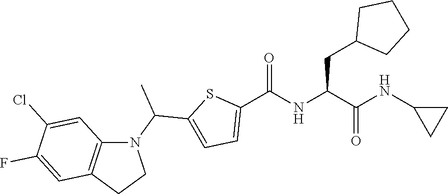 [SMILES: CC(c1ccc(C(=O)N[C@@H](CC2CCCC2)C(=O)NC2CC2)s1)N1CCc2cc(F)c(Cl)cc21] | (2S)-2-({5-[1-(6-chloro-5-fluoro-2,3- dihydro-1H-indol-1- yl)ethyl]thiophen-2-yl}formamido)-3- cyclopentyl-N- cyclopropylpropanamide | 504.06 |
| 47 | 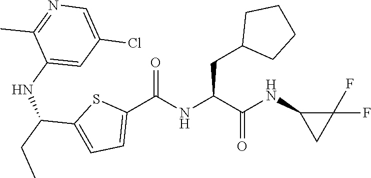 [SMILES: CC[C@H](Nc1cc(Cl)cnc1C)c1ccc(C(=O)N[C@@H](CC2CCCC2)C(=O)N[C@@H]2CC2(F)F)s1] | (2S)-2-({5-[(1S)-1-[(5-chloro-2- methylpyridin-3- yl)amino]propyl]thiophen-2- yl}formamido)-3-cyclopentyl-N- [(1R)-2,2- difluorocyclopropyl]propanamide | 525.06 |
| 48 | 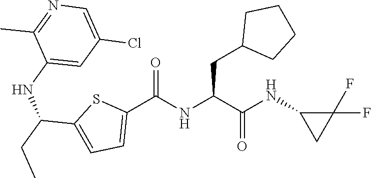 [SMILES: CC[C@H](Nc1cc(Cl)cnc1C)c1ccc(C(=O)N[C@@H](CC2CCCC2)C(=O)N[C@H]2CC2(F)F)s1] | (2S)-2-({5-[(1S)-1-[(5-chloro-2- methylpyridin-3- yl)amino]propyl]thiophen-2- yl}formamido)-3-cyclopentyl-N- [(1S)-2,2- difluorocyclopropyl]propanamide | 525.06 |
| 49 | 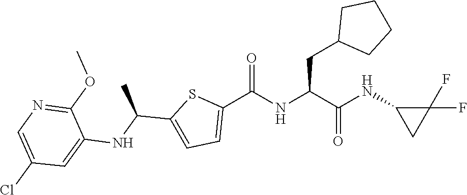 [SMILES: COc1ncc(Cl)cc1N[C@@H](C)c1ccc(C(=O)N[C@@H](CC2CCCC2)C(=O)N[C@H]2CC2(F)F)s1] | (2S)-2-({5-[(1S)-1-[(5-chloro-2- methoxypyridin-3- yl)amino]ethyl]thiophen-2- yl}formamido)-3-cyclopentyl-N- [(1S)-2,2- difluorocyclopropyl]propanamide | 527.03 |
| 50 | 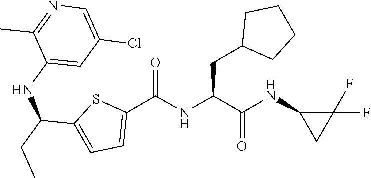 [SMILES: CC[C@@H](Nc1cc(Cl)cnc1C)c1ccc(C(=O)N[C@@H](CC2CCCC2)C(=O)N[C@@H]2CC2(F)F)s1] | (2S)-2-({5-[(1R)-1-[(5-chloro-2- methylpyridin-3- yl)amino]propyl]thiophen-2- yl}formamido)-3-cyclopentyl-N- [(1R)-2,2- difluorocyclopropyl]propanamide | 525.06 |
| 51 | 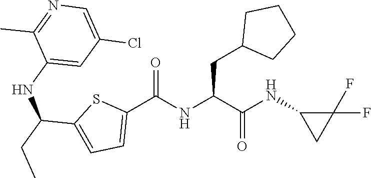 [SMILES: CC[C@@H](Nc1cc(Cl)cnc1C)c1ccc(C(=O)N[C@@H](CC2CCCC2)C(=O)N[C@H]2CC2(F)F)s1] | (2S)-2-({5-[(1R)-1-[(5-chloro-2- methylpyridin-3- yl)amino]propyl]thiophen-2- yl}formamido)-3-cyclopentyl-N- [(1S)-2,2- difluorocyclopropyl]propanamide | 525.06 |
| 52 | 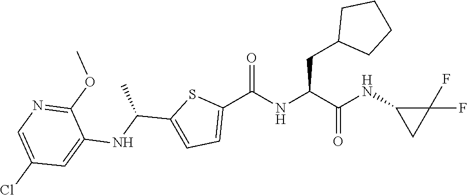 [SMILES: COc1ncc(Cl)cc1N[C@H](C)c1ccc(C(=O)N[C@@H](CC2CCCC2)C(=O)N[C@H]2CC2(F)F)s1] | (2S)-2-({5-[(1S)-1-[(5-chloro-2- methylpyridin-3- yl)amino]propyl]thiophen-2- yl}formamido)-3-cyclopentyl-N- [(1R)-2,2- difluorocyclopropyl]propanamide | 525.06 |
| 54 | 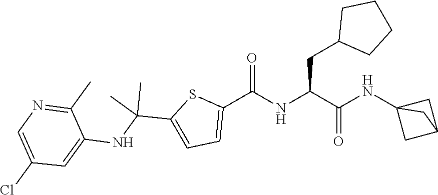 [SMILES: Cc1ncc(Cl)cc1NC(C)(C)c1ccc(C(=O)N[C@@H](CC2CCCC2)C(=O)NC23CC(C2)C3)s1] | (2S)-N-{bicyclo[1.1.1]pentan-1-yl}- 2-[(5-{2-[(5-chloro-2-methylpyridin- 3-yl)amino]propan-2-yl}thiophen-2- yl)formamido]-3- cyclopentylpropanamide | 515.11 |
| 55 | 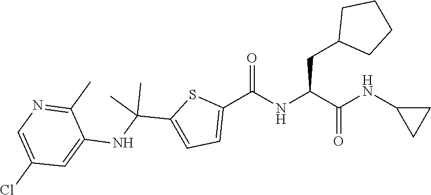 [SMILES: Cc1ncc(Cl)cc1NC(C)(C)c1ccc(C(=O)N[C@@H](CC2CCCC2)C(=O)NC2CC2)s1] | (2S)-2-[(5-{2-[(5-chloro-2- methylpyridin-3-yl)amino]propan-2- yl}thiophen-2-yl)formamido]-3- cyclopentyl-N- cyclopropylpropanamide | 489.08 |
| 56 | 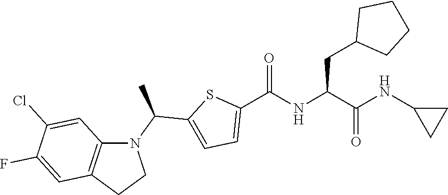 [SMILES: C[C@@H](c1ccc(C(=O)N[C@@H](CC2CCCC2)C(=O)NC2CC2)s1)N1CCc2cc(F)c(Cl)cc21] | (2S)-2-({5-[(1S)-1-(6-chloro-5- fluoro-2,3-dihydro-1H-indol-1- yl)ethyl]thiophen-2-yl}formamido)-3- cyclopentyl-N- cyclopropylpropanamide | 504.06 |
| 57 | 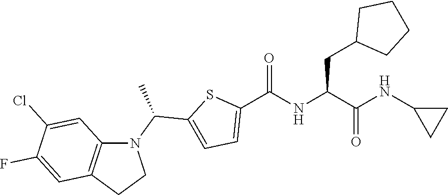 [SMILES: C[C@H](c1ccc(C(=O)N[C@@H](CC2CCCC2)C(=O)NC2CC2)s1)N1CCc2cc(F)c(Cl)cc21] | (2S)-2-({5-[(1R)-1-(6-chloro-5- fluoro-2,3-dihydro-1H-indol-1- yl)ethyl]thiophen-2-yl}formamido)-3- cyclopentyl-N- cyclopropylpropanamide | 504.06 |
| 58 | 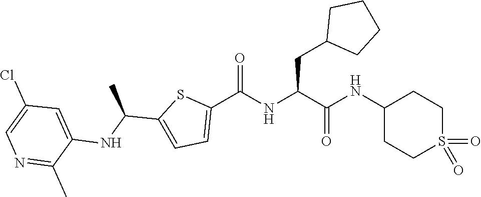 [SMILES: Cc1ncc(Cl)cc1N[C@@H](C)c1ccc(C(=O)N[C@@H](CC2CCCC2)C(=O)NC2CCS(=O)(=O)CC2)s1] | (2S)-2-({5-[(1S)-1-[(5-chloro-2- methylpyridin-3- yl)amino]ethyl]thiophen-2- yl}formamido)-3-cyclopentyl-N-(1,1- dioxo-1λ⁶-thian-4-yl)propanamide | 567.16 |
| 59 | 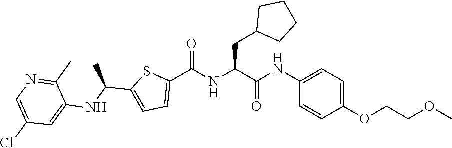 [SMILES: COCCOc1ccc(NC(=O)[C@H](CC2CCCC2)NC(=O)c2ccc([C@H](C)Nc3cc(Cl)cnc3C)s2)cc1] | (2S)-2-({5-[(1S)-1-[(5-chloro-2- methylpyridin-3- yl)amino]ethyl]thiophen-2- yl}formamido)-3-cyclopentyl-N-[4- (2- methoxyethoxy)phenyl]propanamide | 585.16 |
| 60 | 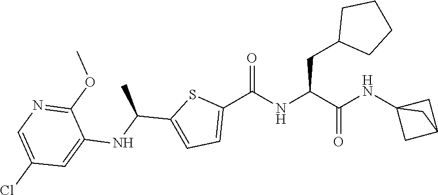 [SMILES: COc1ncc(Cl)cc1N[C@@H](C)c1ccc(C(=O)N[C@@H](CC2CCCC2)C(=O)NC23CC(C2)C3)s1] | (2S)-N-{bicyclo[1.1.1]pentan-1-yl}- 2-({5-[(1S)-1-[(5-chloro-2- methoxypyridin-3- yl)amino]ethyl]thiophen-2- yl}formamido)-3- cyclopentylpropanamide | 517.09 |
| 61 | 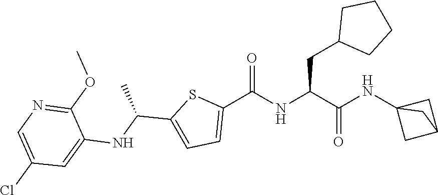 [SMILES: COc1ncc(Cl)cc1N[C@H](C)c1ccc(C(=O)N[C@@H](CC2CCCC2)C(=O)NC23CC(C2)C3)s1] | (2S)-N-{bicyclo[1.1.1]pentan-1-yl}- 2-({5-[(1R)-1-[(5-chloro-2- methoxypyridin-3- yl)amino]ethyl]thiophen-2- yl}formamido)-3- cyclopentylpropanamide | 517.09 |
| 62 | 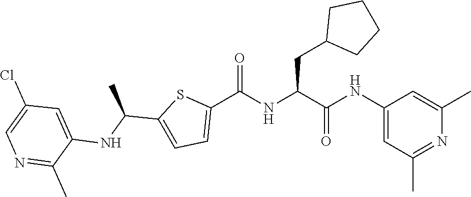 [SMILES: Cc1cc(NC(=O)[C@H](CC2CCCC2)NC(=O)c2ccc([C@H](C)Nc3cc(Cl)cnc3C)s2)cc(C)n1] | (2S)-2-({5-[(1S)-1-[(5-chloro-2- methylpyridin-3- yl)amino]ethyl]thiophen-2- yl}formamido)-3-cyclopentyl-N-(2,6- dimethylpyridin-4-yl)propanamide | 540.12 |
| 63 | 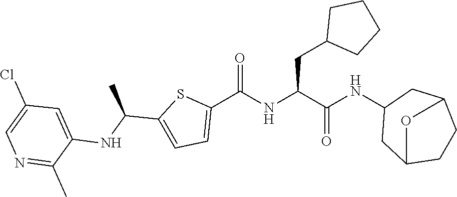 [SMILES: Cc1ncc(Cl)cc1N[C@@H](C)c1ccc(C(=O)N[C@@H](CC2CCCC2)C(=O)NC2CC3CCC(C2)O3)s1] | (2S)-2-({5-[(1S)-1-[(5-chloro-2- methylpyridin-3- yl)amino]ethyl]thiophen-2- yl}formamido)-3-cyclopentyl-N-{8- oxabicyclo[3.2.1]octan-3- yl}propanamide | 545.14 |
| 64 | 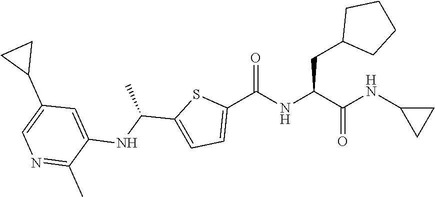 [SMILES: Cc1ncc(C2CC2)cc1N[C@H](C)c1ccc(C(=O)N[C@@H](CC2CCCC2)C(=O)NC2CC2)s1] | (2S)-3-cyclopentyl-N-cyclopropyl-2- ({5-[(1R)-1-[(5-cyclopropyl-2- methylpyridin-3- yl)amino]ethyl]thiophen-2- yl}formamido)propanamide | 480.67 |
| 65 | 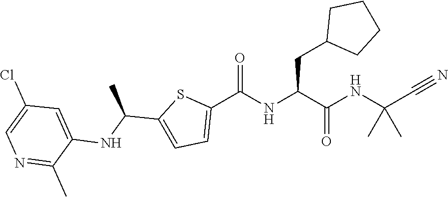 [SMILES: Cc1ncc(Cl)cc1N[C@@H](C)c1ccc(C(=O)N[C@@H](CC2CCCC2)C(=O)NC(C)(C)C#N)s1] | (2S)-2-({5-[(1S)-1-[(5-chloro-2- methylpyridin-3- yl)amino]ethyl]thiophen-2- yl}formamido)-N-(1-cyano-1- methylethyl)-3- cyclopentylpropanamide | 502.07 |
| 66 | 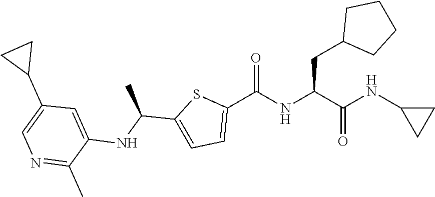 [SMILES: Cc1ncc(C2CC2)cc1N[C@@H](C)c1ccc(C(=O)N[C@@H](CC2CCCC2)C(=O)NC2CC2)s1] | (2S)-3-cyclopentyl-N-cyclopropyl-2- ({5-[(1S)-1-[(5-cyclopropyl-2- methylpyridin-3- yl)amino]ethyl]thiophen-2- yl}formamido)propanamide | 480.67 |
| 67 | 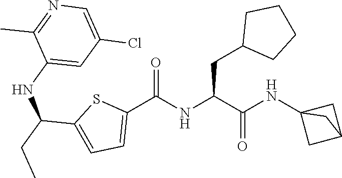 [SMILES: CC[C@@H](Nc1cc(Cl)cnc1C)c1ccc(C(=O)N[C@@H](CC2CCCC2)C(=O)NC23CC(C2)C3)s1] | (2S)-N-{bicyclo[1.1.1]pentan-1-yl}- 2-({5-[(1R)-1-[(5-chloro-2- methylpyridin-3- yl)amino]propyl]thiophen-2- yl}formamido)-3- cyclopentylpropanamide | 515.11 |
| 68 | 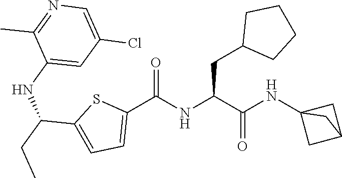 [SMILES: CC[C@H](Nc1cc(Cl)cnc1C)c1ccc(C(=O)N[C@@H](CC2CCCC2)C(=O)NC23CC(C2)C3)s1] | (2S)-N-{bicyclo[1.1.1]pentan-1-yl}- 2-({5-[(1S)-1-[(5-chloro-2- methylpyridin-3- yl)amino]propyl]thiophen-2- yl}formamido)-3- cyclopentylpropanamide | 515.11 |
| 69 | 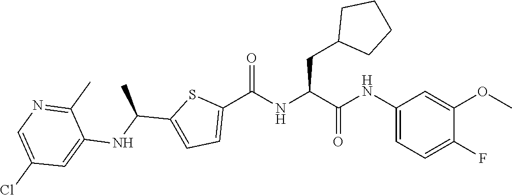 [SMILES: COc1cc(NC(=O)[C@H](CC2CCCC2)NC(=O)c2ccc([C@H](C)Nc3cc(Cl)cnc3C)s2)ccc1F] | (2S)-2-({5-[(1S)-1-[(5-chloro-2- methylpyridin-3- yl)amino]ethyl]thiophen-2- yl}formamido)-3-cyclopentyl-N-(4- fluoro-3- methoxyphenyl)propanamide | 559.1 |
| 70 | 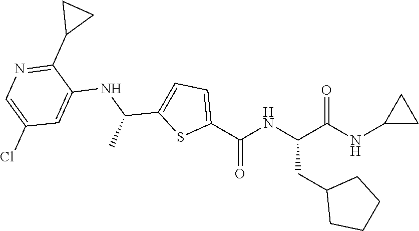 [SMILES: C[C@H](Nc1cc(Cl)cnc1C1CC1)c1ccc(C(=O)N[C@@H](CC2CCCC2)C(=O)NC2CC2)s1] | (2S)-2-({5-[(1S)-1-[(5-chloro-2- cyclopropylpyridin-3- yl)amino]ethyl]thiophen-2- yl}formamido)-3-cyclopentyl-N- cyclopropylpropanamide | 501.09 |
| 71 | 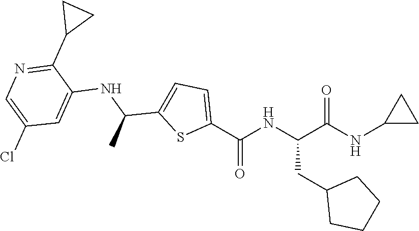 [SMILES: C[C@@H](Nc1cc(Cl)cnc1C1CC1)c1ccc(C(=O)N[C@@H](CC2CCCC2)C(=O)NC2CC2)s1] | (2S)-2-({5-[(1R)-1-[(5-chloro-2- cyclopropylpyridin-3- yl)amino]ethyl]thiophen-2- yl}formamido)-3-cyclopentyl-N- cyclopropylpropanamide | 501.09 |
| 72 | 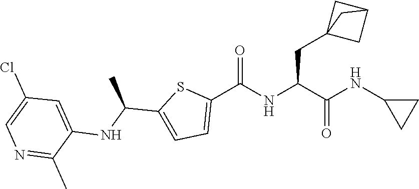 [SMILES: Cc1ncc(Cl)cc1N[C@@H](C)c1ccc(C(=O)N[C@@H](CC23CC(C2)C3)C(=O)NC2CC2)s1] | (2S)-3-{bicyclo[1.1.1]pentan-1-yl}-2- ({5-[(1S)-1-[(5-chloro-2- methylpyridin-3- yl)amino]ethyl]thiophen-2- yl}formamido)-N- cyclopropylpropanamide | 473.03 |
| 77 | 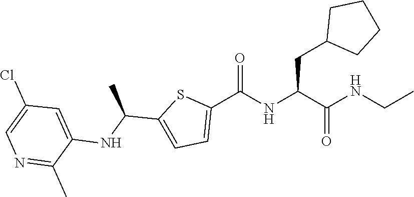 [SMILES: CCNC(=O)[C@H](CC1CCCC1)NC(=O)c1ccc([C@H](C)Nc2cc(Cl)cnc2C)s1] | (2S)-2-({5-[(1S)-1-[(5-chloro-2- methylpyridin-3- yl)amino]ethyl]thiophen-2- yl}formamido)-3-cyclopentyl-N- ethylpropanamide | 463.04 |
| 78 | 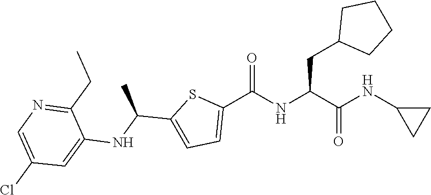 [SMILES: CCc1ncc(Cl)cc1N[C@@H](C)c1ccc(C(=O)N[C@@H](CC2CCCC2)C(=O)NC2CC2)s1] | (2S)-2-({5-[(1S)-1-[(5-chloro-2- ethylpyridin-3- yl)amino]ethyl]thiophen-2- yl}formamido)-3-cyclopentyl-N- cyclopropylpropanamide | 489.08 |
| 79 | 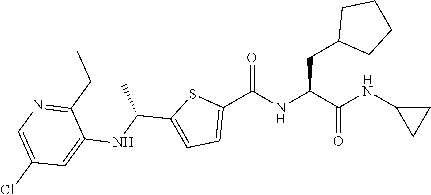 [SMILES: CCc1ncc(Cl)cc1N[C@H](C)c1ccc(C(=O)N[C@@H](CC2CCCC2)C(=O)NC2CC2)s1] | (2S)-2-({5-[(1R)-1-[(5-chloro-2- ethylpyridin-3- yl)amino]ethyl]thiophen-2- yl}formamido)-3-cyclopentyl-N- cyclopropylpropanamide | 489.08 |
| 80 | 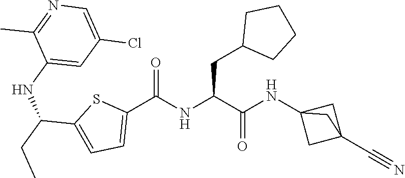 [SMILES: CC[C@H](Nc1cc(Cl)cnc1C)c1ccc(C(=O)N[C@@H](CC2CCCC2)C(=O)NC23CC(C#N)(C2)C3)s1] | (2S)-2-({5-[(1S)-1-[(5-chloro-2- methylpyridin-3- yl)amino]propyl]thiophen-2- yl}formamido)-N-{3- cyanobicyclo[1.1.1]pentan-1-yl}-3- cyclopentylpropanamide | 540.12 |
| 81 | 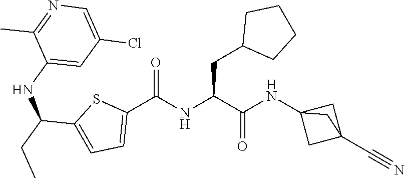 [SMILES: CC[C@@H](Nc1cc(Cl)cnc1C)c1ccc(C(=O)N[C@@H](CC2CCCC2)C(=O)NC23CC(C#N)(C2)C3)s1] | (2S)-2-({5-[(1R)-1-[(5-chloro-2- methylpyridin-3- yl)amino]propyl]thiophen-2- yl}formamido)-N-{3- cyanobicyclo[1.1.1]pentan-1-yl}-3- cyclopentylpropanamide | 540.12 |
| 82 | 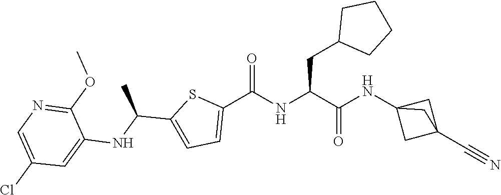 [SMILES: COc1ncc(Cl)cc1N[C@@H](C)c1ccc(C(=O)N[C@@H](CC2CCCC2)C(=O)NC23CC(C#N)(C2)C3)s1] | (2S)-2-({5-[(1S)-1-[(5-chloro-2- methoxypyridin-3- yl)amino]ethyl]thiophen-2- yl}formamido)-N-{3- cyanobicyclo[1.1.1]pentan-1-yl}-3- cyclopentylpropanamide | 542.1 |
| 83 | 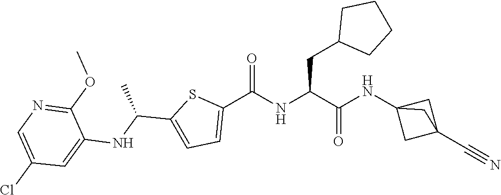 [SMILES: COc1ncc(Cl)cc1N[C@H](C)c1ccc(C(=O)N[C@@H](CC2CCCC2)C(=O)NC23CC(C#N)(C2)C3)s1] | (2S)-2-({5-[(1R)-1-[(5-chloro-2- methoxypyridin-3- yl)amino]ethyl]thiophen-2- yl}formamido)-N-{3- cyanobicyclo[1.1.1]pentan-1-yl}-3- cyclopentylpropanamide | 542.1 |
| 84 | 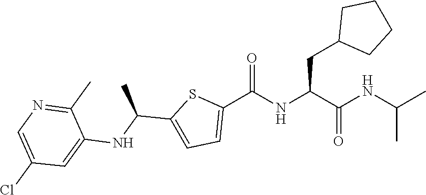 [SMILES: Cc1ncc(Cl)cc1N[C@@H](C)c1ccc(C(=O)N[C@@H](CC2CCCC2)C(=O)NC(C)C)s1] | (2S)-2-({5-[(1S)-1-[(5-chloro-2- methylpyridin-3- yl)amino]ethyl]thiophen-2- yl}formamido)-3-cyclopentyl-N- (propan-2-yl)propanamide | 477.06 |
| 85 | 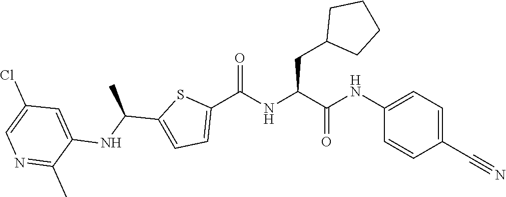 [SMILES: Cc1ncc(Cl)cc1N[C@@H](C)c1ccc(C(=O)N[C@@H](CC2CCCC2)C(=O)Nc2ccc(C#N)cc2)s1] | (2S)-2-({5-[(1S)-1-[(5-chloro-2- methylpyridin-3- yl)amino]ethyl]thiophen-2- yl}formamido)-N-(4-cyanophenyl)-3- cyclopentylpropanamide | 536.09 |
| 89 | 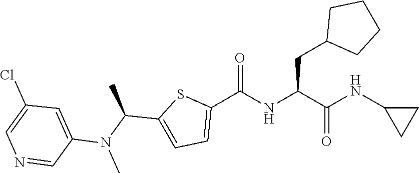 [SMILES: C[C@@H](c1ccc(C(=O)N[C@@H](CC2CCCC2)C(=O)NC2CC2)s1)N(C)c1cncc(Cl)c1] | (2S)-2-({5-[(1S)-1-[(5-chloropyridin- 3-yl)(methyl)amino]ethyl]thiophen-2- yl}formamido)-3-cyclopentyl-N- cyclopropylpropanamide | 475.05 |
| 90 | 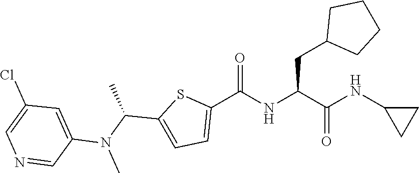 [SMILES: C[C@H](c1ccc(C(=O)N[C@@H](CC2CCCC2)C(=O)NC2CC2)s1)N(C)c1cncc(Cl)c1] | (2S)-2-({5-[(1R)-1-[(5-chloropyridin- 3-yl)(methyl)amino]ethyl]thiophen-2- yl}formamido)-3-cyclopentyl-N- cyclopropylpropanamide | 475.05 |
| 91 | 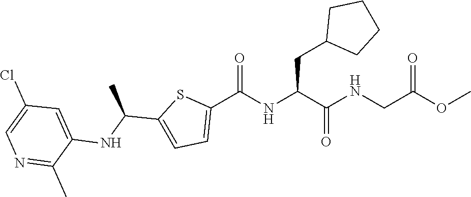 [SMILES: COC(=O)CNC(=O)[C@H](CC1CCCC1)NC(=O)c1ccc([C@H](C)Nc2cc(Cl)cnc2C)s1] | methyl 2-[(2S)-2-({5-[(1S)-1-[(5- chloro-2-methylpyridin-3- yl)amino]ethyl]thiophen-2- yl}formamido)-3- cyclopentylpropanamido]acetate | 507.05 |
| 92 | 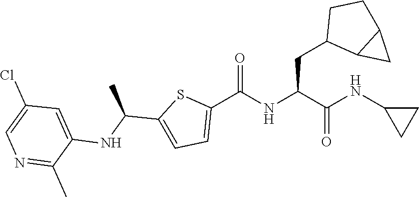 [SMILES: Cc1ncc(Cl)cc1N[C@@H](C)c1ccc(C(=O)N[C@@H](CC2CCC3CC32)C(=O)NC2CC2)s1] | (2S)-3-{bicyclo[3.1.0]hexan-2-yl}-2- ({5-[(1S)-1-[(5-chloro-2- methylpyridin-3- yl)amino]ethyl]thiophen-2- yl}formamido)-N- cyclopropylpropanamide | 487.06 |
| 93 | 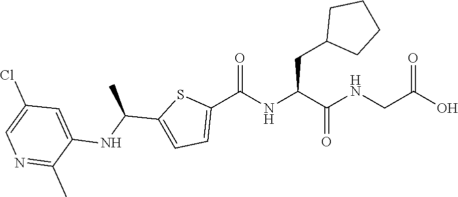 [SMILES: Cc1ncc(Cl)cc1N[C@@H](C)c1ccc(C(=O)N[C@@H](CC2CCCC2)C(=O)NCC(=O)O)s1] | 2-[(2S)-2-({5-[(1S)-1-[(5-chloro-2- methylpyridin-3- yl)amino]ethyl]thiophen-2- yl}formamido)-3- cyclopentylpropanamido]acetic acid | 493.02 |
| 96 | 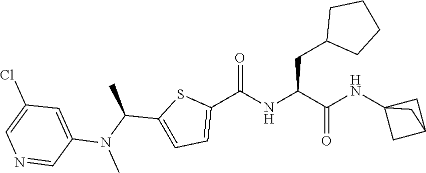 [SMILES: C[C@@H](c1ccc(C(=O)N[C@@H](CC2CCCC2)C(=O)NC23CC(C2)C3)s1)N(C)c1cncc(Cl)c1] | (2S)-N-{bicyclo[1.1.1]pentan-1-yl}- 2-({5-[(1S)-1-[(5-chloropyridin-3- yl)(methyl)amino]ethyl]thiophen-2- yl}formamido)-3- cyclopentylpropanamide | 501.09 |
| 97 | 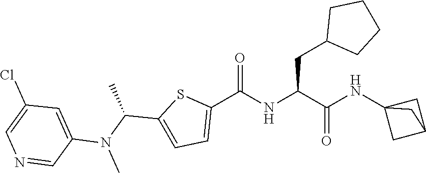 [SMILES: C[C@H](c1ccc(C(=O)N[C@@H](CC2CCCC2)C(=O)NC23CC(C2)C3)s1)N(C)c1cncc(Cl)c1] | (2S)-N-{bicyclo[1.1.1]pentan-1-yl}- 2-({5-[(1R)-1-[(5-chloropyridin-3- yl)(methyl)amino]ethyl]thiophen-2- yl}formamido)-3- cyclopentylpropanamide | 501.09 |
| 100 | 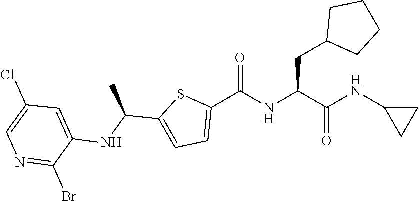 [SMILES: C[C@H](Nc1cc(Cl)cnc1Br)c1ccc(C(=O)N[C@@H](CC2CCCC2)C(=O)NC2CC2)s1] | (2S)-2-({5-[(1S)-1-[(2-bromo-5- chloropyridin-3- yl)amino]ethyl]thiophen-2- yl}formamido)-3-cyclopentyl-N- cyclopropylpropanamide | 539.92 |
| 101 | 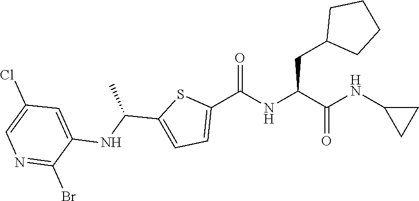 [SMILES: C[C@@H](Nc1cc(Cl)cnc1Br)c1ccc(C(=O)N[C@@H](CC2CCCC2)C(=O)NC2CC2)s1] | (2S)-2-({5-[(1R)-1-[(2-bromo-5- chloropyridin-3- yl)amino]ethyl]thiophen-2- yl}formamido)-3-cyclopentyl-N- cyclopropylpropanamide | 539.92 |
| 102 | 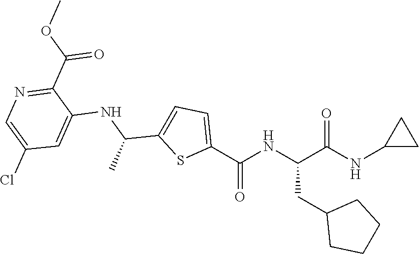 [SMILES: COC(=O)c1ncc(Cl)cc1N[C@@H](C)c1ccc(C(=O)N[C@@H](CC2CCCC2)C(=O)NC2CC2)s1] | methyl 5-chloro-3-{[(1S)-1-(5-{[(1S)- 2-cyclopentyl-1- (cyclopropylcarbamoyl)ethyl]carba- moyl}thiophen-2- yl)ethyl]amino}pyridine-2- carboxylate | 519.06 |
| 103 | 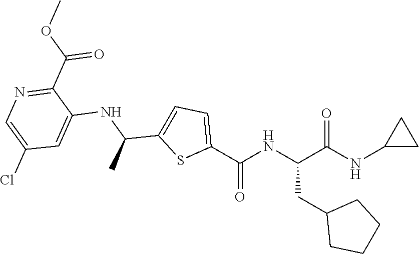 [SMILES: COC(=O)c1ncc(Cl)cc1N[C@H](C)c1ccc(C(=O)N[C@@H](CC2CCCC2)C(=O)NC2CC2)s1] | methyl 5-chloro-3-{[(1R)-1-(5- {[(1S)-2-cyclopentyl-1- (cyclopropylcarbamoyl)ethyl]carba- moyl}thiophen-2- yl)ethyl]amino}pyridine-2- carboxylate | 519.06 |
| 104 | 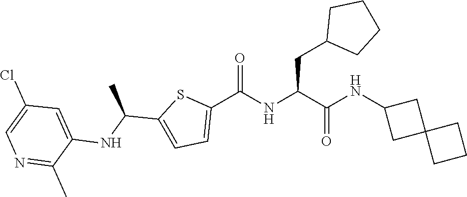 [SMILES: Cc1ncc(Cl)cc1N[C@@H](C)c1ccc(C(=O)N[C@@H](CC2CCCC2)C(=O)NC2CC3(CCC3)C2)s1] | (2S)-2-({5-[(1S)-1-[(5-chloro-2- methylpyridin-3- yl)amino]ethyl]thiophen-2- yl}formamido)-3-cyclopentyl-N- {spiro[3.3]heptan-2-yl}propanamide | 529.14 |
| 105 | 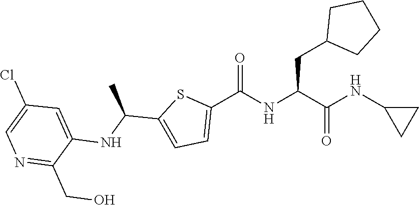 [SMILES: C[C@H](Nc1cc(Cl)cnc1CO)c1ccc(C(=O)N[C@@H](CC2CCCC2)C(=O)NC2CC2)s1] | (2S)-2-({5-[(1S)-1-{[5-chloro-2- (hydroxymethyl)129yridine-3- yl]amino}ethyl]thiophen-2- yl}formamido)-3-cyclopentyl-N- cyclopropylpropanamide | 491.05 |
| 106 | 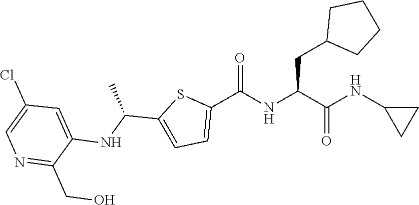 [SMILES: C[C@@H](Nc1cc(Cl)cnc1CO)c1ccc(C(=O)N[C@@H](CC2CCCC2)C(=O)NC2CC2)s1] | (2S)-2-({5-[(1R)-1-{[5-chloro-2- (hydroxymethyl)129yridine-3- yl]amino}ethyl]thiophen-2- yl}formamido)-3-cyclopentyl-N- cyclopropylpropanamide | 491.05 |
| 107 | 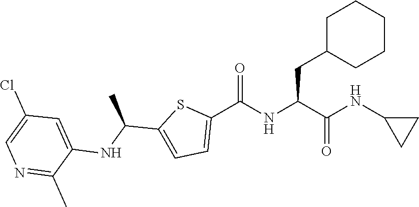 [SMILES: Cc1ncc(Cl)cc1N[C@@H](C)c1ccc(C(=O)N[C@@H](CC2CCCCC2)C(=O)NC2CC2)s1] | (2S)-2-({5-[(1S)-1-[(5-chloro-2- methylpyridin-3- yl)amino]ethyl]thiophen-2- yl}formamido)-3-cyclohexyl-N- cyclopropylpropanamide | 489.08 |
| 108 | 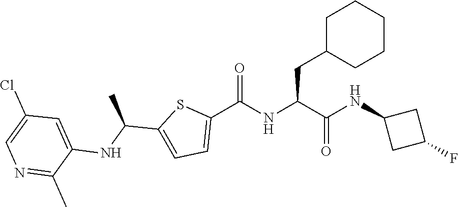 [SMILES: Cc1ncc(Cl)cc1N[C@@H](C)c1ccc(C(=O)N[C@@H](CC2CCCCC2)C(=O)N[C@H]2C[C@H](F)C2)s1] | (2S)-2-({5-[(1S)-1-[(5-chloro-2- methylpyridin-3- yl)amino]ethyl]thiophen-2- yl}formamido)-3-cyclohexyl-N- [(1r,3r)-3- fluorocyclobutyl]propanamide | 521.09 |
| 109 | 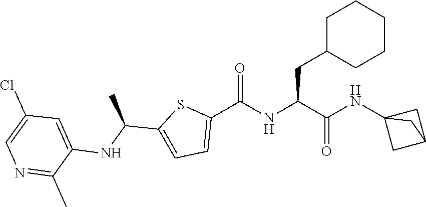 [SMILES: Cc1ncc(Cl)cc1N[C@@H](C)c1ccc(C(=O)N[C@@H](CC2CCCCC2)C(=O)NC23CC(C2)C3)s1] | (2S)-N-{bicyclo[1.1.1]pentan-1-yl}- 2-({5-[(1S)-1-[(5-chloro-2- methylpyridin-3- yl)amino]ethyl]thiophen-2- yl}formamido)-3- cyclohexylpropanamide | 515.11 |
| 110 | 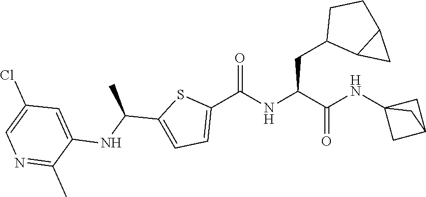 [SMILES: Cc1ncc(Cl)cc1N[C@@H](C)c1ccc(C(=O)N[C@@H](CC2CCC3CC32)C(=O)NC23CC(C2)C3)s1] | (2S)-N-{bicyclo[1.1.1]pentan-1-yl}- 3-{bicyclo[3.1.0]hexan-2-yl}-2-({5- [(1S)-1-[(5-chloro-2-methylpyridin-3- yl)amino]ethyl]thiophen-2- yl}formamido)propanamide | 513.1 |
| 111 | 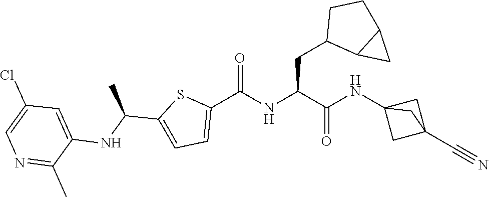 [SMILES: Cc1ncc(Cl)cc1N[C@@H](C)c1ccc(C(=O)N[C@@H](CC2CCC3CC32)C(=O)NC23CC(C#N)(C2)C3)s1] | (2S)-3-{bicyclo[3.1.0]hexan-2-yl}-2- ({5-[(1S)-1-[(5-chloro-2- methylpyridin-3- yl)amino]ethyl]thiophen-2- yl}formamido)-N-{3- cyanobicyclo[1.1.1]pentan-1- yl}propanamide | 538.11 |
| 112 | 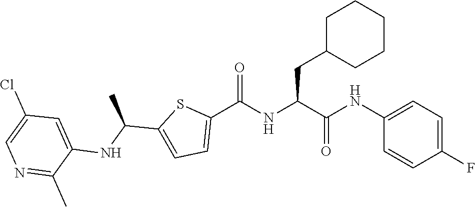 [SMILES: Cc1ncc(Cl)cc1N[C@@H](C)c1ccc(C(=O)N[C@@H](CC2CCCCC2)C(=O)Nc2ccc(F)cc2)s1] | (2S)-2-({5-[(1S)-1-[(5-chloro-2- methylpyridin-3- yl)amino]ethyl]thiophen-2- yl}formamido)-3-cyclohexyl-N-(4- fluorophenyl)propanamide | 543.1 |
| 113 | 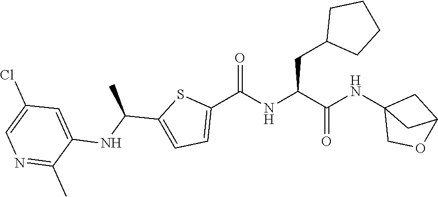 [SMILES: Cc1ncc(Cl)cc1N[C@@H](C)c1ccc(C(=O)N[C@@H](CC2CCCC2)C(=O)NC23COC(C2)C3)s1] | (2S)-2-({5-[(1S)-1-[(5-chloro-2- methylpyridin-3- yl)amino]ethyl]thiophen-2- yl}formamido)-3-cyclopentyl-N-{2- oxabicyclo[2.1.1]hexan-4- yl}propanamide | 517.09 |
| 114 | 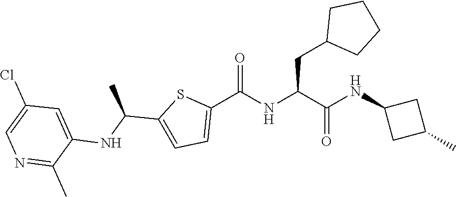 [SMILES: Cc1ncc(Cl)cc1N[C@@H](C)c1ccc(C(=O)N[C@@H](CC2CCCC2)C(=O)N[C@H]2C[C@H](C)C2)s1] | (2S)-2-({5-[(1S)-1-[(5-chloro-2- methylpyridin-3- yl)amino]ethyl]thiophen-2- yl}formamido)-3-cyclopentyl-N- [(1s,3r)-3- methylcyclobutyl]propanamide | 503.1 |
| 115 | 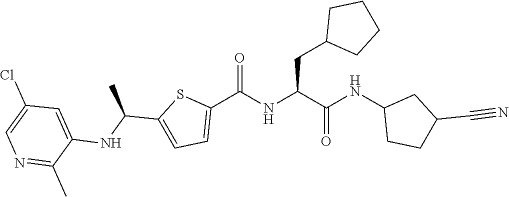 [SMILES: Cc1ncc(Cl)cc1N[C@@H](C)c1ccc(C(=O)N[C@@H](CC2CCCC2)C(=O)NC2CCC(C#N)C2)s1] | (2S)-2-({5-[(1S)-1-[(5-chloro-2- methylpyridin-3- yl)amino]ethyl]thiophen-2- yl}formamido)-N-(3- cyanocyclopentyl)-3- cyclopentylpropanamide | 528.11 |
| 116 | 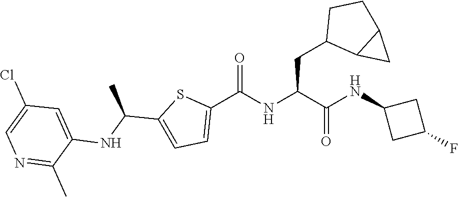 [SMILES: Cc1ncc(Cl)cc1N[C@@H](C)c1ccc(C(=O)N[C@@H](CC2CCC3CC32)C(=O)N[C@H]2C[C@H](F)C2)s1] | (2S)-3-{bicyclo[3.1.0]hexan-2-yl}-2- ({5-[(1S)-1-[(5-chloro-2- methylpyridin-3- yl)amino]ethyl]thiophen-2- yl}formamido)-N-[(1r,3r)-3- fluorocyclobutyl]propanamide | 519.08 |

### Example 16

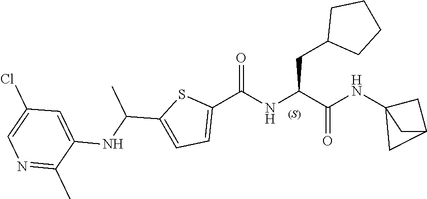

### N-[(1S)-2-(1-bicyclo [1.1.1]pentanylamino)-1-(cyclopentylmethyl)-2-oxo-ethyl]-5-[1-[(5-chloro-2-methyl-3-pyridyl) amino]ethyl]thiophene-2-carboxamide

### Step 1—methyl 5-(1-hydroxyethyl) thiophene-2-carboxylate

To a mixture of methyl 5-formylthiophene-2-carboxylate (3.00 g, 17.63 mmol, 1.00 eq) in THF (30 ml) was added MeMgBr (3 M, 7.64 mL, 1.30 eq) at 0° C. for 0.5 hr. The reaction mixture was added to water (30 mL). The aqueous phase was extracted with EtOAc (3*30 mL). The combined organic phase was washed with brine (3*30 mL), dried with anhydrous Na₂SO₄ filtered and concentrated in vacuum. The residue was purified by column chromatography (SiO₂, Petroleum ether:EtOAc=100:1 to 0:1, TM/R_{f}=0.6) to afford the title compound methyl 5-(1-hydroxyethyl) thiophene-2-carboxylate (2.30 g, 12.35 mmol, 70.06% yield) as a yellow solid. LC-MS (ES⁺, m/z): 187.2 [(M+H)⁺].

### Step 2—methyl 5-(1-bromoethyl) thiophene-2-carboxylate

To a mixture methyl 5-(1-hydroxyethyl) thiophene-2-carboxylate (2.30 g, 12.35 mmol, 1.00 eq) in DCM (23 mL) was added PBr₃ (10.03 g, 37.05 mmol, 3.00 eq) at 25° C., the reaction was stirred at 25° C. for 2 br. The reaction mixture was added to DCM (15 mL) The mixture was purified by column chromatography (SiO2, Petroleum ether:EtOAc=100:1) to afford the title compound methyl 5-(1-bromoethyl) thiophene-2-carboxylate (3.40 g, crude) as yellow solid.

### Step 3—methyl 5-[1-[(5-chloro-2-methyl-3-pyridyl) amino]ethyl]thiophene-2-carboxylate

To a mixture of methyl 5-(1-bromoethyl)thiophene-2-carboxylate (3.30 g, 13.25 mmol, 1.00 eq) and 5-chloro-2-methyl-pyridin-3-amine (2.08 g, 14.57 mmol, 1.10 eq) in DMF (30 mL) was added CaCO₃ (6.63 g, 66.23 mmol, 500 eq) at 70° C. for 4 hr. The reaction mixture was added to water (10 mL), the aqueous phase was extracted with EtOAc (3*10 mL). The combined organic phase was dried with anhydrous Na₂SO₄, filtered and concentrated in vacuum. The residue was purified by column chromatography (SiO₂, PE:EA=100:1 to 0:1, TM/R_{f}=0.7) to afford the title compound methyl 5-[1-[(5-chloro-2-methyl-3-pyridyl) amino]ethyl]thiophene-2-carboxylate (2.50 g, crude) as yellow solid, LC-MS (ES⁺, m/z): 311.1 [(M+H)⁺].

### Step 4—5-[1-[(5-chloro-2-methyl-3-pyridyl) amino]ethyl]thiophene-2-carboxylic acid

To a mixture methyl 5-[1-[(5-chloro-2-methyl-3-pyridyl)amino]ethyl]thiophene-2-carboxylate (1.60 g, 5.15 mmol, 1.00 eq) in THE (8 mL), H₂O (4 mL) were added LiOH·H₂O (432.10 mg, 10.30 mmnol, 2.00 eq) at 25° C., the reaction was stirred at 25° C. for 2 hour. The reaction mixture was added to water (20 mL). The aqueous phase was extracted with EtOAc (20 mL) and the aqueous phase PH value was adjusted to pH=3 with aq. HCl (2N). The resulting precipitate was collected by filtration to give product as a white solid to afford the title compound 5-[1-[(5-chloro-2-methyl-3-pyridyl) amino]ethyl]thiophene-2-carboxylic acid 3_5 (630.0 mg, crude) as a yellow solid. LC-MS (ES⁺, m/z): 297.1 [(M+H)⁺].

### Step 5—N-[(1S)-2-(1-bicyclo [1.1.1]pentanylamino)-1-(cyclopentylmethyl)-2-oxo-ethyl]-5-[1-[(5-chloro-2-methyl-3-pyridyl) amino]ethyl]thiophene-2-carboxamide (Compound 10)

To a mixture of 5-[1-[(5-chloro-2-methyl-3-pyridyl)amino]ethyl]thiophene-2-carboxylic acid 3_5 (400.00 mg, 1.35 mmol, 1.00 eq) and (2S)-2-amino-N-(1-bicyclo[1.1.1]pentanyl)-3-cyclopentyl-propanamide hydrochloride (418.60 mg, 1.62 mmol, 1.20 eq) in DMF (4 mL) were added T₃P (1.72 g, 2.70 mmol, 1.60 mL, 50% purity, 2.00 eq) and TEA (818.30 mg, 8.09 mmol, 1.13 mL, 6.00 eq) at 0° C., the reaction was stirred at 25° C. for 0.5 hour. The reaction mixture was added to water (10 mL), the aqueous phase was extracted with EtOAc (3*10 mL). The combined organic phase was dried with anhydrous Na₂SO₄, filtered and concentrated in vacuum. The residue was purified by prep-HPLC (column: Waters Xbridge Prep OBD C18 150*40 mm*10 um; mobile phase: [water (NH₄HCO₃)-ACN]; B %: 40%-75%, 8 min) to afford the title compound N-[(1S)-2(1-bicyclo[1.1.1]pentanylamino)-1-(cyclopentylmethyl)-2-oxo-ethyl]-5-[1-[(5-chloro-2-methyl-3-pyridyl)amino]ethyl]thiophene-2-carboxamide (135.0 mg, 269.42 μmol, 19.99% yield) as a white solid. LC-MS (ES+, m/z) 501.2 [(M+H)⁺], ¹H NMR (400 MHz, DMSO-d₆) δ=8.48 (d, J=4 Hz, 1H), 8.31-8.26 (m, 1H), 7.71 (dd, J=4, 4 Hz, 1H), 7.67 (d, J=4 Hz, 1H), 7.07 (d, J=4 Hz, 1H), 6.84 (m, 1H), 5.85 (t, J=8 Hz, 1H), 4.94 (m, 1H), 4.31-4.23 (m, 1H), 2.39-2.35 (m, 4H), 1.95 (d, J=4 Hz, 6H), 1.82-1.61 (m, 5H), 1.59 (d, J=4 Hz, 3H), 1.55-1.49 (m, 2H), 1.46-1.37 (m, 2H).

Other compounds were made via methods similar to that described in the examples and the

TABLE L

| | | Exact mass | Molecular |
|---|---|---|---|
| # | Structure | M + 1 (g/mol) | weight (g/mol) |
| 129 | 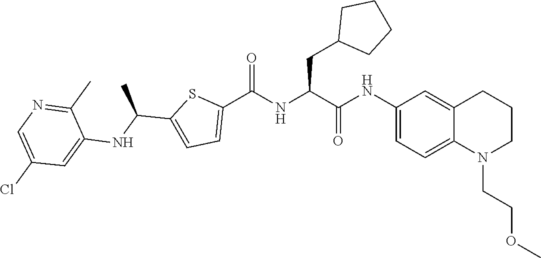 [SMILES: COCCN1CCCc2cc(NC(=O)[C@H](CC3CCCC3)NC(=O)c3ccc([C@H](C)Nc4cc(Cl)cnc4C)s3)ccc21] | 624.24 | 623.2697 |
| | (2S)-2-({5-[(1S)-1-[(5-chloro-2-methylpyridin-3- | | |
| | yl)amino]ethyl]thiophen-2-yl}formamido)-3- | | |
| | cyclopentyl-N-[1-(2-methoxyethyl)-1,2,3,4- | | |
| | tetrahydroquinolin-6-yl]propanamide | | |
| 130 | 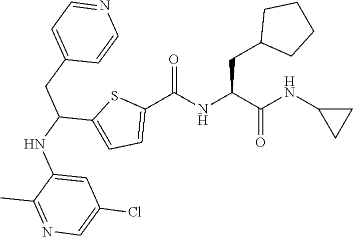 [SMILES: Cc1ncc(Cl)cc1NC(Cc1ccncc1)c1ccc(C(=O)N[C@@H](CC2CCCC2)C(=O)NC2CC2)s1] | 552.13 | 551.2122 |
| | (2S)-2-[(5-{1-[(5-chloro-2-methylpyridin-3- | | |
| | yl)amino]-2-(pyridin-4-yl)ethyl}thiophen-2- | | |
| | yl)formamido]-3-cyclopentyl-N- | | |
| | cyclopropylpropanamide | | |
| 131 | 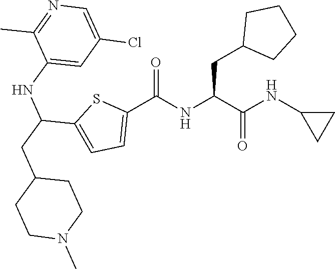 [SMILES: Cc1ncc(Cl)cc1NC(CC1CCN(C)CC1)c1ccc(C(=O)N[C@@H](CC2CCCC2)C(=O)NC2CC2)s1] | 572.21 | 571.2748 |
| | (2S)-2-[(5-{1-[(5-chloro-2-methylpyridin-3- | | |
| | yl)amino]-2-(1-methylpiperidin-4-yl)ethyl}thiophen- | | |
| | 2-yl)formamido]-3-cyclopentyl-N- | | |
| | cyclopropylpropanamide | | |
| 132 | 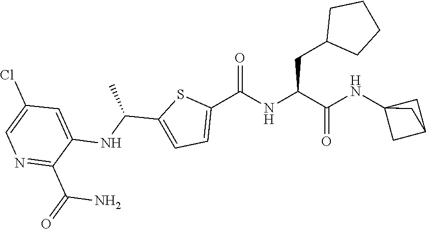 [SMILES: C[C@@H](Nc1cc(Cl)cnc1C(N)=O)c1ccc(C(=O)N[C@@H](CC2CCCC2)C(=O)NC23CC(C2)C3)s1] | 530.08 | 529.1914 |
| | 3-{[(1R)-1-(5-{[(1S)-1-({bicyclo[1.1.1]pentan-1- | | |
| | yl}carbamoyl)-2- | | |
| | cyclopentylethyl]carbamoyl}thiophen-2- | | |
| | yl)ethyl]amino}-5-chloropyridine-2-carboxamide | | |
| 133 | 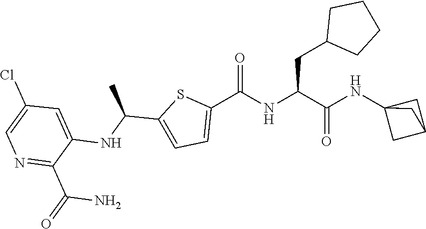 [SMILES: C[C@H](Nc1cc(Cl)cnc1C(N)=O)c1ccc(C(=O)N[C@@H](CC2CCCC2)C(=O)NC23CC(C2)C3)s1] | 530.08 | 529.1914 |
| | 3-{[(1S)-1-(5-{[(1S)-1-({bicyclo[1.1.1]pentan-1- | | |
| | yl}carbamoyl)-2- | | |
| | cyclopentylethyl]carbamoyl}thiophen-2- | | |
| | yl)ethyl]amino}-5-chloropyridine-2-carboxamide | | |
| 134 | 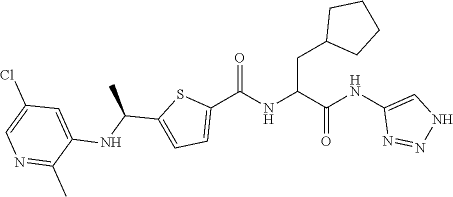 [SMILES: Cc1ncc(Cl)cc1N[C@@H](C)c1ccc(C(=O)NC(CC2CCCC2)C(=O)Nc2c[nH]nn2)s1] | 502.03 | 501.1714 |
| | 2-({5-[(1S)-1-[(5-chloro-2-methylpyridin-3- | | |
| | yl)amino]ethyl]thiophen-2-yl}formamido)-3- | | |
| | cyclopentyl-N-(1H-1,2,3-triazol-4-yl)propanamide | | |
| 135 | 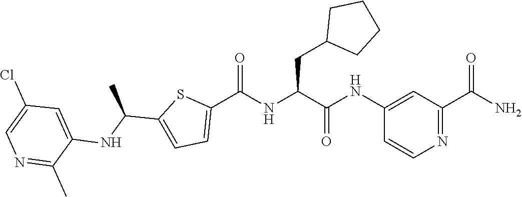 [SMILES: Cc1ncc(Cl)cc1N[C@@H](C)c1ccc(C(=O)N[C@@H](CC2CCCC2)C(=O)Nc2ccnc(C(N)=O)c2)s1] | 555.09 | 554.1867 |
| | 4-[(2S)-2-({5-[(1S)-1-[(5-chloro-2-methylpyridin-3- | | |
| | yl)amino]ethyl]thiophen-2-yl}formamido)-3- | | |
| | cyclopentylpropanamido]pyridine-2-carboxamide | | |
| 136 | 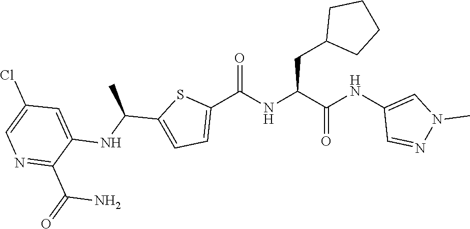 [SMILES: C[C@H](Nc1cc(Cl)cnc1C(N)=O)c1ccc(C(=O)N[C@@H](CC2CCCC2)C(=O)Nc2cnn(C)c2)s1] | 544.07 | 543.1819 |
| | 5-chloro-3-{[(1S)-1-(5-{[(1S)-2-cyclopentyl-1-[(1- | | |
| | methyl-1H-pyrazol-4- | | |
| | yl)carbamoyl]ethyl]carbamoyl}thiophen-2- | | |
| | yl)ethyl]amino}pyridine-2-carboxamide | | |
| 137 | 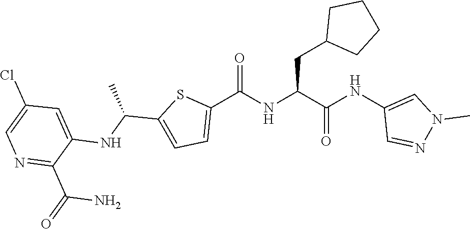 [SMILES: C[C@@H](Nc1cc(Cl)cnc1C(N)=O)c1ccc(C(=O)N[C@@H](CC2CCCC2)C(=O)Nc2cnn(C)c2)s1] | 544.07 | 543.1819 |
| | 5-chloro-3-{[(1R)-1-(5-{[(1S)-2-cyclopentyl-1-[(1- | | |
| | methyl-1H-pyrazol-4- | | |
| | yl)carbamoyl]ethyl]carbamoyl}thiophen-2- | | |
| | yl)ethyl]amino}pyridine-2-carboxamide | | |
| 138 | 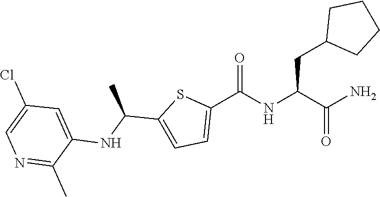 [SMILES: Cc1ncc(Cl)cc1N[C@@H](C)c1ccc(C(=O)N[C@@H](CC2CCCC2)C(N)=O)s1] | 434.98 | 434.1543 |
| | (2S)-2-({5-[(1S)-1-[(5-chloro-2-methylpyridin-3- | | |
| | yl)amino]ethyl]thiophen-2-yl}formamido)-3- | | |
| | cyclopentylpropanamide | | |
| 139 | 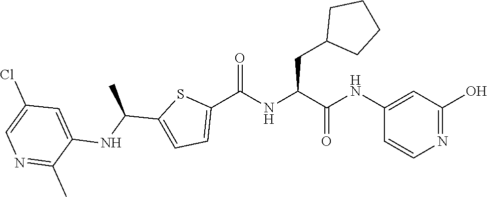 [SMILES: Cc1ncc(Cl)cc1N[C@@H](C)c1ccc(C(=O)N[C@@H](CC2CCCC2)C(=O)Nc2ccnc(O)c2)s1] | 528.07 | 527.1758 |
| | (2S)-2-({5-[(1S)-1-[(5-chloro-2-methylpyridin-3- | | |
| | yl)amino]ethyl]thiophen-2-yl}formamido)-3- | | |
| | cyclopentyl-N-(2-hydroxypyridin-4-yl)propanamide | | |
| 140 | 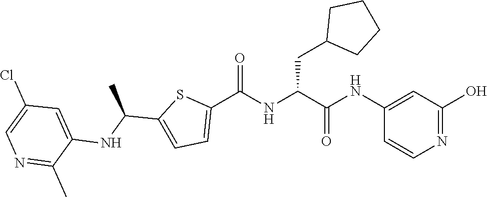 [SMILES: Cc1ncc(Cl)cc1N[C@@H](C)c1ccc(C(=O)N[C@H](CC2CCCC2)C(=O)Nc2ccnc(O)c2)s1] | 528.07 | 527.1758 |
| | (2R)-2-({5-[(1S)-1-[(5-chloro-2-methylpyridin-3- | | |
| | yl)amino]ethyl]thiophen-2-yl}formamido)-3- | | |
| | cyclopentyl-N-(2-hydroxypyridin-4-yl)propanamide | | |
| 141 | 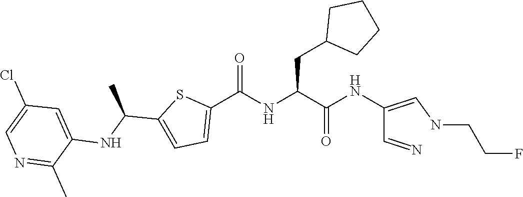 [SMILES: Cc1ncc(Cl)cc1N[C@@H](C)c1ccc(C(=O)N[C@@H](CC2CCCC2)C(=O)N/C(C=N)=C/NCCF)s1] | 547.09 | 546.198 |
| | (2S)-2-({5-[(1S)-1-[(5-chloro-2-methylpyridin-3- | | |
| | yl)amino]ethyl]thiophen-2-yl}formamido)-3- | | |
| | cyclopentyl-N-[1-(2-fluoroethyl)-1H-pyrazol-4- | | |
| | yl]propanamide | | |
| 142 | 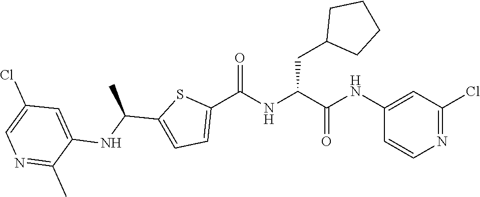 [SMILES: Cc1ncc(Cl)cc1N[C@@H](C)c1ccc(C(=O)N[C@H](CC2CCCC2)C(=O)Nc2ccnc(Cl)c2)s1] | 546.51 | 545.1419 |
| | (2R)-2-({5-[(1S)-1-[(5-chloro-2-methylpyridin-3- | | |
| | yl)amino]ethyl]thiophen-2-yl}formamido)-N-(2- | | |
| | chloropyridin-4-yl)-3-cyclopentylpropanamide | | |
| 143 | 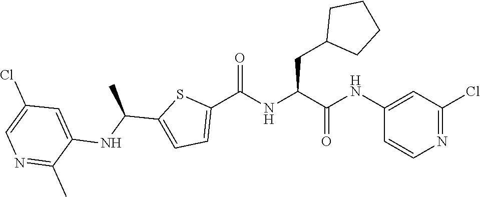 [SMILES: Cc1ncc(Cl)cc1N[C@@H](C)c1ccc(C(=O)N[C@@H](CC2CCCC2)C(=O)Nc2ccnc(Cl)c2)s1] | 546.51 | 545.1419 |
| | (2S)-2-({5-[(1S)-1-[(5-chloro-2-methylpyridin-3- | | |
| | yl)amino]ethyl]thiophen-2-yl}formamido)-N-(2- | | |
| | chloropyridin-4-yl)-3-cyclopentylpropanamide | | |
| 144 | 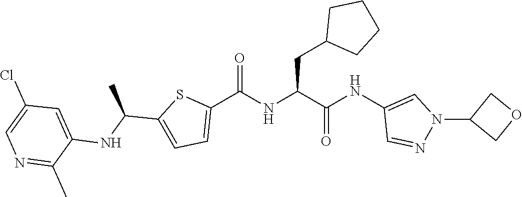 [SMILES: Cc1ncc(Cl)cc1N[C@@H](C)c1ccc(C(=O)N[C@@H](CC2CCCC2)C(=O)Nc2cnn(C3COC3)c2)s1] | 557.11 | 556.2023 |
| | (2S)-2-({5-[(1S)-1-[(5-chloro-2-methylpyridin-3- | | |
| | yl)amino]ethyl]thiophen-2-yl}formamido)-3- | | |
| | cyclopentyl-N-[1-(oxetan-3-yl)-1H-pyrazol-4- | | |
| | yl]propanamide | | |
| 145 | 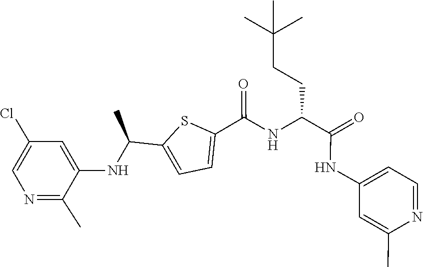 [SMILES: Cc1cc(NC(=O)[C@@H](CCC(C)(C)C)NC(=O)c2ccc([C@H](C)Nc3cc(Cl)cnc3C)s2)ccn1] | 528.11 | 527.2122 |
| | (2R)-2-({5-[(1S)-1-[(5-chloro-2-methylpyridin-3- | | |
| | yl)amino]ethyl]thiophen-2-yl}formamido)-5,5- | | |
| | dimethyl-N-(2-methylpyridin-4-yl)hexanamide | | |
| 146 | 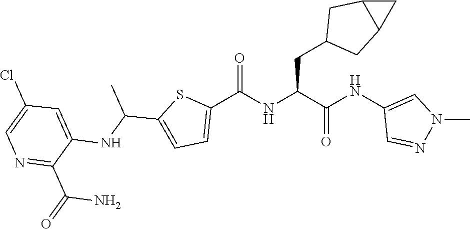 [SMILES: CC(Nc1cc(Cl)cnc1C(N)=O)c1ccc(C(=O)N[C@@H](CC2CC3CC3C2)C(=O)Nc2cnn(C)c2)s1] | 556.08 | 555.1819 |
| | 3-{[1-(5-{[(1S)-2-{bicyclo[3.1.0]hexan-3-yl}-1-[(1- | | |
| | methyl-1H-pyrazol-4- | | |
| | yl)carbamoyl]ethyl]carbamoyl}thiophen-2- | | |
| | yl)ethyl]amino}-5-chloropyridine-2-carboxamide | | |
| 147 3 | 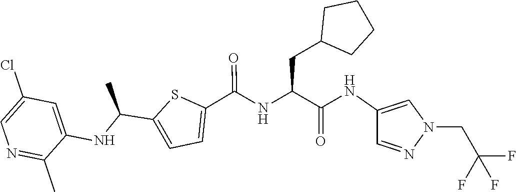 [SMILES: Cc1ncc(Cl)cc1N[C@@H](C)c1ccc(C(=O)N[C@@H](CC2CCCC2)C(=O)Nc2cnn(CC(F)(F)F)c2)s1] | 583.07 | 582.1792 |
| | (2S)-2-({5-[(1S)-1-[(5-chloro-2-methylpyridin-3- | | |
| | yl)amino]ethyl]thiophen-2-yl}formamido)-3- | | |
| | cyclopentyl-N-[1-(2,2,2-trifluoroethyl)-1H-pyrazol-4- | | |
| | yl]propanamide | | |
| 148 | 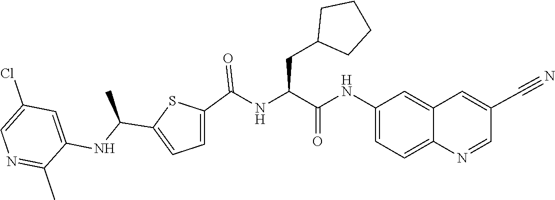 [SMILES: Cc1ncc(Cl)cc1N[C@@H](C)c1ccc(C(=O)N[C@@H](CC2CCCC2)C(=O)Nc2ccc3ncc(C#N)cc3c2)s1] | 587.14 | 586.1918 |
| | (2S)-2-({5-[(1S)-1-[(5-chloro-2-methylpyridin-3- | | |
| | yl)amino]ethyl]thiophen-2-yl}formamido)-N-(3- | | |
| | cyanoquinolin-6-yl)-3-cyclopentylpropanamide | | |
| 149 | 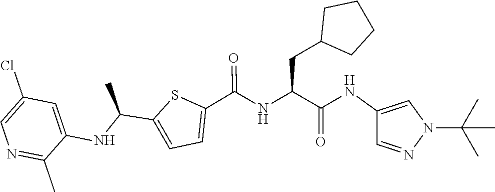 [SMILES: Cc1ncc(Cl)cc1N[C@@H](C)c1ccc(C(=O)N[C@@H](CC2CCCC2)C(=O)Nc2cnn(C(C)(C)C)c2)s1] | 557.15 | 556.2387 |
| | (2S)-N-(1-tert-butyl-1H-pyrazol-4-yl)-2-({5-[(1S)-1- | | |
| | [(5-chloro-2-methylpyridin-3- | | |
| | yl)amino]ethyl]thiophen-2-yl}formamido)-3- | | |
| | cyclopentylpropanamide | | |
| 150 | 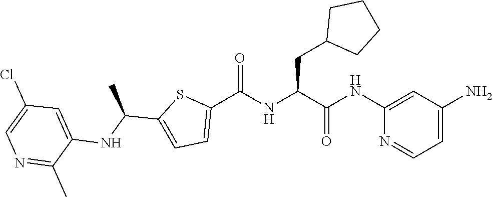 [SMILES: Cc1ncc(Cl)cc1N[C@@H](C)c1ccc(C(=O)N[C@@H](CC2CCCC2)C(=O)Nc2cc(N)ccn2)s1] | 527.08 | 526.1918 |
| | (2S)-N-(4-aminopyridin-2-yl)-2-({5-[(1S)-1-[(5- | | |
| | chloro-2-methylpyridin-3-yl)amino]ethyl]thiophen-2- | | |
| | yl}formamido)-3-cyclopentylpropanamide | | |
| 151 | 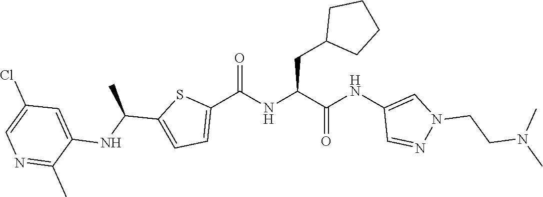 [SMILES: Cc1ncc(Cl)cc1N[C@@H](C)c1ccc(C(=O)N[C@@H](CC2CCCC2)C(=O)Nc2cnn(CCN(C)C)c2)s1] | 572.17 | 571.2496 |
| | (2S)-2-({5-[(1S)-1-[(5-chloro-2-methylpyridin-3- | | |
| | yl)amino]ethyl]thiophen-2-yl}formamido)-3- | | |
| | cyclopentyl-N-{1-[2-(dimethylamino)ethyl]-1H- | | |
| | pyrazol-4-yl}propanamide | | |
| 152 | 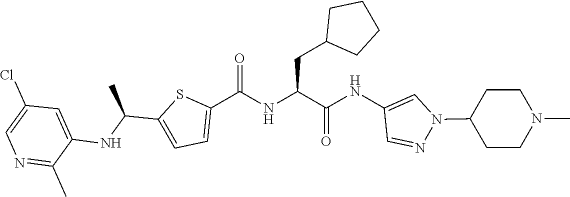 [SMILES: Cc1ncc(Cl)cc1N[C@@H](C)c1ccc(C(=O)N[C@@H](CC2CCCC2)C(=O)Nc2cnn(C3CCN(C)CC3)c2)s1] | 598.21 | 597.2653 |
| | (2S)-2-({5-[(1S)-1-[(5-chloro-2-methylpyridin-3- | | |
| | yl)amino]ethyl]thiophen-2-yl}formamido)-3- | | |
| | cyclopentyl-N-[1-(1-methylpiperidin-4-yl)-1H- | | |
| | pyrazol-4-yl]propanamide | | |
| 153 | 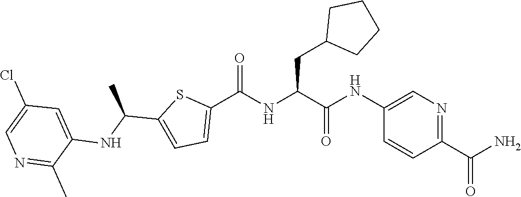 [SMILES: Cc1ncc(Cl)cc1N[C@@H](C)c1ccc(C(=O)N[C@@H](CC2CCCC2)C(=O)Nc2ccc(C(N)=O)nc2)s1] | 555.09 | 554.1867 |
| | 5-[(2S)-2-({5-[(1S)-1-[(5-chloro-2-methylpyridin-3- | | |
| | yl)amino]ethyl]thiophen-2-yl}formamido)-3- | | |
| | cyclopentylpropanamido]pyridine-2-carboxamide | | |
| 154 | 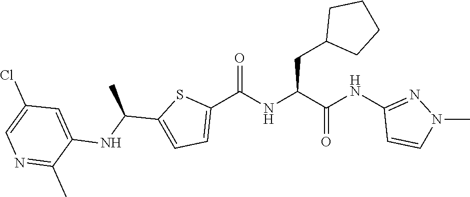 [SMILES: Cc1ncc(Cl)cc1N[C@@H](C)c1ccc(C(=O)N[C@@H](CC2CCCC2)C(=O)Nc2ccn(C)n2)s1] | 515.07 | 514.1918 |
| | (2S)-2-({5-[(1S)-1-[(5-chloro-2-methylpyridin-3- | | |
| | yl)amino]ethyl]thiophen-2-yl}formamido)-3- | | |
| | cyclopentyl-N-(1-methyl-1H-pyrazol-3- | | |
| | yl)propanamide | | |
| 155 | 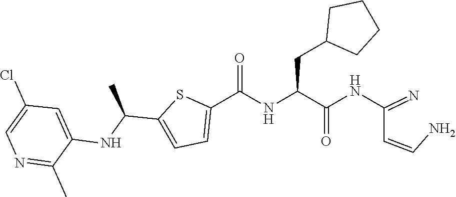 | 501.05 | 500.1761 |
| | (2S)-2-({5-[(1S)-1-[(5-chloro-2-methylpyridin-3- | | |
| | yl)amino]ethyl]thiophen-2-yl}formamido)-3- | | |
| | cyclopentyl-N-(1H-pyrazol-3-yl)propanamide | | |
| 156 | 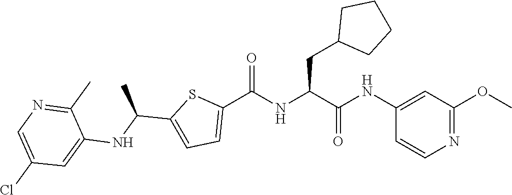 [SMILES: COc1cc(NC(=O)[C@H](CC2CCCC2)NC(=O)c2ccc([C@H](C)Nc3cc(Cl)cnc3C)s2)ccn1] | 542.1 | 541.1914 |
| | (2S)-2-({5-[(1S)-1-[(5-chloro-2-methylpyridin-3- | | |
| | yl)amino]ethyl]thiophen-2-yl}formamido)-3- | | |
| | cyclopentyl-N-(2-methoxypyridin-4-yl)propanamide | | |
| 157 | 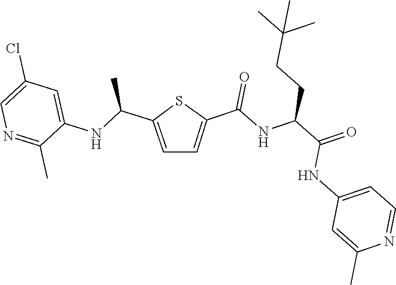 [SMILES: Cc1cc(NC(=O)[C@H](CCC(C)(C)C)NC(=O)c2ccc([C@H](C)Nc3cc(Cl)cnc3C)s2)ccn1] | 528.11 | 527.2122 |
| | (2S)-2-({5-[(1S)-1-[(5-chloro-2-methylpyridin-3- | | |
| | yl)amino]ethyl]thiophen-2-yl}formamido)-5,5- | | |
| | dimethyl-N-(2-methylpyridin-4-yl)hexanamide | | |
| 158 | 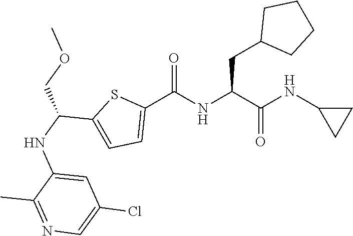 [SMILES: COC[C@@H](Nc1cc(Cl)cnc1C)c1ccc(C(=O)N[C@@H](CC2CCCC2)C(=O)NC2CC2)s1] | 505.07 | 504.1962 |
| | (2S)-2-({5-[(1R)-1-[(5-chloro-2-methylpyridin-3- | | |
| | yl)amino]-2-methoxyethyl]thiophen-2- | | |
| | yl}formamido)-3-cyclopentyl-N- | | |
| | cyclopropylpropanamide | | |
| 159 | 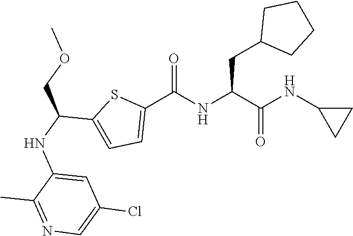 [SMILES: COC[C@H](Nc1cc(Cl)cnc1C)c1ccc(C(=O)N[C@@H](CC2CCCC2)C(=O)NC2CC2)s1] | 505.07 | 504.1962 |
| | (2S)-2-({5-[(1S)-1-[(5-chloro-2-methylpyridin-3- | | |
| | yl)amino]-2-methoxyethyl]thiophen-2- | | |
| | yl}formamido)-3-cyclopentyl-N- | | |
| | cyclopropylpropanamide | | |
| 160 | 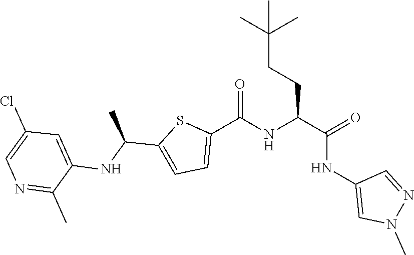 [SMILES: Cc1ncc(Cl)cc1N[C@@H](C)c1ccc(C(=O)N[C@@H](CCC(C)(C)C)C(=O)Nc2cnn(C)c2)s1] | 517.09 | 516.2074 |
| | (2S)-2-({5-[(1S)-1-[(5-chloro-2-methylpyridin-3- | | |
| | yl)amino]ethyl]thiophen-2-yl}formamido)-5,5- | | |
| | dimethyl-N-(1-methyl-1H-pyrazol-4-yl)hexanamide | | |
| 161 | 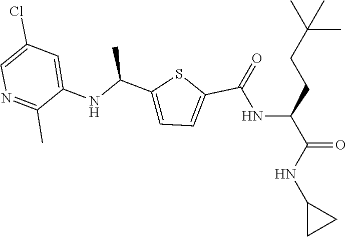 [SMILES: Cc1ncc(Cl)cc1N[C@@H](C)c1ccc(C(=O)N[C@@H](CCC(C)(C)C)C(=O)NC2CC2)s1] | 477.06 | 476.2013 |
| | (2S)-2-({5-[(1S)-1-[(5-chloro-2-methylpyridin-3- | | |
| | yl)amino]ethyl]thiophen-2-yl}formamido)-N- | | |
| | cyclopropyl-5,5-dimethylhexanamide | | |
| 162 | 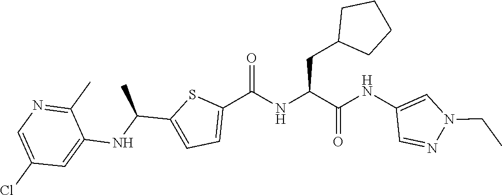 [SMILES: CCn1cc(NC(=O)[C@H](CC2CCCC2)NC(=O)c2ccc([C@H](C)Nc3cc(Cl)cnc3C)s2)cn1] | 529.1 | 528.2074 |
| | (2S)-2-({5-[(1S)-1-[(5-chloro-2-methylpyridin-3- | | |
| | yl)amino]ethyl]thiophen-2-yl}formamido)-3- | | |
| | cyclopentyl-N-(1-ethyl-1H-pyrazol-4-yl)propanamide | | |
| 163 | 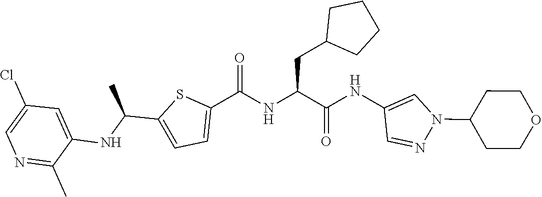 [SMILES: Cc1ncc(Cl)cc1N[C@@H](C)c1ccc(C(=O)N[C@@H](CC2CCCC2)C(=O)Nc2cnn(C3CCOCC3)c2)s1] | 585.16 | 584.2336 |
| | (2S)-2-({5-[(1S)-1-[(5-chloro-2-methylpyridin-3- | | |
| | yl)amino]ethyl]thiophen-2-yl}formamido)-3- | | |
| | cyclopentyl-N-[1-(oxan-4-yl)-1H-pyrazol-4- | | |
| | yl]propanamide | | |
| 164 | 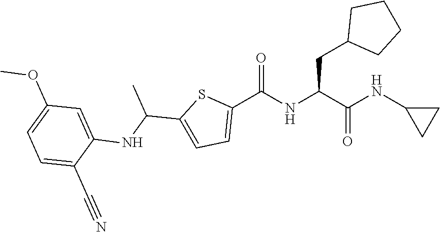 [SMILES: COc1ccc(C#N)c(NC(C)c2ccc(C(=O)N[C@@H](CC3CCCC3)C(=O)NC3CC3)s2)c1] | 480.63 | 480.2195 |
| | (2S)-2-[(5-{1-[(2-cyano-5- | | |
| | methoxyphenyl)amino]ethyl}thiophen-2- | | |
| | yl)formamido]-3-cyclopentyl-N- | | |
| | cyclopropylpropanamide | | |
| 165 | 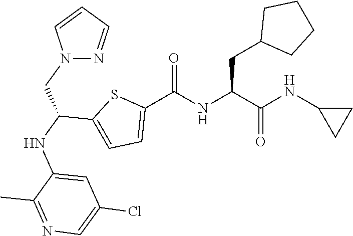 [SMILES: Cc1ncc(Cl)cc1N[C@H](Cn1cccn1)c1ccc(C(=O)N[C@@H](CC2CCCC2)C(=O)NC2CC2)s1] | 541.11 | 540.2074 |
| | (2S)-2-({5-[(1R)-1-[(5-chloro-2-methylpyridin-3- | | |
| | yl)amino]-2-(1H-pyrazol-1-yl)ethyl]thiophen-2- | | |
| | yl}formamido)-3-cyclopentyl-N- | | |
| | cyclopropylpropanamide | | |
| 166 | 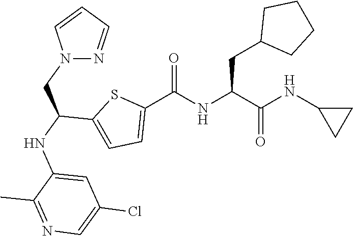 [SMILES: Cc1ncc(Cl)cc1N[C@@H](Cn1cccn1)c1ccc(C(=O)N[C@@H](CC2CCCC2)C(=O)NC2CC2)s1] | 541.11 | 540.2074 |
| | (2S)-2-({5-[(1S)-1-[(5-chloro-2-methylpyridin-3- | | |
| | yl)amino]-2-(1H-pyrazol-1-yl)ethyl]thiophen-2- | | |
| | yl}formamido)-3-cyclopentyl-N- | | |
| | cyclopropylpropanamide | | |
| 167 | 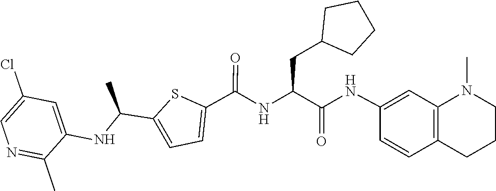 [SMILES: Cc1ncc(Cl)cc1N[C@@H](C)c1ccc(C(=O)N[C@@H](CC2CCCC2)C(=O)Nc2ccc3c(c2)N(C)CCC3)s1] | 580.19 | 579.2435 |
| | (2S)-2-({5-[(1S)-1-[(5-chloro-2-methylpyridin-3- | | |
| | yl)amino]ethyl]thiophen-2-yl}formamido)-3- | | |
| | cyclopentyl-N-(1-methyl-1,2,3,4-tetrahydroquinolin- | | |
| | 7-yl)propanamide | | |
| 168 | 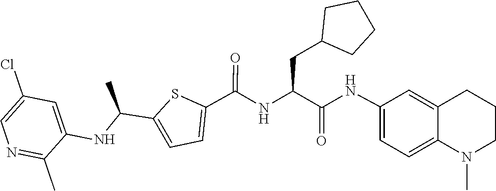 [SMILES: Cc1ncc(Cl)cc1N[C@@H](C)c1ccc(C(=O)N[C@@H](CC2CCCC2)C(=O)Nc2ccc3c(c2)CCCN3C)s1] | 580.19 | 579.2435 |
| | (2S)-2-({5-[(1S)-1-[(5-chloro-2-methylpyridin-3- | | |
| | yl)amino]ethyl]thiophen-2-yl}formamido)-3- | | |
| | cyclopentyl-N-(1-methyl-1,2,3,4-tetrahydroquinolin- | | |
| | 6-yl)propanamide | | |
| 169 | 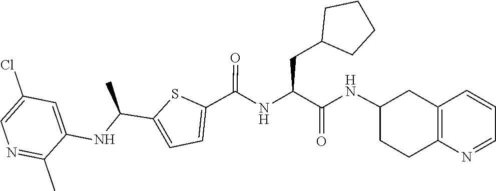 [SMILES: Cc1ncc(Cl)cc1N[C@@H](C)c1ccc(C(=O)N[C@@H](CC2CCCC2)C(=O)NC2CCc3ncccc3C2)s1] | 566.16 | 565.2278 |
| | (2S)-2-({5-[(1S)-1-[(5-chloro-2-methylpyridin-3- | | |
| | yl)amino]ethyl]thiophen-2-yl}formamido)-3- | | |
| | cyclopentyl-N-(5,6,7,8-tetrahydroquinolin-6- | | |
| | yl)propanamide | | |
| 170 | 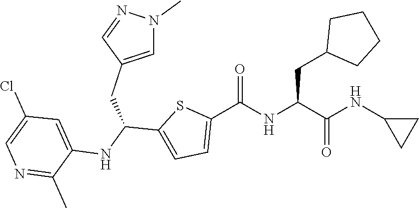 [SMILES: Cc1ncc(Cl)cc1N[C@H](Cc1cnn(C)c1)c1ccc(C(=O)N[C@@H](CC2CCCC2)C(=O)NC2CC2)s1] | 555.14 | 554.2231 |
| | (2S)-2-({5-[(1R)-1-[(5-chloro-2-methylpyridin-3- | | |
| | yl)amino]-2-(1-methyl-1H-pyrazol-4- | | |
| | yl)ethyl]thiophen-2-yl}formamido)-3-cyclopentyl-N- | | |
| | cyclopropylpropanamide | | |
| 171 | 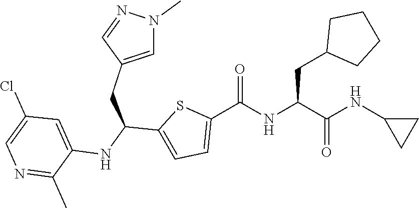 [SMILES: Cc1ncc(Cl)cc1N[C@@H](Cc1cnn(C)c1)c1ccc(C(=O)N[C@@H](CC2CCCC2)C(=O)NC2CC2)s1] | 555.14 | 554.2231 |
| | (2S)-2-({5-[(1S)-1-[(5-chloro-2-methylpyridin-3- | | |
| | yl)amino]-2-(1-methyl-1H-pyrazol-4- | | |
| | yl)ethyl]thiophen-2-yl}formamido)-3-cyclopentyl-N- | | |
| | cyclopropylpropanamide | | |
| 172 | 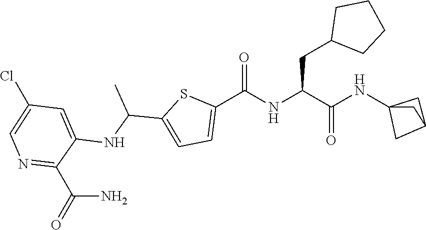 [SMILES: CC(Nc1cc(Cl)cnc1C(N)=O)c1ccc(C(=O)N[C@@H](CC2CCCC2)C(=O)NC23CC(C2)C3)s1] | 530.08 | 529.1914 |
| | 3-{[1-(5-{[(1S)-1-({bicyclo[1.1.1]pentan-1- | | |
| | yl}carbamoyl)-2- | | |
| | cyclopentylethyl]carbamoyl}thiophen-2- | | |
| | yl)ethyl]amino}-5-chloropyridine-2-carboxamide | | |
| 173 | 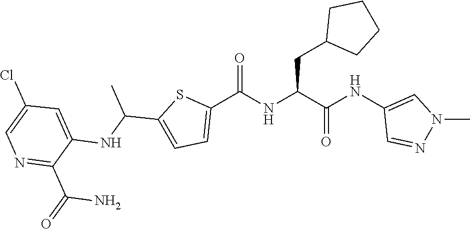 [SMILES: CC(Nc1cc(Cl)cnc1C(N)=O)c1ccc(C(=O)N[C@@H](CC2CCCC2)C(=O)Nc2cnn(C)c2)s1] | 544.07 | 543.1819 |
| | 5-chloro-3-{[1-(5-{[(1S)-2-cyclopentyl-1-[(1-methyl- | | |
| | 1H-pyrazol-4- | | |
| | yl)carbamoyl]ethyl]carbamoyl}thiophen-2- | | |
| | yl)ethyl]amino}pyridine-2-carboxamide | | |
| 174 | 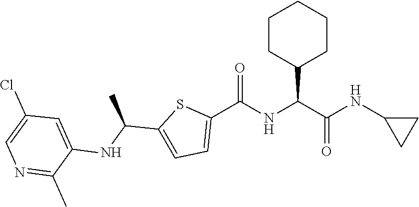 [SMILES: Cc1ncc(Cl)cc1N[C@@H](C)c1ccc(C(=O)N[C@H](C(=O)NC2CC2)C2CCCCC2)s1] | 475.05 | 474.1856 |
| | (2S)-2-({5-[(1S)-1-[(5-chloro-2-methylpyridin-3- | | |
| | yl)amino]ethyl]thiophen-2-yl}formamido)-2- | | |
| | cyclohexyl-N-cyclopropylacetamide | | |
| 175 | 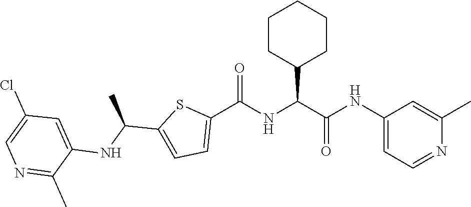 [SMILES: Cc1cc(NC(=O)[C@@H](NC(=O)c2ccc([C@H](C)Nc3cc(Cl)cnc3C)s2)C2CCCCC2)ccn1] | 526.1 | 525.1965 |
| | (2S)-2-({5-[(1S)-1-[(5-chloro-2-methylpyridin-3- | | |
| | yl)amino]ethyl]thiophen-2-yl}formamido)-2- | | |
| | cyclohexyl-N-(2-methylpyridin-4-yl)acetamide | | |
| 176 | 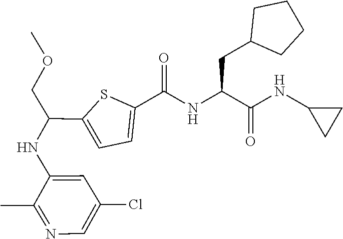 [SMILES: COCC(Nc1cc(Cl)cnc1C)c1ccc(C(=O)N[C@@H](CC2CCCC2)C(=O)NC2CC2)s1] | 505.07 | 504.1962 |
| | (2S)-2-[(5-{1-[(5-chloro-2-methylpyridin-3- | | |
| | yl)amino]-2-methoxyethyl}thiophen-2- | | |
| | yl)formamido]-3-cyclopentyl-N- | | |
| | cyclopropylpropanamide | | |
| 177 | 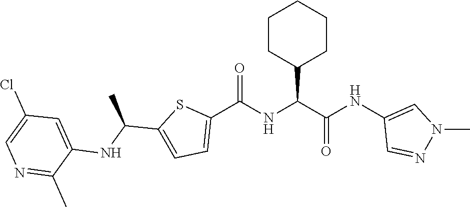 [SMILES: Cc1ncc(Cl)cc1N[C@@H](C)c1ccc(C(=O)N[C@H](C(=O)Nc2cnn(C)c2)C2CCCCC2)s1] | 515.07 | 514.1918 |
| | (2S)-2-({5-[(1S)-1-[(5-chloro-2-methylpyridin-3- | | |
| | yl)amino]ethyl]thiophen-2-yl}formamido)-2- | | |
| | cyclohexyl-N-(1-methyl-1H-pyrazol-4-yl)acetamide | | |
| 178 | 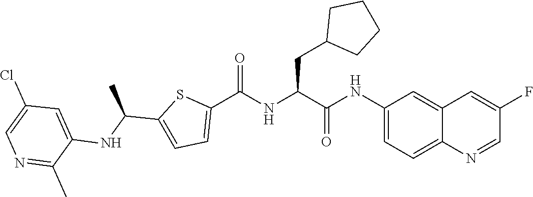 [SMILES: Cc1ncc(Cl)cc1N[C@@H](C)c1ccc(C(=O)N[C@@H](CC2CCCC2)C(=O)Nc2ccc3ncc(F)cc3c2)s1] | 580.12 | 579.1871 |
| | (2S)-2-({5-[(1S)-1-[(5-chloro-2-methylpyridin-3- | | |
| | yl)amino]ethyl]thiophen-2-yl}formamido)-3- | | |
| | cyclopentyl-N-(3-fluoroquinolin-6-yl)propanamide | | |
| 179 | 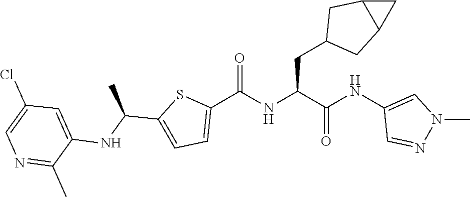 [SMILES: Cc1ncc(Cl)cc1N[C@@H](C)c1ccc(C(=O)N[C@@H](CC2CC3CC3C2)C(=O)Nc2cnn(C)c2)s1] | 527.08 | 526.1918 |
| | (2S)-3-{bicyclo[3.1.0]hexan-3-yl}-2-({5-[(1S)-1-[(5- | | |
| | chloro-2-methylpyridin-3-yl)amino]ethyl]thiophen-2- | | |
| | yl}formamido)-N-(1-methyl-1H-pyrazol-4- | | |
| | yl)propanamide | | |
| 180 | 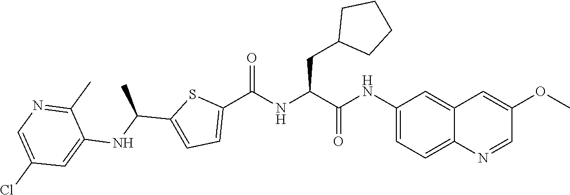 [SMILES: COc1cnc2ccc(NC(=O)[C@H](CC3CCCC3)NC(=O)c3ccc([C@H](C)Nc4cc(Cl)cnc4C)s3)cc2c1] | 592.16 | 591.2071 |
| | (2S)-2-({5-[(1S)-1-[(5-chloro-2-methylpyridin-3- | | |
| | yl)amino]ethyl]thiophen-2-yl}formamido)-3- | | |
| | cyclopentyl-N-(3-methoxyquinolin-6-yl)propanamide | | |
| 181 | 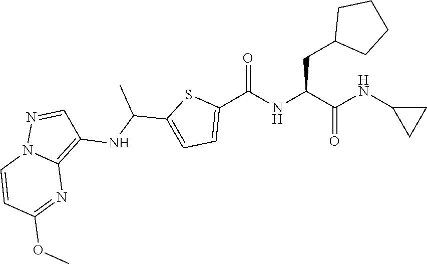 [SMILES: COc1ccn2ncc(NC(C)c3ccc(C(=O)N[C@@H](CC4CCCC4)C(=O)NC4CC4)s3)c2n1] | 496.63 | 496.2257 |
| | (2S)-3-cyclopentyl-N-cyclopropyl-2-({5-[1-({5- | | |
| | methoxypyrazolo[1,5-a]pyrimidin-3- | | |
| | yl}amino)ethyl]thiophen-2- | | |
| | yl}formamido)propanamide | | |
| 182 | 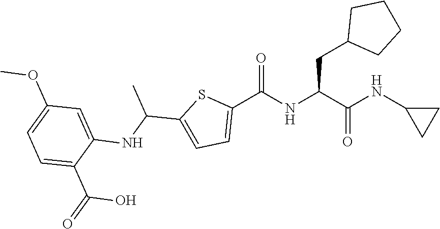 [SMILES: COc1ccc(C(=O)O)c(NC(C)c2ccc(C(=O)N[C@@H](CC3CCCC3)C(=O)NC3CC3)s2)c1] | 499.63 | 499.2141 |
| | 2-{[1-(5-{[(1S)-2-cyclopentyl-1- | | |
| | (cyclopropylcarbamoyl)ethyl]carbamoyl}thiophen-2- | | |
| | yl)ethyl]amino}-4-methoxybenzoic acid | | |
| 183 | 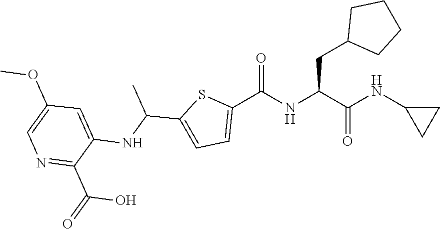 [SMILES: COc1cnc(C(=O)O)c(NC(C)c2ccc(C(=O)N[C@@H](CC3CCCC3)C(=O)NC3CC3)s2)c1] | 500.61 | 500.2093 |
| | 3-{[1-(5-{[(1S)-2-cyclopentyl-1- | | |
| | (cyclopropylcarbamoyl)ethyl]carbamoyl}thiophen-2- | | |
| | yl)ethyl]amino}-5-methoxypyridine-2-carboxylic acid | | |
| 184 | 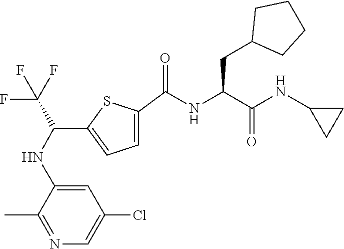 | 529.02 | 528.1574 |
| | (2S)-2-({5-[(1R)-1-[(5-chloro-2-methylpyridin-3- | | |
| | yl)amino]-2,2,2-trifluoroethyl]thiophen-2- | | |
| | yl}formamido)-3-cyclopentyl-N- | | |
| | cyclopropylpropanamide | | |
| 185 | 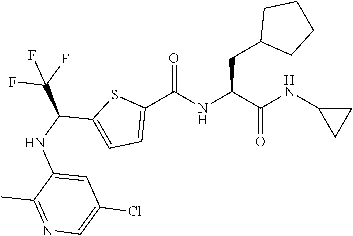 [SMILES: Cc1ncc(Cl)cc1N[C@H](c1ccc(C(=O)N[C@@H](CC2CCCC2)C(=O)NC2CC2)s1)C(F)(F)F] | 529.02 | 528.1574 |
| | (2S)-2-({5-[(1S)-1-[(5-chloro-2-methylpyridin-3- | | |
| | yl)amino]-2,2,2-trifluoroethyl]thiophen-2- | | |
| | yl}formamido)-3-cyclopentyl-N- | | |
| | cyclopropylpropanamide | | |
| 186 | 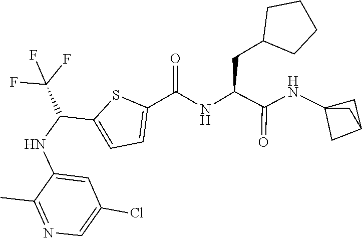 | 555.06 | 554.173 |
| | (2S)-N-{bicyclo[1.1.1]pentan-1-yl}-2-({5-[(1R)-1-[(5- | | |
| | chloro-2-methylpyridin-3-yl)amino]-2,2,2- | | |
| | trifluoroethyl]thiophen-2-yl}formamido)-3- | | |
| | cyclopentylpropanamide | | |
| 187 | 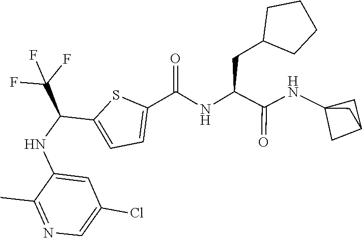 [SMILES: Cc1ncc(Cl)cc1N[C@H](c1ccc(C(=O)N[C@@H](CC2CCCC2)C(=O)NC23CC(C2)C3)s1)C(F)(F)F] | 555.06 | 554.173 |
| | (2S)-N-{bicyclo[1.1.1]pentan-1-yl}-2-({5-[(1S)-1-[(5- | | |
| | chloro-2-methylpyridin-3-yl)amino]-2,2,2- | | |
| | trifluoroethyl]thiophen-2-yl}formamido)-3- | | |
| | cyclopentylpropanamide | | |
| 188 | 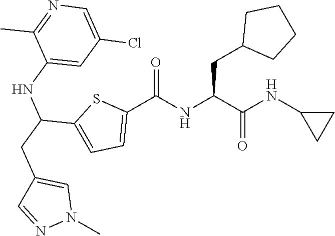 [SMILES: Cc1ncc(Cl)cc1NC(Cc1cnn(C)c1)c1ccc(C(=O)N[C@@H](CC2CCCC2)C(=O)NC2CC2)s1] | 555.14 | 554.2231 |
| | (2S)-2-[(5-{1-[(5-chloro-2-methylpyridin-3- | | |
| | yl)amino]-2-(1-methyl-1H-pyrazol-4- | | |
| | yl)ethyl}thiophen-2-yl)formamido]-3-cyclopentyl-N- | | |
| | cyclopropylpropanamide | | |
| 189 | 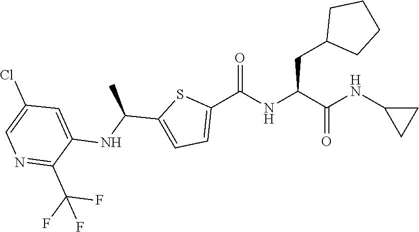 [SMILES: C[C@H](Nc1cc(Cl)cnc1C(F)(F)F)c1ccc(C(=O)N[C@@H](CC2CCCC2)C(=O)NC2CC2)s1] | 529.02 | 528.1574 |
| | (2S)-2-({5-[(1S)-1-{[5-chloro-2- | | |
| | (trifluoromethyl)pyridin-3-yl]amino}ethyl]thiophen-2- | | |
| | yl}formamido)-3-cyclopentyl-N- | | |
| | cyclopropylpropanamide | | |
| 190 | 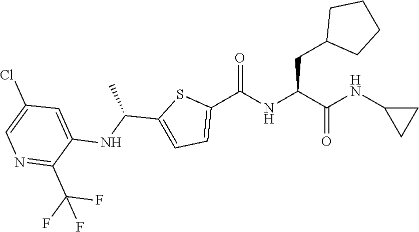 [SMILES: C[C@@H](Nc1cc(Cl)cnc1C(F)(F)F)c1ccc(C(=O)N[C@@H](CC2CCCC2)C(=O)NC2CC2)s1] | 529.02 | 528.1574 |
| | (2S)-2-({5-[(1R)-1-{[5-chloro-2- | | |
| | (trifluoromethyl)pyridin-3-yl]amino}ethyl]thiophen-2- | | |
| | yl}formamido)-3-cyclopentyl-N- | | |
| | cyclopropylpropanamide | | |
| 191 | 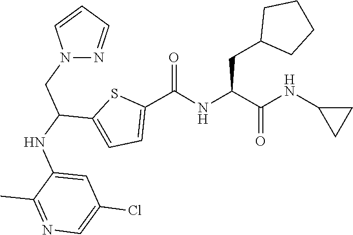 [SMILES: Cc1ncc(Cl)cc1NC(Cn1cccn1)c1ccc(C(=O)N[C@@H](CC2CCCC2)C(=O)NC2CC2)s1] | 541.11 | 540.2074 |
| | (2S)-2-[(5-{1-[(5-chloro-2-methylpyridin-3- | | |
| | yl)amino]-2-(1H-pyrazol-1-yl)ethyl}thiophen-2- | | |
| | yl)formamido]-3-cyclopentyl-N- | | |
| | cyclopropylpropanamide | | |
| 192 | 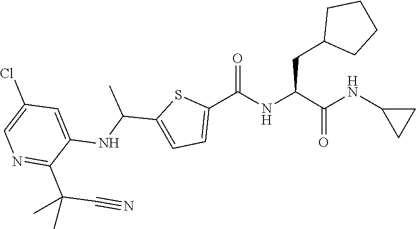 [SMILES: CC(Nc1cc(Cl)cnc1C(C)(C)C#N)c1ccc(C(=O)N[C@@H](CC2CCCC2)C(=O)NC2CC2)s1] | 528.11 | 527.2122 |
| | (2S)-2-{[5-(1-{[5-chloro-2-(1-cyano-1- | | |
| | methylethyl)pyridin-3-yl]amino}ethyl)thiophen-2- | | |
| | yl]formamido}-3-cyclopentyl-N- | | |
| | cyclopropylpropanamide | | |
| 193 | 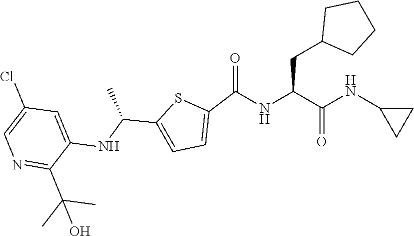 [SMILES: C[C@@H](Nc1cc(Cl)cnc1C(C)(C)O)c1ccc(C(=O)N[C@@H](CC2CCCC2)C(=O)NC2CC2)s1] | 519.1 | 518.2118 |
| | (2S)-2-({5-[(1R)-1-{[5-chloro-2-(2-hydroxypropan-2- | | |
| | yl)pyridin-3-yl]amino}ethyl]thiophen-2- | | |
| | yl}formamido)-3-cyclopentyl-N- | | |
| | cyclopropylpropanamide | | |
| 194 | 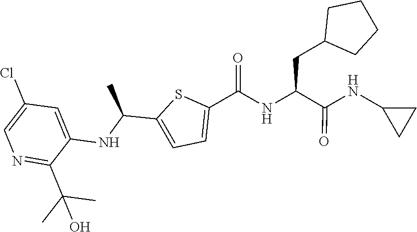 [SMILES: C[C@H](Nc1cc(Cl)cnc1C(C)(C)O)c1ccc(C(=O)N[C@@H](CC2CCCC2)C(=O)NC2CC2)s1] | 519.1 | 518.2118 |
| | (2S)-2-({5-[(1S)-1-{[5-chloro-2-(2-hydroxypropan-2- | | |
| | yl)pyridin-3-yl]amino}ethyl]thiophen-2- | | |
| | yl}formamido)-3-cyclopentyl-N- | | |
| | cyclopropylpropanamide | | |
| 195 | 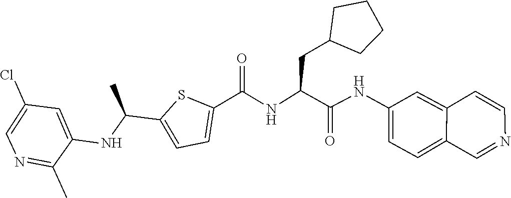 [SMILES: Cc1ncc(Cl)cc1N[C@@H](C)c1ccc(C(=O)N[C@@H](CC2CCCC2)C(=O)Nc2ccc3cnccc3c2)s1] | 562.13 | 561.1965 |
| | (2S)-2-({5-[(1S)-1-[(5-chloro-2-methylpyridin-3- | | |
| | yl)amino]ethyl]thiophen-2-yl}formamido)-3- | | |
| | cyclopentyl-N-(isoquinolin-6-yl)propanamide | | |
| 196 | 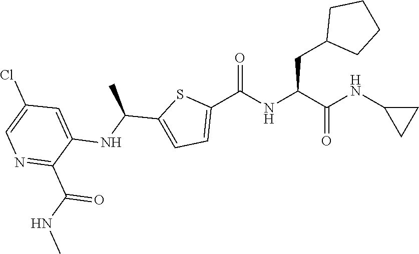 [SMILES: CNC(=O)c1ncc(Cl)cc1N[C@@H](C)c1ccc(C(=O)N[C@@H](CC2CCCC2)C(=O)NC2CC2)s1] | 518.07 | 517.1914 |
| | 5-chloro-3-{[(1S)-1-(5-{[(1S)-2-cyclopentyl-1- | | |
| | (cyclopropylcarbamoyl)ethyl]carbamoyl}thiophen-2- | | |
| | yl)ethyl]amino}-N-methylpyridine-2-carboxamide | | |
| 197 | 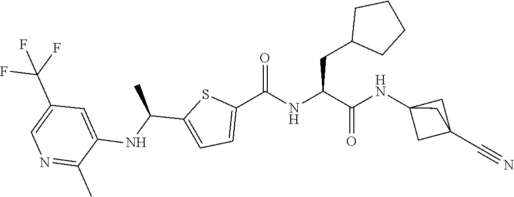 [SMILES: Cc1ncc(C(F)(F)F)cc1N[C@@H](C)c1ccc(C(=O)N[C@@H](CC2CCCC2)C(=O)NC23CC(C#N)(C2)C3)s1] | 559.65 | 559.2229 |
| | (2S)-N-{3-cyanobicyclo[1.1.1]pentan-1-yl}-3- | | |
| | cyclopentyl-2-({5-[(1S)-1-{[2-methyl-5- | | |
| | (trifluoromethyl)pyridin-3-yl]amino}ethyl]thiophen-2- | | |
| | yl}formamido)propanamide | | |
| 198 | 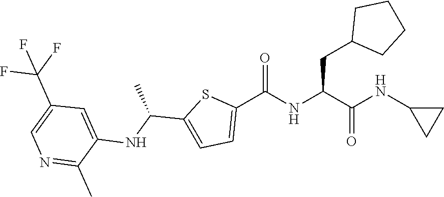 [SMILES: Cc1ncc(C(F)(F)F)cc1N[C@H](C)c1ccc(C(=O)N[C@@H](CC2CCCC2)C(=O)NC2CC2)s1] | 508.6 | 508.212 |
| | (2S)-3-cyclopentyl-N-cyclopropyl-2-({5-[(1R)-1-{[2- | | |
| | methyl-5-(trifluoromethyl)pyridin-3- | | |
| | yl]amino}ethyl]thiophen-2- | | |
| | yl}formamido)propanamide | | |
| 199 | 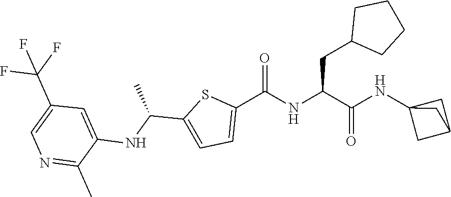 [SMILES: Cc1ncc(C(F)(F)F)cc1N[C@H](C)c1ccc(C(=O)N[C@@H](CC2CCCC2)C(=O)NC23CC(C2)C3)s1] | 534.64 | 534.2276 |
| | (2S)-N-{bicyclo[1.1.1]pentan-1-yl}-3-cyclopentyl-2- | | |
| | ({5-[(1R)-1-{[2-methyl-5-(trifluoromethyl)pyridin-3- | | |
| | yl]amino}ethyl]thiophen-2- | | |
| | yl}formamido)propanamide | | |
| 200 | 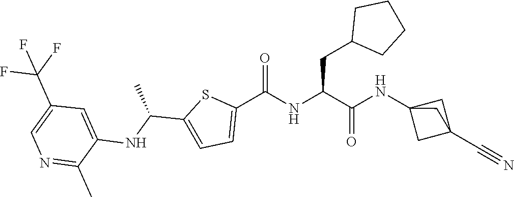 [SMILES: Cc1ncc(C(F)(F)F)cc1N[C@H](C)c1ccc(C(=O)N[C@@H](CC2CCCC2)C(=O)NC23CC(C#N)(C2)C3)s1] | 559.65 | 559.2229 |
| | (2S)-N-{3-cyanobicyclo[1.1.1]pentan-1-yl}-3- | | |
| | cyclopentyl-2-({5-[(1R)-1-{[2-methyl-5- | | |
| | (trifluoromethyl)pyridin-3-yl]amino}ethyl]thiophen-2- | | |
| | yl}formamido)propanamide | | |
| 201 | 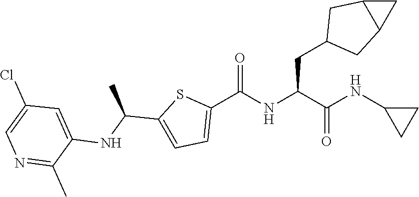 [SMILES: Cc1ncc(Cl)cc1N[C@@H](C)c1ccc(C(=O)N[C@@H](CC2CC3CC3C2)C(=O)NC2CC2)s1] | 487.06 | 486.1856 |
| | (2S)-3-{bicyclo[3.1.0]hexan-3-yl}-2-({5-[(1S)-1-[(5- | | |
| | chloro-2-methylpyridin-3-yl)amino]ethyl]thiophen-2- | | |
| | yl}formamido)-N-cyclopropylpropanamide | | |
| 202 | 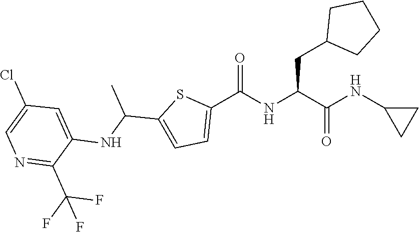 [SMILES: CC(Nc1cc(Cl)cnc1C(F)(F)F)c1ccc(C(=O)N[C@@H](CC2CCCC2)C(=O)NC2CC2)s1] | 529.02 | 528.1574 |
| | (2S)-2-{[5-(1-{[5-chloro-2-(trifluoromethyl)pyridin-3- | | |
| | yl]amino}ethyl)thiophen-2-yl]formamido}-3- | | |
| | cyclopentyl-N-cyclopropylpropanamide | | |
| 203 | 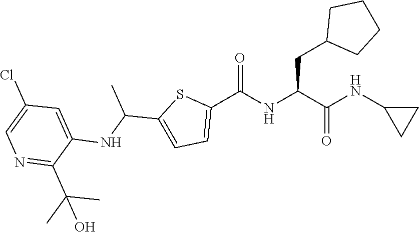 [SMILES: CC(Nc1cc(Cl)cnc1C(C)(C)O)c1ccc(C(=O)N[C@@H](CC2CCCC2)C(=O)NC2CC2)s1] | 519.1 | 518.2118 |
| | (2S)-2-{[5-(1-{[5-chloro-2-(2-hydroxypropan-2- | | |
| | yl)pyridin-3-yl]amino}ethyl)thiophen-2- | | |
| | yl]formamido}-3-cyclopentyl-N- | | |
| | cyclopropylpropanamide | | |
| 204 | 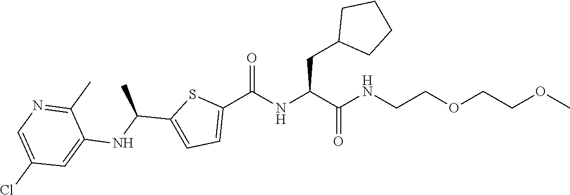 [SMILES: COCCOCCNC(=O)[C@H](CC1CCCC1)NC(=O)c1ccc([C@H](C)Nc2cc(Cl)cnc2C)s1] | 537.12 | 536.2224 |
| | (2S)-2-({5-[(1S)-1-[(5-chloro-2-methylpyridin-3- | | |
| | yl)amino]ethyl]thiophen-2-yl}formamido)-3- | | |
| | cyclopentyl-N-[2-(2- | | |
| | methoxyethoxy)ethyl]propanamide | | |
| 205 | 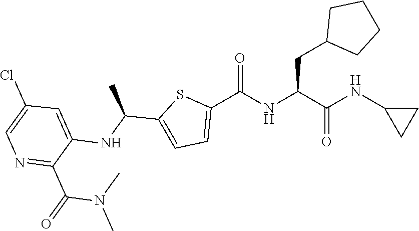 [SMILES: C[C@H](Nc1cc(Cl)cnc1C(=O)N(C)C)c1ccc(C(=O)N[C@@H](CC2CCCC2)C(=O)NC2CC2)s1] | 532.1 | 531.2071 |
| | 5-chloro-3-{[(1S)-1-(5-{[(1S)-2-cyclopentyl-1- | | |
| | (cyclopropylcarbamoyl)ethyl]carbamoyl}thiophen-2- | | |
| | yl)ethyl]amino}-N,N-dimethylpyridine-2- | | |
| | carboxamide | | |
| 206 | 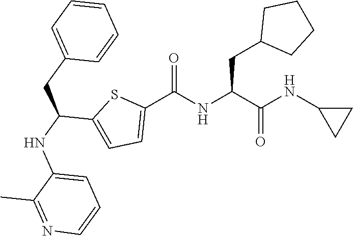 [SMILES: Cc1ncccc1N[C@@H](Cc1ccccc1)c1ccc(C(=O)N[C@@H](CC2CCCC2)C(=O)NC2CC2)s1] | 516.7 | 516.2559 |
| | (2S)-3-cyclopentyl-N-cyclopropyl-2-({5-[(1S)-1-[(2- | | |
| | methylpyridin-3-yl)amino]-2-phenylethyl]thiophen-2- | | |
| | yl}formamido)propanamide | | |
| 207 | 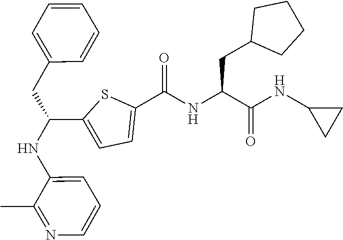 [SMILES: Cc1ncccc1N[C@H](Cc1ccccc1)c1ccc(C(=O)N[C@@H](CC2CCCC2)C(=O)NC2CC2)s1] | 516.7 | 516.2559 |
| | (2S)-3-cyclopentyl-N-cyclopropyl-2-({5-[(1R)-1-[(2- | | |
| | methylpyridin-3-yl)amino]-2-phenylethyl]thiophen-2- | | |
| | yl}formamido)propanamide | | |
| 208 | 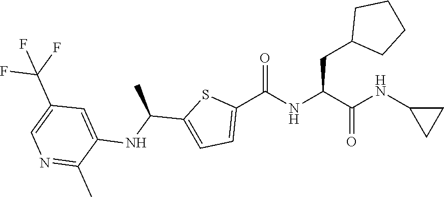 [SMILES: Cc1ncc(C(F)(F)F)cc1N[C@@H](C)c1ccc(C(=O)N[C@@H](CC2CCCC2)C(=O)NC2CC2)s1] | 508.6 | 508.212 |
| | (2S)-3-cyclopentyl-N-cyclopropyl-2-({5-[(1S)-1-{[2- | | |
| | methyl-5-(trifluoromethyl)pyridin-3- | | |
| | yl]amino}ethyl]thiophen-2- | | |
| | yl}formamido)propanamide | | |
| 209 | 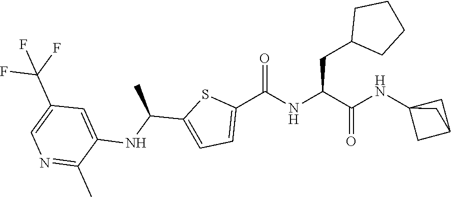 [SMILES: Cc1ncc(C(F)(F)F)cc1N[C@@H](C)c1ccc(C(=O)N[C@@H](CC2CCCC2)C(=O)NC23CC(C2)C3)s1] | 534.64 | 534.2276 |
| | (2S)-N-{bicyclo[1.1.1]pentan-1-yl}-3-cyclopentyl-2- | | |
| | ({5-[(1S)-1-{[2-methyl-5-(trifluoromethyl)pyridin-3- | | |
| | yl]amino}ethyl]thiophen-2- | | |
| | yl}formamido)propanamide | | |
| 210 | 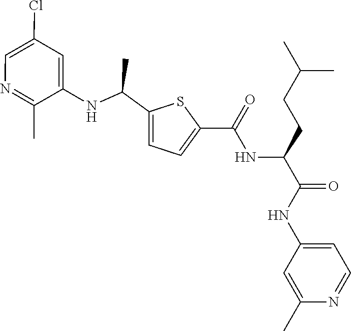 [SMILES: Cc1cc(NC(=O)[C@H](CCC(C)C)NC(=O)c2ccc([C@H](C)Nc3cc(Cl)cnc3C)s2)ccn1] | 514.09 | 513.1965 |
| | (2S)-2-({5-[(1S)-1-[(5-chloro-2-methylpyridin-3- | | |
| | yl)amino]ethyl]thiophen-2-yl}formamido)-5-methyl- | | |
| | N-(2-methylpyridin-4-yl)hexanamide | | |
| 211 | 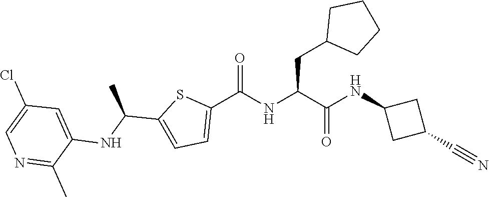 [SMILES: Cc1ncc(Cl)cc1N[C@@H](C)c1ccc(C(=O)N[C@@H](CC2CCCC2)C(=O)N[C@H]2C[C@H](C#N)C2)s1] | 514.09 | 513.1965 |
| | (2S)-2-({5-[(1S)-1-[(5-chloro-2-methylpyridin-3- | | |
| | yl)amino]ethyl]thiophen-2-yl}formamido)-3- | | |
| | cyclopentyl-N-[(1r,3r)-3- | | |
| | cyanocyclobutyl]propanamide | | |
| 212 | 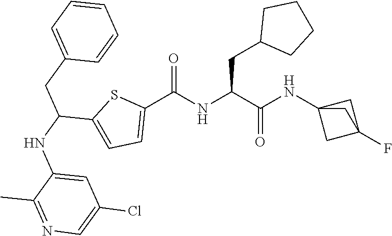 [SMILES: Cc1ncc(Cl)cc1NC(Cc1ccccc1)c1ccc(C(=O)N[C@@H](CC2CCCC2)C(=O)NC23CC(F)(C2)C3)s1] | 595.17 | 594.2232 |
| | (2S)-2-[(5-{1-[(5-chloro-2-methylpyridin-3- | | |
| | yl)amino]-2-phenylethyl}thiophen-2-yl)formamido]- | | |
| | 3-cyclopentyl-N-{3-fluorobicyclo[1.1.1]pentan-1- | | |
| | yl}propanamide | | |
| 213 | 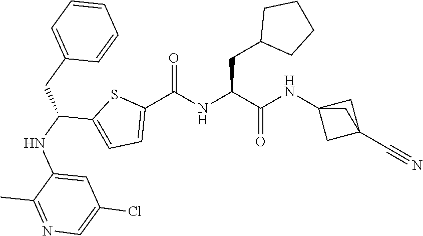 [SMILES: Cc1ncc(Cl)cc1N[C@H](Cc1ccccc1)c1ccc(C(=O)N[C@@H](CC2CCCC2)C(=O)NC23CC(C#N)(C2)C3)s1] | 602.19 | 601.2278 |
| | (2S)-2-({5-[(1R)-1-[(5-chloro-2-methylpyridin-3- | | |
| | yl)amino]-2-phenylethyl]thiophen-2-yl}formamido)- | | |
| | N-{3-cyanobicyclo[1.1.1]pentan-1-yl}-3- | | |
| | cyclopentylpropanamide | | |
| 214 | 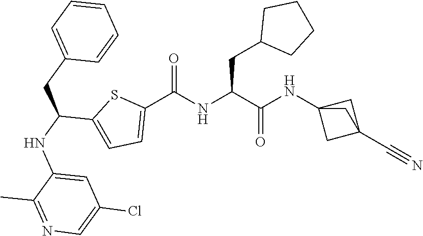 [SMILES: Cc1ncc(Cl)cc1N[C@@H](Cc1ccccc1)c1ccc(C(=O)N[C@@H](CC2CCCC2)C(=O)NC23CC(C#N)(C2)C3)s1] | 602.19 | 601.2278 |
| | (2S)-2-({5-[(1S)-1-[(5-chloro-2-methylpyridin-3- | | |
| | yl)amino]-2-phenylethyl]thiophen-2-yl}formamido)- | | |
| | N-{3-cyanobicyclo[1.1.1]pentan-1-yl}-3- | | |
| | cyclopentylpropanamide | | |
| 215 | 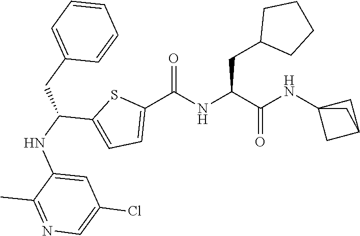 [SMILES: Cc1ncc(Cl)cc1N[C@H](Cc1ccccc1)c1ccc(C(=O)N[C@@H](CC2CCCC2)C(=O)NC23CC(C2)C3)s1] | 577.18 | 576.2326 |
| | (2S)-N-{bicyclo[1.1.1]pentan-1-yl}-2-({5-[(1R)-1-[(5- | | |
| | chloro-2-methylpyridin-3-yl)amino]-2- | | |
| | phenylethyl]thiophen-2-yl}formamido)-3- | | |
| | cyclopentylpropanamide | | |
| 216 | 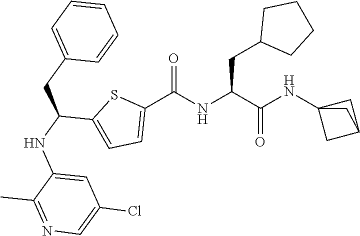 [SMILES: Cc1ncc(Cl)cc1N[C@@H](Cc1ccccc1)c1ccc(C(=O)N[C@@H](CC2CCCC2)C(=O)NC23CC(C2)C3)s1] | 577.18 | 576.2326 |
| | (2S)-N-{bicyclo[1.1.1]pentan-1-yl}-2-({5-[(1S)-1-[(5- | | |
| | chloro-2-methylpyridin-3-yl)amino]-2- | | |
| | phenylethyl]thiophen-2-yl}formamido)-3- | | |
| | cyclopentylpropanamide | | |
| 217 | 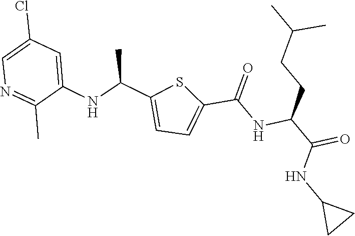 [SMILES: Cc1ncc(Cl)cc1N[C@@H](C)c1ccc(C(=O)N[C@@H](CCC(C)C)C(=O)NC2CC2)s1] | 463.04 | 462.1856 |
| | (2S)-2-({5-[(1S)-1-[(5-chloro-2-methylpyridin-3- | | |
| | yl)amino]ethyl]thiophen-2-yl}formamido)-N- | | |
| | cyclopropyl-5-methylhexanamide | | |
| 218 | 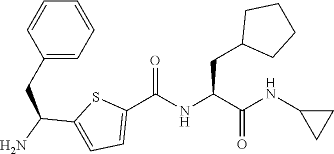 [SMILES: N[C@@H](Cc1ccccc1)c1ccc(C(=O)N[C@@H](CC2CCCC2)C(=O)NC2CC2)s1] | 425.59 | 425.2137 |
| | (2S)-2-({5-[(1S)-1-amino-2-phenylethyl]thiophen-2- | | |
| | yl}formamido)-3-cyclopentyl-N- | | |
| | cyclopropylpropanamide | | |
| 219 | 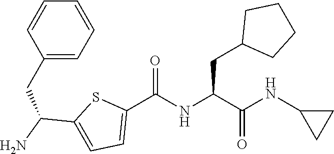 [SMILES: N[C@H](Cc1ccccc1)c1ccc(C(=O)N[C@@H](CC2CCCC2)C(=O)NC2CC2)s1] | 425.59 | 425.2137 |
| | (2S)-2-({5-[(1R)-1-amino-2-phenylethyl]thiophen-2- | | |
| | yl}formamido)-3-cyclopentyl-N- | | |
| | cyclopropylpropanamide | | |
| 220 | 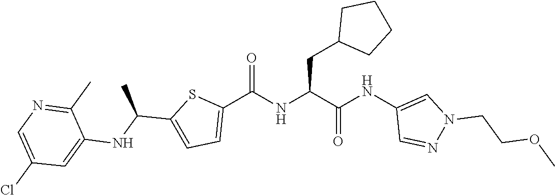 [SMILES: COCCn1cc(NC(=O)[C@H](CC2CCCC2)NC(=O)c2ccc([C@H](C)Nc3cc(Cl)cnc3C)s2)cn1] | 559.13 | 558.218 |
| | (2S)-2-({5-[(1S)-1-[(5-chloro-2-methylpyridin-3- | | |
| | yl)amino]ethyl]thiophen-2-yl}formamido)-3- | | |
| | cyclopentyl-N-[1-(2-methoxyethyl)-1H-pyrazol-4- | | |
| | yl]propanamide | | |
| 221 | 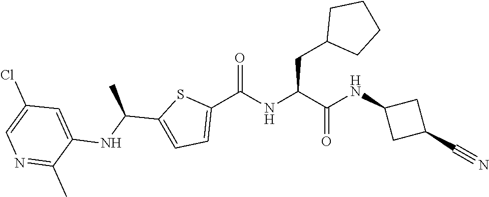 [SMILES: Cc1ncc(Cl)cc1N[C@@H](C)c1ccc(C(=O)N[C@@H](CC2CCCC2)C(=O)N[C@H]2C[C@@H](C#N)C2)s1] | 514.09 | 513.1965 |
| | (2S)-2-({5-[(1S)-1-[(5-chloro-2-methylpyridin-3- | | |
| | yl)amino]ethyl]thiophen-2-yl}formamido)-3- | | |
| | cyclopentyl-N-[(1s,3s)-3- | | |
| | cyanocyclobutyl]propanamide | | |
| 222 | 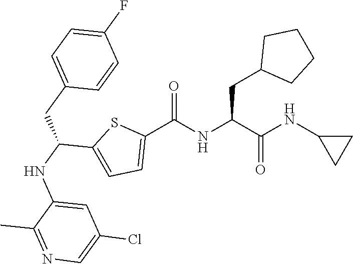 [SMILES: Cc1ncc(Cl)cc1N[C@H](Cc1ccc(F)cc1)c1ccc(C(=O)N[C@@H](CC2CCCC2)C(=O)NC2CC2)s1] | 569.14 | 568.2075 |
| | (2S)-2-({5-[(1R)-1-[(5-chloro-2-methylpyridin-3- | | |
| | yl)amino]-2-(4-fluorophenyl)ethyl]thiophen-2- | | |
| | yl}formamido)-3-cyclopentyl-N- | | |
| | cyclopropylpropanamide | | |
| 223 | 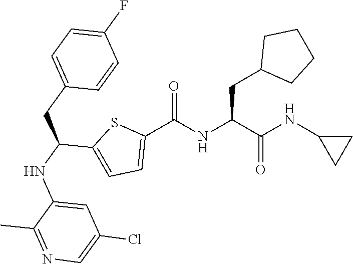 [SMILES: Cc1ncc(Cl)cc1N[C@@H](Cc1ccc(F)cc1)c1ccc(C(=O)N[C@@H](CC2CCCC2)C(=O)NC2CC2)s1] | 569.14 | 568.2075 |
| | (2S)-2-({5-[(1S)-1-[(5-chloro-2-methylpyridin-3- | | |
| | yl)amino]-2-(4-fluorophenyl)ethyl]thiophen-2- | | |
| | yl}formamido)-3-cyclopentyl-N- | | |
| | cyclopropylpropanamide | | |
| 224 | 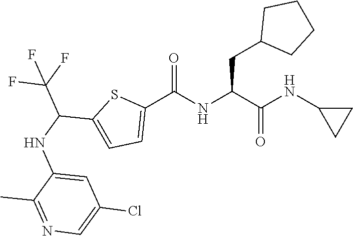 [SMILES: Cc1ncc(Cl)cc1NC(c1ccc(C(=O)N[C@@H](CC2CCCC2)C(=O)NC2CC2)s1)C(F)(F)F] | 529.02 | 528.1574 |
| | (2S)-2-[(5-{1-[(5-chloro-2-methylpyridin-3- | | |
| | yl)amino]-2,2,2-trifluoroethyl}thiophen-2- | | |
| | yl)formamido]-3-cyclopentyl-N- | | |
| | cyclopropylpropanamide | | |
| 225 | 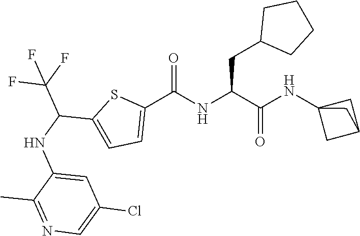 [SMILES: Cc1ncc(Cl)cc1NC(c1ccc(C(=O)N[C@@H](CC2CCCC2)C(=O)NC23CC(C2)C3)s1)C(F)(F)F] | 555.06 | 554.173 |
| | (2S)-N-{bicyclo[1.1.1]pentan-1-yl}-2-[(5-{1-[(5- | | |
| | chloro-2-methylpyridin-3-yl)amino]-2,2,2- | | |
| | trifluoroethyl}thiophen-2-yl)formamido]-3- | | |
| | cyclopentylpropanamide | | |
| 226 | 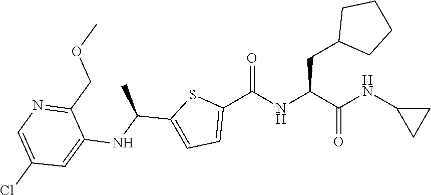 [SMILES: COCc1ncc(Cl)cc1N[C@@H](C)c1ccc(C(=O)N[C@@H](CC2CCCC2)C(=O)NC2CC2)s1] | 505.07 | 504.1962 |
| | (2S)-2-({5-[(1S)-1-{[5-chloro-2- | | |
| | (methoxymethyl)pyridin-3-yl]amino}ethyl]thiophen- | | |
| | 2-yl}formamido)-3-cyclopentyl-N- | | |
| | cyclopropylpropanamide | | |
| 227 | 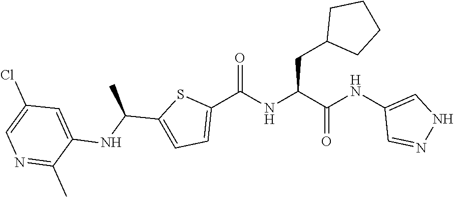 [SMILES: Cc1ncc(Cl)cc1N[C@@H](C)c1ccc(C(=O)N[C@@H](CC2CCCC2)C(=O)Nc2cn[nH]c2)s1] | 501.05 | 500.1761 |
| | (2S)-2-({5-[(1S)-1-[(5-chloro-2-methylpyridin-3- | | |
| | yl)amino]ethyl]thiophen-2-yl}formamido)-3- | | |
| | cyclopentyl-N-(1H-pyrazol-4-yl)propanamide | | |
| 228 | 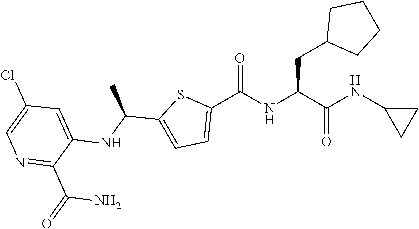 [SMILES: C[C@H](Nc1cc(Cl)cnc1C(N)=O)c1ccc(C(=O)N[C@@H](CC2CCCC2)C(=O)NC2CC2)s1] | 504.05 | 503.1758 |
| | 5-chloro-3-{[(1S)-1-(5-{[(1S)-2-cyclopentyl-1- | | |
| | (cyclopropylcarbamoyl)ethyl]carbamoyl}thiophen-2- | | |
| | yl)ethyl]amino}pyridine-2-carboxamide | | |
| 229 | 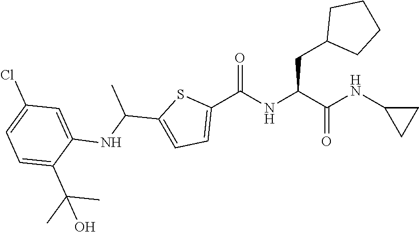 [SMILES: CC(Nc1cc(Cl)ccc1C(C)(C)O)c1ccc(C(=O)N[C@@H](CC2CCCC2)C(=O)NC2CC2)s1] | 518.11 | 517.2166 |
| | (2S)-2-{[5-(1-{[5-chloro-2-(2-hydroxypropan-2- | | |
| | yl)phenyl]amino}ethyl)thiophen-2-yl]formamido}-3- | | |
| | cyclopentyl-N-cyclopropylpropanamide | | |
| 230 | 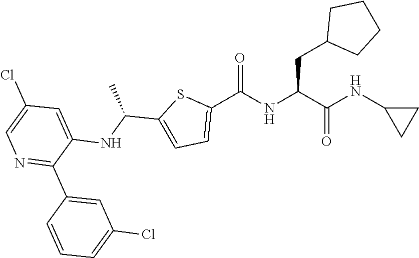 [SMILES: C[C@@H](Nc1cc(Cl)cnc1-c1cccc(Cl)c1)c1ccc(C(=O)N[C@@H](CC2CCCC2)C(=O)NC2CC2)s1] | 571.56 | 570.1623 |
| | (2S)-2-({5-[(1R)-1-{[5-chloro-2-(3- | | |
| | chlorophenyl)pyridin-3-yl]amino}ethyl]thiophen-2- | | |
| | yl}formamido)-3-cyclopentyl-N- | | |
| | cyclopropylpropanamide | | |
| 231 | 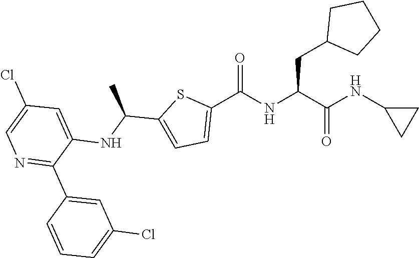 [SMILES: C[C@H](Nc1cc(Cl)cnc1-c1cccc(Cl)c1)c1ccc(C(=O)N[C@@H](CC2CCCC2)C(=O)NC2CC2)s1] | 571.56 | 570.1623 |
| | (2S)-2-({5-[(1S)-1-{[5-chloro-2-(3- | | |
| | chlorophenyl)pyridin-3-yl]amino}ethyl]thiophen-2- | | |
| | yl}formamido)-3-cyclopentyl-N- | | |
| | cyclopropylpropanamide | | |
| 232 | 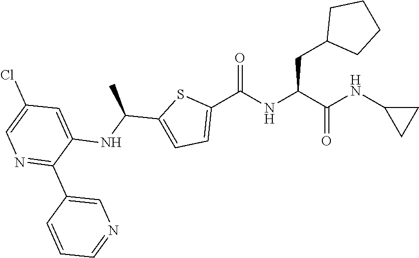 [SMILES: C[C@H](Nc1cc(Cl)cnc1-c1cccnc1)c1ccc(C(=O)N[C@@H](CC2CCCC2)C(=O)NC2CC2)s1] | 538.11 | 537.1965 |
| | (2S)-2-({5-[(1S)-1-({5-chloro-[2,3′-bipyridin]-3- | | |
| | yl}amino)ethyl]thiophen-2-yl}formamido)-3- | | |
| | cyclopentyl-N-cyclopropylpropanamide | | |
| 233 | 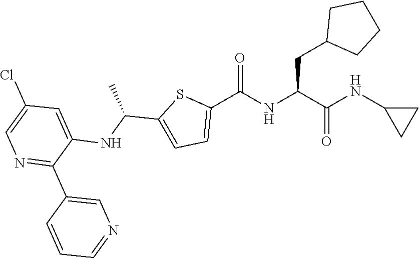 [SMILES: C[C@@H](Nc1cc(Cl)cnc1-c1cccnc1)c1ccc(C(=O)N[C@@H](CC2CCCC2)C(=O)NC2CC2)s1] | 538.11 | 537.1965 |
| | (2S)-2-({5-[(1R)-1-({5-chloro-[2,3′-bipyridin]-3- | | |
| | yl}amino)ethyl]thiophen-2-yl}formamido)-3- | | |
| | cyclopentyl-N-cyclopropylpropanamide | | |
| 234 | 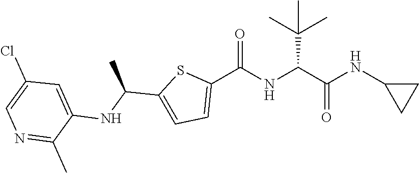 [SMILES: Cc1ncc(Cl)cc1N[C@@H](C)c1ccc(C(=O)N[C@@H](C(=O)NC2CC2)C(C)(C)C)s1] | 449.01 | 448.17 |
| | (2R)-2-({5-[(1S)-1-[(5-chloro-2-methylpyridin-3- | | |
| | yl)amino]ethyl]thiophen-2-yl}formamido)-N- | | |
| | cyclopropyl-3,3-dimethylbutanamide | | |
| 235 | 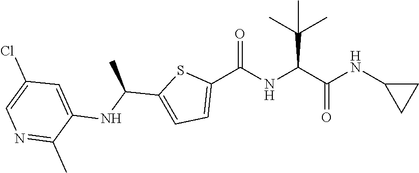 [SMILES: Cc1ncc(Cl)cc1N[C@@H](C)c1ccc(C(=O)N[C@H](C(=O)NC2CC2)C(C)(C)C)s1] | 449.01 | 448.17 |
| | (2S)-2-({5-[(1S)-1-[(5-chloro-2-methylpyridin-3- | | |
| | yl)amino]ethyl]thiophen-2-yl}formamido)-N- | | |
| | cyclopropyl-3,3-dimethylbutanamide | | |
| 236 | 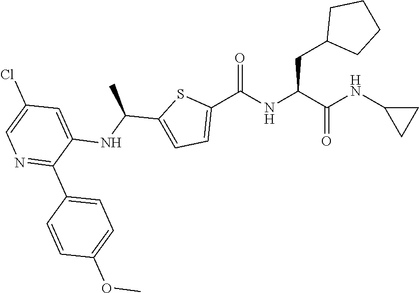 [SMILES: COc1ccc(-c2ncc(Cl)cc2N[C@@H](C)c2ccc(C(=O)N[C@@H](CC3CCCC3)C(=O)NC3CC3)s2)cc1] | 567.15 | 566.2118 |
| | (2S)-2-({5-[(1S)-1-{[5-chloro-2-(4- | | |
| | methoxyphenyl)pyridin-3-yl]amino}ethyl]thiophen-2- | | |
| | yl}formamido)-3-cyclopentyl-N- | | |
| | cyclopropylpropanamide | | |
| 237 | 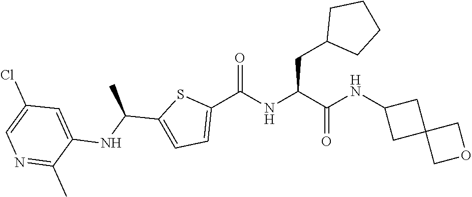 [SMILES: Cc1ncc(Cl)cc1N[C@@H](C)c1ccc(C(=O)N[C@@H](CC2CCCC2)C(=O)NC2CC3(COC3)C2)s1] | 531.11 | 530.2118 |
| | (2S)-2-({5-[(1S)-1-[(5-chloro-2-methylpyridin-3- | | |
| | yl)amino]ethyl]thiophen-2-yl}formamido)-3- | | |
| | cyclopentyl-N-{2-oxaspiro[3.3]heptan-6- | | |
| | yl}propanamide | | |
| 238 | 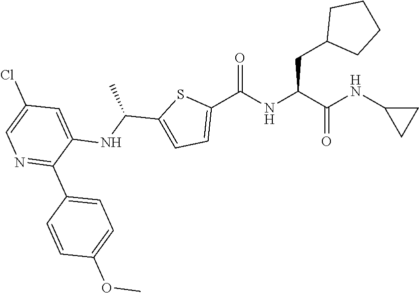 [SMILES: COc1ccc(-c2ncc(Cl)cc2N[C@H](C)c2ccc(C(=O)N[C@@H](CC3CCCC3)C(=O)NC3CC3)s2)cc1] | 567.15 | 566.2118 |
| | (2S)-2-({5-[(1R)-1-{[5-chloro-2-(4- | | |
| | methoxyphenyl)pyridin-3-yl]amino}ethyl]thiophen-2- | | |
| | yl}formamido)-3-cyclopentyl-N- | | |
| | cyclopropylpropanamide | | |
| 239 | 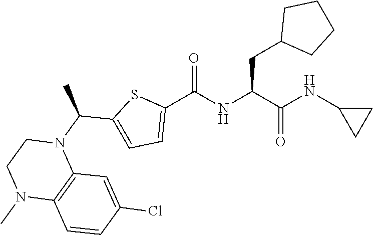 [SMILES: C[C@@H](c1ccc(C(=O)N[C@@H](CC2CCCC2)C(=O)NC2CC2)s1)N1CCN(C)c2ccc(Cl)cc21] | 515.11 | 514.2169 |
| | (2S)-2-({5-[(1S)-1-(7-chloro-4-methyl-1,2,3,4- | | |
| | tetrahydroquinoxalin-1-yl)ethyl]thiophen-2- | | |
| | yl}formamido)-3-cyclopentyl-N- | | |
| | cyclopropylpropanamide | | |
| 240 | 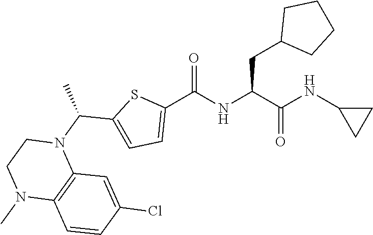 [SMILES: C[C@H](c1ccc(C(=O)N[C@@H](CC2CCCC2)C(=O)NC2CC2)s1)N1CCN(C)c2ccc(Cl)cc21] | 515.11 | 514.2169 |
| | (2S)-2-({5-[(1R)-1-(7-chloro-4-methyl-1,2,3,4- | | |
| | tetrahydroquinoxalin-1-yl)ethyl]thiophen-2- | | |
| | yl}formamido)-3-cyclopentyl-N- | | |
| | cyclopropylpropanamide | | |
| 241 | 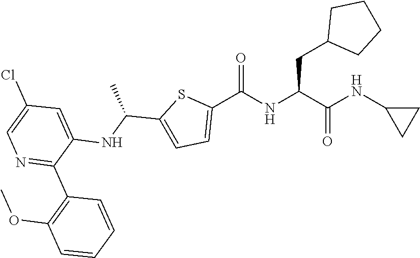 [SMILES: COc1ccccc1-c1ncc(Cl)cc1N[C@H](C)c1ccc(C(=O)N[C@@H](CC2CCCC2)C(=O)NC2CC2)s1] | 567.15 | 566.2118 |
| | (2S)-2-({5-[(1R)-1-{[5-chloro-2-(2- | | |
| | methoxyphenyl)pyridin-3-yl]amino}ethyl]thiophen-2- | | |
| | yl}formamido)-3-cyclopentyl-N- | | |
| | cyclopropylpropanamide | | |
| 242 | 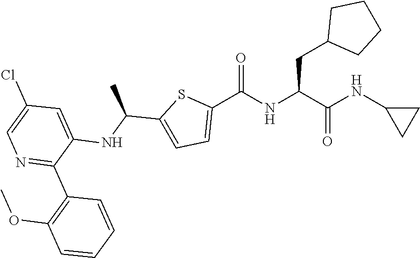 [SMILES: COc1ccccc1-c1ncc(Cl)cc1N[C@@H](C)c1ccc(C(=O)N[C@@H](CC2CCCC2)C(=O)NC2CC2)s1] | 567.15 | 566.2118 |
| | (2S)-2-({5-[(1S)-1-{[5-chloro-2-(2- | | |
| | methoxyphenyl)pyridin-3-yl]amino}ethyl]thiophen-2- | | |
| | yl}formamido)-3-cyclopentyl-N- | | |
| | cyclopropylpropanamide | | |
| 243 | 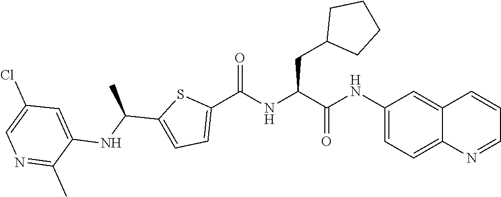 [SMILES: Cc1ncc(Cl)cc1N[C@@H](C)c1ccc(C(=O)N[C@@H](CC2CCCC2)C(=O)Nc2ccc3ncccc3c2)s1] | 562.13 | 561.1965 |
| | (2S)-2-({5-[(1S)-1-[(5-chloro-2-methylpyridin-3- | | |
| | yl)amino]ethyl]thiophen-2-yl}formamido)-3- | | |
| | cyclopentyl-N-(quinolin-6-yl)propanamide | | |
| 244 | 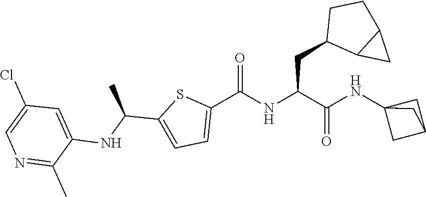 [SMILES: Cc1ncc(Cl)cc1N[C@@H](C)c1ccc(C(=O)N[C@@H](C[C@H]2CCC3CC32)C(=O)NC23CC(C2)C3)s1] | 513.1 | 512.2013 |
| | (2S)-N-{bicyclo[1.1.1]pentan-1-yl}-3-[(2R)- | | |
| | bicyclo[3.1.0]hexan-2-yl]-2-({5-[(1S)-1-[(5-chloro-2- | | |
| | methylpyridin-3-yl)amino]ethyl]thiophen-2- | | |
| | yl}formamido)propanamide | | |
| 245 | 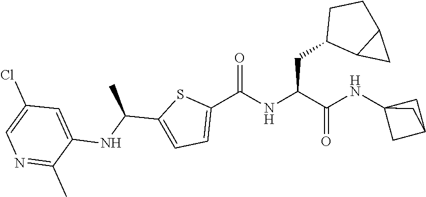 [SMILES: Cc1ncc(Cl)cc1N[C@@H](C)c1ccc(C(=O)N[C@@H](C[C@@H]2CCC3CC32)C(=O)NC23CC(C2)C3)s1] | 513.1 | 512.2013 |
| | (2S)-N-{bicyclo[1.1.1]pentan-1-yl}-3-[(2S)- | | |
| | bicyclo[3.1.0]hexan-2-yl]-2-({5-[(1S)-1-[(5-chloro-2- | | |
| | methylpyridin-3-yl)amino]ethyl]thiophen-2- | | |
| | yl}formamido)propanamide | | |
| 246 | 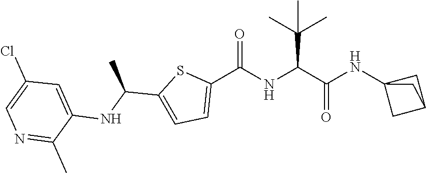 [SMILES: Cc1ncc(Cl)cc1N[C@@H](C)c1ccc(C(=O)N[C@H](C(=O)NC23CC(C2)C3)C(C)(C)C)s1] | 475.05 | 474.1856 |
| | (2S)-N-{bicyclo[1.1.1]pentan-1-yl}-2-({5-[(1S)-1-[(5- | | |
| | chloro-2-methylpyridin-3-yl)amino]ethyl]thiophen-2- | | |
| | yl}formamido)-3,3-dimethylbutanamide | | |
| 247 | 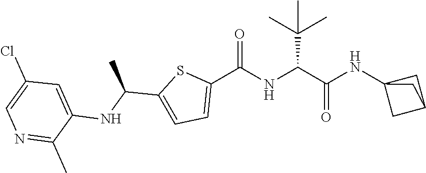 [SMILES: Cc1ncc(Cl)cc1N[C@@H](C)c1ccc(C(=O)N[C@@H](C(=O)NC23CC(C2)C3)C(C)(C)C)s1] | 475.05 | 474.1856 |
| | (2R)-N-{bicyclo[1.1.1]pentan-1-yl}-2-({5-[(1S)-1-[(5- | | |
| | chloro-2-methylpyridin-3-yl)amino]ethyl]thiophen-2- | | |
| | yl}formamido)-3,3-dimethylbutanamide | | |
| 248 | 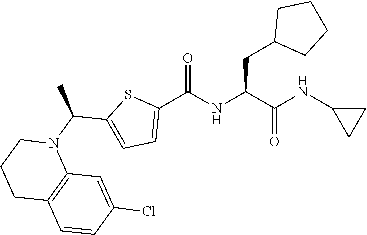 [SMILES: C[C@@H](c1ccc(C(=O)N[C@@H](CC2CCCC2)C(=O)NC2CC2)s1)N1CCCc2ccc(Cl)cc21] | 500.1 | 499.206 |
| | (2S)-2-({5-[(1S)-1-(7-chloro-1,2,3,4- | | |
| | tetrahydroquinolin-1-yl)ethyl]thiophen-2- | | |
| | yl}formamido)-3-cyclopentyl-N- | | |
| | cyclopropylpropanamide | | |
| 249 | 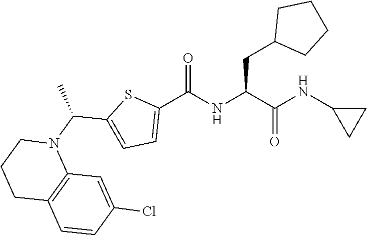 [SMILES: C[C@H](c1ccc(C(=O)N[C@@H](CC2CCCC2)C(=O)NC2CC2)s1)N1CCCc2ccc(Cl)cc21] | 500.1 | 499.206 |
| | (2S)-2-({5-[(1R)-1-(7-chloro-1,2,3,4- | | |
| | tetrahydroquinolin-1-yl)ethyl]thiophen-2- | | |
| | yl}formamido)-3-cyclopentyl-N- | | |
| | cyclopropylpropanamide | | |
| 250 | 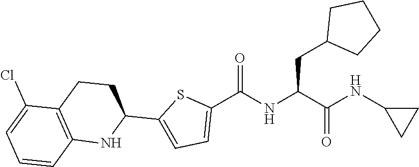 [SMILES: O=C(N[C@@H](CC1CCCC1)C(=O)NC1CC1)c1ccc([C@@H]2CCc3c(Cl)cccc3N2)s1] | 472.04 | 471.1747 |
| | (2S)-2-({5-[(2S)-5-chloro-1,2,3,4-tetrahydroquinolin- | | |
| | 2-yl]thiophen-2-yl}formamido)-3-cyclopentyl-N- | | |
| | cyclopropylpropanamide | | |
| 251 | 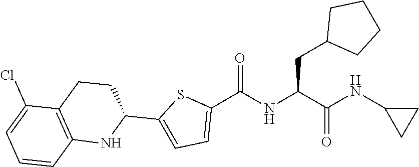 [SMILES: O=C(N[C@@H](CC1CCCC1)C(=O)NC1CC1)c1ccc([C@H]2CCc3c(Cl)cccc3N2)s1] | 472.04 | 471.1747 |
| | (2S)-2-({5-[(2R)-5-chloro-1,2,3,4-tetrahydroquinolin- | | |
| | 2-yl]thiophen-2-yl}formamido)-3-cyclopentyl-N- | | |
| | cyclopropylpropanamide | | |
| 252 | 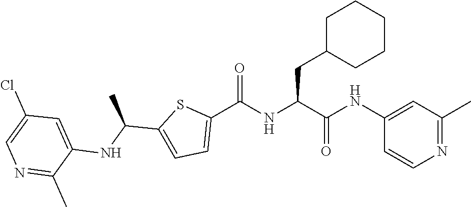 [SMILES: Cc1cc(NC(=O)[C@H](CC2CCCCC2)NC(=O)c2ccc([C@H](C)Nc3cc(Cl)cnc3C)s2)ccn1] | 540.12 | 539.2122 |
| | (2S)-2-({5-[(1S)-1-[(5-chloro-2-methylpyridin-3- | | |
| | yl)amino]ethyl]thiophen-2-yl}formamido)-3- | | |
| | cyclohexyl-N-(2-methylpyridin-4-yl)propanamide | | |
| 253 | 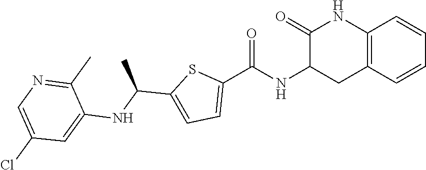 [SMILES: Cc1ncc(Cl)cc1N[C@@H](C)c1ccc(C(=O)NC2Cc3ccccc3NC2=O)s1] | 440.95 | 440.1074 |
| | 5-[(1S)-1-[(5-chloro-2-methylpyridin-3- | | |
| | yl)amino]ethyl]-N-(2-oxo-1,2,3,4-tetrahydroquinolin- | | |
| | 3-yl)thiophene-2-carboxamide | | |
| 254 | 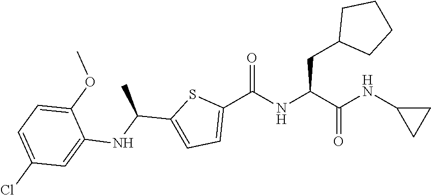 [SMILES: COc1ccc(Cl)cc1N[C@@H](C)c1ccc(C(=O)N[C@@H](CC2CCCC2)C(=O)NC2CC2)s1] | 490.06 | 489.1853 |
| | (2S)-2-({5-[(1S)-1-[(5-chloro-2- | | |
| | methoxyphenyl)amino]ethyl]thiophen-2- | | |
| | yl}formamido)-3-cyclopentyl-N- | | |
| | cyclopropylpropanamide | | |
| 255 | 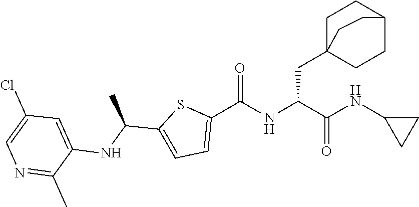 [SMILES: Cc1ncc(Cl)cc1N[C@@H](C)c1ccc(C(=O)N[C@H](CC23CCC(CC2)CC3)C(=O)NC2CC2)s1] | 515.11 | 514.2169 |
| | (2R)-3-{bicyclo[2.2.2]octan-1-yl}-2-({5-[(1S)-1-[(5- | | |
| | chloro-2-methylpyridin-3-yl)amino]ethyl]thiophen-2- | | |
| | yl}formamido)-N-cyclopropylpropanamide | | |
| 256 | 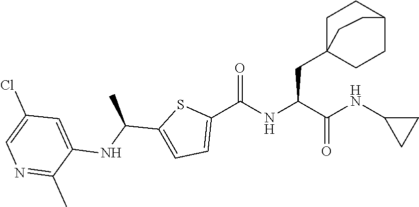 [SMILES: Cc1ncc(Cl)cc1N[C@@H](C)c1ccc(C(=O)N[C@@H](CC23CCC(CC2)CC3)C(=O)NC2CC2)s1] | 515.11 | 514.2169 |
| | (2S)-3-{bicyclo[2.2.2]octan-1-yl}-2-({5-[(1S)-1-[(5- | | |
| | chloro-2-methylpyridin-3-yl)amino]ethyl]thiophen-2- | | |
| | yl}formamido)-N-cyclopropylpropanamide | | |
| 257 | 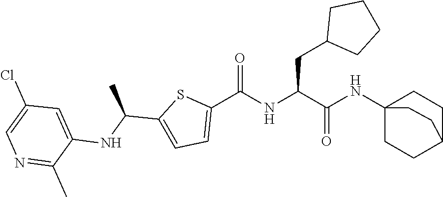 [SMILES: Cc1ncc(Cl)cc1N[C@@H](C)c1ccc(C(=O)N[C@@H](CC2CCCC2)C(=O)NC23CCC(CC2)CC3)s1] | 543.17 | 542.2482 |
| | (2S)-N-{bicyclo[2.2.2]octan-1-yl}-2-({5-[(1S)-1-[(5- | | |
| | chloro-2-methylpyridin-3-yl)amino]ethyl]thiophen-2- | | |
| | yl}formamido)-3-cyclopentylpropanamide | | |
| 258 | 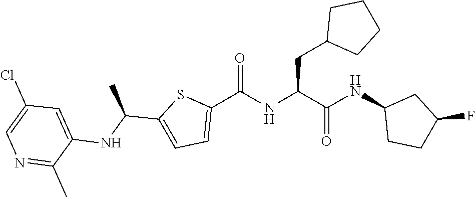 [SMILES: Cc1ncc(Cl)cc1N[C@@H](C)c1ccc(C(=O)N[C@@H](CC2CCCC2)C(=O)N[C@@H]2CC[C@H](F)C2)s1] | 521.09 | 520.2075 |
| | (2S)-2-({5-[(1S)-1-[(5-chloro-2-methylpyridin-3- | | |
| | yl)amino]ethyl]thiophen-2-yl}formamido)-3- | | |
| | cyclopentyl-N-[(1R,3S)-3- | | |
| | fluorocyclopentyl]propanamide | | |
| 259 | 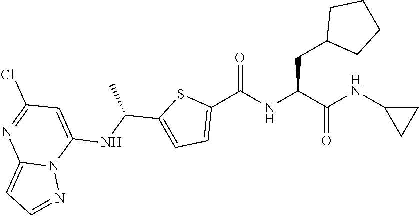 [SMILES: C[C@@H](Nc1cc(Cl)nc2ccnn12)c1ccc(C(=O)N[C@@H](CC2CCCC2)C(=O)NC2CC2)s1] | 501.05 | 500.1761 |
| | (2S)-2-({5-[(1R)-1-({5-chloropyrazolo[1,5- | | |
| | a]pyrimidin-7-yl}amino)ethyl]thiophen-2- | | |
| | yl}formamido)-3-cyclopentyl-N- | | |
| | cyclopropylpropanamide | | |
| 260 | 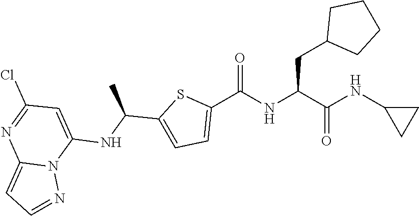 [SMILES: C[C@H](Nc1cc(Cl)nc2ccnn12)c1ccc(C(=O)N[C@@H](CC2CCCC2)C(=O)NC2CC2)s1] | 501.05 | 500.1761 |
| | (2S)-2-({5-[(1S)-1-({5-chloropyrazolo[1,5- | | |
| | a]pyrimidin-7-yl}amino)ethyl]thiophen-2- | | |
| | yl}formamido)-3-cyclopentyl-N- | | |
| | cyclopropylpropanamide | | |
| 261 | 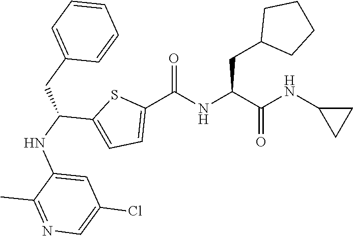 [SMILES: Cc1ncc(Cl)cc1N[C@H](Cc1ccccc1)c1ccc(C(=O)N[C@@H](CC2CCCC2)C(=O)NC2CC2)s1] | 551.15 | 550.2169 |
| | (2S)-2-({5-[(1R)-1-[(5-chloro-2-methylpyridin-3- | | |
| | yl)amino]-2-phenylethyl]thiophen-2-yl}formamido)- | | |
| | 3-cyclopentyl-N-cyclopropylpropanamide | | |
| 262 | 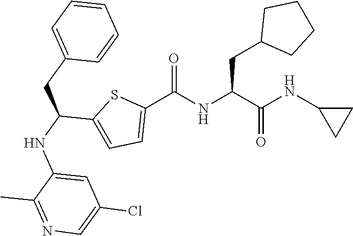 [SMILES: Cc1ncc(Cl)cc1N[C@@H](Cc1ccccc1)c1ccc(C(=O)N[C@@H](CC2CCCC2)C(=O)NC2CC2)s1] | 551.15 | 550.2169 |
| | (2S)-2-({5-[(1S)-1-[(5-chloro-2-methylpyridin-3- | | |
| | yl)amino]-2-phenylethyl]thiophen-2-yl}formamido)- | | |
| | 3-cyclopentyl-N-cyclopropylpropanamide | | |
| 263 | 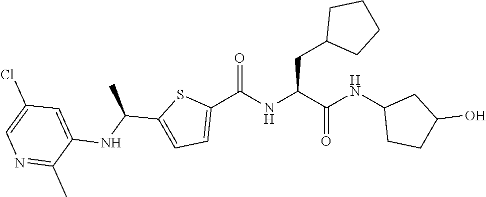 [SMILES: Cc1ncc(Cl)cc1N[C@@H](C)c1ccc(C(=O)N[C@@H](CC2CCCC2)C(=O)NC2CCC(O)C2)s1] | 519.1 | 518.2118 |
| | (2S)-2-({5-[(1S)-1-[(5-chloro-2-methylpyridin-3- | | |
| | yl)amino]ethyl]thiophen-2-yl}formamido)-3- | | |
| | cyclopentyl-N-(3-hydroxycyclopentyl)propanamide | | |
| 264 | 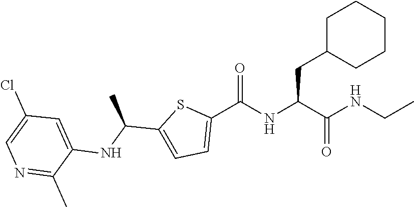 [SMILES: CCNC(=O)[C@H](CC1CCCCC1)NC(=O)c1ccc([C@H](C)Nc2cc(Cl)cnc2C)s1] | 477.06 | 476.2013 |
| | (2S)-2-({5-[(1S)-1-[(5-chloro-2-methylpyridin-3- | | |
| | yl)amino]ethyl]thiophen-2-yl}formamido)-3- | | |
| | cyclohexyl-N-ethylpropanamide | | |
| 265 | 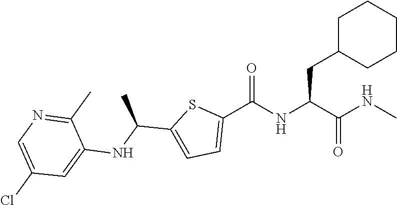 [SMILES: CNC(=O)[C@H](CC1CCCCC1)NC(=O)c1ccc([C@H](C)Nc2cc(Cl)cnc2C)s1] | 463.04 | 462.1856 |
| | (2S)-2-({5-[(1S)-1-[(5-chloro-2-methylpyridin-3- | | |
| | yl)amino]ethyl]thiophen-2-yl}formamido)-3- | | |
| | cyclohexyl-N-methylpropanamide | | |
| 266 | 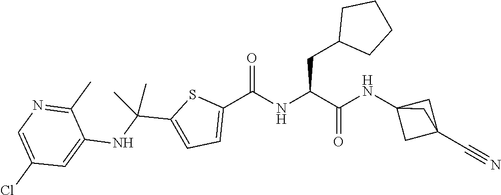 [SMILES: Cc1ncc(Cl)cc1NC(C)(C)c1ccc(C(=O)N[C@@H](CC2CCCC2)C(=O)NC23CC(C#N)(C2)C3)s1] | 540.12 | 539.2122 |
| | (2S)-2-[(5-{2-[(5-chloro-2-methylpyridin-3- | | |
| | yl)amino]propan-2-yl}thiophen-2-yl)formamido]-N- | | |
| | {3-cyanobicyclo[1.1.1]pentan-1-yl}-3- | | |
| | cyclopentylpropanamide | | |
| 267 | 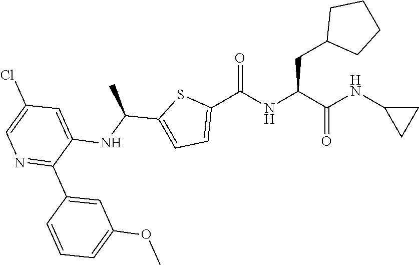 [SMILES: COc1cccc(-c2ncc(Cl)cc2N[C@@H](C)c2ccc(C(=O)N[C@@H](CC3CCCC3)C(=O)NC3CC3)s2)c1] | 567.15 | 566.2118 |
| | (2S)-2-({5-[(1S)-1-{[5-chloro-2-(3- | | |
| | methoxyphenyl)pyridin-3-yl]amino}ethyl]thiophen-2- | | |
| | yl}formamido)-3-cyclopentyl-N- | | |
| | cyclopropylpropanamide | | |
| 268 | 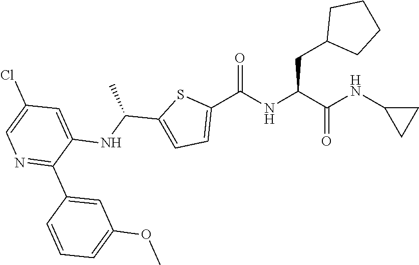 [SMILES: COc1cccc(-c2ncc(Cl)cc2N[C@H](C)c2ccc(C(=O)N[C@@H](CC3CCCC3)C(=O)NC3CC3)s2)c1] | 567.15 | 566.2118 |
| | (2S)-2-({5-[(1R)-1-{[5-chloro-2-(3- | | |
| | methoxyphenyl)pyridin-3-yl]amino}ethyl]thiophen-2- | | |
| | yl}formamido)-3-cyclopentyl-N- | | |
| | cyclopropylpropanamide | | |
| 269 | 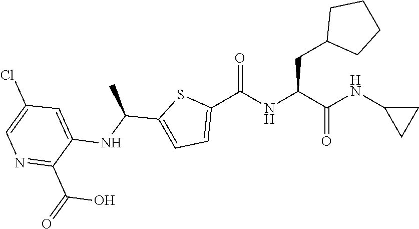 [SMILES: C[C@H](Nc1cc(Cl)cnc1C(=O)O)c1ccc(C(=O)N[C@@H](CC2CCCC2)C(=O)NC2CC2)s1] | 505.03 | 504.1598 |
| | 5-chloro-3-{[(1S)-1-(5-{[(1S)-2-cyclopentyl-1- | | |
| | (cyclopropylcarbamoyl)ethyl]carbamoyl}thiophen-2- | | |
| | yl)ethyl]amino}pyridine-2-carboxylic acid | | |
| 270 | 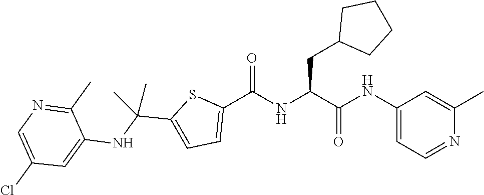 [SMILES: Cc1cc(NC(=O)[C@H](CC2CCCC2)NC(=O)c2ccc(C(C)(C)Nc3cc(Cl)cnc3C)s2)ccn1] | 540.12 | 539.2122 |
| | (2S)-2-[(5-{2-[(5-chloro-2-methylpyridin-3- | | |
| | yl)amino]propan-2-yl}thiophen-2-yl)formamido]-3- | | |
| | cyclopentyl-N-(2-methylpyridin-4-yl)propanamide | | |
| 271 | 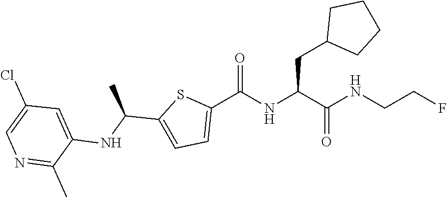 [SMILES: Cc1ncc(Cl)cc1N[C@@H](C)c1ccc(C(=O)N[C@@H](CC2CCCC2)C(=O)NCCF)s1] | 481.03 | 480.1762 |
| | (2S)-2-({5-[(1S)-1-[(5-chloro-2-methylpyridin-3- | | |
| | yl)amino]ethyl]thiophen-2-yl}formamido)-3- | | |
| | cyclopentyl-N-(2-fluoroethyl)propanamide | | |
| 272 | 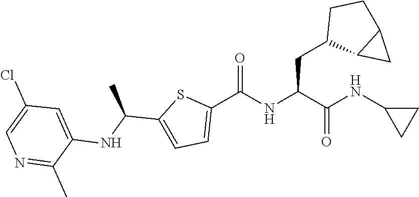 | 487.06 | 486.1856 |
| | (2S)-3-[(2R)-bicyclo[3.1.0]hexan-2-yl]-2-({5-[(1S)-1- | | |
| | [(5-chloro-2-methylpyridin-3- | | |
| | yl)amino]ethyl]thiophen-2-yl}formamido)-N- | | |
| | cyclopropylpropanamide | | |
| 273 | 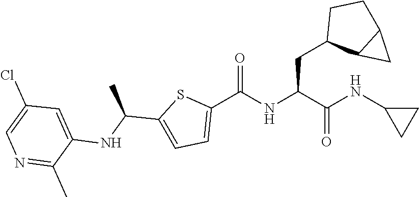 | 487.06 | 486.1856 |
| | (2S)-3-[(2S)-bicyclo[3.1.0]hexan-2-yl]-2-({5-[(1S)-1- | | |
| | [(5-chloro-2-methylpyridin-3- | | |
| | yl)amino]ethyl]thiophen-2-yl}formamido)-N- | | |
| | cyclopropylpropanamide | | |
| 274 | 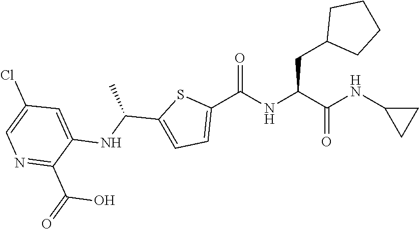 [SMILES: C[C@@H](Nc1cc(Cl)cnc1C(=O)O)c1ccc(C(=O)N[C@@H](CC2CCCC2)C(=O)NC2CC2)s1] | 505.03 | 504.1598 |
| | 5-chloro-3-{[(1R)-1-(5-{[(1S)-2-cyclopentyl-1- | | |
| | (cyclopropylcarbamoyl)ethyl]carbamoyl}thiophen-2- | | |
| | yl)ethyl]amino}pyridine-2-carboxylic acid | | |
| 275 | 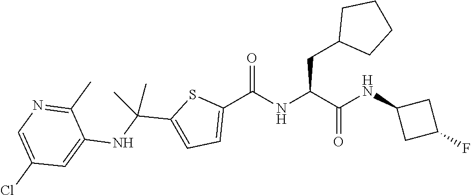 [SMILES: Cc1ncc(Cl)cc1NC(C)(C)c1ccc(C(=O)N[C@@H](CC2CCCC2)C(=O)N[C@H]2C[C@H](F)C2)s1] | 521.09 | 520.2075 |
| | (2S)-2-[(5-{2-[(5-chloro-2-methylpyridin-3- | | |
| | yl)amino]propan-2-yl}thiophen-2-yl)formamido]-3- | | |
| | cyclopentyl-N-[(1r,3r)-3- | | |
| | fluorocyclobutyl]propanamide | | |
| 276 | 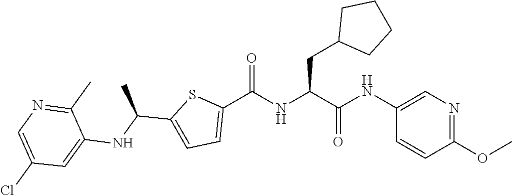 [SMILES: COc1ccc(NC(=O)[C@H](CC2CCCC2)NC(=O)c2ccc([C@H](C)Nc3cc(Cl)cnc3C)s2)cn1] | 542.1 | 541.1914 |
| | (2S)-2-({5-[(1S)-1-[(5-chloro-2-methylpyridin-3- | | |
| | yl)amino]ethyl]thiophen-2-yl}formamido)-3- | | |
| | cyclopentyl-N-(6-methoxypyridin-3-yl)propanamide | | |
| 277 | 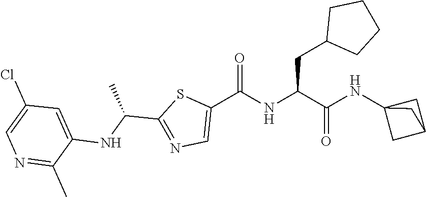 [SMILES: Cc1ncc(Cl)cc1N[C@H](C)c1ncc(C(=O)N[C@@H](CC2CCCC2)C(=O)NC23CC(C2)C3)s1] | 502.07 | 501.1965 |
| | (2S)-N-{bicyclo[1.1.1]pentan-1-yl}-2-({2-[(1R)-1-[(5- | | |
| | chloro-2-methylpyridin-3-yl)amino]ethyl]-1,3-thiazol- | | |
| | 5-yl}formamido)-3-cyclopentylpropanamide | | |
| 278 | 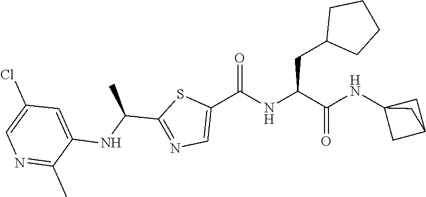 [SMILES: Cc1ncc(Cl)cc1N[C@@H](C)c1ncc(C(=O)N[C@@H](CC2CCCC2)C(=O)NC23CC(C2)C3)s1] | 502.07 | 501.1965 |
| | (2S)-N-{bicyclo[1.1.1]pentan-1-yl}-2-({2-[(1S)-1-[(5- | | |
| | chloro-2-methylpyridin-3-yl)amino]ethyl]-1,3-thiazol- | | |
| | 5-yl}formamido)-3-cyclopentylpropanamide | | |
| 279 | 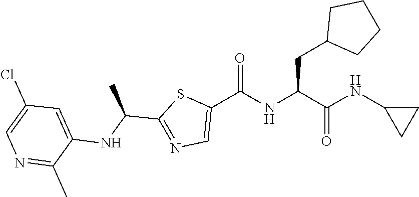 [SMILES: Cc1ncc(Cl)cc1N[C@@H](C)c1ncc(C(=O)N[C@@H](CC2CCCC2)C(=O)NC2CC2)s1] | 476.04 | 475.1809 |
| | (2S)-2-({2-[(1S)-1-[(5-chloro-2-methylpyridin-3- | | |
| | yl)amino]ethyl]-1,3-thiazol-5-yl}formamido)-3- | | |
| | cyclopentyl-N-cyclopropylpropanamide | | |
| 280 | 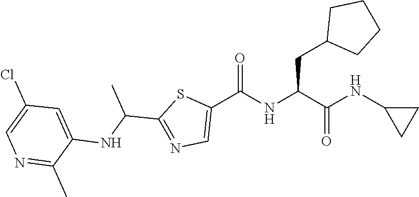 [SMILES: Cc1ncc(Cl)cc1NC(C)c1ncc(C(=O)N[C@@H](CC2CCCC2)C(=O)NC2CC2)s1] | 476.04 | 475.1809 |
| | (2S)-2-[(2-{1-[(5-chloro-2-methylpyridin-3- | | |
| | yl)amino]ethyl}-1,3-thiazol-5-yl)formamido]-3- | | |
| | cyclopentyl-N-cyclopropylpropanamide | | |
| 281 | 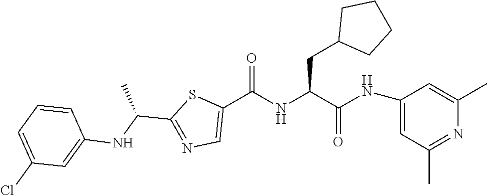 [SMILES: Cc1cc(NC(=O)[C@H](CC2CCCC2)NC(=O)c2cnc([C@@H](C)Nc3cccc(Cl)c3)s2)cc(C)n1] | 526.1 | 525.1965 |
| | (2S)-2-({2-[(1R)-1-[(3-chlorophenyl)amino]ethyl]- | | |
| | 1,3-thiazol-5-yl}formamido)-3-cyclopentyl-N-(2,6- | | |
| | dimethylpyridin-4-yl)propanamide | | |
| 282 | 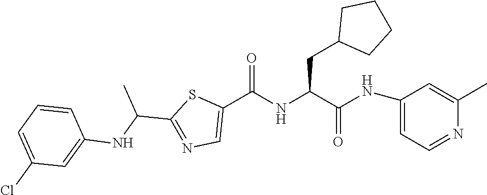 [SMILES: Cc1cc(NC(=O)[C@H](CC2CCCC2)NC(=O)c2cnc(C(C)Nc3cccc(Cl)c3)s2)ccn1] | 512.07 | 511.1809 |
| | (2S)-2-[(2-{1-[(3-chlorophenyl)amino]ethyl}-1,3- | | |
| | thiazol-5-yl)formamido]-3-cyclopentyl-N-(2- | | |
| | methylpyridin-4-yl)propanamide | | |
| 283 | 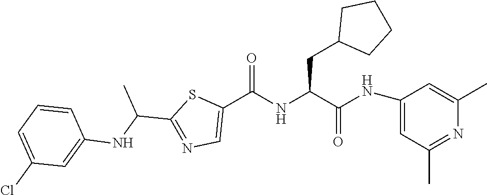 [SMILES: Cc1cc(NC(=O)[C@H](CC2CCCC2)NC(=O)c2cnc(C(C)Nc3cccc(Cl)c3)s2)cc(C)n1] | 526.1 | 525.1965 |
| | (2S)-2-[(2-{1-[(3-chlorophenyl)amino]ethyl}-1,3- | | |
| | thiazol-5-yl)formamido]-3-cyclopentyl-N-(2,6- | | |
| | dimethylpyridin-4-yl)propanamide | | |
| 284 | 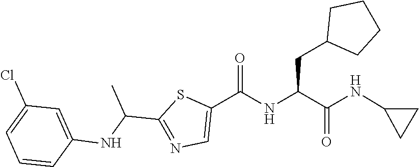 [SMILES: CC(Nc1cccc(Cl)c1)c1ncc(C(=O)N[C@@H](CC2CCCC2)C(=O)NC2CC2)s1] | 461.02 | 460.17 |
| | (2S)-2-[(2-{1-[(3-chlorophenyl)amino]ethyl}-1,3- | | |
| | thiazol-5-yl)formamido]-3-cyclopentyl-N- | | |
| | cyclopropylpropanamide | | |
| 285 | 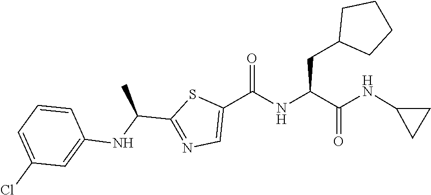 [SMILES: C[C@H](Nc1cccc(Cl)c1)c1ncc(C(=O)N[C@@H](CC2CCCC2)C(=O)NC2CC2)s1] | 461.02 | 460.17 |
| | (2S)-2-({2-[(1S)-1-[(3-chlorophenyl)amino]ethyl]-1,3- | | |
| | thiazol-5-yl}formamido)-3-cyclopentyl-N- | | |
| | cyclopropylpropanamide | | |
| 286 | 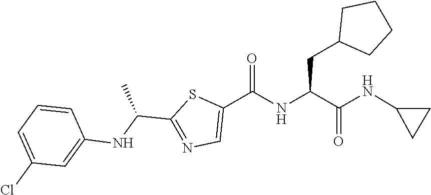 [SMILES: C[C@@H](Nc1cccc(Cl)c1)c1ncc(C(=O)N[C@@H](CC2CCCC2)C(=O)NC2CC2)s1] | 461.02 | 460.17 |
| | (2S)-2-({2-[(1R)-1-[(3-chlorophenyl)amino]ethyl]- | | |
| | 1,3-thiazol-5-yl}formamido)-3-cyclopentyl-N- | | |
| | cyclopropylpropanamide | | |
| 293 | 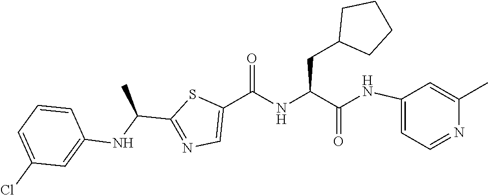 [SMILES: Cc1cc(NC(=O)[C@H](CC2CCCC2)NC(=O)c2cnc([C@H](C)Nc3cccc(Cl)c3)s2)ccn1] | 512.07 | 511.1809 |
| | (2S)-2-({2-[(1S)-1-[(3-chlorophenyl)amino]ethyl]-1,3- | | |
| | thiazol-5-yl}formamido)-3-cyclopentyl-N-(2- | | |
| | methylpyridin-4-yl)propanamide | | |
| 295 | 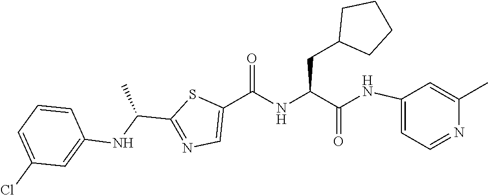 [SMILES: Cc1cc(NC(=O)[C@H](CC2CCCC2)NC(=O)c2cnc([C@@H](C)Nc3cccc(Cl)c3)s2)ccn1] | 512.07 | 511.1809 |
| | (2S)-2-({2-[(1R)-1-[(3-chlorophenyl)amino]ethyl]- | | |
| | 1,3-thiazol-5-yl}formamido)-3-cyclopentyl-N-(2- | | |
| | methylpyridin-4-yl)propanamide | | |
| 296 | 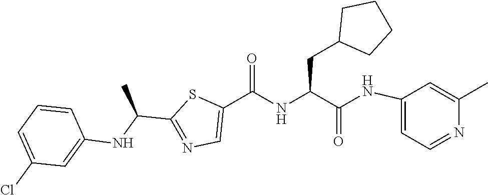 [SMILES: Cc1cc(NC(=O)[C@H](CC2CCCC2)NC(=O)c2cnc([C@H](C)Nc3cccc(Cl)c3)s2)ccn1] | 512.07 | 511.1809 |
| | (2S)-2-({2-[(1S)-1-[(3-chlorophenyl)amino]ethyl]-1,3- | | |
| | thiazol-5-yl}formamido)-3-cyclopentyl-N-(2- | | |
| | methylpyridin-4-yl)propanamide | | |

### Example 17

### Evaluation of Compounds as WIP Inhibitors

### Plasmid Construction, Exogenous Protein Expression and Purification of Human Wip1(2-420) and WIp1 Full Length.

The Human Wip1 (2-420, aa 2-420) or Wip1 full length (aa 1-605) DNA sequence was codon optimized and synthesized by Genscript for insect cell expression. The open reading frame of Wip1(2-420) with a N-terminal-His6 affinity tag followed by a TEV cleavage site was subcloned sites into the pFastBac1 expression vector. The recombinant bacmid DNA was transfected in Sf9 insect cells for baculovirus production. After 72 hours infection, the Sf9 cells were harvested by centrifugation and resuspended in lysis buffer containing 50 mM Tris/HCl, pH 8.0, 500 mM NaCl, 20% glycerol, Benzonase 0.5% CHAPS and Roche cOmplete™ EDTA free protease inhibitors. Cells were disrupted with microfluidizer and the debris was removed by centrifugation at 185,000 g for 1 hour. The supernatant was subjected to HisTrap FF for affinity purification. Proteins were eluted with a linear gradient of 0-500 mM imidazole in HisTrap buffer containing 50 mM Tris/HCl, pH 8.0, 500 mM NaCl and 20% glycerol, 0.05% CHAPS. The eluted protein was dialyzed against dialysis buffer (20 mM MES, 100 mM NaCl, 20% glycerol, 0.5 mM TCEP, 0.05% CHAPS) followed purification via HiTrap SP HP column. The protein was eluted with a gradient of 100 mM-2 M NaCl in dialysis buffer. His₆-Wip1(2-20) was finally concentrated and loaded onto a size-exclusion column (NiLoad 16/600 Superdex 200 pg, GE Healthcare) pre-equilibrated with 50 mM MOPS (pH 7.5), 200 mM NaCl, 20% glycerol, 1 mM TCEP, 0.05% CHAPS and 0.1 mM EGTA. The peak fractions containing purified Nis₆-Wip1(2-420) were pooled and concentrated to 1.0 mg/mL and flash frozen at −80° C.

### Wip1 Length and Wip1(2-420) Phosphatase Enzyme FDP Assays.

Test compounds (252 mM stock in DMSO) were diluted 3 folds in series in DMSO and 1.2 μl per well were added into 384-well polypropylene black plates (Nunc). Twelve μl per well of Assay Buffer (100 mM NaCl, 50 mM MOPS, pH 7.5, 0.8 mM CHAPS, 0.1 mM EGTA, 30 mM MgCl2, 0.3 mM TCEP, 0.2 mg/mL BSA) was added followed by 12 ml per well of 50.4 nM recombinant His-tag Wip1 full length protein (amino acids #1-605) or recombinant His-tag Wip1(2-420) protein (amino acids #2-420) in Assay Buffer and the plate was incubated at RT (22° C.) for 5 min. Ten mM of fluorescein diphosphate (FDP) in DMSO was diluted to 40 mM in Assay Buffer and 5 ml per well was added to the plate and incubated at RT (22° C.) for 2 hours. Four ml per well of 64.1 mM EDTA in water were added to stop the enzyme reaction. The plate was centrifuged at 1,200 rpm (Eppendorf 5810R Plate centrifuge) for 1 minute and fluorescence intensity (Ex485/Em535) was measured on Perkin Elmer Victor Reader. EC50 values were calculated using either Prism (GraphPad) or ActivityBase software (IDBS), The IC50 results are provided in Table M.

TABLE M

| | WIP1 (Truncated 2-420) | WIP1 (Full-Length) |
|---|---|---|
| # | FDP Assay: IC50 (μM) | FDP Assay: IC50 (μM) |
| 1 | 0.006 | 0.009 |
| 2 | 0.232 | 0.117 |
| 3 | 0.06 | 0.028 |
| 4 | 0.079 | 0.015 |
| 5 | 0.013 | 0.024 |
| 6 | 0.006 | 0.009 |
| 7 | 0.08 | 0.068 |
| 8 | 0.008 | 0.011 |
| 9 | 0.411 | |
| 10 | 0.01 | 0.018 |
| 11 | 0.006 | 0.009 |
| 12 | 0.148 | 0.356 |
| 13 | 0.01 | 0.011 |
| 14 | 0.007 | 0.006 |
| 15 | 0.08 | 0.071 |
| 16 | 0.007 | 0.01 |
| 17 | 0.007 | 0.006 |
| 18 | 0.007 | 0.008 |
| 19 | 0.011 | 0.013 |
| 20 | 0.008 | 0.01 |
| 21 | 0.109 | 0.139 |
| 22 | 0.314 | |
| 23 | 0.005 | 0.008 |
| 24 | 0.008 | 0.008 |
| 25 | 0.005 | 0.008 |
| 26 | 0.007 | 0.009 |
| 27 | 0.006 | 0.01 |
| 28 | 0.004 | 0.007 |
| 29 | 0.003 | 0.007 |
| 30 | 0.006 | 0.01 |
| 31 | 0.01 | 0.016 |
| 32 | 0.048 | 0.073 |
| 33 | 0.008 | 0.011 |
| 34 | 0.017 | 0.034 |
| 35 | 0.301 | |
| 36 | 0.008 | 0.01 |
| 37 | 0.006 | 0.009 |
| 38 | 0.007 | 0.009 |
| 39 | 0.122 | 0.332 |
| 40 | 0.149 | 0.181 |
| 41 | 0.005 | 0.007 |
| 42 | 0.012 | 0.015 |
| 43 | 0.013 | 0.016 |
| 44 | 0.012 | 0.014 |
| 45 | 0.007 | 0.009 |
| 46 | 0.016 | 0.023 |
| 47 | 0.011 | 0.011 |
| 48 | 0.005 | 0.008 |
| 49 | 0.104 | 0.164 |
| 50 | 0.122 | 0.17 |
| 51 | 0.074 | 0.113 |
| 52 | 0.006 | 0.008 |
| 53 | 0.005 | 0.008 |
| 54 | 0.008 | 0.011 |
| 55 | 0.029 | 0.036 |
| 56 | 0.039 | 0.05 |
| 57 | 0.012 | 0.016 |
| 58 | 0.009 | 0.012 |
| 59 | 0.005 | 0.007 |
| 60 | 0.006 | 0.007 |
| 61 | 0.5 | |
| 62 | 0.006 | 0.01 |
| 63 | 0.015 | 0.022 |
| 64 | 0.008 | 0.011 |
| 65 | 0.008 | 0.01 |
| 66 | 0.434 | |
| 67 | 0.27 | |
| 68 | 0.006 | 0.008 |
| 69 | 0.004 | 0.007 |
| 70 | 0.006 | 0.008 |
| 71 | 0.127 | 0.215 |
| 72 | 0.103 | 0.136 |
| 73 | 0.004 | 0.008 |
| 74 | 0.021 | 0.027 |
| 75 | 0.006 | 0.009 |
| 76 | 0.007 | 0.009 |
| 77 | 0.006 | 0.01 |
| 78 | 0.006 | 0.007 |
| 79 | 0.223 | 0.308 |
| 80 | 0.01 | 0.018 |
| 81 | 0.693 | |
| 82 | 0.013 | 0.02 |
| 83 | 0.484 | |
| 84 | 0.008 | |
| 85 | 0.007 | |
| 86 | 0.008 | |
| 87 | 0.005 | |
| 88 | 0.006 | |
| 89 | 0.102 | |
| 90 | 0.011 | |
| 91 | 0.019 | |
| 92 | 0.01 | |
| 93 | 0.208 | |
| 94 | 0.005 | |
| 95 | 0.01 | |
| 96 | 0.076 | |
| 97 | 0.011 | |
| 98 | 0.006 | |
| 99 | 0.007 | |
| 100 | 0.086 | |
| 101 | 0.006 | |
| 102 | 0.01 | |
| 103 | 0.52 | |
| 104 | 0.006 | |
| 105 | 0.018 | |
| 106 | 0.472 | |
| 107 | 0.004 | |
| 108 | 0.005 | |
| 109 | 0.008 | |
| 110 | 0.006 | |
| 111 | 0.012 | |
| 112 | 0.004 | |
| 113 | 0.011 | |
| 114 | 0.006 | |
| 115 | 0.011 | |
| 116 | 0.01 | |
| 117 | 0.041 | 0.07 |
| 118 | 0.135 | 0.196 |
| 119 | 0.07 | 0.115 |
| 120 | 0.007 | 0.011 |
| 121 | 0.066 | 0.019 |
| 122 | 0.13 | 0.062 |
| 123 | 0.159 | 0.039 |
| 124 | 0.905 | |
| 125 | 0.059 | 0.016 |
| 126 | 1.023 | |
| 127 | 1.551 | |
| 128 | 3.909 | |
| 129 | 0.007 | |
| 130 | 0.022 | |
| 131 | 0.08 | |
| 132 | 0.076 | |
| 133 | 0.004 | |
| 134 | 0.004 | |
| 135 | 0.005 | |
| 136 | 0.005 | |
| 137 | 0.054 | |
| 138 | 0.019 | |
| 139 | 0.005 | |
| 140 | 0.402 | |
| 141 | 0.005 | |
| 142 | 2.495 | |
| 143 | 0.005 | |
| 144 | 0.006 | |
| 145 | 1.422 | |
| 146 | 0.015 | |
| 147 | 0.006 | |
| 148 | 0.006 | |
| 149 | 0.006 | |
| 150 | 0.008 | |
| 151 | 0.006 | |
| 152 | 0.006 | |
| 153 | 0.005 | |
| 154 | 0.01 | |
| 155 | 0.007 | |
| 156 | 0.004 | 0.009 |
| 157 | 0.009 | |
| 158 | 1.005 | |
| 159 | 0.013 | |
| 160 | 0.009 | 0.013 |
| 161 | 0.011 | |
| 162 | 0.006 | 0.008 |
| 163 | 0.005 | 0.008 |
| 164 | 0.037 | |
| 165 | 0.378 | |
| 166 | 0.007 | |
| 167 | 0.011 | |
| 168 | 0.011 | 0.015 |
| 169 | 0.009 | |
| 170 | 0.217 | |
| 171 | 0.007 | 0.01 |
| 172 | 0.01 | |
| 173 | 0.008 | |
| 174 | 0.037 | |
| 175 | 0.013 | 0.015 |
| 176 | 0.021 | |
| 177 | 0.018 | 0.023 |
| 178 | 0.005 | |
| 179 | 0.004 | |
| 180 | 0.004 | |
| 181 | 1.949 | |
| 182 | 0.148 | |
| 183 | 0.416 | |
| 184 | 0.131 | |
| 185 | 0.061 | |
| 186 | 0.195 | |
| 187 | 0.037 | |
| 188 | 0.011 | |
| 189 | 0.01 | |
| 190 | 0.224 | |
| 191 | 0.013 | |
| 192 | 0.408 | |
| 193 | 0.316 | |
| 194 | 0.006 | |
| 195 | 0.003 | |
| 196 | 0.006 | |
| 197 | 0.013 | |
| 198 | 0.204 | |
| 199 | 0.255 | |
| 200 | 0.321 | |
| 201 | 0.007 | |
| 202 | 0.019 | |
| 203 | 0.017 | |
| 204 | 0.102 | |
| 205 | 0.029 | |
| 206 | 0.098 | |
| 207 | 0.171 | |
| 208 | 0.007 | |
| 209 | 0.008 | |
| 210 | 0.007 | 0.01 |
| 211 | 0.01 | |
| 212 | 0.023 | |
| 213 | 0.166 | |
| 214 | 0.02 | |
| 215 | 0.101 | |
| 216 | 0.012 | |
| 217 | 0.027 | |
| 218 | 0.907 | |
| 219 | 4.219 | |
| 220 | 0.006 | |
| 221 | 0.006 | |
| 222 | 0.068 | |
| 223 | 0.011 | |
| 224 | 0.132 | |
| 225 | 0.089 | |
| 226 | 0.011 | |
| 227 | 0.008 | |
| 228 | 0.005 | |
| 229 | 0.02 | |
| 230 | 0.196 | |
| 231 | 0.008 | |
| 232 | 0.013 | |
| 233 | 0.293 | |
| 234 | >10.000 | |
| 235 | 5.66 | |
| 236 | 0.007 | |
| 237 | 0.008 | |
| 238 | 0.188 | |
| 239 | 0.038 | |
| 240 | 0.154 | |
| 241 | 0.33 | |
| 242 | 0.007 | |
| 243 | 0.004 | |
| 244 | 0.005 | |
| 245 | 0.01 | |
| 246 | 2.21 | |
| 247 | >10.000 | |
| 248 | 0.016 | |
| 249 | 0.156 | |
| 250 | 0.301 | |
| 251 | 0.678 | |
| 252 | 0.008 | |
| 253 | >10.000 | |
| 254 | 0.011 | |
| 255 | 4.866 | |
| 256 | 0.006 | |
| 257 | 0.008 | |
| 258 | 0.007 | |
| 259 | 0.013 | |
| 260 | 0.21 | |
| 261 | 0.04 | |
| 262 | 0.006 | |
| 263 | 0.013 | |
| 264 | 0.007 | |
| 265 | 0.008 | |
| 266 | 0.02 | |
| 267 | 0.01 | |
| 268 | 0.482 | |
| 269 | 0.014 | |
| 270 | 0.006 | |
| 271 | 0.01 | |
| 272 | 0.014 | |
| 273 | 0.007 | |
| 274 | 0.811 | |
| 275 | 0.01 | |
| 276 | 0.005 | |
| 277 | 1.023 | |
| 278 | 0.019 | 0.03 |
| 279 | 0.041 | 0.053 |
| 280 | 0.041 | 0.07 |
| 281 | 0.135 | 0.196 |
| 282 | 0.066 | 0.019 |
| 283 | 0.13 | 0.062 |
| 284 | 0.159 | 0.039 |
| 285 | 0.905 | |
| 286 | 0.059 | 0.016 |
| 287 | 0.006 | 0.014 |
| 293 | 0.07 | 0.115 |
| 295 | 0.007 | 0.011 |
| 296 | 0.07 | 0.115 |
| 297 | 0.013 | |
| 298 | 0.031 | 0.011 |
| 299 | 0.047 | 0.023 |
| 300 | 0.006 | 0.015 |
| 301 | 0.042 | 0.023 |
| 302 | 0.055 | 0.015 |
| 303 | 0.067 | 0.067 |
| 304 | 0.105 | 0.05 |
| 305 | 0.09 | 0.031 |
| 306 | 0.083 | 0.038 |
| 307 | 0.008 | |
| 308 | 0.008 | |
| 309 | 0.007 | |
| 310 | 1.023 | |
| 311 | 1.551 | |

1-24. (canceled)

25. A compound of Formula 2: 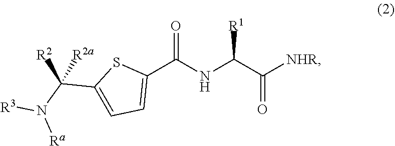
wherein
R is selected from H, unsubstituted or substituted C₁-C₄ alkoxy, unsubstituted or substituted C₁-C₄ alkyl, C₃-C₇ cycloalkyl, C₅-C₁₀ bicycloalkyl, C₅-C₁₀ spirocycloalkyl, C₆-C₁₀ aryl, 5-10 membered heteroaryl, 4-10 membered heterocyclyl, 5-10 membered bicycloheterocyclyl, and 7-12 membered spiroheterocyclyl;
wherein each of said cycloalkyl, bicycloalkyl and spirocycloalkyl is independently unsubstituted or substituted with one, two, or three substituents selected from heterocyclyl, C₁-C₄ alkyl, carboxy, aminocarbonyl, C₁-C₄ alkylaminocarbonyl, C₁-C₄ dialkylaminocarbonyl, C₁-C₄ alkoxycarbonyl, hydroxyl, amino, and C₁-C₄ alkoxy, each of which is substituted or unsubstituted, and halo, cyano, and hydroxyl;
wherein each of said heterocyclyl, bicycloheterocyclyl, and spiroheterocyclyl is independently unsubstituted or substituted with one, two, or three substituents selected from heterocyclyl, C₁-C₄ alkyl, carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, cyano, and hydroxyl;
wherein said heteroaryl is unsubstituted or substituted with one, two, or three substituents selected from heterocyclyl, C₁-C₄ alkyl, C₁-C₄ alkylsulfonyl, carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, cyano, and hydroxyl; and
wherein said aryl is unsubstituted or substituted with one, two, or three substituents selected from heterocyclyl, C₁-C₄ alkyl, C₁-C₄ alkylsulfonyl, carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, cyano, and hydroxyl;
R¹ is selected from C₃-C₈ alkyl, cyclohexyl, cyclopentyl-C₁₋₂-alkyl, cyclohexyl-C₁₋₂-alkyl, bicyclo[2.2.2]octan-1-yl-C₁₋₂-alkyl, bicyclo[3.1.0]hexan-2-yl-C₁₋₂-alkyl and bicyclo[1.1.1]pentan-1-yl-C₁₋₂-alkyl, each of which is unsubstituted;
R² is selected from substituted or unsubstituted C₁-C₄ alkyl, C₁-C₂ haloalkyl, substituted or unsubstituted C₁-C₄ alkoxy-C₁-C₄ alkyl, substituted or unsubstituted 5-6 membered heterocyclyl-C₁-C₄ alkyl, substituted or unsubstituted aryl-C₁-C₄ alkyl, and substituted or unsubstituted 5-6 membered heteroaryl-C₁-2 alkyl;
R^{2a} is H or substituted or unsubstituted C₁-C₄ alkyl; and
R³ is selected from H, C₆-C₁₀ aryl, and 5-10 membered heteroaryl, wherein each heteroaryl and aryl is unsubstituted or substituted with one, two, or three substituents selected from C₁-C₄ alkyl, C₃-C₆ heterocycloalkyl, aralkyl, C₁-C₄ alkylsulfonyl, carboxy, amino, substituted amino, C₃-C₇ cycloalkyl, phenyl, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl; and R^{a} is H or substituted or unsubstituted C₁-C₆ alkyl; or
R³ and R^{a} together with the nitrogen atom to which R³ and R^{a} are bound form 5-10 membered heterocyclyl that is unsubstituted or substituted with one or two substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl,
or a pharmaceutically acceptable salt thereof.

26. The compound of claim 25, wherein R is selected from H, substituted or unsubstituted C₁-C₄ alkyl, C₃-C₇ cycloalkyl, phenyl, 5-10 membered heteroaryl, 4-10 membered heterocyclyl, C₅-C₁₀ bicycloalkyl, C₅-C₁₀ spirocycloalkyl, 5-10 membered bicycloheterocyclyl, and 7-12 membered spiroheterocyclyl;
wherein each of said cycloalkyl, bicycloalkyl and spirocycloalkyl is independently unsubstituted or substituted with one, two, or three substituents selected from C₁-C₄ alkyl, carboxy, aminocarbonyl, C₁-C₄ alkylaminocarbonyl, C₁-C₄ alkoxycarbonyl, and C₁-C₄alkoxy, each of which is substituted or unsubstituted, and halo, cyano, and hydroxyl;
wherein each of said heterocyclyl, bicycloheterocyclyl and spiroheterocyclyl is independently unsubstituted or substituted with one, two, or three substituents selected from C₁-C₄ alkyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl;
wherein said heteroaryl is unsubstituted or substituted with one, two, or three substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl; and
wherein said phenyl is unsubstituted or substituted with one, two, or three substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl.

27. The compound of claim 26, wherein R is selected from H, methyl, ethyl, fluoroethyl, isopropyl, 1-cyano-1-methylethyl, CH₃OCH₂CH₂OCH₂—, CH₃O(C═O)CH₂—, carboxymethyl, 3-fluorophenylmethyl, 4-fluorophenylmethyl, 3-pyridylmethyl, and 4-pyridylmethyl.

28. The compound of claim 26, wherein R is selected from 3-pyrazolyl, 4-pyrazolyl, 1H-1,2,3-triazol-4-yl, 3-pyridinyl, 4-pyridinyl, 2-pyridinyl, 3-thienyl, phenyl, 5-indolyl, 6-indolyl, 5-indazolyl, 5-benzimidazolyl, 1H-pyrrolo[2,3-b]pyridin-5-yl, 1,2,3,4-tetrahydroquinolin-7-yl, 5,6,7,8-tetrahydroquinolin-6-yl, 1,2,3,4-tetrahydroquinolin-6-yl, 6-isoquinolinyl, and quinolin-6-yl, each of which is independently unsubstituted or substituted with one, two, or three substituents selected from methyl, methoxy, ethoxy, isopropoxy, carboxy, (2-methoxyeth-1-yl), methoxycarbonyl, methylaminocarbonyl, aminocarbonyl, methylsulfonyl, hydroxy, chloro, fluoro, and cyano.

29. The compound of claim 26, wherein R is bicyclo[1.1.1]pentan-1-yl that is unsubstituted or substituted with one, two, or three substituents selected from methyl, methoxy, carboxy, methoxycarbonyl, methylaminocarbonyl, aminocarbonyl hydroxy, fluoro, and cyano.

30. The compound of claim 29, wherein R is selected from bicyclo[1.1.1]pentan-1-yl, 3-methyl-bicyclo[1.1.1]pentan-1-yl, 3-methoxycarbonyl-bicyclo[1.1.1]pentan-1-yl, 3-carboxyl-bicyclo[1.1.1]pentan-1-yl, 3-aminocarbonyl-bicyclo[1.1.1]pentan-1-yl, 3-methylaminocarbonyl-bicyclo[1.1.1]pentan-1-yl, 3-hydroxy-bicyclo[1.1.1]pentan-1-yl, 3-fluoro-bicyclo[1.1.1]pentan-1-yl, 3-cyano-bicyclo[1.1.1]pentan-1-yl, and bicyclo[2.2.2]octan-1-yl.

31. The compound of claim 26, wherein R is spiro[3.3]heptan-2-yl or 2-oxaspiro[3.3]heptan-6-yl.

32. The compound of claim 26, wherein R is selected from 1,1-dioxo-1λ⁶-thian-4-yl, 2-oxabicyclo[2.1.1]hexan-4-yl and 8-oxabicyclo[3.2.1]octan-3-yl.

33. The compound of claim 26, wherein R is selected from cyclopropyl, 1-cyanocyclopropyl, 1-trifluoromethyl-cyclopropyl, 2-fluorocyclopropyl, 2,2-difluorocyclopropyl, cyclobutyl, 3-fluorocyclobutyl, 2-methylcyclobutyl 3-methylcyclobutyl, 3,3-difluorocyclobutyl, 3-methoxycarbonylcyclobutyl, 3-carboxy-cyclobutyl, 3,3-difluorocyclopentyl, 3-cyanocyclobutyl, 3-hydroxycyclopentyl, cyclopentyl, 3-fluorocyclopentyl, and 3-cyanocyclopentyl.

34. The compound of claim 26, wherein R is piperidin-4-yl or 1-methylpiperidin-4-yl.

35. The compound of claim 28, wherein R is selected from 1-methyl-3-pyrazolyl, 3-pyrazolyl, 4-pyrazolyl, 1-methyl-4-pyrazolyl, 1-(2-methoxyeth-1-yl)-4-pyrazolyl, 1-ethyl-4-pyrazolyl, 1-fluoroethyl-4-pyrazolyl, 1-trifluoroethyl-4-pyrazolyl, 1-dimethylaminoethyl-4-pyrazolyl, 1-tert-butyl-4-pyrazolyl, 1-(1-methyl-4-piperidinyl)-4-pyrazolyl, 1-oxan-4-yl-4-pyrazolyl, 1-oxetan-3-yl-4-pyrazolyl, 1H-1,2,3-triazol-4-yl, 2-methylpyridin-4-yl, 2,6-dimethylpyridin-4-yl, 2-methylpyridin-5-yl, 2-methylpyridin-6-yl, 2-hydroxy-4-pyridinyl, 2-methoxy-4-pyridinyl, 2-chloro-4-pyridinyl, 2-aminocarbonyl-4-pyridinyl, 2-methoxy-5-pyridinyl, 2-aminocarbonyl-5-pyridinyl, 4-amino-2-pyridinyl, 3-thienyl, 4-cyanophenyl, 3-cyanophenyl, 3-methylsulfonylphenyl, 4-methylsulfonylphenyl, 3,4-difluorophenyl, 4-fluorophenyl, 3-chlorophenyl, 4-chlorophenyl, 3-methoxyphenyl, 3-ethoxyphenyl, 3-isopropoxyphenyl, 4-methoxyethoxyphenyl, 4-fluorophenyl, 4-fluoro-3-methoxyphenyl, 5-indolyl, 1-methyl-5-indolyl, 6-indolyl, 1-methyl-indol-6-yl, 4-chloro-6-indolyl, 7-fluoro-5-indolyl, 3-cyano-5-indolyl, 1-(2-methoxyeth-1-yl)-5-indolyl, 5-indazolyl, 5-benzimidazolyl, 2-methyl-5-benzimidazolyl, 1H-pyrrolo[2,3-b]pyridin-5-yl, 1-methyl-1,2,3,4-tetrahydroquinolin-7-yl, 5,6,7,8-tetrahydroquinolin-6-yl, 1-methyl-1,2,3,4-tetrahydroquinolin-6-yl, 1-(2-methoxyeth-1-yl)-1,2,3,4-tetrahydroquinolin-6-yl, 6-isoquinolinyl, 3-cyano-quinolin-6-yl, 3-fluoroquinolin-6-yl, 3-methoxyquinolin-6-yl, and 8-methoxyquinolin-6-yl.

36. The compound of claim 25, wherein R² is selected from methyl, ethyl, benzyl, trifluoromethyl, 4-fluorobenzyl, methoxymethyl, 1-methyl-4-piperidinylmethyl 4-pyridinylmethyl, 1-pyrazolylmethyl, and 1-methyl-4-pyrazolylmethyl; and R^{2a} is H.

37. The compound of claim 25, wherein R² is methyl; and R^{2a} is H.

38. The compound of claim 25, wherein R² is methyl; and R^{2a} is methyl.

39. The compound of claim 25, wherein R³ is pyrazolo[1,5-a]pyrimidin-7-yl, phenyl or pyridyl, each of which is independently unsubstituted or substituted with one, two, or three substituents selected from chloro, fluoro, bromo, cyano, C₁₋₃ alkyl, hydroxymethyl, C₁₋₃ haloalkyl, cyclopropyl, aminocarbonyl, di[C₁₋₃ alkyl]aminocarbonyl, di[C₁₋₃ alkyl]aminocarbonyl, methoxycarbonyl, carboxy, hydroxyl, C₁₋₃ alkoxy, C₁₋₃ alkoxyalkyl, pyridyl, and phenyl that is unsubstituted or substituted with one or two substituents selected from chloro, fluoro, bromo, cyano, methyl, ethyl, hydroxymethyl, trifluoromethyl, methoxymethyl, methoxypropyl, cyanopropyl, cyclopropyl, carboxy, methoxycarbonyl, aminocarbonyl, methylaminocarbonyl, dimethylaminocarbonyl, hydroxyl, and methoxy.

40. The compound of claim 39, wherein R³ is 3-pyridyl that is unsubstituted or substituted with one or two substituents selected from chloro, fluoro, bromo, cyano, methyl, ethyl, hydroxymethyl 2-hydroxy-2-methylethyl, trifluoromethyl, methoxy methyl, methoxypropyl, 2-cyano-2-methylethyl, cyclopropyl, carboxy, methoxycarbonyl, aminocarbonyl, methylaminocarbonyl, dimethylaminocarbonyl, hydroxyl, methoxy, pyridyl and phenyl that is unsubstituted or substituted with one or two substituents selected from chloro, fluoro, and methoxy.

41. The compound of claim 25, wherein R¹ is tert-butyl, 1-methylpropyl, 2-methylbutyl, 2,2-dimethylbutyl, cyclohexyl, bicyclo[2.2.2]octan-1-yl-CH₂—, bicyclo[3.1.0]hexan-2-yl-CH₂— or bicyclo[1.1.1]pentan-1-yl-CH₂—.

42. The compound of claim 25, wherein R¹ is cyclopentyl-CH₂— or cyclohexyl-CH₂—.

43. The compound of claim 25, wherein R³ and R^{a} together with the nitrogen atom forms dihydroindol-1-yl, 4-methyl-7-chloro-tetrahydroquinoxalin-1-yl, 5-chloro-1,2,3,4-tetrahydroquinolin-2-yl or 7-chloro-1,2,3,4-tetrahydroquinolin-1-yl, wherein the dihydroindol-1-yl or tetrahydroquinoline-1-yl each are unsubstituted or substituted with one or two substituents selected from halo and methoxy.

44. The compound of claim 25, wherein R^{a} is H.

45. The compound of claim 25, wherein R³ and R^{a} together with the nitrogen atom to which R³ and R^{a} are bound form 1,2,3,4-tetrahydroquinoline-1-yl that is unsubstituted or substituted with one or two substituents selected from C₁-C₄ alkyl, C₁-C₄ alkylsulfonyl, carboxy, aminocarbonyl, C₁-C₄ alkylamidocarbonyl, C₁-C₄ alkoxycarbonyl, and C₁-C₄ alkoxy, each of which is substituted or unsubstituted, and halo, cyano, and hydroxy.

46-66. (canceled)

67. A pharmaceutical composition comprising a compound of Formula 2 or a pharmaceutically acceptable salt thereof: 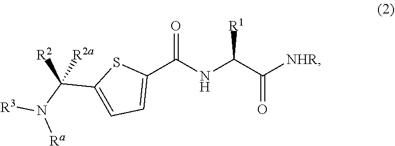
and a pharmaceutically acceptable carrier,
wherein
R is selected from H, unsubstituted or substituted C₁-C₄ alkoxy, unsubstituted or substituted C₁-C₄ alkyl, C₃-C₇ cycloalkyl, C₅-C₁₀ bicycloalkyl, C₅-C₁₀ spirocycloalkyl, C₆-C₁₀ aryl, 5-10 membered heteroaryl, 4-10 membered heterocyclyl, 5-10 membered bicycloheterocyclyl, and 7-12 membered spiroheterocyclyl:
wherein each of said cycloalkyl, bicycloalkyl and spirocycloalkyl is independently unsubstituted or substituted with one, two, or three substituents selected from heterocyclyl, C₁-C₄ alkyl, carboxy, aminocarbonyl, C₁-C₄ alkylaminocarbonyl, C₁-C₄ dialkylaminocarbonyl, C₁-C₄ alkoxycarbonyl, hydroxyl, amino, and C₁-C₄ alkoxy, each of which is substituted or unsubstituted, and halo, cyano, and hydroxyl;
wherein each of said heterocyclyl, bicycloheterocyclyl, and spiroheterocyclyl is independently unsubstituted or substituted with one, two, or three substituents selected from heterocyclyl, C₁-C₄ alkyl, carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, cyano, and hydroxyl;
wherein said heteroaryl is unsubstituted or substituted with one, two, or three substituents selected from heterocyclyl, C₁-C₄ alkyl, C₁-C₄ alkylsulfonyl, carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, cyano, and hydroxyl; and
wherein said aryl is unsubstituted or substituted with one, two, or three substituents selected from heterocyclyl, C₁-C₄ alkyl, C₁-C₄ alkylsulfonyl, carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, cyano, and hydroxyl:
R¹ is selected from C₃-C₈ alkyl, cyclohexyl, Cyclopentyl-C₁₋₂-alkyl, cyclohexyl-C₁₋₂-alkyl, bicyclo[2.2.2]octan-1-yl-C₁₋₂-alkyl, bicyclo[3.1.0]hexan-2-yl-C₁₋₂-alkyl and bicyclo[1.1.1]pentan-1-yl-C₁₋₂-alkyl, each of which is unsubstituted;
R² is selected from substituted or unsubstituted C₁-C₄ alkyl, C₁-C₂ haloalkyl, substituted or unsubstituted C₁-C₄ alkoxy-C₁-C₄ alkyl, substituted or unsubstituted 5-6 membered heterocyclyl-C₁-C₄ alkyl, substituted or unsubstituted aryl-C₁-C₄ alkyl, and substituted or unsubstituted 5-6 membered heteroaryl-C₁₋₂ alkyl;
R^{2a} is H or substituted or unsubstituted C₁-C₄ alkyl; and
R³ is selected from H, C₆-C₁₀ aryl, and 5-10 membered heteroaryl, wherein each heteroaryl and aryl is unsubstituted or substituted with one, two, or three substituents selected from C₁-C₄ alkyl, C₃-C₆ heterocycloalkyl, aralkyl, C₁-C₄ alkylsulfonyl, carboxy, amino, substituted amino, C₃-C₇ cycloalkyl, phenyl, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl; and R^{a} is H or substituted or unsubstituted C₁-C₆ alkyl; or
R³ and R^{a} together with the nitrogen atom to which R³ and R^{a} are bound form 5-10 membered heterocyclyl that is unsubstituted or substituted with one or two substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl.

68. A method of treating cancer, the method comprising administering to a human in need thereof an effective amount of a compound of Formula 2: 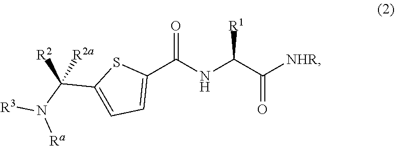
wherein
R is selected from H, unsubstituted or substituted C₁-C₄ alkoxy, unsubstituted or substituted C₁-C₄ alkyl, C₃-C₇ cycloalkyl, C₅-C₁₀ bicycloalkyl, C₅-C₁₀ spirocycloalkyl, C₆-C₁₀ aryl, 5-10 membered heteroaryl, 4-10 membered heterocyclyl, 5-10 membered bicycloheterocyclyl, and 7-12 membered spiroheterocyclyl;
wherein each of said cycloalkyl, bicycloalkyl and spirocycloalkyl is independently unsubstituted or substituted with one, two, or three substituents selected from heterocyclyl, C₁-C₄ alkyl, carboxy, aminocarbonyl, C₁-C₄ alkylaminocarbonyl, C₁-C₄ dialkylaminocarbonyl, C₁-C₄ alkoxycarbonyl, hydroxyl, amino, and C₁-C₄ alkoxy, each of which is substituted or unsubstituted, and halo, cyano, and hydroxyl;
wherein each of said heterocyclyl, bicycloheterocyclyl, and spiroheterocyclyl is independently unsubstituted or substituted with one, two, or three substituents selected from heterocyclyl, C₁-C₄ alkyl, carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, cyano, and hydroxyl;
wherein said heteroaryl is unsubstituted or substituted with one, two, or three substituents selected from heterocyclyl, C₁-C₄ alkyl, C₁-C₄ alkylsulfonyl, carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, cyano, and hydroxyl; and
wherein said aryl is unsubstituted or substituted with one, two, or three substituents selected from heterocyclyl, C₁-C₄ alkyl, C₁-C₄ alkylsulfonyl, carboxy, aminocarbonyl, C₁-C₄ alkylaminocarbonyl, C₁-C₄ dialkylaminocarbonyl, C₁-C₄ alkoxycarbonyl, amino, and C₁-C₄ alkoxy, each of which is substituted or unsubstituted, and halo, cyano, and hydroxyl:
R¹ is selected from C₃-C₈ alkyl, cyclohexyl, cyclopentyl-C₁₋₂-alkyl, cyclohexyl-C₁₋₂-alkyl, bicyclo[2.2.2]octan-1-yl-C₁₋₂-alkyl, bicyclo[3.1.0]hexan-2-yl-C₁₋₂-alkyl and bicyclo[1.1.1]pentan-1-yl-C₁₋₂-alkyl, each of which is unsubstituted;
R² is selected from substituted or unsubstituted C₁-C₄ alkyl, C₁-C₂ haloalkyl, substituted or unsubstituted C₁-C₄ alkoxy-C₁-C₄ alkyl, substituted or unsubstituted 5-6 membered heterocyclyl-C₁-C₄ alkyl, substituted or unsubstituted aryl-C₁-C₄ alkyl, and substituted or unsubstituted 5-6 membered heteroaryl-C₁-2 alkyl;
R^{2a} is H or substituted or unsubstituted C₁-C₄ alkyl; and
R³ is selected from H, C₆-C₁₀ aryl, and 5-10 membered heteroaryl, wherein each heteroaryl and aryl is unsubstituted or substituted with one, two, or three substituents selected from C₁-C₄ alkyl, C₃-C₆ heterocycloalkyl, aralkyl, C₁-C₄ alkylsulfonyl, carboxy, amino, substituted amino, C₃-C₇ cycloalkyl, phenyl, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl; and R^{a} is H or substituted or unsubstituted C₁-C₆ alkyl; or
R³ and R^{a} together with the nitrogen atom to which R³ and R^{a} are bound form 5-10 membered heterocyclyl that is unsubstituted or substituted with one or two substituents selected from C₁-C₄ alkyl, C₁-C₄ alkylsulfonyl, carboxy, aminocarbonyl, C₁-C₄ alkylaminocarbonyl, C₁-C₄ alkoxycarbonyl, and C₁-C₄ alkoxy, each of which is substituted or unsubstituted, and halo, cyano, and hydroxyl,
or a pharmaceutically acceptable salt thereof.

69. The method of claim 68, wherein the cancer is selected from the group consisting of brain, glioblastomas, leukemias, lymphomas, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, neuroblastoma, small cell lung, endometrium, cervix, esophagus, colon, gastric, bladder, head and neck, kidney, lung, liver, melanoma, ovarian, pancreatic, prostate, mesothelioma, sarcoma, osteosarcoma, giant cell tumor of bone, and thyroid.
